SEQUENCE LISTING (239 sequences)

SEQ ID NO: 1 - protein, 423 aa, synthetic construct
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 2 - protein, 500 aa, synthetic construct [sequence omitted]

SEQ ID NO: 3 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 4 - protein, 485 aa, synthetic construct
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 5 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 6 - protein, 380 aa, synthetic construct
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 7 - protein, 582 aa, synthetic construct
MTNPASRPKAYSYIRMSSAIQIKGDSFRRQAEASAKYAAEHDLDLIDDYKLADLGVSAFKSDNLTTGALGRFVAECEAGEIEAGSFLLIESLDRLSRDKILDAFSLFARILKTGVKIVTLSDGQVYDGSSDQVGSIYYAISVMIRSNDESKIKSTRGLANWSQKRKLAAEHGVKMSSQCPAWLKLSVDRKSYLIDKERAKIVQRIFEASASGKGANLITKELNRDKVPTFGRGALWAEAFVSKTLRNRAVLGEFQPGQYVSGKRQPAGDPIPGYFPPVIEEELFDIVQASLRGRLLAGGRRGEGQSNIFTHVAFCGYCGSKMRHRSKGSRVKGNPPHRYLTCFNRFNGPGCDCKPLPYAAFERSFLTFVRDVDLRGLLEGAKRKSEAKTIADRITVNEEKVRKADERIRDYLIKIEGAPDLAEIFMERIRELKAEKDDLVRSIEESNDALSKIKSDNVTDEELASLISTFQNPCGENRIRLADRIKSIIERIDVYPNGEIRKDDPAIDLVRASGDPDAEKIIAAMNAGSRLKDDPYFIVTFRNGAVQTVVPNPSNPDDIRVSVYAGEKTRRVEGSAYEYESD

SEQ ID NO: 8 - protein, 509 aa, synthetic construct
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 9 - protein, 471 aa, synthetic construct
MRKVAIYSRVSTINQAEEGYSIQGQIEALTKYCEAMEWKIYKNYSDAGFSGGKLERPAITELIEDGKNNKFDTILVYKLDRLSRNVKDTLYLVKDVFTANNIHFVSLKENIDTSSAMGNLFLTLLSAIAEFEREQIKERMQFGVMNRAKSGKTTAWKTPPYGYRYNKDEKTLSVNELEAANVRQMFDMIISGCSIMSITNYARDNFVGNTWTHVKVKRILENETYKGLVKYREQTFSGDHQAIIDEKTYNKAQIALAHRTDTKTNTRPFQGKYMLSHIAKCGYCGAPLKVCTGRAKNDGTRRQTYVCVNKTESLARRSVNNYNNQKICNTGRYEKKHIEKYVIDVLYKLQHDKEYLKKIKKDDNIIDITPLKKEIEIIDKKINRLNDLYINDLIDLPKLKKDIEELNHLKDDYNKAIKLNYLDKKNEDSLGMLMDNLDIRKSSYDVQSRIVKQLIDRVEVTMDNIDIIFKF

SEQ ID NO: 10 - protein, 477 aa, synthetic construct
MITTRKVAIYVRVSTTNQAEEGYSIQGQIDSLIKYCEAMGWIIYEEYTDAGFSGGKIDRPAMSKLITDAKHKRFDTILVYKLDRLSRSVRDTLYLVKDVFNQNNIHFVSLQENIDTSSAMGNLFLTLLSAIAEFEREQITERMTMGKIGRAKSGKTMAWTYTPFGYDYNKEKGELILDPAKAPIVKMIYTDYLKGMSIQKIVDKLNKMDYNGKDCTWFPHGVKHLLDNPVYYGMTRYNNKLFPGNHQPIITKELFDKTQRERQRRRLGIEENHYTIPFQAKYMLSKFLRCRQCGSRMGLELGRPRKKEGKRSKKYYCLNSRPKRTASCDTPLYDAETLEDYVLHEIAKIQKDPSIASRQKHIEDHELKYKRERIEANINKTVNQLSKLNNLYLNDLITLEDLKTQTNTLIAKKRLLENELDKTCDNDDELDRQETIADFLALPDVWTMDYEGQKYAVELLVQRVKVDRDNIDIHWTF

SEQ ID NO: 11 - protein, 552 aa, synthetic construct
MPGMTTETGPDPAGLIDLFCRKSKAVKSRANGAGQRRKQEISIAAQETLGRKVAALLGMQVRHVWKEVGSASRFRKGKARDDQSKALKALESGEVGALWCYRLDRWDRGGAGAILKIIEPEDGMPRRLLFGWDEDTGRPVLDSTNKRDRGELIRRAEEAREEAEKLSERVRDTKAHQRENGEWVNARAPYGLRVVLVTVSDEEGDEYDERKLAADDEDAGGPDGLTKAEAARLVFTLPVTDRLSYAGTAHAMNTREIPSPTGGPWIAVTVRDMIQNPAYAGWQTTGRQDGKQRRLTFYNGEGKRVSVMHGPPLVTDEEQEAAKAAVKGEDGVGVPLDGSDHDTRRKHLLSGRMRCPGCGGSCSYSGNGYRCWRSSVKGGCPAPTYVARKSVEEYVAFRWAAKLAASEPDDPFVIAVADRWAALTHPQASEDEKYAKAAVREAEKNLGRLLRDRQNGVYDGPAEQFFAPAYQEALSTLQAAKDAVSESSASAAVDVSWIVDSSDYEELWLRATPTMRNAIIDTCIDEIWVAKGQRGRPFDGDERVKIKWAART

SEQ ID NO: 12 - protein, 519 aa, synthetic construct
MQLDATLTLRDEGLSAFHQRHIKQGALGVFLRAIEDGRIQPGSVLIVEGLDRLSRAEPIQAQAQLAQIINAGITVVTASDGREYNRERLKAQPMDLVYSLLVMIRAHEESDTKSKRVKAAIRRQCEGWVAGTWRGIIRNGKDPHWVRLGEHGKFEHVPERVLAVRTMIDLFLEGHGAIEITRRLTEQNLYVSNAGNYSVHMYRIVRNQALIGEKRISVDGEEFRLDGYYPPILTREEFAELQQTMSERGRRKGKGEIPNIITGLSITVCGYCGRAMTTQNSKARAPKGKSVVRRLSCPMNSFNEGCPIGGSCESEIVERALMRYCSDQFNLSRLLEGDDGTARRTAQLAVARQRASDIEAQIQRVTDALLSDDGKAPAAFTRRARELETQLEEQRREIEALEHQIAASSAHGIPAAAEAWAQLVDGVLALDYDARMKARQLVADTFRKIVVYQRGFAPIDDAAADRWKRSGTIGLMLVTKRGGMRLLNVDRRTGCWQAEDDLDPSLIPSDGLPMLPLDA

SEQ ID NO: 13 - protein, 462 aa, synthetic construct
MKVAIYVRVSTDEQAKEGFSIPAQRERLRAFCASQGWEIVQEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEEEADVVRMIYRMYCDGYGYRSIADRLNELMVKPRIAKEWNHNSVRDILTNDIYIGTYRWGDKVVPNNHPPIISETLFKKAQKEKEKRGVDRKRVGKFLFTGLLQCGNCGGHKMQGHFDKREQKTYYRCTKCHRITNEKNILEPLLDEIQLLITSKEYFMSKFSDRYDQQEVVDVSALTKELEKIKRQKEKWYDLYMDDRNPIPKEELFAKINELNKKEEEIYSKLSEVEEDKEPVEEKYNRLSKMIDFKQQFEQANDFTKKELLFSIFEKIVIYREKGKLKKITLDYTLK

SEQ ID NO: 14 - protein, 461 aa, synthetic construct
MKVAVYCRVSTLEQKEHGHSIEEQERKLKSFCDINDWTVYDTYIDAGYSGAKRDRPELQRLMNDINKFDLVLVYKLDRLTRNVRDLLDLLEIFEKNDVSFRSATEVYDTTTAMGRLFVTLVGAMAEWERETIRERTQMGKLAALRKGIMLTTPPFYYDRVDNKFVPNKYKDVILWAYDEAMKGQSAKAIARKLNNSDIPPPNNTQWQGRTITHALRNPFTRGHFDWGGVHIENNHEPIITDEMYEKVKDRLNERVNTKKVRHTSIFRGKLVCPVCNARLTLNSHKKKSNSGYIFVKQYYCNNCKVTPNLKPVYIKEKEVIKVFYNYLKRFDLEKYEVTQKQNEPEITIDINKVMEQRKRYHKLYASGLMQEDELFDLIKETDQTIAEYEKQNENREVKQYDIEDIKQYKDLLLEMWDISSDEDKEDFIKMAIKNIYFEYIIGTGNTSRKRNSLKITSIEFY

SEQ ID NO: 15 - protein, 461 aa, synthetic construct
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 16 - protein, 481 aa, synthetic construct
MITTNKVAIYVRVSTTNQVEEGYSIDEQKDKLSSYCDIKDWNVYKVYTDGGFSGSNTDRPALESLIKDAKKRKFDTVLVYKLDRLSRSQKDTLHLIEDVFIKNGIEFLSLQENFDTSTPFGKAMIGLLSVFAQLEREQIKERMQLGKLGRAKSGKSMMWAKTSYGYDYHKETGTVTINPAQALTIKFIFESYLRGRSITKLRDDLNEKYPKHVPWSYRAVRTILDNPVYCGFNQYKGEIYPGNHEPIISKEEYDKTQSELKIRQRTAAENVNPRPFQAKYILSGIAQCGYCGAPLKIMLGVKRKDGSRLKKYECHQRHPRTLRGVTTYNDNKKCDSGFYYKDKLEAYVLKEISKLQDDADYLDKIFSGDNAETIDRESYKKQIEELSKKLSRLNDLYIDDRITLEELQSKSAEFISMRGTLETELENDPALRKNKRKADMRKLLNAEKVFSMDYESQKVLVRRLINKVKVTAEDIVINWKI

SEQ ID NO: 17 - protein, 447 aa, synthetic construct
MLRCAIYIRVSTEEQAMHGLSMDAQKADLTDYAKKHNYEIIDYYVDSGKTARKRLSKRKDLQRMIEDVKLNKIDIIIFTKLDRWFRNVRDYYKIQEVLEDHNVDWKTIFENYDTSTANGRLHINIMLSVAQDEADRTSERIKRVFENKLKNNEPTSGSLPIGYKIKEKSIIIDEEKAPIAKDVFDFYYYHQSQTKVFKEILNKYNLSLCEKTIRRMLENKLYIGIYREHENFCPPLIDKNKFDEVQLILKRRNIKYIPTKRIFLFTSLLICKECRHKMIGNAQIRNTKAGKIEYILYRCNQSYARHTCNHRKVIYENKIETYLLNNIESELKKFIYDYELEDIPKVKNKVNKTNIKRKLEKLKELYINDLIDIDMYKEDYKKYTEILNTKEEKIEQRNLQPLKDFLNSDFKSLYSSISREEKRLLWRGIISEIQIDCNNDITIIPHP

SEQ ID NO: 18 - protein, 460 aa, synthetic construct
MKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKF

SEQ ID NO: 19 - protein, 474 aa, synthetic construct
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 20 - protein, 471 aa, synthetic construct
MTVGIYIRVSTEEQVKEGFSISAQKEKLKAYCTAQGWEDFKFYVDEGKSAKDMHRPLLQEMISHIKKGLIDTVLVYKLDRLTRSVVDLHNLLSIFDEFNCAFKSATEVYDTSSAMGRFFITIISSVAQFERENTSERVSFGMAEKVRQGEYIPLAPFGYTKGTDGKLIVNKIEKEIFLQVVEMVSTGYSLRQTCEYLTNIGLKTRRSNDVWKVSTLIWMLKNPAVYGAIKWNNEIYENTHEPLIDKATFNKVAKILSIRSKSTTSRRGHVHHIFKNRLICPACGKRLSGLRTKYINKNKETFYNNNYRCATCKEHRRPAVQISEQKIEKAFIDYISNYTLNKANISSKKLDNNLRKQEMIQKEIISLQRKREKFQKAWAADLMNDDEFSKLMIDTKMEIDAAEDRKKEYDVSLFVSPEDIAKRNNILRELKINWTSLSPTEKTDFISMFIEGIEYVKDDENKAVITKISFL

SEQ ID NO: 21 - protein, 452 aa, synthetic construct
MKAAIYIRVSTQEQIENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQMIYDIFEEEKSITTLQKRLKKLGFKVKSYSSYNNWLTNDLYCGYVSYADKVHTKGVHEPIISEEQFYRVQEIFSRMGKNPNMNRDSASLLNNLVVCGKCGLGFVHRRKDTISRGKKYHYRYYSCKTYKHTHELEKCGNKIWRADKLEELIIDRVNNYSFASRNVDKEDELDNLNEKLKTEHKKKKRLFDLYISGSYEVSELDAMMADIDAQINYYEAQIEANEELKKNKKIQENLADLATVDFDSLEFREKQLYLKSLINKIYIDGEQVTIEWL

SEQ ID NO: 22 - protein, 470 aa, synthetic construct
MKGESELDKKAAIYIRVSTQEQATEGYSIQAQTDRLIKYVEAKDFILYKKYIDAGYSASKLERPAMQDLIQDVQSKKVDVVIVYKLDRLSRSQKDTMYLIEDIFRPNDVELISMQESFDTSTAFGSATVGMLSVFAQLERKSISERMITGRVERAKKGFYHTGGQDRPPAGYQFNSDNQLIINEYEAAAIKDLFRLYNDGLGKSSISEYLKKNYPGKNKWLPSSIDRMLKNSLYIGKVKFSGAEYDGIHEPIIDEVTFYKTQKEIARRKQTNTKRYNYVALLGGLCECGICGAKMANRRAVGRKGKVYRYYRCYSKKGSPKHMMKTDGCSSKAQQQFIIDEAVINNLKNIDVEAELKRRSAPQTNTSLISSQIESIDKQINKLIDLFQVDSMPLDVISEKIDKLNKEKQSMEKLLERKNKLDKTELQHRFDVLKSFDWDNSSIESKRVVIEMLVQKVIIHDNSIEIILVE

SEQ ID NO: 23 - protein, 512 aa, synthetic construct
MRTNEHNFHNIEEEIKHVAVYLRLSRGEDESELDNHKTRLLNRCELNNWSYELYKEIGSGSTIDDRPVMQKLLTDVEKNLYDAVLVVDLDRLSRGNGTDNDRILYSMKVSETLIVVESPYQVLDANNESDEEIILFKGFFARFEFKQINKRMREGKKLAQSRGQWVNSVTPYGYIVNKTTKKLTPSEEEAKVVIMIKDFFFEGKSTSDIAWELNKRKIKPRRATEWRSSSIANILQNEVYVGNIVYNKSVGNKKPSKSKTRVTTPYRRLPEEEWRRVYNAHQPLYSKEEFDRIKQYFECNVKSHKGSEVRTYALTGLCKTPDGKTMRVTQGKKGTDDDLYLFPKKNKHGDSSIYKGISYNVVYETLKEVILQVKDYLDSVLDQNENKDLVEELKEELMKKEDELETIQKAKNRIVQGFLIGLYDEQDSIELKVEKEKEIDEKEKEIEAIKMKIDNAKTVNNSIKKTKIERLLSDVQSAESEKEINRFYKTLIKEIIVDRTDENEAKIKVNFL

SEQ ID NO: 24 - protein, 475 aa, synthetic construct
MITTNKVAIYVRVSTTNQVEEGYSIDEQKDKLEAYCKIKDWKIYDVYVDGGFSGANTQRPELERLISDVKRKKVDIVLVYKLDRLSRSQKDTLFLIEDVFAKNDVAFISLQENFDTSTPFGKASIGMLSVFAQLEREQIKERMMLGKEGRAKNGKSMSWTTIAFGYDYSKETGVLSVNPTQALIVNRIFTEYLNGKPVVKIIRDLNAEGHVGRKRPWGETITKYLLKNETYLGKVKYKDKVYEGQHEPIITQELFDLVQLEVERRQISAYEKYNNPRPFRAKYMLSGLMKCGYCGASLGLRYTRKDKNGISHHKYQCRNRHSKDLEKRCESGWYSKEELERGVIKELERIKFDPKYKNETLAKKEETIKVEEIKKQLERINNQVSKLTELYLDEIITRKELDEKNDKIKTERQFLEEQLENQKSNVLSIRKRKLTRLLKDFDVEKLSYEDASKIVKNIIKEIIVTKDGMSITLDF

SEQ ID NO: 25 - protein, 462 aa, synthetic construct
MGKSITVIPAKKVQTSVLHQDRKKIKVAAYCRVSTDQEEQLSSYENQVNYYREFISKHEDYELVDIYADEGISATNTKKRDAFNRLIQDCRAGKVDRILVKSISRFARNTLDCIKYVRELKELGVGVTFEKENIDSLDSKGEVLLTILSSLAQDESRSISENATWGIRKKFERGEVRVNTTKFMGYDKDENGRLIINPQQAETVKFIYEKFLEGYSPESIAKYLNDNEIPGWTGKANWYPSAIQKMLQNEKYKGDALLQKTFTVDFLTKKRVQNDGQVNQYYVENSHEAIIDEETWETVQLEMARRKTYRDEHQLKSYIMQSEDNPFTTKVFCGACGSAFGRKNWATSRGKRKVWQCNNRYRIKGVEGCYSSHLDEATLEQIFLKALELLSENIDLLDGKWEKILAENRLLDKHYSMALSDLLRQEQIDFNPSDMCRVLDHIRIGLDGEITVCLLEGTEVDL

SEQ ID NO: 26 - protein, 411 aa, synthetic construct
MRTVRRIQPIKSPCKPRFKVAAYARVSDSRLHHSLSTQISYYNRLIQAHPDWELVGIYYDEGISGKEQSNRQGFLNLIKDCEDGKIDRIITKSIARFGRNTVELLTTVRQLRLKNIGVTFEKENIDSLSSEGELMLTLLASVAQEESQNLSENIRWRIQKKFEKGIPHTPQDMYGYRWDGEQYQIEPNEAKVIRKVFKWYLDGDSVQQIVDKLNQEQVLTRLGNPFTVASIREFFKQEAYFGRLVLQKTYREAFSRNPKRNKGQRNKYIIENAHEPIVTKEYFDLVLHEKERRNQLMHQESHLNKGIFRDKISCSECGCLMIVKVDSKQVNKTVRYYCRTRNRFGASSCSCRTLGEKRLLASFKSKLGIVPDKEWVENNIKHIEYDFGYRILRVTPVKGRKYLIEIREGRY

SEQ ID NO: 27 - protein, 473 aa, synthetic construct
MRNKVAIYVRVSTASQADEGYSIDEQKSKLEAYCEIKDWKIYDTYIDGGFSGANTQRPELERLISDAKRKKIDIVLVYKLDRLSRSQKDTLFLIEDVFAKNDVAFISLQENFDTSTPFGKASIGMLSVFAQLEREQIKERMMLGKEGRAKNGKSMSWTTIPFGYDYSKETGILSVNPTQALIVKRIFTEYLNGKSVVKIIRDLNAEGHVGRKRPWGETITKYLLKNETYLGKSKYKGKVFEGQHDAIISQELFDLVQLEVEKRQISAFEKYNNPRPFRAKYMLSGLMKCGYCGASLGLYVAPKNKNGVSKYKYQCRHRYHKDKAIRCNSGWYSKDELEKRVIKELERLKFDPKYKKETLAKKDETIKVEDIKKQLERINKQVSKLTELYLDEVITRKDLDEKNAKIKTERQYLEEQLENQKSNVMSIRKRKLSRLLKDFDIEKLSYEEASKIVKSVIKEIVVTKDDMTITLDF

SEQ ID NO: 28 - protein, 463 aa, synthetic construct
MKVATYVRVSTDEQAKEGFSIPAQRERLRAFCESQGWEIVEEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEDEANTVRMIYRMYCDGYGYHSIAKRLNELGIKPRIAKEWNHNSVRDILTNDIYIGTYRWGNKVVLNNHPPIISETLFRKVQKEKEKRRVDRTRVGKFLLTGLLYCGNCNGHKMQGTFDKREQKTYYRCLKCNRITNEKNILEPLLDEIQLLITSKEYFMSKFSDQYDQKEEVDVSALKKELEKIKRQKEKWYDLYMDDRNPIPKEDLFAKINELNKKEEEIYNKLNEVEPEDKEPVEEKYNRLSKMIDFKQQFEQANDFTKKELLFSIFEKIVIYREKGKLKKITLDYTLK

SEQ ID NO: 29 - protein, 452 aa, synthetic construct
MLRVALYIRVSTEEQALNGDSIRTQIEALEQYSKENDFNIVGKYIDEGCSATNLKRPNLQRLLRDVEKDKVDLVLMTKIDRLSRGVKNYYKIMETLEKHKCDWKTILENYDSSTAAGRLHINIMLSVAENEAAQTSERIKFVFQDKLRRKEVISGTIPIGYKIENKHLVIDKEKKYIVKAIFDEYEKSGSVRTLIETINNLHGELYSYNKIKNILRNELYIGIYNKRGFYVEDYCEPIISKKQFKQIQRILEKNKKTTPNKNIHYHIFSGLLKCKECGYTLKGNSSNVGEKLYLSYRCSTFYLNKNCVHNVTHNEKHIENYLLTNLKPQLHKHMVKLEAQNEKIRRNKKSNKKDEKKKIMKKLDKIKDLYLEDLIDKETYRKDYEKLQSQLDNITEEQESQIIDTSHIKKFLDIDINEMYSDLSRVERRRFWLSIIDYIEIDNNKNITINFI

SEQ ID NO: 30 - protein, 592 aa, synthetic construct
MKITLLYYIKKFNIYCNRYLSQQINISVDIIGFYQFKNVTNSVTDVLKRGDNLDRICIYLRKSRADEELEKTIGVGETLSKHRKALLKFAKEKKLNIMEIKEEIVSADSIFFRPKMIELLKEVENNQYTGVLVMDIQRLGRGDTEDQGIIARIFKESHTKIITPMKTYDLDDDLDEDYFEFESFMGRKEYKMIKKRMQGGRVRSVEDGNYIATNPPFGYDIHWINKSRTLKFNSKESEIVKLIFKLYTEGNGAGTISNYLNSLGYKTKFGNNFSNSSIIFILKNPVYIGKITWKKKDIRKSKDPHKVKDTRTRDKSEWIIADGKHEPIIDEKIWNKAQEILNNKYHIPYKIANGPANPLAGVVICSKCNSKMVMRKYGKKLPHLICNNKECNNKSARFDYIEKAVLEGLDEYLKNYKVNVKANNKTSDIEPYEQQSNALNKELILLNEQKLKLFDFLEREIYTEEIFLERSKNLDERINTTTLAINKIKKILDNEKKKNNKNDIVKFEKILEGYKKTNDIQKKNELMKSLVFKIEYKKEQHQRNDGLLYIYFLSFCVRCISYLTQFISFFVYPYRILEIYLTFSFFIISYEH

SEQ ID NO: 31 - protein, 504 aa, synthetic construct
MRICMYLRKSRADEELEKTLGEGETLSKHRKALLKFAKEKNLNIVEIKEEIVSGESLFFRPKMLELLKEIENKQYSGVLVMDMQRLGRGNMQDQGIILETFKKSNTKIITPMKTYDLSNDFDEEYSEFEAFMSRKELKMINRRMQGGRVRSVEDGNYIATNAPYGYDIHWINKARTLKPNQKESEIVKLIFKLYIEGNGAGTIAKHLNSLGYKTKFGNSFNNSSIIFILKNPVYIGKITWKKKDIRKSKDPNKVKDTRTRDKSEWIIVDGKHDPIIDQITWKQAQEILNNRYHVPYKLVNGPANPLAGLIICTTCKSKMVMRKLRGTDRILCKNNKCNNISNRFDAVEKSVVESLENYLKAYKVNLPELNKTSNLKLYEQQISTLKKELKILNEQKLKLFDFLERGIYDEDTFLKRSKNLDERIEITNESLSNLNQIIAKENKAIKKEDIIKFEKVLDSYKSTADIRLKNELMKTLIFKIEYTKNKKGNDFKIKVFPKLKPLNI

SEQ ID NO: 32 - protein, 545 aa, synthetic construct
MIAAIYSRKSKFTGKGESVENQIEMCKEYLKRNFNNIDDIEIYEDEGFSGKDTNRPKFKKMIKAAKNKKFNILICYRLDRISRNVADFSNTIEELQKYNIDFISIKEQFDTSTPMGRAMMNIAAVFAQLERETIAERIKDNMVELAKTGRWLGGTSPLGYKSEPIEYSNEDGKSKKMYKLTEVENEMNIVKLIYKLYLEKRGFSSVATYLCKNKYKGKNGGEFSRETARQIVINPVYCISDKTIFKWFKSKGATTYGTPDGIHGLMVYNKREGGKKDKPINEWIIAVGKHRGVISSDIWLKCQNLIQQNNAKSSPRSGTGEKFLLSGMVVCKECGSGMSSWSHFNKKTNFMERYYRCNLRNRASNRCSTKMLNAYKAEEYVANYLKELDINAIKKMYHSNKKNIIDYDAKYEVNKLNKSIEENKKIIQGIIKKIALFDDLDILGMLKNELERLKKENDEMKIKLKELKSILELEDEEEIFLSTMEENISNFKKFYDFVNITQKRILIKGLVESIVWDTGGEEKILEINLIGSNTKLPSGKVKRRE

SEQ ID NO: 33 - protein, 485 aa, synthetic construct
MSKKVAIYTRVSTTNQAEEGYSIDEQIDKLKMYCEAMDWKVSEIYTDAGFTGSKLTRPAMEKMITDIGLKKFDTVIVYKLDRLSRSVRDTLYLVKDVFTKNEIDFISLSESIDTSSAMGSLFLTILSAINEFERENIKERMTMGKIGRAKSGKSMMWAKTAFGYSHNQETGILEINPLEASIVEQIFNEYLKGTSITKLRDKLNEDGHIAKELPWSYRTIRQTLDNPVYCGYIKYKNNTFEGLHKPIISHETYLSVQKELEARQQQTYEKNNNPRPFQAKYLLSGIARCGYCGAPLRIVLGHRRKDGSRTMKYQCVNRFPRKTKGVTTYNDNKKCDSGAYDMQWIEDIVLKTLNGFQKSDKKLRKILNIKEESKVDTSGFQKQLKSINNKIQKNSDLYLNDFITMDDLKKRTEMLQGEKKLIQARINEVDKPSTSEIFDLVKSELGETTISKISYEDKKKIVNNLISKVDVTADNIDIIFKFQLA

SEQ ID NO: 34 - protein, 458 aa, synthetic construct
MNEQKDKLKKYCEIKDWTIVKEYVDPGRSGSNINRPSMQQLIKDADTGLYDAVLVYKLDRLSRSQKDTLYLIEDVFQKNNIHFISLSENFDTSTAFGKAMIGILSVFAQLEREQIKERMSMGRVGRAKSGKIMEFNNPAFGYEVDGDNYKVDPLRAEIVKRIYKMYLSGTSINKIKETLNLEGHIGNKKNWSDTRIRYILSNPTYLGKIRYDGKTYDGKFSPIIDEETFNKTQNELKERQTATYKRFNMKLRPFQSKYMLSGLLRCGYCGATLFVNSYVYNGKRKLRYNCPSTYKSKQKTRTYKIMDPNCPFKLVYAKDLEPAVINEIKNLALNPQSIQKPVKKKPDIDVEAIQKELAKVRKQQQRLIDLYVISDDVNIDNISKKSADLKLQEETLKKQLAPLEEPNDDDKIVAFNEILAQIKDIDSLDYDKQKFIVKKLIKKIDVWNDNKIKIHWNI

SEQ ID NO: 35 - protein, 515 aa, synthetic construct
MKTAIYLRKSRADLEAEARGEGETLAKHRSTLLKIAKEMNLNVLSVREEIVSGESLVKRPEMLALLEEIEDNKYDAVLCMDMDRLGRGGMKEQGIILETFKRSNTKIMTPRKTYDLNDEWDEEYSEFEAFMARKELKIITRRMQRGRIASVEAGNYLGTHAPFGYDIHRLNKRERTLTINSEEASVVRMIFDWYANEDMGASAIRNKLNDLGYKSKLGNDWNPYSILDILKNNIYIGKVTWQKRKEVKRPDAVKRSCARQDKSDWIIADGKHEPIIPESLFEQAQEKLNSRYHVPYNTNGIKNPLAGIIKCSKCGYSMVQRYPKNRKETMDCKHRGCENKSSYTELIEKRLLEALKEWYINYKADFEAHKQGDKLKETQVIQMNEAALRKLEKELVDVQKQKNNLHDLLERGVYTVDMFLERSQVISDRINEITSTMENLKKEIKTEIKKEKVKKDTIPQVEHVLDLYFKTDDPKKKNSLLKSVLEKAVYKKEKWQRLDDFELVLYPKLPQDGDI

SEQ ID NO: 36 - protein, 516 aa, synthetic construct
MYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKI

SEQ ID NO: 37 - protein, 513 aa, synthetic construct
MKYLALHENSRIAVYSRKSREDRDSEDTLAKHRNELEYLIKRENFKNVQWFEKVVSGETIDERPMFSLLLPRIENGEFDAVCAVAMDRLSRGSQIDSGRILEAFKQSGTLFITPKKTYDLSIEGDEMLSEFESIIARSEYRAIKRRTINGKKNATREGRLHSGSVPYGYKWDKNLKAAVVVEEKKKIYRMMIKWFLEEEYSCTVIAEMLNELKVPSPSGRSIWYGEVVSEILSNDFHRGYVWFGKYKKSKSNNSIVQNKNLDEVLIAKGHHETMKTDEEHALILNRIEKLRTYKVAGRRLNMNTHRLSGIVRCPYCHKAQAIEQPKGRRKHVRKCLRKSAERTKECEETKGIHEEVLFQSIMKEIKKYNESLFSPTEQDVNDDSYTAQLIGLREKAVKKAKGRIERIKEMYLDGDISKTEYKEKLKISQETLQKAENELAELIASTEFQNALSAETKKEKWSHHKVQEMIESTDGMSNSEINLILKMLISHVTYTVEDLGDGTKNLNIKVYYN

SEQ ID NO: 38 - protein, 580 aa, synthetic construct
MDPQHKPTRALIVIRLSRLTDETTSPERQLEACERFCAARGWEVVGVAEDLDVSAGTTSPFERPSLSQWIGDGKDNPGRIGEFDTVVFYRVDRLVRRVRHLHDVIAWSERFDVNMVSATESHFDLSTTIGALIAQLVASFAEMELEGISQRATSAHRHNVQLGKFVGGSPPFGYMPEETPDGWRLVHDPDVVPIILEVVDRVLEGEPLRRITDDLNARGATTARDLVKQRKGKETEGHKWHSNVLKRRLMSPAMLGYALRREPLTDSKGKPKLSAKGAKLYGPEEIVRGPDGLPVQRAEPILPKPLFDRVVAELEARELQKEPTKRINSMLLRVLYCGVCGQPVYRAKGQGGRSDRYRCRSIQDGANCGNPSVLTYELDDLVEESILVLMGDSERLAHVWNPGEDNASELAEVEARLADRTGLIGVGAYKAGTPQRATLDTLIEADAKLYERLKAATPRPAGWTWEPTGETFAEWWAALDTGARNVYLRNMGVRVTYDKRPVPEQVSAGEKPRVHLELGEVRKMAEQVAVTGTIGTLTRNYTRLGEIGITHVDIDAGSGKAVFVTKSGERFELPLNIPEE

SEQ ID NO: 39 - protein, 540 aa, synthetic construct
MKAIAIYARKSLFTGKGDSIGAQVDTCKRFIDYKFANEDYEIRTFKDEGWSGKTTDRPDFTNMVNLIKSKKIDYVITYKLDRIGRTARDLHNFLYELDNLGIVYLSATEPYDTTTSAGRFMISILAAMAQMERERLAERVKSGMIQIAKKGRWLGGQCPLGFDSKREIYIDDMGKERQMMRLTPNKEEIKIVKLIYDKYLEMGSMSQVRKYCLENSIRGKNGGDFSTNTLKQLLTSPIYVKSSDNIFKYLESQNINVFGTPNGNGMLTFNKTKEIRIERDKSEWIAAVGKHKGIIDDNKWLQIQQQLQQQSEKQIKSSGRQGTTSTGLLSGIIKCSKCGNNLLIKTGHKSKKNPGTTYSYYVCGKKDNSYGHKCDNKNVRTDEADSAVITQLKLYNKELLIKNLKEALIQNEKTDTDNIEILESKLKEKEKAVSNLVKKLSLIDDESISNIILNEVTNINKEINDIKLQLSNETLKINEVTKATLDTEIYIKILENFNKKIDDITDPIEKMNLLKSALESVEWNGDSGEFKINLIGSKKK

SEQ ID NO: 40 - protein, 460 aa, synthetic construct
MKVAIYTRVSTLEQREKGHSIDEQERKLRSFCDINDWTVKDVYVDAGFSGAKRDRPELTRLLDDISEFDLVLVYKLDRLTRSVRDLLDLLEVFENNNVAFRSATEVYDTTTAIGRLFVTLVGAMAEWERETIRERSLMGKRAAIKKGMILTAPPFYYDRVNNTYIPNQYKDVVLDVYNKVKKGYSIAHIARLYNNSDVKPPNGNEEWTTRMLMHALRNPVTRGHYQWGEIYIEDSHEPIITDEMYNTIIDRLDKHTNTKVVAHTSVFRGKLICPNCGYALTLNSQKRKRKNDTIVYKTYYCNNCKITKGMKPHHITETETLRVFKDHLSKIDLKQYETQEKEKQSHVTIDLSKVMEQRKRYHKLYASGMMQENELFELIKETDEMIEEYEKQRKQVDVKEFDICKIKEIKDVLLKSWDIFTLEDKADFIQMSIKAINIEYTKLKRGKSSNSMKIKDIEFY

SEQ ID NO: 41 - protein, 27 aa, synthetic construct
MSQFDILCKTPPKVLVRQFVERFERPS

SEQ ID NO: 42 - protein, 396 aa, synthetic construct
GEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 43 - protein, 396 aa, synthetic construct
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEG

SEQ ID NO: 44 - protein, 27 aa, synthetic construct
SIRYPAWNGIISQEVLDYLSSYINRRI

SEQ ID NO: 45 - protein, 37 aa, synthetic construct
MRALVVIRLSRVTDATTSPERQLESCQQLCAQRGWDV

SEQ ID NO: 46 - protein, 463 aa, synthetic construct
VGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 47 - protein, 169 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVD

SEQ ID NO: 48 - protein, 331 aa, synthetic construct
GEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 49 - protein, 208 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRR

SEQ ID NO: 50 - protein, 292 aa, synthetic construct
GVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 51 - protein, 222 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQG

SEQ ID NO: 52 - protein, 278 aa, synthetic construct
REPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 53 - protein, 259 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDD

SEQ ID NO: 54 - protein, 241 aa, synthetic construct
GAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 55 - protein, 262 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAP

SEQ ID NO: 56 - protein, 238 aa, synthetic construct
LVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 57 - protein, 363 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERL

SEQ ID NO: 58 - protein, 137 aa, synthetic construct
EKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 59 - protein, 370 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAG

SEQ ID NO: 60 - protein, 130 aa, synthetic construct
SDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 61 - protein, 399 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAG

SEQ ID NO: 62 - protein, 101 aa, synthetic construct
SPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 63 - protein, 400 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGS

SEQ ID NO: 64 - protein, 100 aa, synthetic construct
PQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 65 - protein, 468 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRG

SEQ ID NO: 66 - protein, 32 aa, synthetic construct
GLTRTIDFGDLQEYEQHLRLGSVVERLHTGMS

SEQ ID NO: 67 - protein, 233 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKP

SEQ ID NO: 68 - protein, 372 aa, synthetic construct
GSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 69 - protein, 571 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRG

SEQ ID NO: 70 - protein, 34 aa, synthetic construct
QGTPIEKRASITWAKPPTDDDEDDAQDGTEDVAA

SEQ ID NO: 71 - protein, 326 aa, synthetic construct
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGI

SEQ ID NO: 72 - protein, 159 aa, synthetic construct
TVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 73 - protein, 229 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSG

SEQ ID NO: 74 - protein, 114 aa, synthetic construct
VADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 75 - protein, 269 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATH

SEQ ID NO: 76 - protein, 74 aa, synthetic construct
RLIYGAKDDSGQRYLAWSGHSARVGAARDMARAGVSIPEIMQAGGWTNVNIVMNYIRNLDSETGAMVRLLEDGD

SEQ ID NO: 77 - protein, 269 aa, synthetic construct
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCI

SEQ ID NO: 78 - protein, 111 aa, synthetic construct
KPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 79 - protein, 345 aa, synthetic construct
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 80 - protein, 82 aa, synthetic construct
QDKKTMMMNEEDDGNGMDELLAVLGYKVRSSEMADVAQKLEQLEVMMSNVQEDDLSQLATETVHYNPAELYTWLDSMLTDLN

SEQ ID NO: 81 - protein, 297 aa, synthetic construct
PLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIGSVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 82 - protein, 177 aa, synthetic construct
TQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 83 - protein, 92 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRIS

SEQ ID NO: 84 - protein, 107 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 85 - DNA, 557 bp, synthetic construct
  source 1..557: mol_type = other DNA
ccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 86 - DNA, 497 bp, synthetic construct
  source 1..497: mol_type = other DNA
agttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcga

SEQ ID NO: 87 - DNA, 462 bp, synthetic construct
  source 1..462: mol_type = other DNA
aagcaggtttccccaactgacacaaaccgtgcaatttggaactccgcctggtctttccaggtctagaggggtgacactttgtactgtgtttggctccacgctcggtccactggcgagtgttagtaacagcaccgttgcttcgtagcggagcatgatggccgtgggaactcctccttggtaacaaggacccacggggccgaaagccacgtccaatcggacccatcatgtgtgcaaccccagcacagcaacttttctgcgaaactcacttcaaggtgacactgatactggtactcaaacactggtgacaggctaaggatgcccttcaggtaccccgaggtaacacgcgtcactcgggatctgagaaggggactggggcttctataaaagcgtccagtttaaaaagcttctatgcctgaataggtgaccggaggccggcaccttttctttacagccactgacttt

SEQ ID NO: 88 - protein, 19 aa, synthetic construct
ATNFSLLKQAGDVEENPGP

SEQ ID NO: 89 - protein, 18 aa, synthetic construct
EGRGSLLTCGDVEENPGP

SEQ ID NO: 90 - protein, 921 aa, synthetic construct
MSQFDILCKTPPKVLVRQFVERFERPSSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRIPKKKRKV

SEQ ID NO: 91 - protein, 978 aa, synthetic construct
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSGGSGSGSSGGSGTPLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIGSVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKDYKDDDDKPKKKRKVATNFSLLKQAGDVEENPGPMAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNYPYDVPDYASGGSGSGSSGGSGTSIRYPAWNGIISQEVLDYLSSYINRRIPKKKRKV

SEQ ID NO: 92 - protein, 1000 aa, synthetic construct
MRALVVIRLSRVTDATTSPERQLESCQQLCAQRGWDVSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 93 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 94 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 95 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 96 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 97 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 98 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 99 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 100 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 101 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 102 - protein, 1000 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTDWWREQDTAAKNTWLRSMNVRLTFDVRGGLTRTIDFGDLQEYEQHLRLGSVVERLHTGMSGSPKKKRKV

SEQ ID NO: 103 - protein, 998 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGLTRTIDFGDLQEYEQHLRLGSVVERLHTGMSPKKKRKV

SEQ ID NO: 104 - protein, 1103 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 105 - protein, 868 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGSGGSGSGSSGGSGTILWHEMWHEGLEEASRLYFGERNVKGMFEVLEPLHAMMERGPQTLKETSFNQAYGRDLMEAQEWCRKYMKSGNVKDLLQAWDLYYHVFRRISPKKKRKVATNFSLLKQAGDVEENPGPMSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGSGSGSSGGSGTQGTPIEKRASITWAKPPTDDDEDDAQDGTEDVAAPKKKRKV

SEQ ID NO: 106 - protein, 976 aa, synthetic construct
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGISGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTTVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 107 - protein, 843 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGSGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPATMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDPKKKRKV

SEQ ID NO: 108 - protein, 898 aa, synthetic construct
MAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNYPYDVPDYASGGSGSGSSGGSGTLIYGAKDDSGQRYLAWSGHSARVGAARDMARAGVSIPEIMQAGGWTNVNIVMNYIRNLDSETGAMVRLLEDGDPKKKRKVATNFSLLKQAGDVEENPGPMSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRSGGSGSGSSGGSGTPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKDYKDDDDKPKKKRKV

SEQ ID NO: 109 - protein, 898 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRSGGSGSGSSGGSGTPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKDYKDDDDKPKKKRKVATNFSLLKQAGDVEENPGPMAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNYPYDVPDYASGGSGSGSSGGSGTLIYGAKDDSGQRYLAWSGHSARVGAARDMARAGVSIPEIMQAGGWTNVNIVMNYIRNLDSETGAMVRLLEDGDPKKKRKV

SEQ ID NO: 110 - protein, 878 aa, synthetic construct
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCISGGSGSGSSGGSGTMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECPKKKRKVATNFSLLKQAGDVEENPGPMKRDHHHHHHQDKKTMMMNEEDDGNGMDELLAVLGYKVRSSEMADVAQKLEQLEVMMSNVQEDDLSQLATETVHYNPAELYTWLDSMLTDLNSGGSGSGSSGGSGTKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKDPKKKRKV

SEQ ID NO: 111 - DNA, 2766 bp, synthetic construct
  source 1..2766: mol_type = other DNA
atgagccagttcgacatcctgtgcaagaccccccccaaggtgctggtgcggcagttcgtggagagattcgagaggcccagctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgaaaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatccccaagaaaaagcggaaggtgtga

SEQ ID NO: 112 - DNA, 2937 bp, synthetic construct
  source 1..2937: mol_type = other DNA
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgaaaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggctccggagggtctggctccggatcaagtggtggcagcggtacccctttgtatggttttacttcgatttgtggaagaaggcctgagatggaagatgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgggtcagttgcgccggaaacggttgggtcaacgtcggtggttgccgttgttttcccatctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtggccaccaactttagcctgctgaaacaggctggcgacgtggaagagaaccctggacctatggcgccaactcaagacgagttcacccaactctcccaatcaatcgccgagttccacacgtaccaactcggtaacggccgttgctcatctctcctagctcagcgaatccacgcgccgccggaaacagtatggtccgtggtgaggcgtttcgataggccacagatttacaaacacttcatcaaaagctgtaacgtgagtgaagatttcgagatgcgagtgggatgcacgcgcgacgtgaacgtgataagtggattaccggcgaatacctctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccagcatcagataccccgcctggaacggcatcatcagccaggaggtgctggactacctgagcagctacatcaacaggcggatccccaagaaaaagcggaaggtgtga

SEQ ID NO: 113 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgtttccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 114 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggattccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 115 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccactccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 116 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagatccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 117 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 118 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgattccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctaggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 119 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgccccttccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtacccttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 120 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactgtccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 121 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtacctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 122 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtacctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 123 - DNA, 3003 bp, synthetic construct
  source 1..3003: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtga

SEQ ID NO: 124 - DNA, 2997 bp, synthetic construct
  source 1..2997: mol_type = other DNA
atgcgagccctggtggtcattcgcctgagcagagtcacagacgctactacaagccctgagcggcagctggagtcctgtcagcagctgtgcgcacagcgaggatgggatgtggtcggagtggcagaggatctggacgtgagcggggctgtcgatccattcgaccgaaagcggaggcccaacctggcacgatggctggctttcgaggaacagccctttgatgtgatcgtcgcctacagagtggacaggctgacacgctcaattcgacatctgcagcagctggtgcattgggccgaggatcacaagaaactggtggtcagcgcaactgaagcccacttcgacaccacaactccttttgccgctgtggtcatcgcactgatgggcaccgtggcccagatggagctggaagctatcaaggagcgaaaccggagcgcagcccatttcaatattcgggccgggaaatacagaggcagcctgcccccttggggctatctgcctacccgggtggatggggagtggagactggtgccagaccccgtccagagagagaggattctggaagtgtaccacagagtggtggacaaccacgaaccactgcatctggtggcccacgatctgaataggcgcggagtcctgtctccaaaggactattttgctcagctgcagggaagggagccacagggacgagaatggagtgctaccgcactgaagcggtctatgatcagtgaggctatgctgggctatgcaactctgaatgggaaaaccgtgagagatgatgacggagcaccactggtgcgggctgagcctattctgacaagagagcagctggaagctctgagggcagaactggtgaaaaccagtagggccaagcctgctgtgtcaacaccaagcctgctgctgcgagtgctgttctgcgcagtctgtggcgagccagcatacaaatttgccggcgggggaaggaagcatccccgctatcgatgccggagcatggggttccctaagcactgtggaaacggcactgtggctatggccgaatgggacgccttttgtgaggaacaggtgctggatctgctgggggacgcagagcgcctggaaaaagtgtgggtcgctggaagcgattccgctgtggagctggcagaagtcaatgccgagctggtggacctgacctccctgatcggatctcctgcatacagggcaggctccccacagcgagaagctctggatgcacgaattgctgcactggcagctcgacaggaggaactggaggggctggaagccagaccctctggatgggagtggcgagaaacaggccagcggtttggggattggtggagggagcaggacacagcagccaagaacacttggctgagatccatgaatgtcaggctgactttcgacgtgcgaggatccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggactgacccgaacaatcgattttggcgacctgcaggagtatgaacagcatctgcgcctgggaagtgtggtcgagcgactgcacaccggcatgtcacccaagaaaaagcggaaggtgtga

SEQ ID NO: 125 - DNA, 3312 bp, synthetic construct
  source 1..3312: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagaggcccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaaaagcgccaagatcctggacaccaagaacctgcagagggagctgggcggctatgtgggcggcaaggccccctacggcttcgagctggtgtccgaaaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagccctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcagccaggccgccatccaccccggcagcatcaccggcctgtgtaagagaatggacgccgacgccgtgcccaccagaggcgaaaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagggaccccatcaccctgaggcctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggaaaccctggccctgctgtgggaggccgccagaagattcggcaagctgaccgaggcccccgaaaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccccaagaaaaagcggaaggtgtga

SEQ ID NO: 126 - DNA, 2607 bp, synthetic construct
  source 1..2607: mol_type = other DNA
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagaggcccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaaaagcgccaagatcctggacaccaagaacctgcagagggagctgggcggctatgtgggcggcaaggccccctacggcttcgagctggtgtccgaaaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgaaaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagggaccccatcaccctgaggcctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggaaaccctggccctgctgtgggaggccgccagaagattcggcaagctgaccgaggcccccgaaaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggctccggagggtctggctccggatcaagtggtggcagcggtaccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcacccaagaaaaagcggaaggtggccaccaactttagcctgctgaaacaggctggcgacgtggaagagaaccctggacctatgtctagaggagtgcaggtggaaaccatctccccaggggacggacgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtgttcgatgtggagcttctaaaactggaatccggagggtctggctccggatcaagtggtggcagcggtacccagggcacccccatcgagaagagagccagcatcacctgggccaagcctcccaccgacgacgacgaggatgacgcccaggacggcaccgaggacgtggccgcccccaagaaaaagcggaaggtgtga

SEQ ID NO: 127 - DNA, 2931 bp, synthetic construct
  source 1..2931: mol_type = other DNA
atgaccaagaaggtggccatctacaccagagtgtccaccaccaaccaggccgaggaaggcttcagcatcgacgagcagatcgaccggctgaccaaatacgccgaggccatgggatggcaggtgtccgatacctacaccgacgccggctttagcggcgccaagctggaaagacccgccatgcagcggctgatcaacgacatcgagaacaaggccttcgacaccgtgctggtgtacaagctggacaggctgagcagaagcgtgcgggacaccctgtacctcgtgaaggacgtgttcaccaagaacaagatcgacttcatcagcctgaacgagagcatcgacaccagcagcgctatgggcagcctgttcctgaccatcctgagcgccatcaacgagttcgagcgcgagaacatcaaagaacggatgaccatgggcaagctgggcagagccaagagcggcaagagcatgatgtggaccaagaccgccttcggctactaccacaacagaaagaccggcatcctggaaatagtgccactgcaggccaccatcgtggaacagatcttcaccgactacctgagcggcatctccctgaccaagctgagagacaagctgaacgagtccggccacatcggcaaggacatcccttggagctaccggaccctgcggcagaccctggacaaccctgtgtactgcggctacatcaagttcaaggactccctgttcgagggcatgcacaagcccatcatcccttacgagacatacctgaaggtgcagaaagagctggaagagagacagcagcagacctacgagcggaacaacaaccccagacccttccaggccaagtacatgctgtccggcatggccagatgcggctactgtggcgcccctctgaagatcgtgctgggccacaagagaaaggacggcagccggaccatgaagtaccactgcgccaaccggttccctagaaagaccaagggcatctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccaccgtgtacaacgacaacaagaagtgcgacagcggcacctacgacctgagcaacctggaaaacaccgtgatcgacaacctgatcggcttccaggaaaacaacgacagcctgctgaagatcatcaacggcaacaaccagcccatcctggacacctccagcttcaagaagcagatcagccagatcgacaagaagatccagaagaacagcgacctgtacctgaacgatttcatcaccatggacgagctgaaggaccggaccgactctctgcaggccgagaagaagctgctgaaggccaagatctctgagaacaagttcaacgatagcaccgacgtgttcgagctcgtgaaaacacagctgggctccatccccatcaatgagctgagctacgataacaagaaaaagattgtgaacaacctggtgtctaaggtggacgtgaccgccgacaacgtggacatcatcttcaagttccagctggcctga

SEQ ID NO: 128 - DNA, 2535 bp, synthetic construct
  source 1..2535: mol_type = other DNA
atgtccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggttccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtgtgagccaccaactttagcctgctgaaacaggctggcgacgtggaagagaaccctggacctgccaccatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggaccccaagaaaaagcggaaggtgtga

SEQ ID NO: 129 - DNA, 2697 bp, synthetic construct
  source 1..2697: mol_type = other DNA
atggcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccctgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgaccccaagaaaaagcggaaggtggccaccaactttagcctgctgaaacaggctggcgacgtggaagagaaccctggacctatgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagatccggagggtctggctccggatcaagtggtggcagcggtacccctttgtatggttttacttcgatttgtggaagaaggcctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtgtga

SEQ ID NO: 130 - DNA, 2697 bp, synthetic construct
  source 1..2697: mol_type = other DNA
atgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagatccggagggtctggctccggatcaagtggtggcagcggtacccctttgtatggttttacttcgatttgtggaagaaggcctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtggccaccaactttagcctgctgaaacaggctggcgacgtggaagagaaccctggacctatggcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccctgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgaccccaagaaaaagcggaaggtgtga

SEQ ID NO: 131 - DNA, 2637 bp, synthetic construct
  source 1..2637: mol_type = other DNA
atgatcgagaaccagctgagcctgctgggcgacttttctggcgtgcggcccgacgatgtgaaaaccgccattcaggccgcccagaaaaagggcatcaacgtggccgagaacgagcagttcaaggccgccttcgagcatctgctgaacgagttcaagaagcgggaagagagatacagccccaacaccctgcggcggctggaaagcgcctggacctgcttcgtggattggtgcctggccaaccacagacacagcctgcctgccacccccgataccgtggaagccttcttcatcgagcgggccgaggaactgcaccggaacaccctgagcgtgtacagatgggccatcagccgggtgcacagagtggccggatgccctgatccctgcctggacatctacgtggaagatcggctgaaggccattgcccggaagaaagtgcgggaaggcgaggccgtgaagcaggccagccctttcaacgagcagcatctgctgaagctgaccagcctgtggtacagaagcgacaagctgctgctgcggcggaacctggctctgctggctgtggcctacgagagcatgctgagagccagcgagctggccaacatccgggtgtccgatatggaactggccggcgacggaaccgccatcctgaccatccctatcaccaagaccaaccactccggcgagcccgatacctgcatcctgtcccaggatgtggtgtccctgctgatggactacaccgaggccggcaagctggatatgagcagcgacggcttcctgttcgtgggcgtgtccaagcacaacacctgtatctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccaagcccaagaaggacaagcagaccggcgaggtgctgcacaagcccatcaccaccaagacagtggaaggcgtgttctacagcgcctgggagacactggacctgggcagacagggcgtgaagcctttcacagcccacagcgccagagtgggagccgctcaggacctgctgaagaagggctacaataccctgcagatccagcagtccggccggtggtctagcggagccatggtggccagatacggcagagccatcctggctagggatggcgctatggcccacagcagagtgaaaaccagatccgcccccatgcagtggggcaaggacgagaaggaccccaagaaaaagcggaaggtgtga

SEQ ID NO: 132 - DNA, 4424 bp, synthetic construct
  source 1..4424: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgaaaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggctccggagggtctggctccggatcaagtggtggcagcggtacccctttgtatggttttacttcgatttgtggaagaaggcctgagatggaagatgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgggtcagttgcgccggaaacggttgggtcaacgtcggtggttgccgttgttttcccatctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtggccaccaactttagcctgctgaaacaggctggcgacgtggaagagaaccctggacctatggcgccaactcaagacgagttcacccaactctcccaatcaatcgccgagttccacacgtaccaactcggtaacggccgttgctcatctctcctagctcagcgaatccacgcgccgccggaaacagtatggtccgtggtgaggcgtttcgataggccacagatttacaaacacttcatcaaaagctgtaacgtgagtgaagatttcgagatgcgagtgggatgcacgcgcgacgtgaacgtgataagtggattaccggcgaatacctctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccagcatcagataccccgcctggaacggcatcatcagccaggaggtgctggactacctgagcagctacatcaacaggcggatccccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 133 - DNA, 4253 bp, synthetic construct
  source 1..4253: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgagccagttcgacatcctgtgcaagaccccccccaaggtgctggtgcggcagttcgtggagagattcgagaggcccagctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgaaaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgaaaccaaccccatcgaggagtggcagcacatcgagcagctgaagggcagcgccgagggcagcatcagataccccgcctggaacggcatcatcagccaggaggtgctggactacctgagcagctacatcaacaggcggatccccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 134 - DNA, 4484 bp, synthetic construct
  source 1..4484: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgcgagccctggtggtcattcgcctgagcagagtcacagacgctactacaagccctgagcggcagctggagtcctgtcagcagctgtgcgcacagcgaggatgggatgtggtcggagtggcagaggatctggacgtgagcggggctgtcgatccattcgaccgaaagcggaggcccaacctggcacgatggctggctttcgaggaacagccctttgatgtgatcgtcgcctacagagtggacaggctgacacgctcaattcgacatctgcagcagctggtgcattgggccgaggatcacaagaaactggtggtcagcgcaactgaagcccacttcgacaccacaactccttttgccgctgtggtcatcgcactgatgggcaccgtggcccagatggagctggaagctatcaaggagcgaaaccggagcgcagcccatttcaatattcgggccgggaaatacagaggcagcctgcccccttggggctatctgcctacccgggtggatggggagtggagactggtgccagaccccgtccagagagagaggattctggaagtgtaccacagagtggtggacaaccacgaaccactgcatctggtggcccacgatctgaataggcgcggagtcctgtctccaaaggactattttgctcagctgcagggaagggagccacagggacgagaatggagtgctaccgcactgaagcggtctatgatcagtgaggctatgctgggctatgcaactctgaatgggaaaaccgtgagagatgatgacggagcaccactggtgcgggctgagcctattctgacaagagagcagctggaagctctgagggcagaactggtgaaaaccagtagggccaagcctgctgtgtcaacaccaagcctgctgctgcgagtgctgttctgcgcagtctgtggcgagccagcatacaaatttgccggcgggggaaggaagcatccccgctatcgatgccggagcatggggttccctaagcactgtggaaacggcactgtggctatggccgaatgggacgccttttgtgaggaacaggtgctggatctgctgggggacgcagagcgcctggaaaaagtgtgggtcgctggaagcgattccgctgtggagctggcagaagtcaatgccgagctggtggacctgacctccctgatcggatctcctgcatacagggcaggctccccacagcgagaagctctggatgcacgaattgctgcactggcagctcgacaggaggaactggaggggctggaagccagaccctctggatgggagtggcgagaaacaggccagcggtttggggattggtggagggagcaggacacagcagccaagaacacttggctgagatccatgaatgtcaggctgactttcgacgtgcgaggatccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggactgacccgaacaatcgattttggcgacctgcaggagtatgaacagcatctgcgcctgggaagtgtggtcgagcgactgcacaccggcatgtcacccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 135 - DNA, 4812 bp, synthetic construct
  source 1..4812: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgctctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaactaagtatgtactatacagaggcaagccaccatgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggattccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtgtgacccaccactggtttctacatttacacccatgctagcgcggccgcatcgataagcttgtcgacgatatcttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagat

SEQ ID NO: 136 - DNA, 4799 bp, synthetic construct
  source 1..4799: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagaggcccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaaaagcgccaagatcctggacaccaagaacctgcagagggagctgggcggctatgtgggcggcaaggccccctacggcttcgagctggtgtccgaaaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagccctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccggcagccaggccgccatccaccccggcagcatcaccggcctgtgtaagagaatggacgccgacgccgtgcccaccagaggcgaaaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagggaccccatcaccctgaggcctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggaaaccctggccctgctgtgggaggccgccagaagattcggcaagctgaccgaggcccccgaaaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 137 - DNA, 4094 bp, synthetic construct
  source 1..4094: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagaggcccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaaaagcgccaagatcctggacaccaagaacctgcagagggagctgggcggctatgtgggcggcaaggccccctacggcttcgagctggtgtccgaaaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgaaaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagggaccccatcaccctgaggcctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggaaaccctggccctgctgtgggaggccgccagaagattcggcaagctgaccgaggcccccgaaaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggctccggagggtctggctccggatcaagtggtggcagcggtaccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcacccaagaaaaagcggaaggtggccaccaactttagcctgctgaaacaggctggcgacgtggaagagaaccctggacctatgtctagaggagtgcaggtggaaaccatctccccaggggacggacgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtgttcgatgtggagcttctaaaactggaatccggagggtctggctccggatcaagtggtggcagcggtacccagggcacccccatcgagaagagagccagcatcacctgggccaagcctcccaccgacgacgacgaggatgacgcccaggacggcaccgaggacgtggccgcccccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 138 - DNA, 4418 bp, synthetic construct
  source 1..4418: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgaccaagaaggtggccatctacaccagagtgtccaccaccaaccaggccgaggaaggcttcagcatcgacgagcagatcgaccggctgaccaaatacgccgaggccatgggatggcaggtgtccgatacctacaccgacgccggctttagcggcgccaagctggaaagacccgccatgcagcggctgatcaacgacatcgagaacaaggccttcgacaccgtgctggtgtacaagctggacaggctgagcagaagcgtgcgggacaccctgtacctcgtgaaggacgtgttcaccaagaacaagatcgacttcatcagcctgaacgagagcatcgacaccagcagcgctatgggcagcctgttcctgaccatcctgagcgccatcaacgagttcgagcgcgagaacatcaaagaacggatgaccatgggcaagctgggcagagccaagagcggcaagagcatgatgtggaccaagaccgccttcggctactaccacaacagaaagaccggcatcctggaaatagtgccactgcaggccaccatcgtggaacagatcttcaccgactacctgagcggcatctccctgaccaagctgagagacaagctgaacgagtccggccacatcggcaaggacatcccttggagctaccggaccctgcggcagaccctggacaaccctgtgtactgcggctacatcaagttcaaggactccctgttcgagggcatgcacaagcccatcatcccttacgagacatacctgaaggtgcagaaagagctggaagagagacagcagcagacctacgagcggaacaacaaccccagacccttccaggccaagtacatgctgtccggcatggccagatgcggctactgtggcgcccctctgaagatcgtgctgggccacaagagaaaggacggcagccggaccatgaagtaccactgcgccaaccggttccctagaaagaccaagggcatctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccaccgtgtacaacgacaacaagaagtgcgacagcggcacctacgacctgagcaacctggaaaacaccgtgatcgacaacctgatcggcttccaggaaaacaacgacagcctgctgaagatcatcaacggcaacaaccagcccatcctggacacctccagcttcaagaagcagatcagccagatcgacaagaagatccagaagaacagcgacctgtacctgaacgatttcatcaccatggacgagctgaaggaccggaccgactctctgcaggccgagaagaagctgctgaaggccaagatctctgagaacaagttcaacgatagcaccgacgtgttcgagctcgtgaaaacacagctgggctccatccccatcaatgagctgagctacgataacaagaaaaagattgtgaacaacctggtgtctaaggtggacgtgaccgccgacaacgtggacatcatcttcaagttccagctggcctgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 139 - DNA, 4511 bp, synthetic construct
  source 1..4511: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagatccggagggtctggctccggatcaagtggtggcagcggtacccctttgtatggttttacttcgatttgtggaagaaggcctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtggccaccaactttagcctgctgaaacaggctggcgacgtggaagagaaccctggacctatggcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccctgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgaccccaagaaaaagcggaaggtgtgatgacccaccactggtttctacatttacgagtatgctagcgcggccgcatcgataagcttgtcgacgatatcttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagat

SEQ ID NO: 140 - DNA, 4508 bp, synthetic construct
  source 1..4508: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatggcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccctgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgaccccaagaaaaagcggaaggtggccaccaactttagcctgctgaaacaggctggcgacgtggaagagaaccctggacctatgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagatccggagggtctggctccggatcaagtggtggcagcggtacccctttgtatggttttacttcgatttgtggaagaaggcctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtgtgacccaccactggtttctacatttacgagtatgctagcgcggccgcatcgataagcttgtcgacgatatcttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagat

SEQ ID NO: 141 - DNA, 3625 bp, synthetic construct
  source 1..3625: mol_type = other DNA
tccctatcagtgatagagatccatgtgcagtctactccctatcagtgatagagaagctatgtccagcttactccctatcagtgatagagatggtatgtccagtactctccctatcagtgatagagaacatatgtggagtgtatccctatcagtgatagagaaactatctgcagattactccctatcagtgatagagtatgtatgtcgaggtaggcgtgtacggtgggaggcctatataagcagagctcgtttagtgaaccgtcagatcgctggagaattcgagctcggtacccggggacaacatcataaggttacgataggagagcaagccaccatgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagatccggagggtctggctccggatcaagtggtggcagcggtacccctttgtatggttttacttcgatttgtggaagaaggcctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggattacaaggacgatgacgataagcccaagaaaaagcggaaggtggccaccaactttagcctgctgaaacaggctggcgacgtggaagagaaccctggacctatggcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaactacccatacgatgttccagattacgcttccggagggtctggctccggatcaagtggtggcagcggtaccctgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgaccccaagaaaaagcggaaggtgtgacccaccactggtttctacatttacgagtatgctagcgcggccgcatcgataagcttgtcgacgatatcttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagat

SEQ ID NO: 142 - DNA, 4346 bp, synthetic construct
  source 1..4346: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgtccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggttccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtgtgagccaccaactttagcctgctgaaacaggctggcgacgtggaagagaaccctggacctgccaccatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggaccccaagaaaaagcggaaggtgtgacccaccactggtttctacatttacgagtatgctagcgcggccgcatcgataagcttgtcgacgatatcttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagat

SEQ ID NO: 143 - DNA, 4124 bp, synthetic construct
  source 1..4124: mol_type = other DNA
ttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaacatcataaggttacgataggagagcaagccaccatgatcgagaaccagctgagcctgctgggcgacttttctggcgtgcggcccgacgatgtgaaaaccgccattcaggccgcccagaaaaagggcatcaacgtggccgagaacgagcagttcaaggccgccttcgagcatctgctgaacgagttcaagaagcgggaagagagatacagccccaacaccctgcggcggctggaaagcgcctggacctgcttcgtggattggtgcctggccaaccacagacacagcctgcctgccacccccgataccgtggaagccttcttcatcgagcgggccgaggaactgcaccggaacaccctgagcgtgtacagatgggccatcagccgggtgcacagagtggccggatgccctgatccctgcctggacatctacgtggaagatcggctgaaggccattgcccggaagaaagtgcgggaaggcgaggccgtgaagcaggccagccctttcaacgagcagcatctgctgaagctgaccagcctgtggtacagaagcgacaagctgctgctgcggcggaacctggctctgctggctgtggcctacgagagcatgctgagagccagcgagctggccaacatccgggtgtccgatatggaactggccggcgacggaaccgccatcctgaccatccctatcaccaagaccaaccactccggcgagcccgatacctgcatcctgtcccaggatgtggtgtccctgctgatggactacaccgaggccggcaagctggatatgagcagcgacggcttcctgttcgtgggcgtgtccaagcacaacacctgtatctccggagggtctggctccggatcaagtggtggcagcggtaccatggccgccagcgacgaagtgaacctgatcgagagcagaaccgtggtgcccctgaacacctgggtgctgatctccaacttcaaggtggcctacaacatcctgcggaggcccgacggcaccttcaacagacacctggccgagtacctggaccggaaagtgaccgccaacgccaaccctgtggacggcgtgttcagcttcgacgtgctgatcgaccggcggatcaacctgctgagccgggtgtacagacccgcctacgccgatcaggaacagcccccctctatcctggatctggaaaagcccgtggatggcgacatcgtgcccgtgatcctgttcttccacggcggcagctttgcccacagcagcgccaatagcgccatctacgacaccctgtgcagacggctcgtgggcctgtgcaaatgcgtggtggtgtccgtgaactaccgcagagcccccgagaacccttacccctgcgcctacgatgatggctggatcgccctgaactgggtcaacagcagaagctggctgaagtccaagaaagacagcaaggtgcacatctttctggccggcgatagcagcggcggcaatatcgcccataacgtggccctgagagccggcgagtctggcatcgatgtgctgggcaatatcctgctgaaccccatgttcggcggcaacgagcggaccgagagcgagaagtctctggacggcaagtacttcgtgaccgtgcgggaccgggactggtactggaaggcctttctgcccgagggcgaggacagagagcaccccgcctgcaatcccttcagccccagaggcaaaagcctggaaggcgtgtccttcccaaagtccctggtggtggtggccggcctggacctgatcagagattggcagctggcctatgccgagggcctgaagaaagccggccaggaagtgaagctgatgcacctggaaaaggccaccgtgggcttctacctgctgcccaacaacaaccacttccacaacgtgatggacgagatcagcgccttcgtgaacgccgagtgccccaagaaaaagcggaaggtggccaccaactttagcctgctgaaacaggctggcgacgtggaagagaaccctggacctatgaagcgggaccaccaccatcaccatcatcaggacaagaaaaccatgatgatgaacgaagaggacgacggcaacggcatggacgagctgctggctgtgctgggctacaaagtgcggagcagcgagatggccgacgtggcccagaaactggaacagctggaagtgatgatgagcaacgtgcaggaagatgacctgtcccagctggccaccgagacagtgcactacaaccccgccgagctgtacacctggctggactccatgctgaccgacctgaactccggagggtctggctccggatcaagtggtggcagcggtaccaagcccaagaaggacaagcagaccggcgaggtgctgcacaagcccatcaccaccaagacagtggaaggcgtgttctacagcgcctgggagacactggacctgggcagacagggcgtgaagcctttcacagcccacagcgccagagtgggagccgctcaggacctgctgaagaagggctacaataccctgcagatccagcagtccggccggtggtctagcggagccatggtggccagatacggcagagccatcctggctagggatggcgctatggcccacagcagagtgaaaaccagatccgcccccatgcagtggggcaaggacgagaaggaccccaagaaaaagcggaaggtgtgatgacccaccactggattgtacaattacgagtctaagtaaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggatgacaatagcaggcatgctggggatgcggtgggctctatgg

SEQ ID NO: 144 - DNA, 34 bp, synthetic construct
  source 1..34: mol_type = other DNA
gaagttcctattctctagaaagtataggaacttc

SEQ ID NO: 145 - DNA, 34 bp, synthetic construct
  source 1..34: mol_type = other DNA
gaagttcctattctctagatagtataggaacttc

SEQ ID NO: 146 - DNA, 34 bp, synthetic construct
  source 1..34: mol_type = other DNA
gaagttcctattctctacttagtataggaacttc

SEQ ID NO: 147 - DNA, 34 bp, synthetic construct
  source 1..34: mol_type = other DNA
gaagttcctattcttcaaatagtataggaacttc

SEQ ID NO: 148 - DNA, 34 bp, synthetic construct
  source 1..34: mol_type = other DNA
gaagttcctattctctagaaggtataggaacttc

SEQ ID NO: 149 - DNA, 34 bp, synthetic construct
  source 1..34: mol_type = other DNA
gaagttcctattcttcaaaaggtataggaacttc

SEQ ID NO: 150 - DNA, 34 bp, synthetic construct
  source 1..34: mol_type = other DNA
gaagttcctattcactagaatgtataggaacttc

SEQ ID NO: 151 - DNA, 34 bp, synthetic construct
  source 1..34: mol_type = other DNA
gaagttcctattctgaactaagtataggaacttc

SEQ ID NO: 152 - DNA, 34 bp, synthetic construct
  source 1..34: mol_type = other DNA
gaagttcctattctttctgaagtataggaacttc

SEQ ID NO: 153 - DNA, 34 bp, synthetic construct
  source 1..34: mol_type = other DNA
gaagttcctattctcatataagtataggaacttc

SEQ ID NO: 154 - DNA, 34 bp, synthetic construct
  source 1..34: mol_type = other DNA
gaagttcctattctatcagaagtataggaacttc

SEQ ID NO: 155 - DNA, 34 bp, synthetic construct
  source 1..34: mol_type = other DNA
gaagttcctattcttataggagtataggaacttc

SEQ ID NO: 156 - DNA, 34 bp, synthetic construct
  source 1..34: mol_type = other DNA
gaagttcctattctccgggcagtataggaacttc

SEQ ID NO: 157 - DNA, 38 bp, synthetic construct
  source 1..38: mol_type = other DNA
ggcttgtcgacgacggcgaactccgtcgtcaggatcat

SEQ ID NO: 158 - DNA, 38 bp, synthetic construct
  source 1..38: mol_type = other DNA
ggcttgtcgacgacggcgacctccgtcgtcaggatcat

SEQ ID NO: 159 - DNA, 38 bp, synthetic construct
  source 1..38: mol_type = other DNA
ggcttgtcgacgacggcgagctccgtcgtcaggatcat

SEQ ID NO: 160 - DNA, 38 bp, synthetic construct
  source 1..38: mol_type = other DNA
ggcttgtcgacgacggcgatctccgtcgtcaggatcat

SEQ ID NO: 161 - DNA, 38 bp, synthetic construct
  source 1..38: mol_type = other DNA
ggcttgtcgacgacggcgcactccgtcgtcaggatcat

SEQ ID NO: 162 - DNA, 38 bp, synthetic construct
  source 1..38: mol_type = other DNA
ggcttgtcgacgacggcgccctccgtcgtcaggatcat

SEQ ID NO: 163 - DNA, 38 bp, synthetic construct
  source 1..38: mol_type = other DNA
ggcttgtcgacgacggcgcgctccgtcgtcaggatcat

SEQ ID NO: 164 - DNA, 38 bp, synthetic construct
  source 1..38: mol_type = other DNA
ggcttgtcgacgacggcgctctccgtcgtcaggatcat

SEQ ID NO: 165 - DNA, 38 bp, synthetic construct
  source 1..38: mol_type = other DNA
ggcttgtcgacgacggcggactccgtcgtcaggatcat

SEQ ID NO: 166 - DNA, 38 bp, synthetic construct
  source 1..38: mol_type = other DNA
ggcttgtcgacgacggcggcctccgtcgtcaggatcat

SEQ ID NO: 167 - DNA, 38 bp, synthetic construct
  source 1..38: mol_type = other DNA
ggcttgtcgacgacggcgggctccgtcgtcaggatcat

SEQ ID NO: 168 - DNA, 38 bp, synthetic construct
  source 1..38: mol_type = other DNA
ggcttgtcgacgacggcggtctccgtcgtcaggatcat

SEQ ID NO: 169 - DNA, 38 bp, synthetic construct
  source 1..38: mol_type = other DNA
ggcttgtcgacgacggcgtactccgtcgtcaggatcat

SEQ ID NO: 170 - DNA, 38 bp, synthetic construct
  source 1..38: mol_type = other DNA
ggcttgtcgacgacggcgtcctccgtcgtcaggatcat

SEQ ID NO: 171 - DNA, 38 bp, synthetic construct
  source 1..38: mol_type = other DNA
ggcttgtcgacgacggcgtgctccgtcgtcaggatcat

SEQ ID NO: 172 - DNA, 38 bp, synthetic construct
  source 1..38: mol_type = other DNA
ggcttgtcgacgacggcgttctccgtcgtcaggatcat

SEQ ID NO: 173 - DNA, 48 bp, synthetic construct
  source 1..48: mol_type = other DNA
ggtttgtctggtcaaccaccgcgaactcagtggtgtacggtacaaacc

SEQ ID NO: 174 - DNA, 48 bp, synthetic construct
  source 1..48: mol_type = other DNA
ggtttgtctggtcaaccaccgcgacctcagtggtgtacggtacaaacc

SEQ ID NO: 175 - DNA, 48 bp, synthetic construct
  source 1..48: mol_type = other DNA
ggtttgtctggtcaaccaccgcgagctcagtggtgtacggtacaaacc

SEQ ID NO: 176 - DNA, 48 bp, synthetic construct
  source 1..48: mol_type = other DNA
ggtttgtctggtcaaccaccgcgatctcagtggtgtacggtacaaacc

SEQ ID NO: 177 - DNA, 48 bp, synthetic construct
  source 1..48: mol_type = other DNA
ggtttgtctggtcaaccaccgcgcactcagtggtgtacggtacaaacc

SEQ ID NO: 178 - DNA, 48 bp, synthetic construct
  source 1..48: mol_type = other DNA
ggtttgtctggtcaaccaccgcgccctcagtggtgtacggtacaaacc

SEQ ID NO: 179 - DNA, 48 bp, synthetic construct
  source 1..48: mol_type = other DNA
ggtttgtctggtcaaccaccgcgcgctcagtggtgtacggtacaaacc

SEQ ID NO: 180 - DNA, 48 bp, synthetic construct
  source 1..48: mol_type = other DNA
ggtttgtctggtcaaccaccgcgctctcagtggtgtacggtacaaacc

SEQ ID NO: 181 - DNA, 48 bp, synthetic construct
  source 1..48: mol_type = other DNA
ggtttgtctggtcaaccaccgcggactcagtggtgtacggtacaaacc

SEQ ID NO: 182 - DNA, 48 bp, synthetic construct
  source 1..48: mol_type = other DNA
ggtttgtctggtcaaccaccgcggcctcagtggtgtacggtacaaacc

SEQ ID NO: 183 - DNA, 48 bp, synthetic construct
  source 1..48: mol_type = other DNA
ggtttgtctggtcaaccaccgcgggctcagtggtgtacggtacaaacc

SEQ ID NO: 184 - DNA, 48 bp, synthetic construct
  source 1..48: mol_type = other DNA
ggtttgtctggtcaaccaccgcggtctcagtggtgtacggtacaaacc

SEQ ID NO: 185 - DNA, 48 bp, synthetic construct
  source 1..48: mol_type = other DNA
ggtttgtctggtcaaccaccgcgtactcagtggtgtacggtacaaacc

SEQ ID NO: 186 - DNA, 48 bp, synthetic construct
  source 1..48: mol_type = other DNA
ggtttgtctggtcaaccaccgcgtcctcagtggtgtacggtacaaacc

SEQ ID NO: 187 - DNA, 48 bp, synthetic construct
  source 1..48: mol_type = other DNA
ggtttgtctggtcaaccaccgcgtgctcagtggtgtacggtacaaacc

SEQ ID NO: 188 - DNA, 48 bp, synthetic construct
  source 1..48: mol_type = other DNA
ggtttgtctggtcaaccaccgcgttctcagtggtgtacggtacaaacc

SEQ ID NO: 189 - DNA, 46 bp, synthetic construct
  source 1..46: mol_type = other DNA
gtgcgggtgccagggcgtgcccaagggctccccgggcgcgtactcc

SEQ ID NO: 190 - DNA, 46 bp, synthetic construct
  source 1..46: mol_type = other DNA
gtgcgggtgccagggcgtgcccacgggctccccgggcgcgtactcc

SEQ ID NO: 191 - DNA, 46 bp, synthetic construct
  source 1..46: mol_type = other DNA
gtgcgggtgccagggcgtgcccaggggctccccgggcgcgtactcc

SEQ ID NO: 192 - DNA, 46 bp, synthetic construct
  source 1..46: mol_type = other DNA
gtgcgggtgccagggcgtgcccatgggctccccgggcgcgtactcc

SEQ ID NO: 193 - DNA, 46 bp, synthetic construct
  source 1..46: mol_type = other DNA
gtgcgggtgccagggcgtgccccagggctccccgggcgcgtactcc

SEQ ID NO: 194 - DNA, 46 bp, synthetic construct
  source 1..46: mol_type = other DNA
gtgcgggtgccagggcgtgcccccgggctccccgggcgcgtactcc

SEQ ID NO: 195 - DNA, 46 bp, synthetic construct
  source 1..46: mol_type = other DNA
gtgcgggtgccagggcgtgccccggggctccccgggcgcgtactcc

SEQ ID NO: 196 - DNA, 46 bp, synthetic construct
  source 1..46: mol_type = other DNA
gtgcgggtgccagggcgtgcccctgggctccccgggcgcgtactcc

SEQ ID NO: 197 - DNA, 46 bp, synthetic construct
  source 1..46: mol_type = other DNA
gtgcgggtgccagggcgtgcccgagggctccccgggcgcgtactcc

SEQ ID NO: 198 - DNA, 46 bp, synthetic construct
  source 1..46: mol_type = other DNA
gtgcgggtgccagggcgtgcccgcgggctccccgggcgcgtactcc

SEQ ID NO: 199 - DNA, 46 bp, synthetic construct
  source 1..46: mol_type = other DNA
gtgcgggtgccagggcgtgcccgggggctccccgggcgcgtactcc

SEQ ID NO: 200 - DNA, 46 bp, synthetic construct
  source 1..46: mol_type = other DNA
gtgcgggtgccagggcgtgcccgtgggctccccgggcgcgtactcc

SEQ ID NO: 201 - DNA, 46 bp, synthetic construct
  source 1..46: mol_type = other DNA
gtgcgggtgccagggcgtgccctagggctccccgggcgcgtactcc

SEQ ID NO: 202 - DNA, 46 bp, synthetic construct
  source 1..46: mol_type = other DNA
gtgcgggtgccagggcgtgccctcgggctccccgggcgcgtactcc

SEQ ID NO: 203 - DNA, 46 bp, synthetic construct
  source 1..46: mol_type = other DNA
gtgcgggtgccagggcgtgccctggggctccccgggcgcgtactcc

SEQ ID NO: 204 - DNA, 46 bp, synthetic construct
  source 1..46: mol_type = other DNA
gtgcgggtgccagggcgtgcccttgggctccccgggcgcgtactcc

SEQ ID NO: 205 - DNA, 46 bp, synthetic construct
  source 1..46: mol_type = other DNA
agtgccccaactggggtaacctaagagttctctcagttgggggcgt

SEQ ID NO: 206 - DNA, 46 bp, synthetic construct
  source 1..46: mol_type = other DNA
agtgccccaactggggtaacctacgagttctctcagttgggggcgt

SEQ ID NO: 207 - DNA, 46 bp, synthetic construct
  source 1..46: mol_type = other DNA
agtgccccaactggggtaacctaggagttctctcagttgggggcgt

SEQ ID NO: 208 - DNA, 46 bp, synthetic construct
  source 1..46: mol_type = other DNA
agtgccccaactggggtaacctatgagttctctcagttgggggcgt

SEQ ID NO: 209 - DNA, 46 bp, synthetic construct
  source 1..46: mol_type = other DNA
agtgccccaactggggtaacctcagagttctctcagttgggggcgt

SEQ ID NO: 210 - DNA, 46 bp, synthetic construct
  source 1..46: mol_type = other DNA
agtgccccaactggggtaacctccgagttctctcagttgggggcgt

SEQ ID NO: 211 - DNA, 46 bp, synthetic construct
  source 1..46: mol_type = other DNA
agtgccccaactggggtaacctcggagttctctcagttgggggcgt

SEQ ID NO: 212 - DNA, 46 bp, synthetic construct
  source 1..46: mol_type = other DNA
agtgccccaactggggtaacctctgagttctctcagttgggggcgt

SEQ ID NO: 213 - DNA, 46 bp, synthetic construct
  source 1..46: mol_type = other DNA
agtgccccaactggggtaacctgagagttctctcagttgggggcgt

SEQ ID NO: 214 - DNA, 46 bp, synthetic construct
  source 1..46: mol_type = other DNA
agtgccccaactggggtaacctgcgagttctctcagttgggggcgt

SEQ ID NO: 215 - DNA, 46 bp, synthetic construct
  source 1..46: mol_type = other DNA
agtgccccaactggggtaacctgggagttctctcagttgggggcgt

SEQ ID NO: 216 - DNA, 46 bp, synthetic construct
  source 1..46: mol_type = other DNA
agtgccccaactggggtaacctgtgagttctctcagttgggggcgt

SEQ ID NO: 217 - DNA, 46 bp, synthetic construct
  source 1..46: mol_type = other DNA
agtgccccaactggggtaaccttagagttctctcagttgggggcgt

SEQ ID NO: 218 - DNA, 46 bp, synthetic construct
  source 1..46: mol_type = other DNA
agtgccccaactggggtaaccttcgagttctctcagttgggggcgt

SEQ ID NO: 219 - DNA, 46 bp, synthetic construct
  source 1..46: mol_type = other DNA
agtgccccaactggggtaaccttggagttctctcagttgggggcgt

SEQ ID NO: 220 - DNA, 46 bp, synthetic construct
  source 1..46: mol_type = other DNA
agtgccccaactggggtaacctttgagttctctcagttgggggcgt

SEQ ID NO: 221 - DNA, 34 bp, synthetic construct
  source 1..34: mol_type = other DNA
ataacttcgtatagcatacattatacgaacggta

SEQ ID NO: 222 - DNA, 34 bp, synthetic construct
  source 1..34: mol_type = other DNA
taccgttcgtatagcatacattatacgaagttat

SEQ ID NO: 223 - DNA, 34 bp, synthetic construct
  source 1..34: mol_type = other DNA
ataacttcgtataatgtatactatacgaagttat

SEQ ID NO: 224 - DNA, 34 bp, synthetic construct
  source 1..34: mol_type = other DNA
ataacttcgtataggatactttatacgaagttat

SEQ ID NO: 225 - DNA, 34 bp, synthetic construct
  source 1..34: mol_type = other DNA
ataacttcgtataatgtgtactatacgaagttat

SEQ ID NO: 226 - DNA, 34 bp, synthetic construct
  source 1..34: mol_type = other DNA
ataacttcgtatagcatacattataccttgttat

SEQ ID NO: 227 - DNA, 34 bp, synthetic construct
  source 1..34: mol_type = other DNA
taccgttcgtatatggtttcttatacgaagttat

SEQ ID NO: 228 - DNA, 34 bp, synthetic construct
  source 1..34: mol_type = other DNA
ataacttcgtatagtataccttatacgaagttat

SEQ ID NO: 229 - DNA, 34 bp, synthetic construct
  source 1..34: mol_type = other DNA
ataacttcgtatagcatacattatacgaagttat

SEQ ID NO: 230 - DNA, 34 bp, synthetic construct
  source 1..34: mol_type = other DNA
tcaatttctgagaactgtcattctcggaaattga

SEQ ID NO: 231 - DNA, 34 bp, synthetic construct
  source 1..34: mol_type = other DNA
tcaatttctgagaagtgtctttctcggaaattga

SEQ ID NO: 232 - DNA, 34 bp, synthetic construct
  source 1..34: mol_type = other DNA
tcaatttccgagaactgtcattctcggaaattga

SEQ ID NO: 233 - DNA, 34 bp, synthetic construct
  source 1..34: mol_type = other DNA
tcaatttctgagaactgtcattctcagaaattga

SEQ ID NO: 234 - DNA, 34 bp, synthetic construct
  source 1..34: mol_type = other DNA
tcaatttctgagaactgtcattctcggaatacct

SEQ ID NO: 235 - DNA, 34 bp, synthetic construct
  source 1..34: mol_type = other DNA
cgtgattctgagaactgtcattctcggaaattga

SEQ ID NO: 236 - protein, 20 aa, synthetic construct
QCTNYALLKLAGDVESNPGP

SEQ ID NO: 237 - protein, 22 aa, synthetic construct
VKQTLNFDLLKLAGDVESNPGP

SEQ ID NO: 238 - protein, 469 aa, synthetic construct
MNRGGPTVRADIYVRISLDRTGEELGVERQEESCRELCKSLGMEVGQVWVDNDLSATKKNVVRPDFEAMIASNPQAIVCWHTDRLIRVTRDLERVIDLGVNVHAVMAGHLDLSTPAGRAVARTVTAWATYEGEQKAERQKLANIQNARAGKPYTPGIRPFGYGDDHMTIVTAEADAIRDGAKMILDGWSLSAVARYWEELKLQSPRSMAAGGKGWSLRGVKKVLTSPRYVGRSSYLGEVVGDAQWPPILDPDVYYGVVAILNNPDRFSGGPRTGRTPGTLLAGIALCGECGKTVSGRGYRGVLVYGCKDTHTRTPRSIADGRASSSTLARLMFPDFLPGLLASGQAEDGQSAASKHSEAQTLRERLDGLATAYAEGAISLSQMTAGSEALRKKLEVIEADLVGSAGIPPFDPVAGVAGLISGWPTTPLPTRRAWVDFCLVVTLNTQKGRHASSMTVDDHVTIEWRDVAE

SEQ ID NO: 239 - protein, 351 aa, synthetic construct
MPKKKRKVGSSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

(19) United States
(12) Patent Application Publication
(10) Pub. No.: US 2026/0028602 A1
(43) Pub. Date: Jan. 29, 2026

(54) SPLIT RECOMBINASES HAVING INDUCIBLE RECOMBINASE ACTIVITY

(71) Applicant: Asimov Inc., Boston, MA (US)

(72) Inventors: Jeremy J. Gam, Somerville, MA (US); Maya L. Kaul, Boston, MA (US); Alec A.K. Nielsen, Brookline, MA (US)

(73) Assignee: Asimov Inc., Boston, MA (US)

(21) Appl. No.: 19/101,379

(22) PCT Filed: Aug. 8, 2023
(86) PCT No.: PCT/US2023/071850

§ 371 Date: Feb. 5, 2025

Related U.S. Application Data
(60) Provisional application No. 63/370,881, filed on Aug. 9, 2022.

(51) Int. Cl. C12N 9/12; C07K 14/005

(52) U.S. Cl. CPC C12N 9/1241; C07K 14/005; C07K 2319/70; C12N 2750/14122; C12N 2840/203; C12Y 207/07

(57) ABSTRACT

Described herein are split recombinases having inducible recombinase activity and polynucleic acid molecules encoding the same. Also described herein are kits and host cells comprising the split recombinases, as well as methods of their use.

### CROSS-REFERENCE TO RELATED APPLICATIONS

This application claims the benefit under 35 U.S.C. § 119(e) of U.S. Provisional Application No. 63/370,881, filed Aug. 9, 2022, the entire contents of which are hereby incorporated by reference.

### REFERENCE TO AN ELECTRONIC SEQUENCE LISTING

The contents of the electronic sequence listing (A121070011W000-SEQ-CRP.xml; Size: 376,273 bytes; and Date of Creation: Aug. 8, 2023) is hereby incorporated by reference in its entirety.

### FIELD

Described herein are split recombinases having inducible recombinase activity and polynucleic acid molecules encoding the same. Also described herein are kits and host cells comprising the split recombinases, as well as methods of their use.

### BACKGROUND OF INVENTION

Recombinases are enzymes that catalyze site-specific recombination events within DNA. Recombinases are widely used in multicellular organisms to manipulate the structure of genomes and, thereby, to control gene expression. The use of recombinases to manipulate expression in engineered cells has been limited by their toxicity.

### SUMMARY OF INVENTION

Described herein are split recombinases, which in some embodiments have inducible recombinase activity. The inducible split recombinases described herein are expected to have reduced toxicity relative to the recombinases from which they are derived. These split recombinases can be used to regulate gene expression in various applications. For example, an AAV production system may comprise a split recombinase, wherein the split recombinase mediates inducible control of a gene product(s) required for AAV production, including cytostatic or cytotoxic AAV gene products.

In some aspects, the disclosure relates to polynucleic acid molecules encoding a polypeptide dimer having recombinase activity. In some embodiments, the nucleic acid sequence encoding the polypeptide dimer comprises, from 5′ to 3′: (i) a sequence encoding for a first polypeptide comprising a first portion of a recombinase and a first dimerization domain; (ii) a sequence encoding for a viral 2A peptide and/or an internal ribosomal entry site (IRES); and (iii) a sequence encoding for a second polypeptide comprising a second portion of a recombinase and a second dimerization domain; wherein the first polypeptide and the second polypeptide lack recombinase activity in the absence of dimerization; and wherein a recombinase dimer comprising the first polypeptide and the second polypeptide has recombinase activity.

In some embodiments, the polypeptide dimer is derived from a Flp recombinase, a Bxb1 recombinase, a PhiC31 recombinase, a TP901 recombinase, a Cre recombinase, a VCre recombinase, a R4 recombinase, a Dre recombinase, an Int1 recombinase, an Int2 recombinase, an Int3 recombinase, an Int4 recombinase, an Int5 recombinase, an Int6 recombinase, an Int7 recombinase, an Int8 recombinase, an Int9 recombinase, an Int10 recombinase, an Int11 recombinase, an Int 12 recombinase, an Int13 recombinase, an Int14 recombinase, an Int15 recombinase, an Int16 recombinase, an Int17 recombinase, an Int18 recombinase, an Int19 recombinase, an Int20 recombinase, an Int21 recombinase, an Int22 recombinase, an Int23 recombinase, an Int24 recombinase, an Int25 recombinase, an Int26 recombinase, an Int27 recombinase, an Int28 recombinase, an Int29 recombinase, an Int30 recombinase, an Int31 recombinase, an Int32 recombinase, an Int33 recombinase, or an Int34 recombinase.

In some embodiments, the polypeptide dimer is derived from Flp recombinase.

In some embodiments, the first portion of the recombinase corresponds to an N-terminal portion of Flp recombinase and consists of an amino acid sequence having at least 85% identity to SEQ ID NO: 41, and the second portion of the recombinase corresponds to a C-terminal portion of Flp recombinase and consists of an amino acid sequence having at least 85% identity to SEQ ID NO: 42. In some embodiments, the first portion of the recombinase corresponds to a C-terminal portion of Flp recombinase and consists of an amino acid sequence having at least 85% identity to SEQ ID NO: 42, and the second portion of the recombinase corresponds to an N-terminal portion of Flp recombinase and consists of an amino acid sequence having at least 85% identity to SEQ ID NO: 41.

In some embodiments, the first portion of the recombinase corresponds to an N-terminal portion of Flp recombinase and consists of an amino acid sequence having at least 85% identity to SEQ ID NO: 43, and the second portion of the recombinase corresponds to a C-terminal portion of Flp recombinase and consists of an amino acid sequence having at least 85% identity to SEQ ID NO: 44. In some embodiments, the first portion of the recombinase corresponds to a C-terminal portion of Flp recombinase and consists of an amino acid sequence having at least 85% identity to SEQ ID NO: 44, and the second portion of the recombinase corresponds to an N-terminal portion of Flp recombinase and consists of an amino acid sequence having at least 85% identity to SEQ ID NO: 43.

In some embodiments, the polypeptide dimer is derived from Bxb1 recombinase.

In some embodiments, the first portion of the recombinase corresponds to an N-terminal portion of Bxb1 recombinase and consists of an amino acid sequence having at least 85% identity to SEQ ID NO: 65, and the second portion of the recombinase corresponds to a C-terminal portion of Bxb1 recombinase and consists of an amino acid sequence having at least 85% identity to SEQ ID NO: 66. In some embodiments, the first portion of the recombinase corresponds to a C-terminal portion of Bxb1 recombinase and consists of an amino acid sequence having at least 85% identity to SEQ ID NO: 66, and the second portion of the recombinase corresponds to an N-terminal portion of Bxb1 recombinase and consists of an amino acid sequence having at least 85% identity to SEQ ID NO: 65.

In some embodiments, the first portion of the recombinase corresponds to an N-terminal portion of Bxb1 recombinase and consists of an amino acid sequence having at least 85% identity to SEQ ID NO: 47, and the second portion of the recombinase corresponds to a C-terminal portion of Bxb1 recombinase and consists of an amino acid sequence having at least 85% identity to SEQ ID NO: 48. In some embodiments, the first portion of the recombinase corresponds to a C-terminal portion of Bxb1 recombinase and consists of an amino acid sequence having at least 85% identity to SEQ ID NO: 48, and the second portion of the recombinase corresponds to an N-terminal portion of Bxb1 recombinase and consists of an amino acid sequence having at least 85% identity to SEQ ID NO: 47.

In some embodiments, the first portion of the recombinase corresponds to an N-terminal portion of Bxb1 recombinase and consists of an amino acid sequence having at least 85% identity to SEQ ID NO: 49, and the second portion of the recombinase corresponds to a C-terminal portion of Bxb1 recombinase and consists of an amino acid sequence having at least 85% identity to SEQ ID NO: 50. In some embodiments, the first portion of the recombinase corresponds to a C-terminal portion of Bxb1 recombinase and consists of an amino acid sequence having at least 85% identity to SEQ ID NO: 50, and the second portion of the recombinase corresponds to an N-terminal portion of Bxb1 recombinase and consists of an amino acid sequence having at least 85% identity to SEQ ID NO: 49.

In some embodiments, the first portion of the recombinase corresponds to an N-terminal portion of Bxb1 recombinase and consists of an amino acid sequence having at least 85% identity to SEQ ID NO: 53, and the second portion of the recombinase corresponds to a C-terminal portion of Bxb1 recombinase and consists of an amino acid sequence having at least 85% identity to SEQ ID NO: 54. In some embodiments, the first portion of the recombinase corresponds to a C-terminal portion of Bxb1 recombinase and consists of an amino acid sequence having at least 85% identity to SEQ ID NO: 54, and the second portion of the recombinase corresponds to an N-terminal portion of Bxb1 recombinase and consists of an amino acid sequence having at least 85% identity to SEQ ID NO: 53.

In some embodiments, the first portion of the recombinase corresponds to an N-terminal portion of Bxb1 recombinase and consists of an amino acid sequence having at least 85% identity to SEQ ID NO: 59, and the second portion of the recombinase corresponds to a C-terminal portion of Bxb1 recombinase and consists of an amino acid sequence having at least 85% identity to SEQ ID NO: 60. In some embodiments, the first portion of the recombinase corresponds to a C-terminal portion of Bxb1 recombinase and consists of an amino acid sequence having at least 85% identity to SEQ ID NO: 60, and the second portion of the recombinase corresponds to an N-terminal portion of Bxb1 recombinase and consists of an amino acid sequence having at least 85% identity to SEQ ID NO: 59.

In some embodiments, the polypeptide dimer is derived from PhiC31 recombinase.

In some embodiments, the first portion of the recombinase corresponds to an N-terminal portion of PhiC31 recombinase and consists of an amino acid sequence having at least 85% identity to SEQ ID NO: 67, and the second portion of the recombinase corresponds to a C-terminal portion of PhiC31 recombinase and consists of an amino acid sequence having at least 85% identity to SEQ ID NO: 68. In some embodiments, the first portion of the recombinase corresponds to a C-terminal portion of PhiC31 recombinase and consists of an amino acid sequence having at least 85% identity to SEQ ID NO: 68, and the second portion of the recombinase corresponds to an N-terminal portion of PhiC31 recombinase and consists of an amino acid sequence having at least 85% identity to SEQ ID NO: 67.

In some embodiments, the first portion of the recombinase corresponds to an N-terminal portion of PhiC31 recombinase and consists of an amino acid sequence having at least 85% identity to SEQ ID NO: 69, and the second portion of the recombinase corresponds to a C-terminal portion of PhiC31 recombinase and consists of an amino acid sequence having at least 85% identity to SEQ ID NO: 70. In some embodiments, the first portion of the recombinase corresponds to a C-terminal portion of PhiC31 recombinase and consists of an amino acid sequence having at least 85% identity to SEQ ID NO: 70, and the second portion of the recombinase corresponds to an N-terminal portion of PhiC31 recombinase and consists of an amino acid sequence having at least 85% identity to SEQ ID NO: 69.

In some embodiments, the polypeptide dimer is derived from TP901 recombinase.

In some embodiments, the first portion of the recombinase corresponds to an N-terminal portion of TP901 recombinase and consists of an amino acid sequence having at least 85% identity to SEQ ID NO: 71, and the second portion of the recombinase corresponds to a C-terminal portion of TP901 recombinase and consists of an amino acid sequence having at least 85% identity to SEQ ID NO: 72. In some embodiments, the first portion of the recombinase corresponds to a C-terminal portion of TP901 recombinase and consists of an amino acid sequence having at least 85% identity to SEQ ID NO: 72, and the second portion of the recombinase corresponds to an N-terminal portion of TP901 recombinase and consists of an amino acid sequence having at least 85% identity to SEQ ID NO: 71.

In some embodiments, the polypeptide dimer is derived from Cre recombinase.

In some embodiments, the first portion of the recombinase corresponds to an N-terminal portion of Cre recombinase and consists of an amino acid sequence having at least 85% identity to SEQ ID NO: 73, and the second portion of the recombinase corresponds to a C-terminal portion of Cre recombinase and consists of an amino acid sequence having at least 85% identity to SEQ ID NO: 74. In some embodiments, the first portion of the recombinase corresponds to a C-terminal portion of Cre recombinase and consists of an amino acid sequence having at least 85% identity to SEQ ID NO: 74, and the second portion of the recombinase corresponds to an N-terminal portion of Cre recombinase and consists of an amino acid sequence having at least 85% identity to SEQ ID NO: 73.

In some embodiments, the polypeptide dimer is derived from VCre recombinase. In some embodiments, the first portion of the recombinase corresponds to an N-terminal portion of VCre recombinase and consists of an amino acid sequence having at least 85% identity to SEQ ID NO: 77, and the second portion of the recombinase corresponds to a C-terminal portion of VCre recombinase and consists of an amino acid sequence having at least 85% identity to SEQ ID NO: 78. In some embodiments, the first portion of the recombinase corresponds to a C-terminal portion of VCre recombinase and consists of an amino acid sequence having at least 85% identity to SEQ ID NO: 78, and the second portion of the recombinase corresponds to an N-terminal portion of VCre recombinase and consists of an amino acid sequence having at least 85% identity to SEQ ID NO: 77.

In some embodiments, in the first polypeptide, the first dimerization domain is N-terminal to the first portion of the recombinase. In some embodiments, in the first polypeptide, the first dimerization domain is C-terminal to the first portion of the recombinase. In some embodiments, in the second polypeptide, the second dimerization domain is N-terminal to the second portion of the recombinase. In some embodiments, in the second polypeptide, the second dimerization domain is C-terminal to the second portion of the recombinase.

In some embodiments, the dimerization of the first polypeptide and the second polypeptide is dependent on the presence of a small molecule inducer. In some embodiments, the small molecule inducer is selected from the group consisting of gibberellic acid, abscisic acid, and rapalog.

In some embodiments, the first dimerization domain comprises an amino acid sequence having at least 85% identity to SEQ ID NO: 80, and the second dimerization domain comprises an amino acid sequence having at least 85% identity to SEQ ID NO: 79.

In some embodiments, the first dimerization domain comprises an amino acid sequence having at least 85% identity to SEQ ID NO: 79, and the second dimerization domain comprises an amino acid sequence having at least 85% identity to SEQ ID NO: 80.

In some embodiments, the first dimerization domain comprises an amino acid sequence having at least 85% identity to SEQ ID NO: 81, and the second dimerization domain comprises an amino acid sequence having at least 85% identity to SEQ ID NO: 82.

In some embodiments, the first dimerization domain comprises an amino acid sequence having at least 85% identity to SEQ ID NO: 82, and the second dimerization domain comprises an amino acid sequence having at least 85% identity to SEQ ID NO: 81.

In some embodiments, the first dimerization domain comprises an amino acid sequence having at least 85% identity to SEQ ID NO: 83, and the second dimerization domain comprises an amino acid sequence having at least 85% identity to SEQ ID NO: 84.

In some embodiments, the first dimerization domain comprises an amino acid sequence having at least 85% identity to SEQ ID NO: 84, and the second dimerization domain comprises an amino acid sequence having at least 85% identity to SEQ ID NO: 83.

In some embodiments, the nucleic acid sequence encoding the polypeptide dimer comprises a sequence encoding for a viral 2A peptide. In some embodiments, the viral 2A peptide comprises an amino acid sequence having at least 85% identity to any one of SEQ ID NOs: 88-89 and 236-237.

In some embodiments, the nucleic acid sequence encoding the polypeptide dimer comprises a sequence encoding for an IRES. In some embodiments, the IRES comprises a nucleic acid sequence having at least 85% identity to any one of SEQ ID NOs: 85-87.

In some embodiments, the nucleic acid sequence encoding the polypeptide dimer comprises a sequence having at least 85% identity to any one of SEQ ID NOs: 90-110.

In some embodiments, the polynucleic acid molecule encodes a polycistronic mRNA operably linked to a promoter, wherein the polycistronic mRNA comprises the nucleic acid sequence encoding the polypeptide dimer. In some embodiments, the nucleic acid sequence encoding for the polycistronic mRNA is operably linked to a constitutive promoter. In some embodiments, the nucleic acid sequence encoding for the polycistronic mRNA is operably linked to an inducible promoter.

In some embodiments, the polynucleic acid molecule comprises an expression cassette comprising the nucleotide encoding for the polycistronic mRNA operably linked to a promoter, wherein the expression cassette comprises a nucleic acid sequence having at least 85% identity to any one of SEQ ID NOs: 132-143.

In some aspects, the disclosure relates to engineered cells comprising a polynucleic acid molecule encoding a polypeptide having recombinase activity, as provided herein.

In some embodiments, an engineered cell comprises: (a) a first polynucleic acid molecule encoding a polypeptide having recombinase activity, as provided herein; and (b) a second polynucleic acid molecule comprising a nucleic acid sequence encoding, from 5′ to 3′: (i) a first recombinase site; (ii) a gene coding segment; and (iii) a second recombinase site; wherein the first and second recombinase sites correspond to the polypeptide dimer having recombinase activity encoded by the polycistronic mRNA of the first polynucleic acid of (a).

In some embodiments, the first recombinase site of the second polynucleic acid molecule comprises a nucleic acid sequence having at least 85% identity to any one of SEQ ID NOs: 144-235 and the second recombinase site of the second polynucleic acid molecule comprises the nucleic acid sequence having at least 85% identity to any one of SEQ ID NOs: 144-235.

In some embodiments, the gene coding segment comprises a nucleic acid sequence encoding for at least a portion of Rep52, at least a portion of Rep40, at least a portion of Rep78, at least a portion of Rep68, at least a portion of E2A, at least a portion of E4Orf6, at least a portion of VARNA, at least a portion of VP1, at least a portion of VP2, at least a portion of VP3, at least a portion of AAP, or a combination thereof.

In some embodiments, the engineered cell comprises a stable integration of one or more polynucleic acid molecules collectively comprising nucleic acid sequences encoding for: Rep52 or Rep40; Rep78 or Rep68; E2A; E4Orf6; VARNA; VP1; VP2; VP3; and AAP.

In some embodiments, the engineered cell comprises one or more polynucleic acid molecules collectively comprising nucleic acid sequences encoding for: UL5, UL8, UL29, UL30, UL42, UL52, UL12, ICP10, ICP4, and ICP22.

### BRIEF DESCRIPTION OF DRAWINGS

FIG. 1 provides a diagram of an exemplary small molecule-inducible split recombinases. A genetic schematic of a split recombinase is shown (top) containing the N-terminal portion of a recombinase (Rec-N), a dimerization domain (D1), a corresponding dimerization domain (D2), and the C-terminal portion of the same recombinase (Rec-C). The dimerization domains are separated by a 2A peptide or IRES sequence to allow both coding regions to be expressed from the same transcript. The transcript can be driven by a constitutive promoter (e.g., hEFla) or an inducible promoter (e.g., TRE3G). A schematic showing small-molecule-mediated dimerization is also shown (bottom). After both polypeptides of a split recombinase are expressed separately, or otherwise separated by autocatalysis, they remain separated such that minimal recombinase activity is present, until a dimerizing small molecule is added, causing association of the two dimerization domains and reconstituting recombinase function.

FIGS. 2A-2D show the genetic schematics (left) and experimental results (right) for four embodiments of split Cre recombinases (CreN: N-terminal portion of Cre recombinase; CreC: C-terminal portion of Cre recombinase). Experiments were performed in the presence (+SM) and absence (−SM) of small molecule inducers. FIG. 2A provides a genetic schematic (left) and experimental results (right) for a first embodiment (v1) of a split Cre recombinase. FIG. 2G provides a genetic schematic (left) and experimental results (right) for a second (v2) embodiment of a split Cre recombinase. FIG. 2C provides a genetic schematic (left) and experimental results (right) for a third embodiment (v3) of a split Cre recombinase. FIG. 2D provides a genetic schematic (left) and experimental results (right) for a fourth embodiment (v4) of a split Cre recombinase.

FIG. 3 shows experimental results for several split recombinases (e.g., “Flp 27/28-GA” and “Flp 396/397-ABA”) compared to their respective non-split forms (e.g., “Flp”). The amino acid position of the split in each split recombinase is shown (e.g., Flp 27/28 indicates a split between amino acids 27 and 28 of Flp), as well as the small molecule inducer that was used to induce dimerization (e.g., GA: gibberellic acid; ABA: abscisic acid; Rap: rapalog). −SM; minus small molecule inducer; +SM; plus small molecule inducer.

FIG. 4 shows results for eleven embodiments of split Bxb1 recombinases. GID1 and GAI dimerization domains were used, and dimerization was induced in the presence of GA (gibberellic acid). The amino acid position of the split in each split recombinase is shown (e.g., 468/469 indicates a split between amino acids 468 and 469 of Bxb1). −SM; minus small molecule inducer; +SM; plus small molecule inducer.

FIGS. 5A-5L show FACS results for the eleven embodiments of split Bxb1 recombinases of FIG. 4. FIG. 5A: unsplit Bxb1; FIG. 5B: Bxb1 37/38; FIG. 5C: Bxb1 169/170; FIG. 5D: Bxb1 208/209; FIG. 5E: Bxb1 222/223; FIG. 5F: Bxb1 259/260; FIG. 5G: Bxb1 262/263; FIG. 5H: Bxb1 363/364; FIG. 5I: Bxb1 370/371; FIG. 5J: Bxb1 399/400; FIG. 5K: Bxb1 440/441; FIG. 5L: Bxb1 468/469. −SM; minus small molecule inducer; +SM; plus small molecule inducer. X-axis shows iRFP720 transfection marker fluorescence measured in the APC-A700 channel. Y-axis shows EGFP reporter expression measured in the FITC channel.

FIGS. 6A-5H shows FACS results for eight embodiments of split recombinases compared to their respective non-split forms. FIG. 6A: Flp 27/28 (GA small molecule inducer); FIG. 6B: Flp 396/397 (ABA small molecule inducer); FIG. 6C: Cre 229/230 (GA small molecule inducer); FIG. 6D: VCre 269/270 (GA small molecule inducer); FIG. 6E: Phi 233/234 (GA small molecule inducer); FIG. 6F: Phi 571/572 (Rap small molecule inducer);

FIG. 6G: TP901 326/327 (GA small molecule inducer); FIG. 6H: Bxb1 468/469. −SM; minus small molecule inducer; +SM; plus small molecule inducer. All charts are in terms of transfection marker (iRFP measured in APC-700 channel) and fluorescent readout (EGFP measured in FITC channel). VCre plasmid without split had a nonsense mutation and has no data.

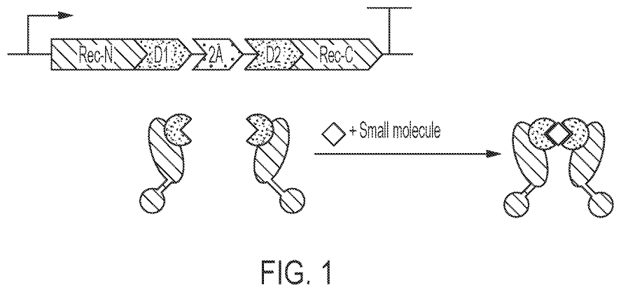

### DETAILED DESCRIPTION OF INVENTION

Split recombinases operate by expressing a recombinase in two parts, each part incapable of independent catalytic activity (see e.g., Weinberg et al., Nat Commun. 2019 Oct 24;10(1):4845. Doi: 10.1038/s41467-019-12800-7). Split recombinases may be expressed linked to a domain capable of dimerizing in the presence of a small molecule, reconstituting the recombinase, and restoring catalytic activity.

In addition to providing previously undescribed split recombinases, the instant disclosure provides polynucleic acid molecules that encode a split recombinase in a single expression cassette. This allows one to express the split recombinase from a single transcription unit, rather than from separate promoters or plasmids.

The split recombinases (and polynucleic acid molecules encoding the same) that are described herein have various applications. For example, disclosed herein are adenovirus (AAV) production systems comprising a split recombinase, wherein the split recombinase mediates inducible control of expression of an AAV gene product(s) required for AAV production, such as a cytostatic or cytotoxic AAV gene product(s) (e.g., Rep, E2A and E4). The cytotoxic and cytostatic nature of these gene products has hampered the development of stable AAV producer cell lines.

Also described herein are engineered cells and kits comprising the split recombinases.

### I. Split Recombinases

In some aspects, the disclosure relates to split recombinases. A “split recombinase” as described herein is a polypeptide dimer comprising a first polypeptide and a second polypeptide, wherein the first polypeptide and the second polypeptide lack recombinase activity in the absence of dimerization, but have recombinase activity when dimerized. In particular, the split recombinases described herein comprise: (i) a first polypeptide comprising a first portion of a recombinase and a first dimerization domain; and (ii) a second polypeptide comprising a second portion of the recombinase and a second dimerization domain; wherein the first polypeptide and the second polypeptide lack recombinase activity in the absence of dimerization; and wherein a recombinase dimer comprising the first polypeptide and the second polypeptide has recombinase activity.

As used herein, the term “recombinase activity” refers to the ability to catalyze site-specific recombination events within DNA. Methods of determining whether a split recombinase has recombinase activity (e.g., in the presence and absence of dimerization) are known to those having skill in the art. Exemplary methods of determining whether a split recombinase has recombinase activity are provided herein in the Examples section.

Recombinase activity of a split recombinase can be controlled (e.g., induced) in various ways. For example, in some embodiments, dimerization of the first polypeptide and the second polypeptide of a split recombinase may depend on the presence of a small molecule inducer. Alternatively, or in addition, in some embodiments, the nucleic acid sequence encoding at least one polypeptide of a split recombinase dimer (and optionally the nucleic acid sequence(s) of both polypeptides of the split recombinase dimer) is operably linked to an inducible promoter.

In some embodiments, the first polypeptide of a split recombinase comprises, from N-terminus to C-terminus: the first portion of the recombinase; and the first dimerization domain. In other embodiments, the first polypeptide of a split recombinase comprises, from N-terminus to C-terminus: the first dimerization domain; and the first portion of the recombinase.

In some embodiments, the second polypeptide of a split recombinase comprises, from N-terminus to C-terminus: the second portion of the recombinase; and the second dimerization domain. In other embodiments, the second polypeptide of a split recombinase comprises, from N-terminus to C-terminus: the second dimerization domain; and the second portion of the recombinase.

In some embodiments, the first portion of the recombinase corresponds to the N-terminal portion of the recombinase from which the split recombinase is derived, and the second portion of the recombinase corresponds to the C-terminal portion of the recombinase from which the split recombinase is derived. In other embodiments, the second portion of the recombinase corresponds to the N-terminal portion of the recombinase from which the split recombinase is derived, and the first portion of the recombinase corresponds to the C-terminal portion of the recombinase from which the split recombinase is derived.

a. First and Second Portions of a Split Recombinase

A split recombinase may be derived from any previously described recombinase (see e.g., Weinberg et al., Nat Commun. 2019 Oct. 24; 10(1):4845. Doi: 10.1038/s41467-019-12800-7). Exemplary recombinase amino acid sequences which have been used herein to derive split recombinases are provided in Table 2.

In some embodiments, a split recombinase described herein comprises a first portion and a second portion of a recombinase selected from the group consisting of a Flp recombinase (e.g., SEQ ID NO: 1), a Bxb1 recombinase (e.g., SEQ ID NO: 2), a PhiC31 recombinase (e.g., SEQ ID NO: 3), a TP901 recombinase (e.g., SEQ ID NO: 4), a Cre recombinase (e.g., SEQ ID NO: 5), a Vcre recombinase (e.g., SEQ ID NO: 6), an Int1 recombinase (e.g., SEQ ID NO: 7), an Int2 recombinase (e.g., SEQ ID NO: 8), an Int3 recombinase (e.g., SEQ ID NO: 9), an Int4 recombinase (e.g., SEQ ID NO: 10), an Int5 recombinase (e.g., SEQ ID NO: 11), an Int6 recombinase (e.g., SEQ ID NO: 12), an Int7 recombinase (e.g., SEQ ID NO: 13), an Int8 recombinase (e.g., SEQ ID NO: 14), an Int9 recombinase (e.g., SEQ ID NO: 15), an Int10 recombinase (e.g., SEQ ID NO: 16), an Int11 recombinase (e.g., SEQ ID NO: 17), an Int12 recombinase (e.g., SEQ ID NO: 18), an Int13 recombinase (e.g., SEQ ID NO: 19), an Int14 recombinase (e.g., SEQ ID NO: 20), an Int15 recombinase (e.g., SEQ ID NO: 21), an Int16 recombinase (e.g., SEQ ID NO: 22), an Int17 recombinase (e.g., SEQ ID NO: 23), an Int18 recombinase (e.g., SEQ ID NO: 24), an Int19 recombinase (e.g., SEQ ID NO: 25), an Int20 recombinase (e.g., SEQ ID NO: 26), an Int21 recombinase (e.g., SEQ ID NO: 27), an Int22 recombinase (e.g., SEQ ID NO: 28), an Int23 recombinase (e.g., SEQ ID NO: 29), an Int24 recombinase (e.g., SEQ ID NO: 30), an Int25 recombinase (e.g., SEQ ID NO: 31), an Int26 recombinase (e.g., SEQ ID NO: 32), an Int27 recombinase (e.g., SEQ ID NO: 33), an Int28 recombinase (e.g., SEQ ID NO: 34), an Int29 recombinase (e.g., SEQ ID NO: 35), an Int30 recombinase (e.g., SEQ ID NO: 36), an Int31 recombinase (e.g., SEQ ID NO: 37), an Int32 recombinase (e.g., SEQ ID NO: 38), an Int33 recombinase (e.g., SEQ ID NO: 39), an Int34 recombinase (e.g., SEQ ID NO: 40), an R4 recombinase (e.g., SEQ ID NO: 238), or a Dre recombinase (e.g., SEQ ID NO: 239), wherein the first portion and the second portion individually lack recombinase activity, but collectively have recombinase activity.

In some embodiments, a split Flp recombinase described herein comprises a first portion and a second portion of a Flp recombinase (e.g., SEQ ID NO: 1), wherein the first portion and the second portion individually lack recombinase activity, but collectively (i.e., when bound together covalently or non-covalently) have recombinase activity. In some embodiments, the first portion of Flp recombinase corresponds to the N-terminal portion of Flp recombinase, and the second portion of Flp recombinase corresponds to the C-terminal portion of Flp recombinase. In other embodiments, the second portion of Flp recombinase corresponds to the N-terminal portion of Flp recombinase, and the first portion of Flp recombinase corresponds to the C-terminal portion of Flp recombinase.

In some embodiments, the first portion of Flp recombinase comprises an amino acid sequence having at least 80%, at least 85%, at least 90%, at least 95%, at least 98%, or at least 99% identity to SEQ ID NO: 41, and the second portion of Flp recombinase comprises an amino acid sequence having at least 80%, at least 85%, at least 90%, at least 95%, at least 98%, or at least 99% identity to SEQ ID NO: 42. In some embodiments, the first portion of Flp recombinase comprises the amino acid sequence of SEQ ID NO: 41, and the second portion of Flp recombinase comprises the amino acid sequence of SEQ ID NO: 42. In some embodiments, the first portion of Flp recombinase comprises an amino acid sequence having at least 80%, at least 85%, at least 90%, at least 95%, at least 98%, or at least 99% identity to SEQ ID NO: 42, and the second portion of Flp recombinase comprises an amino acid sequence having at least 80%, at least 85%, at least 90%, at least 95%, at least 98%, or at least 99% identity to SEQ ID NO: 41. In some embodiments, the first portion of Flp recombinase comprises the amino acid sequence of SEQ ID NO: 42, and the second portion of Flp recombinase comprises the amino acid sequence of SEQ ID NO: 41.

In some embodiments, the first portion of Flp recombinase comprises an amino acid sequence having at least 80%, at least 85%, at least 90%, at least 95%, at least 98%, or at least 99% identity to SEQ ID NO: 43, and the second portion of Flp recombinase comprises an amino acid sequence having at least 80%, at least 85%, at least 90%, at least 95%, at least 98%, or at least 99% identity to SEQ ID NO: 44. In some embodiments, the first portion of Flp recombinase comprises the amino acid sequence of SEQ ID NO: 43, and the second portion of Flp recombinase comprises the amino acid sequence of SEQ ID NO: 44. In some embodiments, the first portion of Flp recombinase comprises an amino acid sequence having at least 80%, at least 85%, at least 90%, at least 95%, at least 98%, or at least 99% identity to SEQ ID NO: 44, and the second portion of Flp recombinase comprises an amino acid sequence having at least 80%, at least 85%, at least 90%, at least 95%, at least 98%, or at least 99% identity to SEQ ID NO: 43. In some embodiments, the first portion of Flp recombinase comprises the amino acid sequence of SEQ ID NO: 44, and the second portion of Flp recombinase comprises the amino acid sequence of SEQ ID NO: 43.

In some embodiments, a split Bxb1 recombinase described herein comprises a first portion and a second portion of a Bxb1 recombinase (e.g., SEQ ID NO: 2), wherein the first portion and the second portion individually lack recombinase activity, but collectively (i.e., when bound together covalently or non-covalently) have recombinase activity. In some embodiments, the first portion of Bxb1 recombinase corresponds to the N-terminal portion of Bxb1 recombinase, and the second portion of Bxb1 recombinase corresponds to the C-terminal portion of Bxb1 recombinase. In other embodiments, the second portion of Bxb1 recombinase corresponds to the N-terminal portion of Bxb1 recombinase, and the first portion of Bxb1 recombinase corresponds to the C-terminal portion of Bxb1 recombinase.

In some embodiments, the first portion of Bxb1 recombinase comprises an amino acid sequence having at least 80%, at least 85%, at least 90%, at least 95%, at least 98%, or at least 99% identity to SEQ ID NO: 45, and the second portion of Bxb1 recombinase comprises an amino acid sequence having at least 80%, at least 85%, at least 90%, at least 95%, at least 98%, or at least 99% identity to SEQ ID NO: 46. In some embodiments, the first portion of Bxb1 recombinase comprises the amino acid sequence of SEQ ID NO: 45, and the second portion of Bxb1 recombinase comprises the amino acid sequence of SEQ ID NO: 46. In some embodiments, the first portion of Bxb1 recombinase comprises an amino acid sequence having at least 80%, at least 85%, at least 90%, at least 95%, at least 98%, or at least 99% identity to SEQ ID NO: 46, and the second portion of Bxb1 recombinase comprises an amino acid sequence having at least 80%, at least 85%, at least 90%, at least 95%, at least 98%, or at least 99% identity to SEQ ID NO: 45. In some embodiments, the first portion of Bxb1 recombinase comprises the amino acid sequence of SEQ ID NO: 46, and the second portion of Bxb1 recombinase comprises the amino acid sequence of SEQ ID NO: 45.

In some embodiments, the first portion of Bxb1 recombinase comprises an amino acid sequence having at least 80%, at least 85%, at least 90%, at least 95%, at least 98%, or at least 99% identity to SEQ ID NO: 47, and the second portion of Bxb1 recombinase comprises an amino acid sequence having at least 80%, at least 85%, at least 90%, at least 95%, at least 98%, or at least 99% identity to SEQ ID NO: 48. In some embodiments, the first portion of Bxb1 recombinase comprises the amino acid sequence of SEQ ID NO: 47, and the second portion of Bxb1 recombinase comprises the amino acid sequence of SEQ ID NO: 48. In some embodiments, the first portion of Bxb1 recombinase comprises an amino acid sequence having at least 80%, at least 85%, at least 90%, at least 95%, at least 98%, or at least 99% identity to SEQ ID NO: 48, and the second portion of Bxb1 recombinase comprises an amino acid sequence having at least 80%, at least 85%, at least 90%, at least 95%, at least 98%, or at least 99% identity to SEQ ID NO: 47. In some embodiments, the first portion of Bxb1 recombinase comprises the amino acid sequence of SEQ ID NO: 48, and the second portion of Bxb1 recombinase comprises the amino acid sequence of SEQ ID NO: 47.

In some embodiments, the first portion of Bxb1 recombinase comprises an amino acid sequence having at least 80%, at least 85%, at least 90%, at least 95%, at least 98%, or at least 99% identity to SEQ ID NO: 49, and the second portion of Bxb1 recombinase comprises an amino acid sequence having at least 80%, at least 85%, at least 90%, at least 95%, at least 98%, or at least 99% identity to SEQ ID NO: 50. In some embodiments, the first portion of Bxb1 recombinase comprises the amino acid sequence of SEQ ID NO: 49, and the second portion of Bxb1 recombinase comprises the amino acid sequence of SEQ ID NO: 50. In some embodiments, the first portion of Bxb1 recombinase comprises an amino acid sequence having at least 80%, at least 85%, at least 90%, at least 95%, at least 98%, or at least 99% identity to SEQ ID NO: 50, and the second portion of Bxb1 recombinase comprises an amino acid sequence having at least 80%, at least 85%, at least 90%, at least 95%, at least 98%, or at least 99% identity to SEQ ID NO: 49. In some embodiments, the first portion of Bxb1 recombinase comprises the amino acid sequence of SEQ ID NO: 50, and the second portion of Bxb1 recombinase comprises the amino acid sequence of SEQ ID NO: 49.

In some embodiments, the first portion of Bxb1 recombinase comprises an amino acid sequence having at least 80%, at least 85%, at least 90%, at least 95%, at least 98%, or at least 99% identity to SEQ ID NO: 51, and the second portion of Bxb1 recombinase comprises an amino acid sequence having at least 80%, at least 85%, at least 90%, at least 95%, at least 98%, or at least 99% identity to SEQ ID NO: 52. In some embodiments, the first portion of Bxb1 recombinase comprises the amino acid sequence of SEQ ID NO: 51, and the second portion of Bxb1 recombinase comprises the amino acid sequence of SEQ ID NO: 52. In some embodiments, the first portion of Bxb1 recombinase comprises an amino acid sequence having at least 80%, at least 85%, at least 90%, at least 95%, at least 98%, or at least 99% identity to SEQ ID NO: 52, and the second portion of Bxb1 recombinase comprises an amino acid sequence having at least 80%, at least 85%, at least 90%, at least 95%, at least 98%, or at least 99% identity to SEQ ID NO: 51. In some embodiments, the first portion of Bxb1 recombinase comprises the amino acid sequence of SEQ ID NO: 52, and the second portion of Bxb1 recombinase comprises the amino acid sequence of SEQ ID NO: 51.

In some embodiments, the first portion of Bxb1 recombinase comprises an amino acid sequence having at least 80%, at least 85%, at least 90%, at least 95%, at least 98%, or at least 99% identity to SEQ ID NO: 53, and the second portion of Bxb1 recombinase comprises an amino acid sequence having at least 80%, at least 85%, at least 90%, at least 95%, at least 98%, or at least 99% identity to SEQ ID NO: 54. In some embodiments, the first portion of Bxb1 recombinase comprises the amino acid sequence of SEQ ID NO: 53, and the second portion of Bxb1 recombinase comprises the amino acid sequence of SEQ ID NO: 54. In some embodiments, the first portion of Bxb1 recombinase comprises an amino acid sequence having at least 80%, at least 85%, at least 90%, at least 95%, at least 98%, or at least 99% identity to SEQ ID NO: 54, and the second portion of Bxb1 recombinase comprises an amino acid sequence having at least 80%, at least 85%, at least 90%, at least 95%, at least 98%, or at least 99% identity to SEQ ID NO: 53. In some embodiments, the first portion of Bxb1 recombinase comprises the amino acid sequence of SEQ ID NO: 54, and the second portion of Bxb1 recombinase comprises the amino acid sequence of SEQ ID NO: 53.

In some embodiments, the first portion of Bxb1 recombinase comprises an amino acid sequence having at least 80%, at least 85%, at least 90%, at least 95%, at least 98%, or at least 99% identity to SEQ ID NO: 55, and the second portion of Bxb1 recombinase comprises an amino acid sequence having at least 80%, at least 85%, at least 90%, at least 95%, at least 98%, or at least 99% identity to SEQ ID NO: 56. In some embodiments, the first portion of Bxb1 recombinase comprises the amino acid sequence of SEQ ID NO: 55, and the second portion of Bxb1 recombinase comprises the amino acid sequence of SEQ ID NO: 56. In some embodiments, the first portion of Bxb1 recombinase comprises an amino acid sequence having at least 80%, at least 85%, at least 90%, at least 95%, at least 98%, or at least 99% identity to SEQ ID NO: 56, and the second portion of Bxb1 recombinase comprises an amino acid sequence having at least 80%, at least 85%, at least 90%, at least 95%, at least 98%, or at least 99% identity to SEQ ID NO: 55. In some embodiments, the first portion of Bxb1 recombinase comprises the amino acid sequence of SEQ ID NO: 56, and the second portion of Bxb1 recombinase comprises the amino acid sequence of SEQ ID NO: 55.

In some embodiments, the first portion of Bxb1 recombinase comprises an amino acid sequence having at least 80%, at least 85%, at least 90%, at least 95%, at least 98%, or at least 99% identity to SEQ ID NO: 57, and the second portion of Bxb1 recombinase comprises an amino acid sequence having at least 80%, at least 85%, at least 90%, at least 95%, at least 98%, or at least 99% identity to SEQ ID NO: 58. In some embodiments, the first portion of Bxb1 recombinase comprises the amino acid sequence of SEQ ID NO: 57, and the second portion of Bxb1 recombinase comprises the amino acid sequence of SEQ ID NO: 58. In some embodiments, the first portion of Bxb1 recombinase comprises an amino acid sequence having at least 80%, at least 85%, at least 90%, at least 95%, at least 98%, or at least 99% identity to SEQ ID NO: 58, and the second portion of Bxb1 recombinase comprises an amino acid sequence having at least 80%, at least 85%, at least 90%, at least 95%, at least 98%, or at least 99% identity to SEQ ID NO: 57. In some embodiments, the first portion of Bxb1 recombinase comprises the amino acid sequence of SEQ ID NO: 58, and the second portion of Bxb1 recombinase comprises the amino acid sequence of SEQ ID NO: 57.

In some embodiments, the first portion of Bxb1 recombinase comprises an amino acid sequence having at least 80%, at least 85%, at least 90%, at least 95%, at least 98%, or at least 99% identity to SEQ ID NO: 59, and the second portion of Bxb1 recombinase comprises an amino acid sequence having at least 80%, at least 85%, at least 90%, at least 95%, at least 98%, or at least 99% identity to SEQ ID NO: 60. In some embodiments, the first portion of Bxb1 recombinase comprises the amino acid sequence of SEQ ID NO: 59, and the second portion of Bxb1 recombinase comprises the amino acid sequence of SEQ ID NO: 60. In some embodiments, the first portion of Bxb1 recombinase comprises an amino acid sequence having at least 80%, at least 85%, at least 90%, at least 95%, at least 98%, or at least 99% identity to SEQ ID NO: 60, and the second portion of Bxb1 recombinase comprises an amino acid sequence having at least 80%, at least 85%, at least 90%, at least 95%, at least 98%, or at least 99% identity to SEQ ID NO: 59. In some embodiments, the first portion of Bxb1 recombinase comprises the amino acid sequence of SEQ ID NO: 60, and the second portion of Bxb1 recombinase comprises the amino acid sequence of SEQ ID NO: 59.

In some embodiments, the first portion of Bxb1 recombinase comprises an amino acid sequence having at least 80%, at least 85%, at least 90%, at least 95%, at least 98%, or at least 99% identity to SEQ ID NO: 61, and the second portion of Bxb1 recombinase comprises an amino acid sequence having at least 80%, at least 85%, at least 90%, at least 95%, at least 98%, or at least 99% identity to SEQ ID NO: 62. In some embodiments, the first portion of Bxb1 recombinase comprises the amino acid sequence of SEQ ID NO: 61, and the second portion of Bxb1 recombinase comprises the amino acid sequence of SEQ ID NO: 62. In some embodiments, the first portion of Bxb1 recombinase comprises an amino acid sequence having at least 80%, at least 85%, at least 90%, at least 95%, at least 98%, or at least 99% identity to SEQ ID NO: 62, and the second portion of Bxb1 recombinase comprises an amino acid sequence having at least 80%, at least 85%, at least 90%, at least 95%, at least 98%, or at least 99% identity to SEQ ID NO: 61. In some embodiments, the first portion of Bxb1 recombinase comprises the amino acid sequence of SEQ ID NO: 62, and the second portion of Bxb1 recombinase comprises the amino acid sequence of SEQ ID NO: 61.

In some embodiments, the first portion of Bxb1 recombinase comprises an amino acid sequence having at least 80%, at least 85%, at least 90%, at least 95%, at least 98%, or at least 99% identity to SEQ ID NO: 63, and the second portion of Bxb1 recombinase comprises an amino acid sequence having at least 80%, at least 85%, at least 90%, at least 95%, at least 98%, or at least 99% identity to SEQ ID NO: 64. In some embodiments, the first portion of Bxb1 recombinase comprises the amino acid sequence of SEQ ID NO: 63, and the second portion of Bxb1 recombinase comprises the amino acid sequence of SEQ ID NO: 64. In some embodiments, the first portion of Bxb1 recombinase comprises an amino acid sequence having at least 80%, at least 85%, at least 90%, at least 95%, at least 98%, or at least 99% identity to SEQ ID NO: 64, and the second portion of Bxb1 recombinase comprises an amino acid sequence having at least 80%, at least 85%, at least 90%, at least 95%, at least 98%, or at least 99% identity to SEQ ID NO: 63. In some embodiments, the first portion of Bxb1 recombinase comprises the amino acid sequence of SEQ ID NO: 64, and the second portion of Bxb1 recombinase comprises the amino acid sequence of SEQ ID NO: 63.

In some embodiments, the first portion of Bxb1 recombinase comprises an amino acid sequence having at least 80%, at least 85%, at least 90%, at least 95%, at least 98%, or at least 99% identity to SEQ ID NO: 65, and the second portion of Bxb1 recombinase comprises an amino acid sequence having at least 80%, at least 85%, at least 90%, at least 95%, at least 98%, or at least 99% identity to SEQ ID NO: 66. In some embodiments, the first portion of Bxb1 recombinase comprises the amino acid sequence of SEQ ID NO: 65, and the second portion of Bxb1 recombinase comprises the amino acid sequence of SEQ ID NO: 66. In some embodiments, the first portion of Bxb1 recombinase comprises an amino acid sequence having at least 80%, at least 85%, at least 90%, at least 95%, at least 98%, or at least 99% identity to SEQ ID NO: 66, and the second portion of Bxb1 recombinase comprises an amino acid sequence having at least 80%, at least 85%, at least 90%, at least 95%, at least 98%, or at least 99% identity to SEQ ID NO: 65. In some embodiments, the first portion of Bxb1 recombinase comprises the amino acid sequence of SEQ ID NO: 66, and the second portion of Bxb1 recombinase comprises the amino acid sequence of SEQ ID NO: 65.

In some embodiments, a split PhiC31 recombinase described herein comprises a first portion and a second portion of a PhiC31 recombinase (e.g., SEQ ID NO: 3), wherein the first portion and the second portion individually lack recombinase activity, but collectively (i.e., when bound together covalently or non-covalently) have recombinase activity. In some embodiments, the first portion of PhiC31 recombinase corresponds to the N-terminal portion of PhiC31 recombinase, and the second portion of PhiC31 recombinase corresponds to the C-terminal portion of PhiC31 recombinase. In other embodiments, the second portion of PhiC31 recombinase corresponds to the N-terminal portion of PhiC31 recombinase, and the first portion of PhiC31 recombinase corresponds to the C-terminal portion of PhiC31 recombinase.

In some embodiments, the first portion of PhiC31 recombinase comprises an amino acid sequence having at least 80%, at least 85%, at least 90%, at least 95%, at least 98%, or at least 99% identity to SEQ ID NO: 67, and the second portion of PhiC31 recombinase comprises an amino acid sequence having at least 80%, at least 85%, at least 90%, at least 95%, at least 98%, or at least 99% identity to SEQ ID NO: 68. In some embodiments, the first portion of PhiC31 recombinase comprises the amino acid sequence of SEQ ID NO: 67, and the second portion of PhiC31 recombinase comprises the amino acid sequence of SEQ ID NO: 68. In some embodiments, the first portion of PhiC31 recombinase comprises an amino acid sequence having at least 80%, at least 85%, at least 90%, at least 95%, at least 98%, or at least 99% identity to SEQ ID NO: 68, and the second portion of PhiC31 recombinase comprises an amino acid sequence having at least 80%, at least 85%, at least 90%, at least 95%, at least 98%, or at least 99% identity to SEQ ID NO: 67. In some embodiments, the first portion of PhiC31 recombinase comprises the amino acid sequence of SEQ ID NO: 68, and the second portion of PhiC31 recombinase comprises the amino acid sequence of SEQ ID NO: 67.

In some embodiments, the first portion of PhiC31 recombinase comprises an amino acid sequence having at least 80%, at least 85%, at least 90%, at least 95%, at least 98%, or at least 99% identity to SEQ ID NO: 69, and the second portion of PhiC31 recombinase comprises an amino acid sequence having at least 80%, at least 85%, at least 90%, at least 95%, at least 98%, or at least 99% identity to SEQ ID NO: 70. In some embodiments, the first portion of PhiC31 recombinase comprises the amino acid sequence of SEQ ID NO: 69, and the second portion of PhiC31 recombinase comprises the amino acid sequence of SEQ ID NO: 70. In some embodiments, the first portion of PhiC31 recombinase comprises an amino acid sequence having at least 80%, at least 85%, at least 90%, at least 95%, at least 98%, or at least 99% identity to SEQ ID NO: 70, and the second portion of PhiC31 recombinase comprises an amino acid sequence having at least 80%, at least 85%, at least 90%, at least 95%, at least 98%, or at least 99% identity to SEQ ID NO: 69. In some embodiments, the first portion of PhiC31 recombinase comprises the amino acid sequence of SEQ ID NO: 70, and the second portion of PhiC31 recombinase comprises the amino acid sequence of SEQ ID NO: 69.

In some embodiments, a split TP901 recombinase described herein comprises a first portion and a second portion of a TP901 recombinase (e.g., SEQ ID NO: 4), wherein the first portion and the second portion individually lack recombinase activity, but collectively (i.e., when bound together covalently or non-covalently) have recombinase activity. In some embodiments, the first portion of TP901 recombinase corresponds to the N-terminal portion of TP901 recombinase, and the second portion of TP901 recombinase corresponds to the C-terminal portion of TP901 recombinase. In other embodiments, the second portion of PhiC31 recombinase corresponds to the N-terminal portion of TP901 recombinase, and the first portion of TP901 recombinase corresponds to the C-terminal portion of TP901 recombinase.

In some embodiments, the first portion of TP901 recombinase comprises an amino acid sequence having at least 80%, at least 85%, at least 90%, at least 95%, at least 98%, or at least 99% identity to SEQ ID NO: 71, and the second portion of TP901 recombinase comprises an amino acid sequence having at least 80%, at least 85%, at least 90%, at least 95%, at least 98%, or at least 99% identity to SEQ ID NO: 72. In some embodiments, the first portion of TP901 recombinase comprises the amino acid sequence of SEQ ID NO: 71, and the second portion of TP901 recombinase comprises the amino acid sequence of SEQ ID NO: 72. In some embodiments, the first portion of TP901 recombinase comprises an amino acid sequence having at least 80%, at least 85%, at least 90%, at least 95%, at least 98%, or at least 99% identity to SEQ ID NO: 72, and the second portion of TP901 recombinase comprises an amino acid sequence having at least 80%, at least 85%, at least 90%, at least 95%, at least 98%, or at least 99% identity to SEQ ID NO: 71. In some embodiments, the first portion of TP901 recombinase comprises the amino acid sequence of SEQ ID NO: 72, and the second portion of TP901 recombinase comprises the amino acid sequence of SEQ ID NO: 71.

In some embodiments, a split Cre recombinase described herein comprises a first portion and a second portion of a Cre recombinase (e.g., SEQ ID NO: 5), wherein the first portion and the second portion individually lack recombinase activity, but collectively (i.e., when bound together covalently or non-covalently) have recombinase activity. In some embodiments, the first portion of Cre recombinase corresponds to the N-terminal portion of Cre recombinase, and the second portion of Cre recombinase corresponds to the C-terminal portion of Cre recombinase. In other embodiments, the second portion of Cre recombinase corresponds to the N-terminal portion of Cre recombinase, and the first portion of Cre recombinase corresponds to the C-terminal portion of Cre recombinase.

In some embodiments, the first portion of Cre recombinase comprises an amino acid sequence having at least 80%, at least 85%, at least 90%, at least 95%, at least 98%, or at least 99% identity to SEQ ID NO: 73, and the second portion of Cre recombinase comprises an amino acid sequence having at least 80%, at least 85%, at least 90%, at least 95%, at least 98%, or at least 99% identity to SEQ ID NO: 74. In some embodiments, the first portion of Cre recombinase comprises the amino acid sequence of SEQ ID NO: 73, and the second portion of Cre recombinase comprises the amino acid sequence of SEQ ID NO: 74. In some embodiments, the first portion of Cre recombinase comprises an amino acid sequence having at least 80%, at least 85%, at least 90%, at least 95%, at least 98%, or at least 99% identity to SEQ ID NO: 74, and the second portion of Cre recombinase comprises an amino acid sequence having at least 80%, at least 85%, at least 90%, at least 95%, at least 98%, or at least 99% identity to SEQ ID NO: 73. In some embodiments, the first portion of Cre recombinase comprises the amino acid sequence of SEQ ID NO: 74, and the second portion of Cre recombinase comprises the amino acid sequence of SEQ ID NO: 73.

In some embodiments, the first portion of Cre recombinase comprises an amino acid sequence having at least 80%, at least 85%, at least 90%, at least 95%, at least 98%, or at least 99% identity to SEQ ID NO: 75, and the second portion of Cre recombinase comprises an amino acid sequence having at least 80%, at least 85%, at least 90%, at least 95%, at least 98%, or at least 99% identity to SEQ ID NO: 76. In some embodiments, the first portion of Cre recombinase comprises the amino acid sequence of SEQ ID NO: 75, and the second portion of Cre recombinase comprises the amino acid sequence of SEQ ID NO: 76. In some embodiments, the first portion of Cre recombinase comprises an amino acid sequence having at least 80%, at least 85%, at least 90%, at least 95%, at least 98%, or at least 99% identity to SEQ ID NO: 76, and the second portion of Cre recombinase comprises an amino acid sequence having at least 80%, at least 85%, at least 90%, at least 95%, at least 98%, or at least 99% identity to SEQ ID NO: 75. In some embodiments, the first portion of Cre recombinase comprises the amino acid sequence of SEQ ID NO: 76, and the second portion of Cre recombinase comprises the amino acid sequence of SEQ ID NO: 75.

In some embodiments, a split Vcre recombinase described herein comprises a first portion and a second portion of a Vcre recombinase (e.g., SEQ ID NO: 6), wherein the first portion and the second portion individually lack recombinase activity, but collectively (i.e., when bound together covalently or non-covalently) have recombinase activity. In some embodiments, the first portion of Vcre recombinase corresponds to the N-terminal portion of Vcre recombinase, and the second portion of Vcre recombinase corresponds to the C-terminal portion of Vcre recombinase. In other embodiments, the second portion of Vcre recombinase corresponds to the N-terminal portion of Vcre recombinase, and the first portion of Vcre recombinase corresponds to the C-terminal portion of Vcre recombinase.

In some embodiments, the first portion of Vcre recombinase comprises an amino acid sequence having at least 80%, at least 85%, at least 90%, at least 95%, at least 98%, or at least 99% identity to SEQ ID NO: 77, and the second portion of Vcre recombinase comprises an amino acid sequence having at least 80%, at least 85%, at least 90%, at least 95%, at least 98%, or at least 99% identity to SEQ ID NO: 78. In some embodiments, the first portion of Vcre recombinase comprises the amino acid sequence of SEQ ID NO: 77, and the second portion of Vcre recombinase comprises the amino acid sequence of SEQ ID NO: 78. In some embodiments, the first portion of Vcre recombinase comprises an amino acid sequence having at least 80%, at least 85%, at least 90%, at least 95%, at least 98%, or at least 99% identity to SEQ ID NO: 78, and the second portion of Vcre recombinase comprises an amino acid sequence having at least 80%, at least 85%, at least 90%, at least 95%, at least 98%, or at least 99% identity to SEQ ID NO: 77. In some embodiments, the first portion of Vcre recombinase comprises the amino acid sequence of SEQ ID NO: 78, and the second portion of Vcre recombinase comprises the amino acid sequence of SEQ ID NO: 77.

b. Dimerization Domains

As described above, the split recombinases described herein comprise: (i) a first polypeptide comprising a first portion of a recombinase and a first dimerization domain; and (ii) a second polypeptide comprising a second portion of the recombinase and a second dimerization domain; wherein the first polypeptide and the second polypeptide lack recombinase activity in the absence of dimerization; and wherein a recombinase dimer comprising the first polypeptide and the second polypeptide has recombinase activity.

Exemplary dimerization domain pairs (i.e., a pair consisting of a first dimerization domain and second dimerization domain) that can be fused to pairs of polypeptides to render the polypeptides capable of dimerization are known to those having ordinary skill in the art.

In some embodiments, a first dimerization domain and a second dimerization domain are capable of dimerizing in the absence of a small molecule inducer.

In some embodiments, dimerization of a first dimerization domain and a second dimerization domain is induced by the presence of a small molecule inducer. For example, in some embodiments, dimerization of a first dimerization domain and a second dimerization domain is induced by the presence of gibberellic acid (GA). In some embodiments, dimerization of a first dimerization domain and a second dimerization domain is induced by the presence of abscisic acid (ABA). In some embodiments, dimerization of a first dimerization domain and a second dimerization domain is induced by the presence of rapalog (Rap).

In some embodiments, a dimerization domain pair comprises a GID1 dimerization domain and a GAI dimerization domain. In some embodiments, the GID1 dimerization domain comprises an amino acid sequence having at least 80%, at least 85%, at least 90%, at least 95%, at least 98%, or at least 99% identity to SEQ ID NO: 79 and the GAI dimerization domain comprises an amino acid sequence having at least 80%, at least 85%, at least 90%, at least 95%, at least 98%, or at least 99% identity to SEQ ID NO: 80. In some embodiments, the GID1 dimerization domain comprises the amino acid sequence of SEQ ID NO: 79, and the GAI dimerization domain comprises the amino acid sequence of SEQ ID NO: 80.

In some embodiments, a dimerization domain pair comprises an ABI dimerization domain and a PYL dimerization domain. In some embodiments, the ABI dimerization domain comprises an amino acid sequence having at least 80%, at least 85%, at least 90%, at least 95%, at least 98%, or at least 99% identity to SEQ ID NO: 81 and the PYL dimerization domain comprises an amino acid sequence having at least 80%, at least 85%, at least 90%, at least 95%, at least 98%, or at least 99% identity to SEQ ID NO: 82. In some embodiments, the ABI dimerization domain comprises the amino acid sequence of SEQ ID NO: 81, and the PYL dimerization domain comprises the amino acid sequence of SEQ ID NO: 82.

In some embodiments, a dimerization domain pair comprises an FRB dimerization domain and a FKBP dimerization domain. In some embodiments, the FRB dimerization domain comprises an amino acid sequence having at least 80%, at least 85%, at least 90%, at least 95%, at least 98%, or at least 99% identity to SEQ ID NO: 83 and the FKBP dimerization domain comprises an amino acid sequence having at least 80%, at least 85%, at least 90%, at least 95%, at least 98%, or at least 99% identity to SEQ ID NO: 84. In some embodiments, the FRB dimerization domain comprises the amino acid sequence of SEQ ID NO: 83, and the FKBP dimerization domain comprises the amino acid sequence of SEQ ID NO: 84.

c. Exemplary Split Recombinases

In some embodiments, a split recombinase comprises a combination of features provided in Table 1.

### Exemplary Split Flp Recombinases

In some embodiments, a split Flp recombinase comprises: (i) a first polypeptide comprising a first portion of a Flp recombinase and a GID1 dimerization domain (as provided in Part Ib, above); and (ii) a second polypeptide comprising a second portion of the Flp recombinase and a GAI dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of Flp recombinase; and the GID1 dimerization domain. In some embodiments, the first polypeptide comprises, from N-terminus to C-terminus: the GID1 dimerization domain; and the first portion of Flp recombinase. In some embodiments, the second polypeptide comprises, from N-terminus to C-terminus: the second portion of Flp recombinase; and the GAI dimerization domain. In some embodiments, the second polypeptide comprises, from N-terminus to C-terminus: the GAI dimerization domain; and the second portion of Flp recombinase. In some embodiments, the first portion of Flp recombinase corresponds to the N-terminal portion of Flp recombinase, and the second portion of Flp recombinase corresponds to the C-terminal portion of Flp recombinase. In other embodiments, the first portion of Flp recombinase corresponds to the C-terminal portion of Flp recombinase, and the second portion of Flp recombinase corresponds to the N-terminal portion of Flp recombinase. In some embodiments, the N-terminal portion of Flp recombinase comprises an amino acid sequence having at least 80%, at least 85%, at least 90%, at least 95%, at least 98%, or at least 99% identity to SEQ ID NO: 41, and the C-terminal portion of Flp recombinase comprises an amino acid sequence having at least 80%, at least 85%, at least 90%, at least 95%, at least 98%, or at least 99% identity to SEQ ID NO: 42. In some embodiments, the N-terminal portion of Flp recombinase comprises the amino acid sequence of SEQ ID NO: 41, and the C-terminal portion of Flp recombinase comprises the amino acid sequence of SEQ ID NO: 42. In some embodiments, the split Flp recombinase comprises an amino acid sequence having at least 80%, at least 85%, at least 90%, at least 95%, at least 98%, or at least 99% identity to SEQ ID NO: 90. In some embodiments, the split Flp recombinase comprises the amino acid sequence of SEQ ID NO: 90.

In some embodiments, a split Flp recombinase comprises: (i) a first polypeptide comprising a first portion of a Flp recombinase and an ABI dimerization domain (as provided in Part Ib, above); and (ii) a second polypeptide comprising a second portion of a Flp recombinase and a PYL dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of Flp recombinase; and the ABI dimerization domain. In some embodiments, the first polypeptide comprises, from N-terminus to C-terminus: the ABI dimerization domain; and the first portion of Flp recombinase. In some embodiments, the second polypeptide comprises, from N-terminus to C-terminus: the second portion of Flp recombinase; and the PYL dimerization domain. In some embodiments, the second polypeptide comprises, from N-terminus to C-terminus: the PYL dimerization domain; and the second portion of Flp recombinase. In some embodiments, the first portion of Flp recombinase corresponds to the N-terminal portion of Flp recombinase, and the second portion of Flp recombinase corresponds to the C-terminal portion of Flp recombinase. In other embodiments, the first portion of Flp recombinase corresponds to the C-terminal portion of Flp recombinase, and the second portion of Flp recombinase corresponds to the N-terminal portion of Flp recombinase. In some embodiments, the N-terminal portion of Flp recombinase comprises an amino acid sequence having at least 80%, at least 85%, at least 90%, at least 95%, at least 98%, or at least 99% identity to SEQ ID NO: 43, and the C-terminal portion of Flp recombinase comprises an amino acid sequence having at least 80%, at least 85%, at least 90%, at least 95%, at least 98%, or at least 99% identity to SEQ ID NO: 44. In some embodiments, the N-terminal portion of Flp recombinase comprises the amino acid sequence of SEQ ID NO: 43, and the C-terminal portion of Flp recombinase comprises the amino acid sequence of SEQ ID NO: 44. In some embodiments, the split Flp recombinase comprises an amino acid sequence having at least 80%, at least 85%, at least 90%, at least 95%, at least 98%, or at least 99% identity to SEQ ID NO: 91. In some embodiments, the split Flp recombinase comprises the amino acid sequence of SEQ ID NO: 91.

### Exemplary Split Bxb1 Recombinases

In some embodiments, a split Bxb1 recombinase comprises: (i) a first polypeptide comprising a first portion of a Bxb1 recombinase and a GID1 dimerization domain (as provided in Part Ib, above); and (ii) a second polypeptide comprising a second portion of the Bxb1 recombinase and a GAI dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of Bxb1 recombinase; and the GID1 dimerization domain. In some embodiments, the first polypeptide comprises, from N-terminus to C-terminus: the GID1 dimerization domain; and the first portion of Bxb1 recombinase. In some embodiments, the second polypeptide comprises, from N-terminus to C-terminus: the second portion of Bxb1 recombinase; and the GAI dimerization domain. In some embodiments, the second polypeptide comprises, from N-terminus to C-terminus: the GAI dimerization domain; and the second portion of Bxb1 recombinase. In some embodiments, the first portion of Bxb1 recombinase corresponds to the N-terminal portion of Bxb1 recombinase, and the second portion of Bxb1 recombinase corresponds to the C-terminal portion of Bxb1 recombinase. In other embodiments, the first portion of Bxb1 recombinase corresponds to the C-terminal portion of Bxb1 recombinase, and the second portion of Bxb1 recombinase corresponds to the N-terminal portion of Bxb1 recombinase.

In some embodiments, the N-terminal portion of Bxb1 recombinase comprises an amino acid sequence having at least 80%, at least 85%, at least 90%, at least 95%, at least 98%, or at least 99% identity to SEQ ID NO: 45, and the C-terminal portion of Bxb1 recombinase comprises an amino acid sequence having at least 80%, at least 85%, at least 90%, at least 95%, at least 98%, or at least 99% identity to SEQ ID NO: 46. In some embodiments, the N-terminal portion of Bxb1 recombinase comprises the amino acid sequence of SEQ ID NO: 45, and the C-terminal portion of Bxb1 recombinase comprises the amino acid sequence of SEQ ID NO: 46.

In some embodiments, the first portion of Bxb1 recombinase comprises an amino acid sequence having at least 80%, at least 85%, at least 90%, at least 95%, at least 98%, or at least 99% identity to SEQ ID NO: 47, and the second portion of Bxb1 recombinase comprises an amino acid sequence having at least 80%, at least 85%, at least 90%, at least 95%, at least 98%, or at least 99% identity to SEQ ID NO: 48. In some embodiments, the N-terminal portion of Bxb1 recombinase comprises the amino acid sequence of SEQ ID NO: 47, and the C-terminal portion of Bxb1 recombinase comprises the amino acid sequence of SEQ ID NO: 48.

In some embodiments, the N-terminal portion of Bxb1 recombinase comprises an amino acid sequence having at least 80%, at least 85%, at least 90%, at least 95%, at least 98%, or at least 99% identity to SEQ ID NO: 49, and the C-terminal portion of Bxb1 recombinase comprises an amino acid sequence having at least 80%, at least 85%, at least 90%, at least 95%, at least 98%, or at least 99% identity to SEQ ID NO: 50. In some embodiments, the N-terminal portion of Bxb1 recombinase comprises the amino acid sequence of SEQ ID NO: 49, and the C-terminal portion of Bxb1 recombinase comprises the amino acid sequence of SEQ ID NO: 50.

In some embodiments, the N-terminal portion of Bxb1 recombinase comprises an amino acid sequence having at least 80%, at least 85%, at least 90%, at least 95%, at least 98%, or at least 99% identity to SEQ ID NO: 51, and the C-terminal portion of Bxb1 recombinase comprises an amino acid sequence having at least 80%, at least 85%, at least 90%, at least 95%, at least 98%, or at least 99% identity to SEQ ID NO: 52. In some embodiments, the N-terminal portion of Bxb1 recombinase comprises the amino acid sequence of SEQ ID NO: 51, and the C-terminal portion of Bxb1 recombinase comprises the amino acid sequence of SEQ ID NO: 52.

In some embodiments, the N-terminal portion of Bxb1 recombinase comprises an amino acid sequence having at least 80%, at least 85%, at least 90%, at least 95%, at least 98%, or at least 99% identity to SEQ ID NO: 53, and the C-terminal portion of Bxb1 recombinase comprises an amino acid sequence having at least 80%, at least 85%, at least 90%, at least 95%, at least 98%, or at least 99% identity to SEQ ID NO: 54. In some embodiments, the N-terminal portion of Bxb1 recombinase comprises the amino acid sequence of SEQ ID NO: 53, and the C-terminal portion of Bxb1 recombinase comprises the amino acid sequence of SEQ ID NO: 54.

In some embodiments, the N-terminal portion of Bxb1 recombinase comprises an amino acid sequence having at least 80%, at least 85%, at least 90%, at least 95%, at least 98%, or at least 99% identity to SEQ ID NO: 55, and the C-terminal portion of Bxb1 recombinase comprises an amino acid sequence having at least 80%, at least 85%, at least 90%, at least 95%, at least 98%, or at least 99% identity to SEQ ID NO: 56. In some embodiments, the N-terminal portion of Bxb1 recombinase comprises the amino acid sequence of SEQ ID NO: 55, and the C-terminal portion of Bxb1 recombinase comprises the amino acid sequence of SEQ ID NO: 56.

In some embodiments, the N-terminal portion of Bxb1 recombinase comprises an amino acid sequence having at least 80%, at least 85%, at least 90%, at least 95%, at least 98%, or at least 99% identity to SEQ ID NO: 57, and the C-terminal portion of Bxb1 recombinase comprises an amino acid sequence having at least 80%, at least 85%, at least 90%, at least 95%, at least 98%, or at least 99% identity to SEQ ID NO: 58. In some embodiments, the N-terminal portion of Bxb1 recombinase comprises the amino acid sequence of SEQ ID NO: 57, and the C-terminal portion of Bxb1 recombinase comprises the amino acid sequence of SEQ ID NO: 58.

In some embodiments, the N-terminal portion of Bxb1 recombinase comprises an amino acid sequence having at least 80%, at least 85%, at least 90%, at least 95%, at least 98%, or at least 99% identity to SEQ ID NO: 59, and the C-terminal portion of Bxb1 recombinase comprises an amino acid sequence having at least 80%, at least 85%, at least 90%, at least 95%, at least 98%, or at least 99% identity to SEQ ID NO: 60. In some embodiments, the N-terminal portion of Bxb1 recombinase comprises the amino acid sequence of SEQ ID NO: 59, and the C-terminal portion of Bxb1 recombinase comprises the amino acid sequence of SEQ ID NO: 60.

In some embodiments, the N-terminal portion of Bxb1 recombinase comprises an amino acid sequence having at least 80%, at least 85%, at least 90%, at least 95%, at least 98%, or at least 99% identity to SEQ ID NO: 61, and the C-terminal portion of Bxb1 recombinase comprises an amino acid sequence having at least 80%, at least 85%, at least 90%, at least 95%, at least 98%, or at least 99% identity to SEQ ID NO: 62. In some embodiments, the N-terminal portion of Bxb1 recombinase comprises the amino acid sequence of SEQ ID NO: 61, and the C-terminal portion of Bxb1 recombinase comprises the amino acid sequence of SEQ ID NO: 62.

In some embodiments, the N-terminal portion of Bxb1 recombinase comprises an amino acid sequence having at least 80%, at least 85%, at least 90%, at least 95%, at least 98%, or at least 99% identity to SEQ ID NO: 63, and the C-terminal portion of Bxb1 recombinase comprises an amino acid sequence having at least 80%, at least 85%, at least 90%, at least 95%, at least 98%, or at least 99% identity to SEQ ID NO: 64. In some embodiments, the N-terminal portion of Bxb1 recombinase comprises the amino acid sequence of SEQ ID NO: 63, and the C-terminal portion of Bxb1 recombinase comprises the amino acid sequence of SEQ ID NO: 64.

In some embodiments, the N-terminal portion of Bxb1 recombinase comprises an amino acid sequence having at least 80%, at least 85%, at least 90%, at least 95%, at least 98%, or at least 99% identity to SEQ ID NO: 65, and the C-terminal portion of Bxb1 recombinase comprises an amino acid sequence having at least 80%, at least 85%, at least 90%, at least 95%, at least 98%, or at least 99% identity to SEQ ID NO: 66. In some embodiments, the N-terminal portion of Bxb1 recombinase comprises the amino acid sequence of SEQ ID NO: 65, and the C-terminal portion of Bxb1 recombinase comprises the amino acid sequence of SEQ ID NO: 66.

In some embodiments, the split Bxb1 recombinase comprises an amino acid sequence having at least 80%, at least 85%, at least 90%, at least 95%, at least 98%, or at least 99% identity to any one of SEQ ID NOs: 92-102. In some embodiments, the split Bxb1 recombinase comprises the amino acid sequence of any one of SEQ ID NOs: 92-102.

### Exemplary Split PhiC31 Recombinases

In some embodiments, a split PhiC31 recombinase comprises: (i) a first polypeptide comprising a first portion of a PhiC31 recombinase and a GID1 dimerization domain (as provided in Part Ib, above); and (ii) a second polypeptide comprising a second portion of the PhiC31 recombinase and a GAI dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of PhiC31 recombinase; and the GID1 dimerization domain. In some embodiments, the first polypeptide comprises, from N-terminus to C-terminus: the GID1 dimerization domain; and the first portion of PhiC31 recombinase. In some embodiments, the second polypeptide comprises, from N-terminus to C-terminus: the second portion of PhiC31 recombinase; and the GAI dimerization domain. In some embodiments, the second polypeptide comprises, from N-terminus to C-terminus: the GAI dimerization domain; and the second portion of PhiC31 recombinase. In some embodiments, the first portion of PhiC31 recombinase corresponds to the N-terminal portion of PhiC31 recombinase, and the second portion of PhiC31 recombinase corresponds to the C-terminal portion of PhiC31 recombinase. In other embodiments, the first portion of PhiC31 recombinase corresponds to the C-terminal portion of PhiC31 recombinase, and the second portion of PhiC31 recombinase corresponds to the N-terminal portion of PhiC31 recombinase. In some embodiments, the N-terminal portion of PhiC31 recombinase comprises an amino acid sequence having at least 80%, at least 85%, at least 90%, at least 95%, at least 98%, or at least 99% identity to SEQ ID NO: 67, and the C-terminal portion of PhiC31 recombinase comprises an amino acid sequence having at least 80%, at least 85%, at least 90%, at least 95%, at least 98%, or at least 99% identity to SEQ ID NO: 68. In some embodiments, the N-terminal portion of PhiC31 recombinase comprises the amino acid sequence of SEQ ID NO: 67, and the C-terminal portion of PhiC31 recombinase comprises the amino acid sequence of SEQ ID NO: 68. In some embodiments, the split PhiC31 recombinase comprises an amino acid sequence having at least 80%, at least 85%, at least 90%, at least 95%, at least 98%, or at least 99% identity to SEQ ID NO: 104. In some embodiments, the split PhiC31 recombinase comprises the amino acid sequence of SEQ ID NO: 104.

In some embodiments, a split PhiC31 recombinase comprises: (i) a first polypeptide comprising a first portion of a PhiC31 recombinase and a FRB dimerization domain (as provided in Part Ib, above); and (ii) a second polypeptide comprising a second portion of a PhiC31 recombinase and a FKBP dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of PhiC31 recombinase; and the FRB dimerization domain. In some embodiments, the first polypeptide comprises, from N-terminus to C-terminus: the FRB dimerization domain; and the first portion of PhiC31 recombinase. In some embodiments, the second polypeptide comprises, from N-terminus to C-terminus: the second portion of PhiC31 recombinase; and the FKBP dimerization domain. In some embodiments, the second polypeptide comprises, from N-terminus to C-terminus: the FKBP dimerization domain; and the second portion of PhiC31 recombinase. In some embodiments, the first portion of PhiC31 recombinase corresponds to the N-terminal portion of PhiC31 recombinase, and the second portion of PhiC31 recombinase corresponds to the C-terminal portion of PhiC31 recombinase. In other embodiments, the first portion of PhiC31 recombinase corresponds to the C-terminal portion of PhiC31 recombinase, and the second portion of PhiC31 recombinase corresponds to the N-terminal portion of PhiC31 recombinase. In some embodiments, the N-terminal portion of PhiC31 recombinase comprises an amino acid sequence having at least 80%, at least 85%, at least 90%, at least 95%, at least 98%, or at least 99% identity to SEQ ID NO: 69, and the C-terminal portion of PhiC31 recombinase comprises an amino acid sequence having at least 80%, at least 85%, at least 90%, at least 95%, at least 98%, or at least 99% identity to SEQ ID NO: 70. In some embodiments, the N-terminal portion of PhiC31 recombinase comprises the amino acid sequence of SEQ ID NO: 69, and the C-terminal portion of PhiC31 recombinase comprises the amino acid sequence of SEQ ID NO: 70. In some embodiments, the split PhiC31 recombinase comprises an amino acid sequence having at least 80%, at least 85%, at least 90%, at least 95%, at least 98%, or at least 99% identity to SEQ ID NO: 105. In some embodiments, the split PhiC31 recombinase comprises the amino acid sequence of SEQ ID NO: 105.

### Exemplary Split TP901 Recombinases

In some embodiments, a split TP901 recombinase comprises: (i) a first polypeptide comprising a first portion of a TP901 recombinase and a GID1 dimerization domain (as provided in Part Ib, above); and (ii) a second polypeptide comprising a second portion of the TP901 recombinase and a GAI dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of TP901 recombinase; and the GID1 dimerization domain. In some embodiments, the first polypeptide comprises, from N-terminus to C-terminus: the GID1 dimerization domain; and the first portion of TP901 recombinase. In some embodiments, the second polypeptide comprises, from N-terminus to C-terminus: the second portion of TP901 recombinase; and the GAI dimerization domain. In some embodiments, the second polypeptide comprises, from N-terminus to C-terminus: the GAI dimerization domain; and the second portion of TP901 recombinase. In some embodiments, the first portion of TP901 recombinase corresponds to the N-terminal portion of TP901 recombinase, and the second portion of TP901 recombinase corresponds to the C-terminal portion of TP901 recombinase. In other embodiments, the first portion of TP901 recombinase corresponds to the C-terminal portion of TP901 recombinase, and the second portion of TP901 recombinase corresponds to the N-terminal portion of TP901 recombinase.

In some embodiments, the N-terminal portion of TP901 recombinase comprises an amino acid sequence having at least 80%, at least 85%, at least 90%, at least 95%, at least 98%, or at least 99% identity to SEQ ID NO: 71, and the C-terminal portion of TP901 recombinase comprises an amino acid sequence having at least 80%, at least 85%, at least 90%, at least 95%, at least 98%, or at least 99% identity to SEQ ID NO: 72. In some embodiments, the N-terminal portion of TP901 recombinase comprises the amino acid sequence of SEQ ID NO: 71, and the C-terminal portion of TP901 recombinase comprises the amino acid sequence of SEQ ID NO: 72. In some embodiments, the split TP901 recombinase comprises an amino acid sequence having at least 80%, at least 85%, at least 90%, at least 95%, at least 98%, or at least 99% identity to SEQ ID NO: 106. In some embodiments, the split TP901 recombinase comprises the amino acid sequence of SEQ ID NO: 106.

### Exemplary Split Cre Recombinases

In some embodiments, a split Cre recombinase comprises: (i) a first polypeptide comprising a first portion of a Cre recombinase and a ABI dimerization domain (as provided in Part Ib, above); and (ii) a second polypeptide comprising a second portion of the Cre recombinase and a PYL dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of Cre recombinase; and the ABI dimerization domain. In some embodiments, the first polypeptide comprises, from N-terminus to C-terminus: the ABI dimerization domain; and the first portion of Cre recombinase. In some embodiments, the second polypeptide comprises, from N-terminus to C-terminus: the second portion of Cre recombinase; and the PYL dimerization domain. In some embodiments, the second polypeptide comprises, from N-terminus to C-terminus: the PYL dimerization domain; and the second portion of Cre recombinase. In some embodiments, the first portion of Cre recombinase corresponds to the N-terminal portion of Cre recombinase, and the second portion of Cre recombinase corresponds to the C-terminal portion of Cre recombinase. In other embodiments, the first portion of Cre recombinase corresponds to the C-terminal portion of Cre recombinase, and the second portion of Cre recombinase corresponds to the N-terminal portion of Cre recombinase.

In some embodiments, the N-terminal portion of Cre recombinase comprises an amino acid sequence having at least 80%, at least 85%, at least 90%, at least 95%, at least 98%, or at least 99% identity to SEQ ID NO: 73, and the C-terminal portion of Cre recombinase comprises an amino acid sequence having at least 80%, at least 85%, at least 90%, at least 95%, at least 98%, or at least 99% identity to SEQ ID NO: 74. In some embodiments, the N-terminal portion of Cre recombinase comprises the amino acid sequence of SEQ ID NO: 73, and the C-terminal portion of Cre recombinase comprises the amino acid sequence of SEQ ID NO: 74.

In some embodiments, the N-terminal portion of Cre recombinase comprises an amino acid sequence having at least 80%, at least 85%, at least 90%, at least 95%, at least 98%, or at least 99% identity to SEQ ID NO: 75, and the C-terminal portion of Cre recombinase comprises an amino acid sequence having at least 80%, at least 85%, at least 90%, at least 95%, at least 98%, or at least 99% identity to SEQ ID NO: 76. In some embodiments, the N-terminal portion of Cre recombinase comprises the amino acid sequence of SEQ ID NO: 75, and the C-terminal portion of Cre recombinase comprises the amino acid sequence of SEQ ID NO: 76.

In some embodiments, the split Cre recombinase comprises an amino acid sequence having at least 80%, at least 85%, at least 90%, at least 95%, at least 98%, or at least 99% identity to any one of SEQ ID NOs: 107-109. In some embodiments, the split Cre recombinase comprises the amino acid sequence of any one of SEQ ID NOs: 107-109.

### Exemplary Split Vcre Recombinases

In some embodiments, a split Vcre recombinase comprises: (i) a first polypeptide comprising a first portion of a Vcre recombinase and a GID1 dimerization domain (as provided in Part Ib, above); and (ii) a second polypeptide comprising a second portion of the Vcre recombinase and a GAI dimerization domain (as provided in Part Ib, above); wherein the first polypeptide and the second polypeptide lack recombinase activity in the absence of dimerization; and wherein a recombinase dimer comprising the first polypeptide and the second polypeptide has recombinase activity. In some embodiments, the first polypeptide comprises, from N-terminus to C-terminus: the first portion of Vcre recombinase; and the GID1 dimerization domain. In some embodiments, the first polypeptide comprises, from N-terminus to C-terminus: the GID1 dimerization domain; and the first portion of VCre recombinase. In some embodiments, the second polypeptide comprises, from N-terminus to C-terminus: the second portion of Vcre recombinase; and the GAI dimerization domain. In some embodiments, the second polypeptide comprises, from N-terminus to C-terminus: the GAI dimerization domain; and the second portion of Vcre recombinase. In some embodiments, the first portion of Vcre recombinase corresponds to the N-terminal portion of Vcre recombinase, and the second portion of Vcre recombinase corresponds to the C-terminal portion of Vcre recombinase. In other embodiments, the first portion of Vcre recombinase corresponds to the C-terminal portion of Vcre recombinase, and the second portion of Vcre recombinase corresponds to the N-terminal portion of Vcre recombinase. In some embodiments, the N-terminal portion of Vcre recombinase comprises an amino acid sequence having at least 80%, at least 85%, at least 90%, at least 95%, at least 98%, or at least 99% identity to SEQ ID NO: 77, and the C-terminal portion of Vcre recombinase comprises an amino acid sequence having at least 80%, at least 85%, at least 90%, at least 95%, at least 98%, or at least 99% identity to SEQ ID NO: 78. In some embodiments, the N-terminal portion of Vcre recombinase comprises the amino acid sequence of SEQ ID NO: 77, and the C-terminal portion of Vcre recombinase comprises the amino acid sequence of SEQ ID NO: 78. In some embodiments, the split Vcre recombinase comprises an amino acid sequence having at least 80%, at least 85%, at least 90%, at least 95%, at least 98%, or at least 99% identity to SEQ ID NO: 110. In some embodiments, the split Vcre recombinase comprises the amino acid sequence of SEQ ID NO: 110.

d. Percent Identity

As used herein, the term “percent identity” (or “% identity”) refers to a relationship between the sequences of two polypeptides or polynucleotides, as determined by sequence comparison (alignment). In some embodiments, identity is determined across the entire length of a sequence. In some embodiments, identity is determined over a region of a sequence. Identity of related polypeptides or nucleic acid sequences can be readily calculated by those having ordinary skill in the art. For example, the percent identity of two sequences (e.g., nucleic acid or amino acid sequences) may be determined using BLAST®, NBLAST®, XBLAST®, Gapped BLAST®, and Clustal Omega programs, using default parameters of the respective programs. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotides and dividing by the length of one of the nucleic acids.

### II. Polynucleic Acid Molecules Encoding a Split Recombinase

In some aspects, the disclosure relates to polynucleic acid molecules (or combinations of polynucleic acid molecules) encoding a split recombinase described herein. In some embodiments, a split recombinase is encoded by a single expression cassette. In other embodiments, a split recombinase is encoded by two expression cassettes.

As used herein, the term “expression cassette” refers to a nucleic acid sequence encoding a gene product (i.e., a mRNA or polypeptide) operably linked to a promoter.

As used herein, the term “promoter” refers to a nucleic acid sequence that is bound by proteins to initiate transcription of RNA from DNA. A promoter may be a constitutive promoter (i.e., an unregulated promoter that allows for continual transcription). Examples of constitutive promoters are known in the art and include, but are not limited to, cytomegalovirus (CMV) promoters, elongation factor 1α (EF1α) promoters, simian vacuolating virus 40 (SV40) promoters, ubiquitin-C(UBC) promoters, U6 promoters, p5 promoters, p19 promoters, p40 promoters, E2A promoters, E4 promoters and phosphoglycerate kinase (PGK) promoters. See e.g., Ferreira et al. Proc. Natl. Acad. Sci. U.S.A. 2013 July; 110(28): 11284-89; Pub. No.: US 2014/377861 A1; Qin et al. PloS one 5.5 (2010): e10611.—the entireties of which are incorporated herein by reference. Alternatively, a promoter may be an inducible promoter (i.e., activates transcription under specific circumstances). An inducible promoter may be a chemically inducible promoter, a temperature inducible promoter, or a light inducible promoter. Additional types of inducible promoters are known to those having ordinary skill in the art. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, pTRE3G promoters, pTREtight promoters, the Gal4 UAS operator sequences and mifepristone inducible promoters, and a promoters containing at least one of VanR, TtgR, PhlF, or CymR operator sequences. See e.g., Stanton et al., ACS Synth. Biol. 2014 Dec. 19; 3(12): 880-91; Liang et al., Sci. Signal. 2011 Mar. 15; 4(164): rs2; U.S. Pat. No. 7,745,592 B2; U.S. Pat. No. 7,935,788 B2—the entireties of which are incorporated herein by reference.

In some embodiments, expression of the first polypeptide and/or the second polypeptide of a split-recombinase is under the control of a constitutive promoter. In some embodiments, expression of the first polypeptide and/or the second polypeptide of a split-recombinase is under the control of an inducible promoter.

a. Single Expression Cassette Embodiments

In some embodiments, a polynucleic acid described herein comprises an expression cassette encoding for a polycistronic mRNA, wherein the polycistronic mRNA comprises: (i) a sequence encoding for a first polypeptide of a split recombinase (e.g., as described above); (ii) a sequence encoding for an intercistronic region; and (iii) a sequence encoding for a second polypeptide of a split recombinase (e.g., as described above); wherein the sequence encoding for the intercistronic region is flanked on one end by the sequence encoding for the first polypeptide and on the other end by the sequence encoding for the second polypeptide.

In some embodiments, the intercistronic region comprises a nucleic acid sequence encoding an internal ribosomal entry site (IRES). Various IRES sequences have been described previously and are known to those having ordinary skill in the art. In some embodiments, an IRES comprises a nucleic acid sequence having at least 80%, at least 85%, at least 90%, at least 95%, at least 98%, or at least 99% identity to any one of SEQ ID NOs: 85-87. In some embodiments, an IRES comprises the nucleic acid sequence of any one of SEQ ID NOs: 85-87.

In some embodiments, the intercistronic region comprises a nucleic acid sequence encoding a 2A peptide. Various 2A peptides have been described previously and are known to those having ordinary skill in the art. In some embodiments, a 2A peptide comprises an amino acid sequence having at least 80%, at least 85%, at least 90%, at least 95%, at least 98%, or at least 99% identity to any one of SEQ ID NOs: 88-89 and 236-237. In some embodiments, a 2A peptide comprises an amino acid sequence of any one of SEQ ID NOs: 88-89 and 236-237.

In some embodiments, a polynucleic acid molecule encoding a split recombinase (or polypeptide dimer having recombinase activity) comprises, from 5′ to 3′: (i) a sequence encoding for a first polypeptide comprising a first portion of a recombinase and a first dimerization domain; (ii) a sequence encoding for a viral 2A peptide and/or an internal ribosomal entry site (IRES); and (iii) a sequence encoding for a second polypeptide comprising a second portion of a recombinase and a second dimerization domain; wherein the first polypeptide and the second polypeptide lack recombinase activity in the absence of dimerization; and wherein a recombinase dimer comprising the first polypeptide and the second polypeptide has recombinase activity.

In some embodiments, the first dimerization domain of the first polypeptide is N-terminal to the first portion of the recombinase. In some embodiments, the first dimerization domain is C-terminal to the first portion of the recombinase. In some embodiments, the second dimerization domain of the second polypeptide is N-terminal to the second portion of the recombinase. In some embodiments, the second dimerization domain is C-terminal to the second portion of the recombinase.

In some embodiments, the first portion of the recombinase corresponds to the N-terminal portion of the recombinase, and the second portion of the recombinase corresponds to the C-terminal portion of the recombinase. In other embodiments, the first portion of the recombinase corresponds to the C-terminal portion of the recombinase, and the second portion of the recombinase corresponds to the N-terminal portion of the recombinase.

In some embodiments, a polynucleic acid molecule comprises a nucleic acid sequence encoding a split recombinase, wherein the nucleic acid sequence has at least 80%, at least 85%, at least 90%, at least 95%, at least 98%, or at least 99% identity to any one of SEQ ID NOs: 111-131. In some embodiments, a polynucleic acid molecule comprises a nucleic acid sequence encoding a split recombinase, wherein the nucleic acid sequence comprises the nucleic acid sequence of any one of SEQ ID NOs: 111-131.

In some embodiments, a polynucleic acid molecule comprises an expression cassette encoding a polycistronic mRNA, wherein the polycistronic mRNA comprises a nucleic acid sequence having at least 80%, at least 85%, at least 90%, at least 95%, at least 98%, or at least 99% identity to any one of SEQ ID NOs: 132-143. In some embodiments, a polynucleic acid molecule comprises an expression cassette encoding a polycistronic mRNA, wherein the polycistronic mRNA comprises a nucleic acid sequence of any one of SEQ ID NOs: 132-143.

b. Two Expression Cassette Embodiments

In other embodiments, a first expression cassette encodes the first polypeptide of a split recombinase, and a second expression cassette encodes the second polypeptide of a split recombinase.

In some embodiments, the first expression cassette comprises a constitutive promoter (as described herein). In some embodiments, the first expression cassette comprises an inducible promoter (as described herein).

In some embodiments, the second expression cassette comprises a constitutive promoter (as described herein). In some embodiments, the second expression cassette comprises an inducible promoter (as described herein).

In some embodiments, a single polynucleic acid comprises the first expression cassette and the second expression cassette. In other embodiments, a first polynucleic acid molecule comprises the first expression cassette, and a second polynucleic acid molecule comprises the second expression cassette.

### III. Adeno-Associated Virus Production Systems

In some aspects, the disclosure relates to adeno-associated virus (AAV) production systems which allow for inducible control of a gene product(s) required for AAV production, including an AAV gene product(s) that is cytotoxic or cytostatic to a cell. In the AAV production systems described herein, this inducible control is mediated by a split-recombinase (e.g., as provided herein). The possibility for near-zero background expression in the absence of dimerization and near-native expression in the presence of dimerization make split recombinases a promising technology for viral platforms which have complex and poorly characterized regulation. In contrast, systems that directly regulate viral genes with synthetic promoters (e.g., Tet-On or cumate) require significant tuning and may result in leaky expression in the off state.

The AAV production systems described herein comprise one or more polynucleic acid molecules comprising: (a) an AAV production component comprising a polynucleic acid molecule encoding an AAV gene product (or a portion thereof) flanked on one end by a first recombinase attachment site and on the other end by a second recombinase attachment site; and (b) a split recombinase (as described herein) corresponding to the first recombinase attachment site and the second recombination attachment site of (a). The one or more polynucleic acid molecules of an AAV production system may further comprise: (c) a transcriptional activator; (d) a transfer polynucleic acid molecule; I a selection marker; or (f) a combination thereof.

a. AAV Production Component

The AAV production systems described herein have an AAV production component comprising an AAV production component comprising a polynucleic acid molecule encoding an AAV gene product (or a portion thereof) flanked on one end by a first recombinase attachment site and on the other end by a second recombinase attachment site. AAV gene products required for generation of an AAV in a recombinant host cell (or an “engineered cell” as described herein) are known to those having ordinary skill in the art. Exemplary AAV gene products include Rep52, Rep40, Rep78, Rep68, E1, E2A, E4Orf6, VARNA, CAP (VP1, VP2, VP3), AAP, and MAAP or functional variants thereof. The Rep gene products (comprising Rep52, Rep40, Rep78 and Rep68) are involved in AAV genome replication and packaging. The E1 genes upregulate transcription of several adenovirus and AAV genes. The E2A gene product is involved in aiding DNA synthesis processivity during AAV replication. The E4Orf6 gene product supports AAV replication. The VARNA gene product plays a role in regulating translation. The CAP gene products (comprising VP1, VP2, VP3) encode viral capsid proteins. The AAP gene product plays a role in capsid assembly. MAAP is a protein residing in an alternate reading from of VP1 and appears to play a role in the viral capsid as described in Ogden et al. Science 366.6469 (2019): 1139-1143, which is incorporated by reference in its entirety.

In some embodiments, an AAV production component comprises a nucleic acid sequence encoding Rep52 (or a portion thereof), wherein the nucleic acid sequence encoding for Rep52 (or a portion thereof), is flanked on one end by a first recombinase attachment site and on the other end by a second recombinase attachment site.

In some embodiments, an AAV production component comprises a nucleic acid sequence encoding Rep40 (or a portion thereof), wherein the nucleic acid sequence encoding for Rep40 (or a portion thereof), is flanked on each end by a recombinase attachment site.

In some embodiments, an AAV production component comprises a nucleic acid sequence encoding Rep78 (or a portion thereof), wherein the nucleic acid sequence encoding for Rep78 (or a portion thereof), is flanked on one end by a first recombinase attachment site and on the other end by a second recombinase attachment site.

In some embodiments, an AAV production component comprises a nucleic acid sequence encoding Rep68 (or a portion thereof), wherein the nucleic acid sequence encoding for Rep68 (or a portion thereof), is flanked on one end by a first recombinase attachment site and on the other end by a second recombinase attachment site.

In some embodiments, an AAV production component comprises a nucleic acid sequence encoding E2A (or a portion thereof), wherein the nucleic acid sequence encoding for E2A (or a portion thereof), is flanked on one end by a first recombinase attachment site and on the other end by a second recombinase attachment site.

In some embodiments, an AAV production component comprises a nucleic acid sequence encoding E40RF6 (or a portion thereof), wherein the nucleic acid sequence encoding for E40RF6 (or a portion thereof), is flanked on one end by a first recombinase attachment site and on the other end by a second recombinase attachment site.

In some embodiments, an AAV production component comprises a nucleic acid sequence encoding VARNA (or a portion thereof), wherein the nucleic acid sequence encoding for VARNA (or a portion thereof), is flanked on one end by a first recombinase attachment site and on the other end by a second recombinase attachment site.

In some embodiments, an AAV production component comprises a nucleic acid sequence encoding VP1 (or a portion thereof), wherein the nucleic acid sequence encoding for VP1 (or a portion thereof), is flanked on one end by a first recombinase attachment site and on the other end by a second recombinase attachment site.

In some embodiments, an AAV production component comprises a nucleic acid sequence encoding VP2 (or a portion thereof), wherein the nucleic acid sequence encoding for VP2 (or a portion thereof), is flanked on one end by a first recombinase attachment site and on the other end by a second recombinase attachment site.

In some embodiments, an AAV production component comprises a nucleic acid sequence encoding VP3 (or a portion thereof), wherein the nucleic acid sequence encoding for VP3 (or a portion thereof), is flanked on one end by a first recombinase attachment site and on the other end by a second recombinase attachment site.

In some embodiments, an AAV production component comprises a nucleic acid sequence encoding AAP (or a portion thereof), wherein the nucleic acid sequence encoding for AAP (or a portion thereof), is flanked on one end by a first recombinase attachment site and on the other end by a second recombinase attachment site.

In some embodiments, an AAV production component comprises a nucleic acid sequence encoding MAAP (or a portion thereof), wherein the nucleic acid sequence encoding for MAAP (or a portion thereof), is flanked on one end by a first recombinase attachment site and on the other end by a second recombinase attachment site.

In some embodiments, an AAV production component is (i.e., the gene products of the AAV component are) encoded on a single nucleic acid molecule. In other embodiments, multiple nucleic acid molecules collectively comprise the AAV production component (i.e., at least two of the gene products of the AAV production component are encoded on different nucleic acid molecules). For example, an AAV production component may comprise at least 2, at least 3, at least 4, at least 5, at least 6, at least 7, at least 8, at least 10, or at least 11 nucleic acid molecules. In some embodiments, an AAV production component comprises 2, 3, 4, 5, 6, 7, 8, 9, 10, or 11 nucleic acid molecules.

In some embodiments, an AAV production system comprises one or more nucleic acid sequences that collectively encode the gene products: Rep52 or Rep40; Rep78 or Rep68; E2A; E4Orf6; VARNA; VP1; VP2; VP3; AAP; and MAAP. In some embodiments, an AAV production system comprises one or more nucleic acid sequences that collectively encode the gene products: Rep52, Rep40, Rep78, Rep68, E2A, E4Orf6, VARNA, VP1, VP2, VP3, and AAP. In some embodiments, the one or more nucleic acid molecules that collectively encode the gene products required for generation of an AAV are each operably linked to a promoter as described herein.

Recombinase attachment sites have been described previously and are known to those having ordinary skill in the art. Exemplary recombinase attachment sites and their corresponding recombinases are provided in Table 10.

In some embodiments, an AAV production system comprise: (i) a nucleic acid sequence encoding for a split Flp recombinase (as described herein); and (ii) a nucleic acid sequence encoding for an AAV gene product (or a portion thereof), wherein the nucleic acid sequence encoding for the AAV gene product (or a portion thereof) is flanked on one end by a first Flp recombinase attachment site and on the other end by a second Flp recombinase attachment site, wherein the first Flp recombinase attachment site and the second Flp recombinase attachment are capable of being bound and recombined by the split Flp recombinase of (i).

In some embodiments, the first Flp recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 144-156. In some embodiments, the first Flp recombinase attachment site comprises the nucleic acid sequence of any one of SEQ ID NOs: 144-156.

In some embodiments, the second Flp recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 144-156. In some embodiments, the second Flp recombinase attachment site comprises the nucleic acid sequence of any one of SEQ ID NOs: 144-156.

In some embodiments, an AAV production system comprise: (i) a nucleic acid sequence encoding for a split Bxb1 recombinase (as described herein); and (ii) a nucleic acid sequence encoding for an AAV gene product (or a portion thereof), wherein the nucleic acid sequence encoding for the AAV gene product (or a portion thereof) is flanked on one end by a first Bxb1 recombinase attachment site and on the other end by a second Bxb1 recombinase attachment site, wherein the first Bxb1 recombinase attachment site and the second Bxb1 recombinase attachment are capable of being bound and recombined by the split Bxb1 recombinase of (i).

In some embodiments, the first Bxb1 recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 157-172. In some embodiments, the first Bxb1 recombinase attachment site comprises the nucleic acid sequence of any one of SEQ ID NOs: 157-172.

In some embodiments, the second Bxb1 recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 173-188. In some embodiments, the second Bxb1 recombinase attachment site comprises the nucleic acid sequence of any one of SEQ ID NOs: 173-188.

In some embodiments, an AAV production system comprise: (i) a nucleic acid sequence encoding for a split PhiC31 recombinase (as described herein); and (ii) a nucleic acid sequence encoding for an AAV gene product (or a portion thereof), wherein the nucleic acid sequence encoding for the AAV gene product (or a portion thereof) is flanked on one end by a first PhiC31 recombinase attachment site and on the other end by a second PhiC31 recombinase attachment site, wherein the first PhiC31 recombinase attachment site and the second PhiC31 recombinase attachment are capable of being bound and recombined by the split PhiC31 recombinase of (i).

In some embodiments, the first PhiC31 recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 189-204. In some embodiments, the first PhiC31 recombinase attachment site comprises the nucleic acid sequence of any one of SEQ ID NOs: 189-204.

In some embodiments, the second PhiC31 recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 205-220. In some embodiments, the second PhiC31 recombinase attachment site comprises the nucleic acid sequence of any one of SEQ ID NOs: 205-220.

In some embodiments, an AAV production system comprise: (i) a nucleic acid sequence encoding for a split Cre recombinase (as described herein); and (ii) a nucleic acid sequence encoding for an AAV gene product (or a portion thereof), wherein the nucleic acid sequence encoding for the AAV gene product (or a portion thereof) is flanked on one end by a first Cre recombinase attachment site and on the other end by a second Cre recombinase attachment site, wherein the first Cre recombinase attachment site and the second Cre recombinase attachment are capable of being bound and recombined by the split Cre recombinase of (i).

In some embodiments, the first Cre recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 221-229. In some embodiments, the first Cre recombinase attachment site comprises the nucleic acid sequence of any one of SEQ ID NOs: 221-229.

In some embodiments, the second Cre recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 221-229. In some embodiments, the second Cre recombinase attachment site comprises the nucleic acid sequence of any one of SEQ ID NOs: 221-229.

In some embodiments, an AAV production system comprise: (i) a nucleic acid sequence encoding for a split Vcre recombinase (as described herein); and (ii) a nucleic acid sequence encoding for an AAV gene product (or a portion thereof), wherein the nucleic acid sequence encoding for the AAV gene product (or a portion thereof) is flanked on one end by a first Vcre recombinase attachment site and on the other end by a second Vcre recombinase attachment site, wherein the first Vcre recombinase attachment site and the second Vcre recombinase attachment are capable of being bound and recombined by the split Vcre recombinase of (i).

In some embodiments, the first Vcre recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 230-235. In some embodiments, the first Vcre recombinase attachment site comprises the nucleic acid sequence of any one of SEQ ID NOs: 230-235.

In some embodiments, the second Vcre recombinase attachment site comprises a nucleic acid sequence having at least 80% (e.g., at least 80%, at least 85%, at least 90%, at least 95%, or at least 99%) identity to the nucleic acid sequence of any one of SEQ ID NOs: 230-235. In some embodiments, the second Vcre recombinase attachment site comprises the nucleic acid sequence of any one of SEQ ID NOs: 230-235.

b. Transcriptional Activator

In some embodiments, an AAV production system further comprises a transcriptional activator (or a polynucleic acid molecule encoding the same). As used herein, the term “transcriptional activator” refers to a transcription factor that binds to and regulates expression of an inducible promoter of an AAV production system (e.g., an inducible promoter operably linked to a nucleic acid sequence encoding for an AAV gene product, an inducible promoter operably linked to a nucleic acid encoding for a polypeptide of a split recombinase, etc.). Exemplary transcriptional activators, and their corresponding promoter recognition sites, are known to those having skill in the art and include, but are not limited to, TetOn-3G, TetOn-V16, TetOff-Advanced, VanR-VP16, TtgR-VP16, PhlF-VP16, and the cumate cTA and rcTA. In some embodiments, the transcriptional activator is operably linked to a promoter (as described herein). In some embodiments, the transcriptional activator binds to its corresponding promoter recognition site when exposed to a small molecule inducer. In some embodiments, the small molecule inducer is selected from the group consisting of doxycycline, vanillate, phloretin, rapamycin, abscisic acid, gibberellic acid, acetoxymethyl ester, and cumate.

In some embodiments, an AAV production system comprises two or more transcriptional activators (or polynucleic acid molecules encoding the same).

c. Transfer Polynucleic Acid Molecule

In some embodiments, an AAV production system further comprises a transfer polynucleic acid molecule. In some embodiments, a transfer polynucleic acid molecule comprises, from 5′ to 3′: (i) a nucleic acid sequence of a 5′ inverted tandem repeat; (ii) a central nucleic acid; and (iii) a nucleic acid sequence of a 3′ inverted tandem repeat. In some embodiments, the nucleic acid sequence is a plasmid or a vector.

In some embodiments, a central nucleic acid of the transfer polynucleic acid molecule comprises a multiple cloning site. Exemplary multiple cloning sites are known to those having ordinary skill in the art. A multiple cloning site can be used for cloning a payload molecule (or gene of interest)—or an expression cassette encoding a payload molecule—into the transfer nucleic acid molecule prior to the generation of viral vectors in a host cell.

In some embodiments, a central nucleic acid of the transfer polynucleic acid molecule comprises a gene product of interest.

d. Selection Marker

An AAV production system may further comprise a nucleic acid sequence encoding for a selection marker. As used herein, the term “selection marker” refers to a protein that—when introduced into or expressed in a cell—confers a trait that is suitable for selection. As used herein, the term “selection cassette” refers to a nucleic acid sequence encoding a selection marker operably linked to a promoter (as described herein) and a terminator.

A selection marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selection marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

Alternatively, or in addition, a selection marker may be an auxotrophic selection marker (e.g., glutamine synthetase).

### IV. Engineered Cells

In some aspects, the disclosure relates to engineered cells comprising a split recombinase described herein or a polynucleic acid molecule encoding a split recombinase described herein (optionally wherein the polynucleic acid molecule is stably integrated).

In some aspects, the disclosure relates to engineered cells for AAV production comprising an AAV production described herein. In some embodiments, the engineered cell may comprise any part (and any combination of parts) of the AAV production systems described herein. An engineered cell may comprise at least a portion of the AAV production component (e.g., one or more nucleic acid sequences encoding Rep52, Rep40, Rep78, Rep68, E2A, E4Orf6, VARNA, VP1, VP2, VP3, and/or AAP). For example, and as described above, an AAV production component may comprise multiple nucleic acid molecules. In such embodiments, an engineered cell comprises one or more of said multiple polynucleic acid molecules—each of which may be located extra-chromosomally or stably integrated into the genome of the engineered cell. In some embodiments, an engineered cell comprises the entire AAV production component. In some embodiments, an engineered cell further comprises one or more polynucleic acid molecules collectively comprising nucleic acid sequences encoding for: UL5, UL8, UL29, UL30, UL42, UL52, UL12, ICP10, ICP4, and ICP22 (optionally wherein one or more of the polynucleic acid molecules is stably integrated).

In some aspects, the disclosure relates to engineered cells comprising: (a) a first polynucleic acid molecule (optionally stably integrated) encoding a split recombinase (as described herein); and (b) a second polynucleic acid molecule (optionally stably integrated) comprising a nucleic acid sequence encoding, from 5′ to 3′: (i) a first recombinase attachment site; (ii) a gene coding segment; and (iii) a second recombinase attachment site; wherein the first recombinase attachment site and the second recombinase attachment site correspond to the split recombinase (or polypeptide dimer having recombinase activity) of (a).

As used herein, the term “stably integrated” refers to an exogenous nucleic acid sequence, nucleic acid molecule, construct, gene, or nucleic acid sequence that has been inserted into the genome of and organism (e.g. the engineered cell as described herein) and is passed on to future generations after cell division. It is to be understood that any nucleic acid sequence, nucleic acid molecule, construct, gene or nucleic acid sequence described herein may be stably integrated. In some embodiments, any nucleic acid sequence, nucleic acid molecule, construct gene or nucleic acid sequence may be integrated into the genome using random integration, targeted integration, or transposon-mediated integration. It is to be understood that any of the stably integrated nucleic acid molecules described herein may comprise IR/DR sequences that are capable of binding the Sleeping Beauty transposase. Stable integration using the Sleeping Beauty transposase is described in Mites, Lajos, et al. Nature genetics 41.6 (2009): 753-761 which is incorporated by reference in its entirety. In some embodiments, a IR/DR sequence comprises a Sleeping Beauty 100X (SB100X) IR/DR.

An engineered cell described herein may further comprise a landing pad. As used herein, the term “landing pad” refers to a heterologous nucleic acid molecule sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; U.S. Pat. No. 8,980,579 B2; U.S. Pat. No. 10,017,786 B2; U.S. Pat. No. 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, COSMIC, H11, CCR5, and LiPS-A3S.

Each of the landing pads described herein comprises at least one recombination site. For example, a landing pad may comprise recombination sites corresponding to a Flp recombinase, a Bxb1 integrase, a PhiC31 recombinase, a TP901 recombinase, a Cre recombinase, a Vcre recombinase, an Int1-Int34 recombinase, an R4 recombinase, or a Dre recombinase.

The landing pads described herein may comprise one or more expression cassettes.

### V. Kits

In some aspects, the disclosure relates to kits comprising a split recombinase described herein, a polynucleic acid encoding a split recombinase described herein, an AAV production system described herein, and/or an engineered cell described herein.

In some embodiments, a kit comprises one or more nucleic acid molecules collectively comprising an AAV production system.

In some embodiments, the kit further comprises a small molecule that induces dimerization of an inducible split recombinase described herein. In some embodiments, the small molecule inducer is gibberellic acid (GA), abscisic acid (ABA), or rapalog (Rap).

In some embodiments, a kit comprises a nucleic acid molecule comprising a nucleic acid sequence of a transcriptional activator operably linked to a nucleic acid sequence of a promoter, wherein the transcriptional activator, when expressed in the presence of the small molecule inducer, binds to a chemically inducible promoter of the AAV production system, optionally wherein an engineered cell comprises the nucleic acid molecule comprising the nucleic acid sequence of the transcriptional activator. In some embodiments, the transcriptional activator is selected from the group consisting of TetOn-3G, TetOn-V16, TetOff-Advanced, VanR-VP16, TtgR-VP16, PhlF-VP16, and the cumate cTA and rcTA.

In some embodiments, the kit may further comprise instructions for use of the cells.

### VI. Methods of Using Engineered Cells for AAV Production

In some aspects, the present disclosure provides methods for producing AAV using an AAV production system described herein, wherein the AAV production system comprises: (a) an AAV production component collectively encode gene products required for generation of an AAV in a recombinant host cell; and (b) a split recombinase described herein (or a polynucleic acid molecule encoding the same, as described herein). In some embodiments, the method of AAV production comprises transfecting or stably integrating into an engineered cell any combination of the one or more nucleic acid molecules collectively comprising the AAV production component and the polynucleic acid molecule encoding the split recombinase. In some embodiments, the method of AAV production further comprises transfecting a nucleic acid molecule comprising a payload for AAV delivery (e.g. a therapeutic DNA sequence) as described above. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises harvesting the AAV produced from the culture of engineered cells using methods that are well known to those of skill in the art.

### Examples

### Example 1: Testing Genetic Designs for Split Recombinases

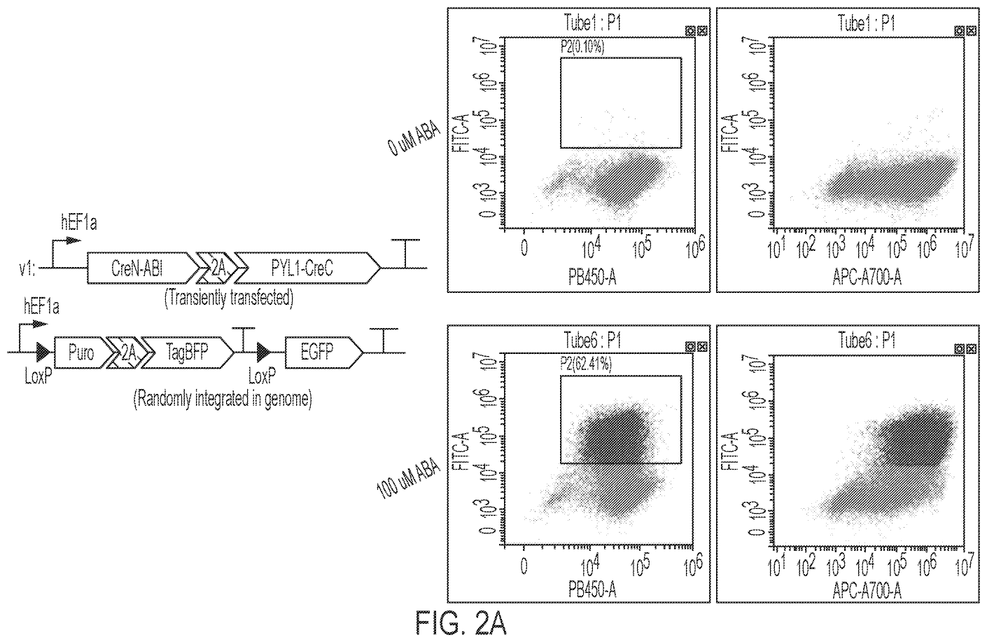

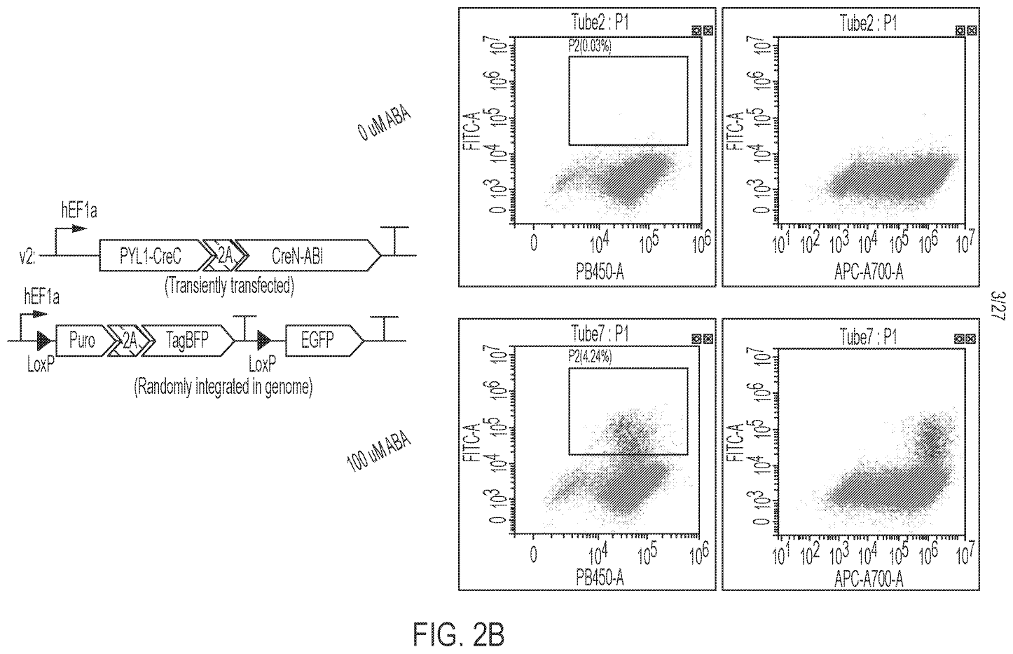

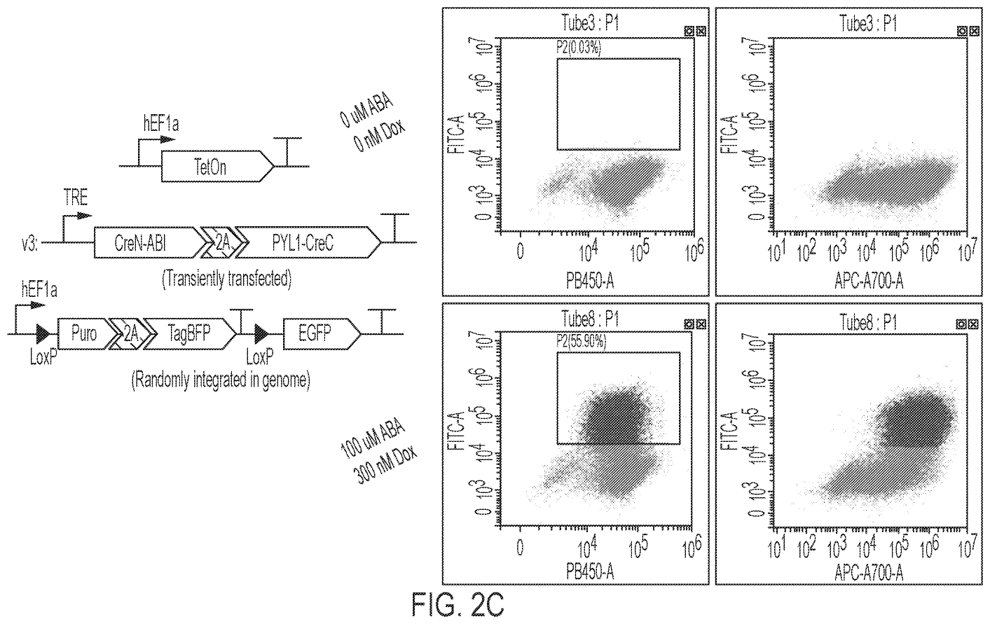

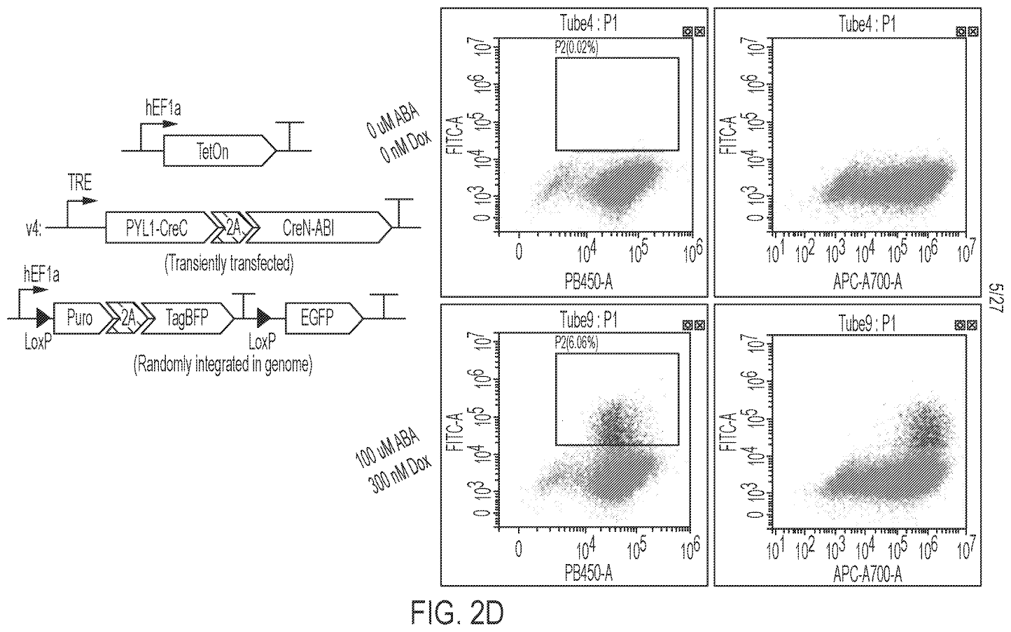

Four genetic designs (V1-V4) were tested for expressing a Cre 270/271 split recombinase (split between amino acids 270 and 271) (FIGS. 2A-2D). The N-terminal portion of the Cre 270/271 split recombinase was fused to an ABI dimerization domain (CreN-ABI) and the C-terminal portion of the Cre 270/271 split recombinase was fused to a PYL1 dimerization domain (PYL1-Cre). In some embodiments, the nucleic acid sequence encoding the split recombinase comprised the structure (from 5′ to 3′): CreN-ABI-P2A-PYL1-CreC (FIGS. 2A and 2C). In other embodiments, the nucleic acid sequence encoding the split recombinase comprised the structure (from 5′ to 3′): PYL1-CreC-P2A-CreN-ABI (FIGS. 2B and 2D). Expression of the Cre 270/271 split recombinase was driven by either a constitutive hEF1a promoter (FIGS. 2A-2B) or an inducible TRE promoter with addition of TetOn (FIGS. 2C-2D). Recombinase constructs were transfected into a reporter HEK293FT cell containing an integrated expression construct that expresses TagBFP prior to recombination and EGFP following recombination. An iRFP720 expression construct was also cotransfected to control for transfection efficiency. TagBFP was measured in the PB450-A channel, EGFP was measured in the FITC-A channel, and iRFP720 was measured in the APC-A700-A channel.

V1 exhibited a low level of background recombination (0.1%) in the absence of a small-molecule inducer. V2-V4 exhibited near zero background recombination in the absence of a small-molecule inducer. In addition, constructs with 5′ CreN-ABI fusion (i.e., V1 and V3) exhibited increased recombination when induced compared to constructs with a 3′ CreN-ABI fusion (i.e., V2 and V4), though the 3′ Cre-N-ABI fusions exhibited lower background recombination. Based on these results, additional split recombinase designs primarily utilize genetic designs in which the sequence encoding the N-terminal half is placed 5′ to the sequence encoding the C-terminal half.

### Example 2: Testing of Split Recombinase/Dimerization Domain Combinations

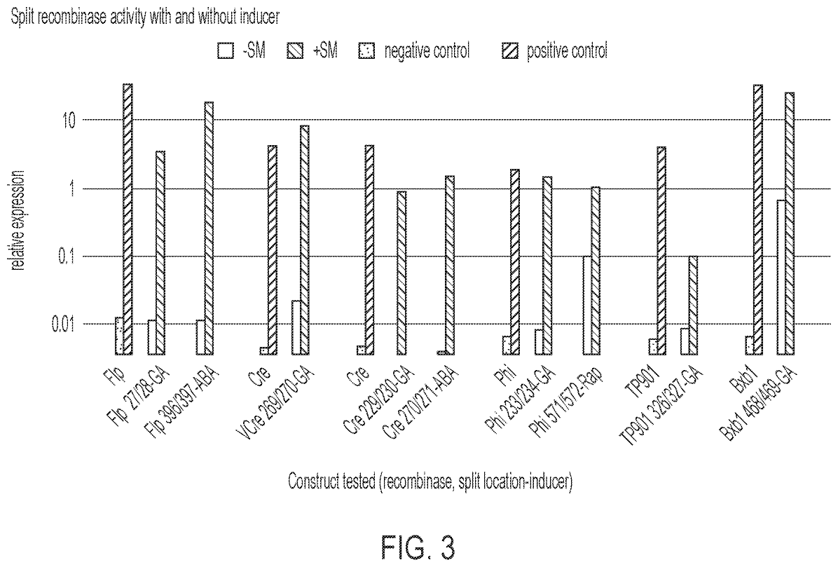

Several split recombinase/dimerization domain combinations were tested (FIG. 3).

Each combination showed induction of recombinase activity with addition of a respective small molecule inducer. Some combinations showed very high induction. Importantly, several combinations had near background levels of recombinase activity in the absence of small molecule. Recombinase/dimerization domains that performed particularly well (high induction and low background) include: F1pN396-ABI/PYL1-F1pC397 (abscisic acid inducible); Vcre_N269-GID1/GAI-Vcre_C279 (gibberellic acid inducible); CreN229-GID1/GAI-CreC230 (gibberellic acid inducible); PhiC31N233-GID1/GAI-PhiC31C234 (gibberellic acid inducible).

### Example 3: Testing of Bxb1 Split Recombinase Split Locations

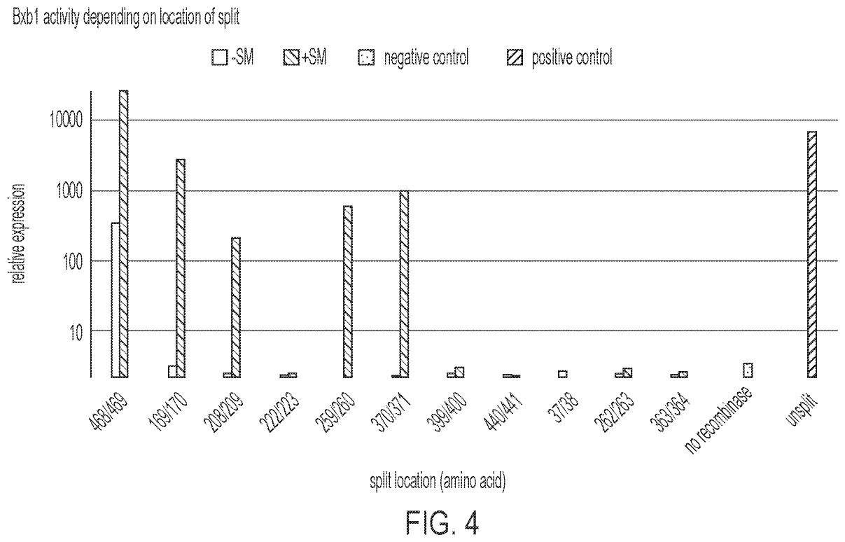

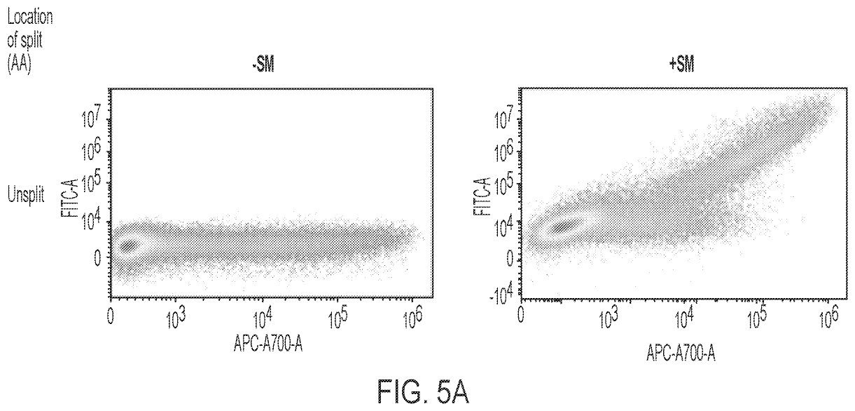

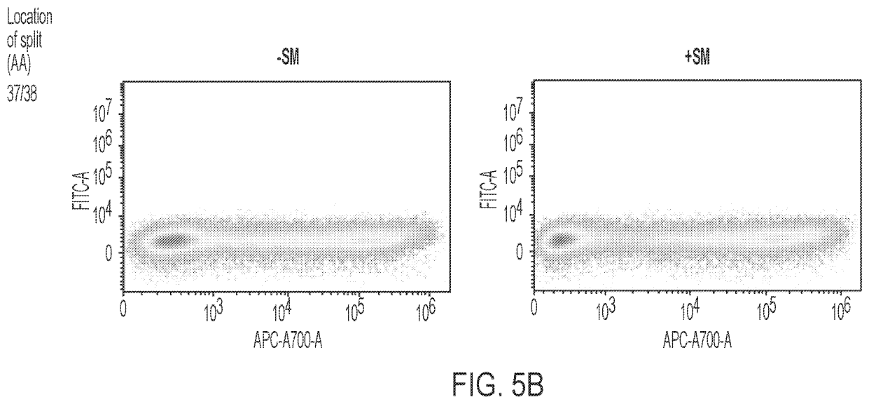

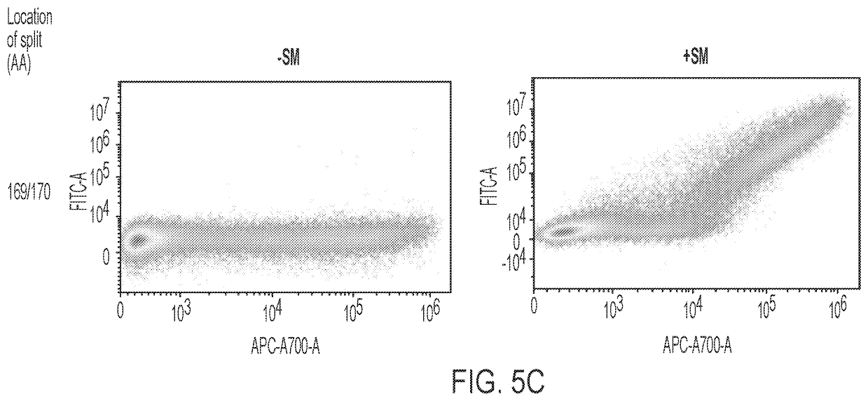

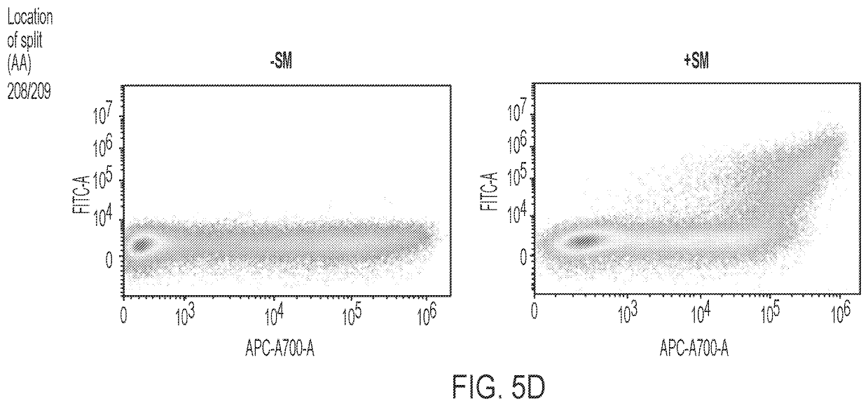

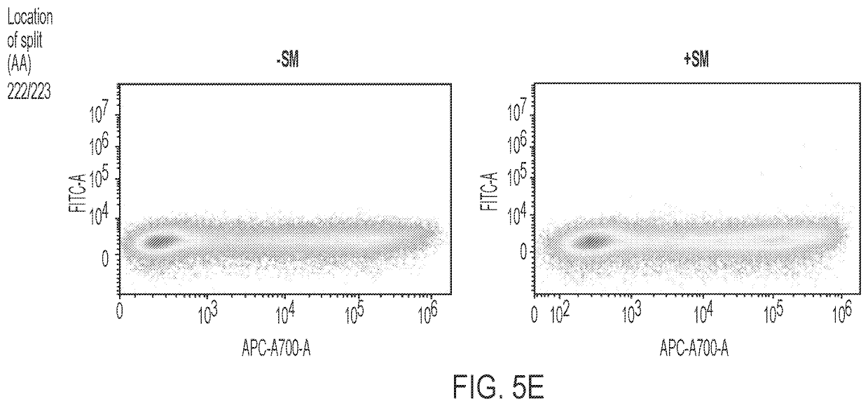

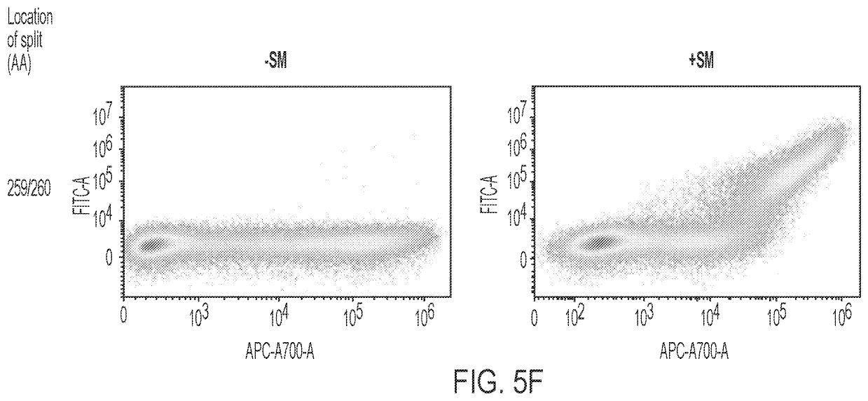

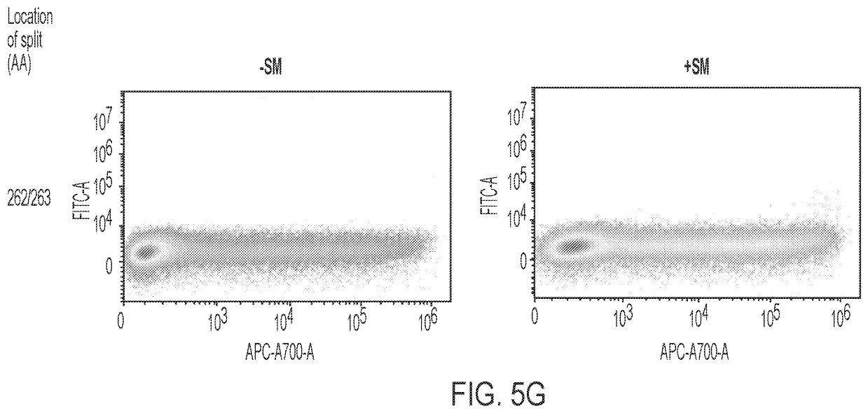

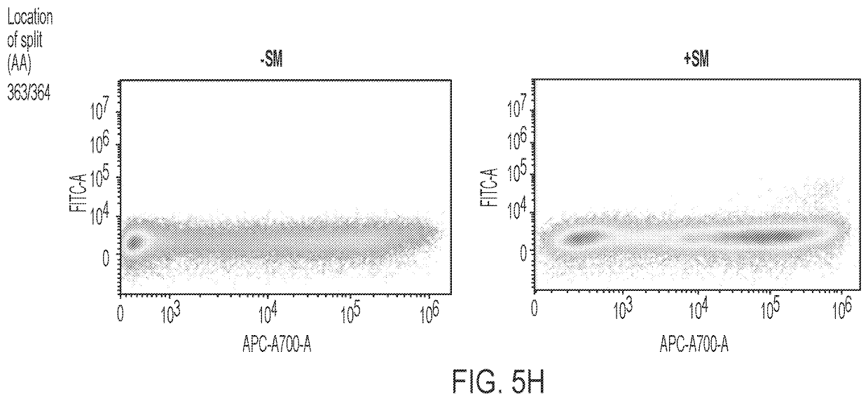

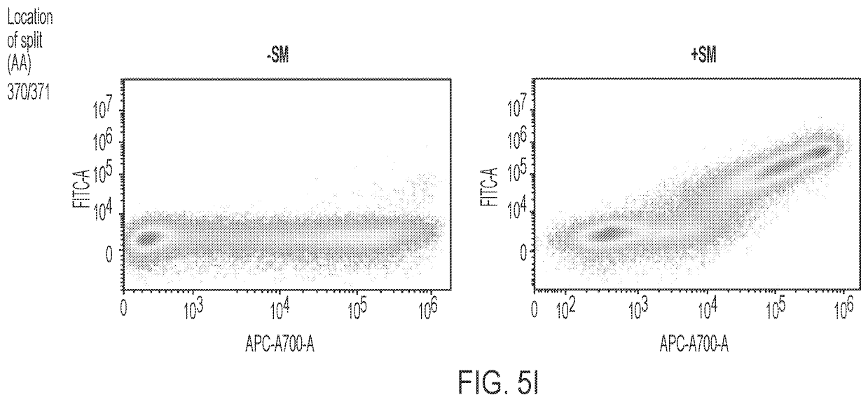

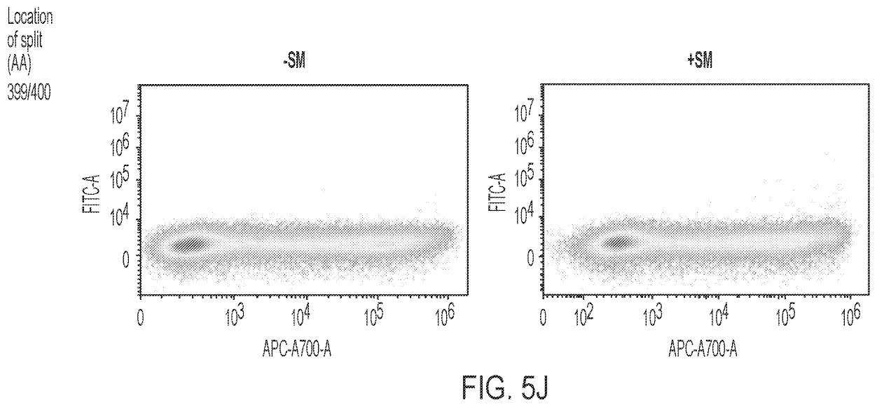

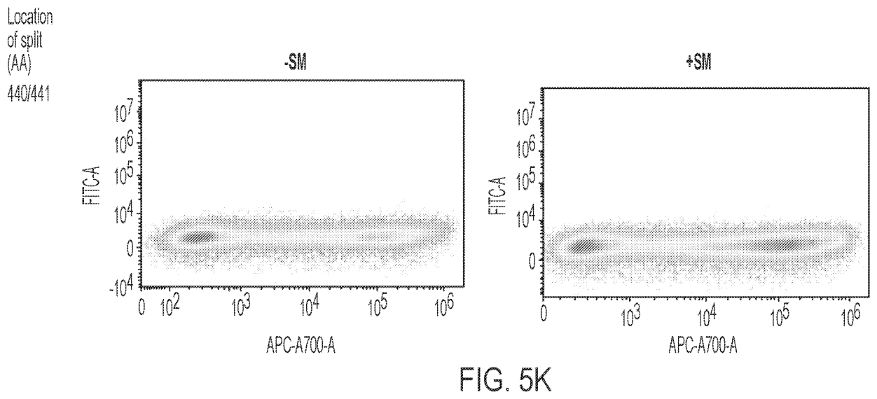

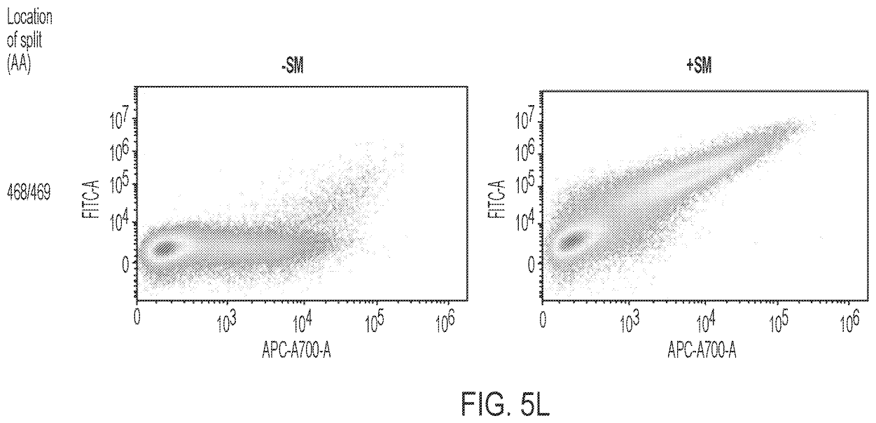

Additional Bxb1 split recombinases were designed by selecting amino acid split locations believed likely to result in successful split recombinases, as well as locations likely to disrupt recombinase function (as a negative control) (FIG. 4 and FIGS. 5A-5L). Each Bxb1 split recombinase showed induction of recombinase activity with addition of a respective small molecule inducer. Some Bxb1 split recombinases showed very high induction (e.g., 169/170, 208/209, 259/260, 370/371, 468/469). Only one Bxb1 split recombinase (468/469) showed high basal activity.

### Example 4: Testing of Additional Split Recombinases

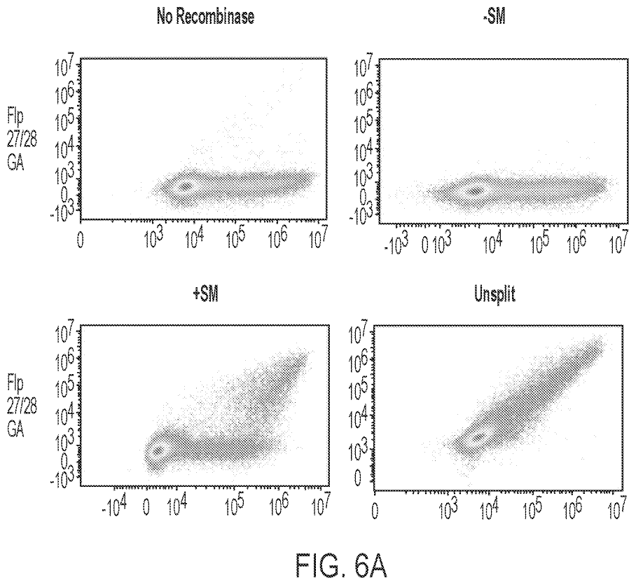

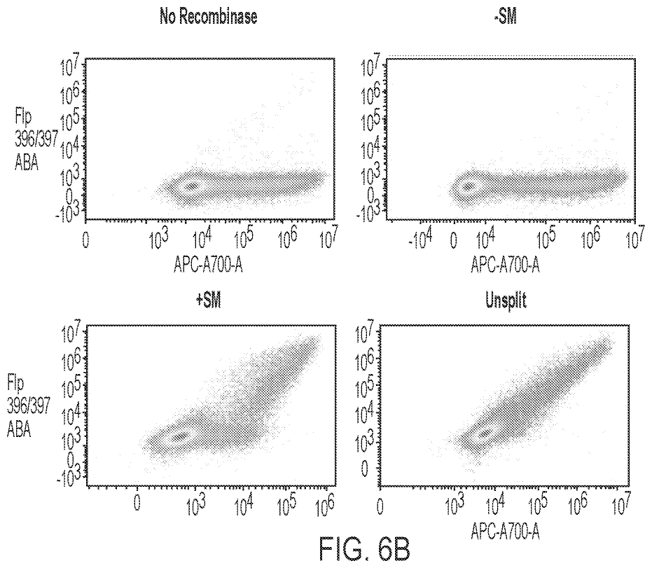

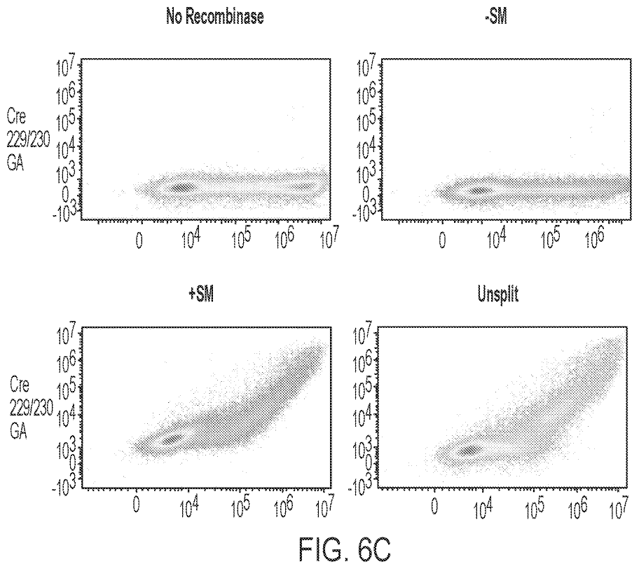

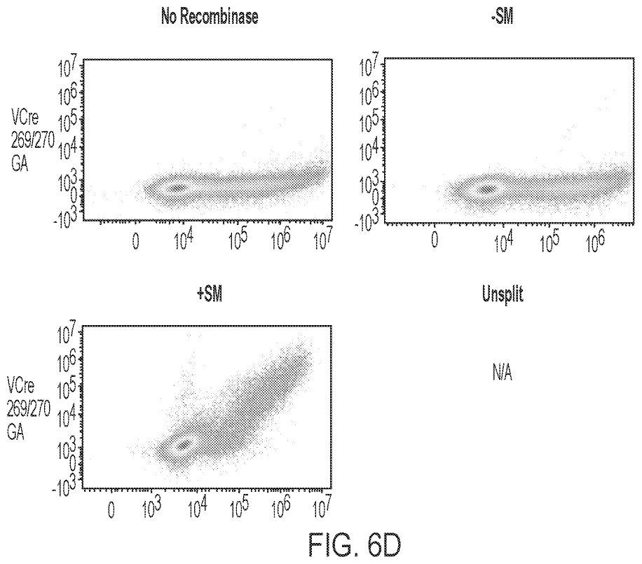

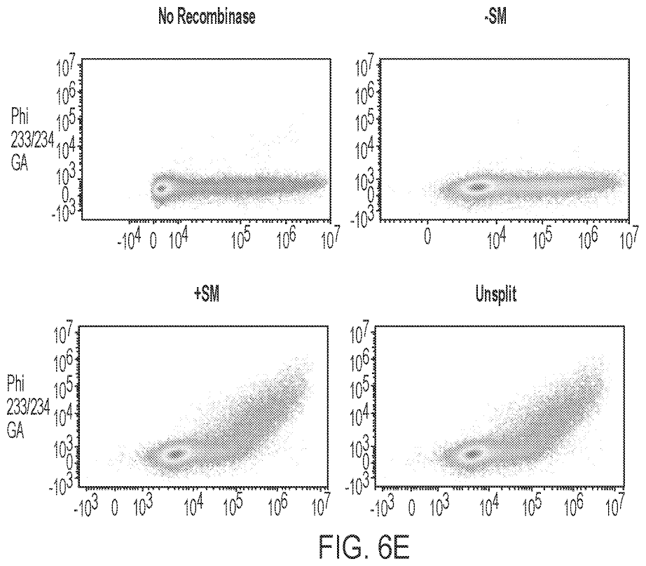

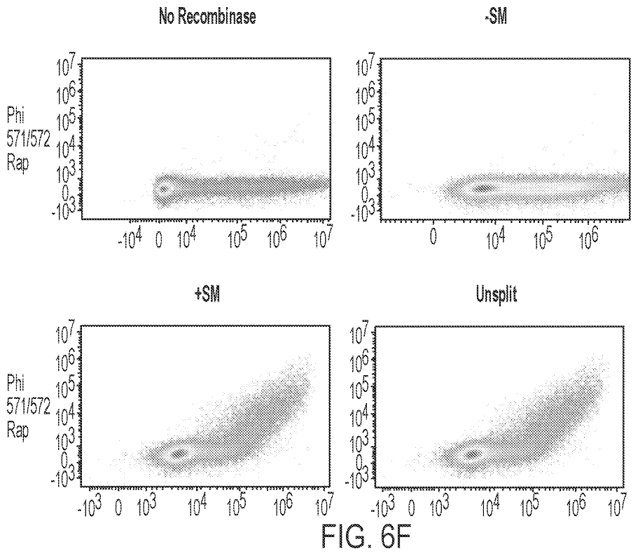

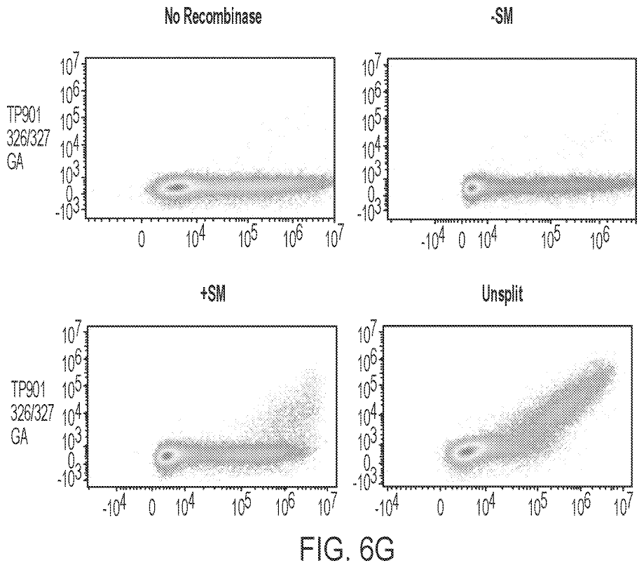

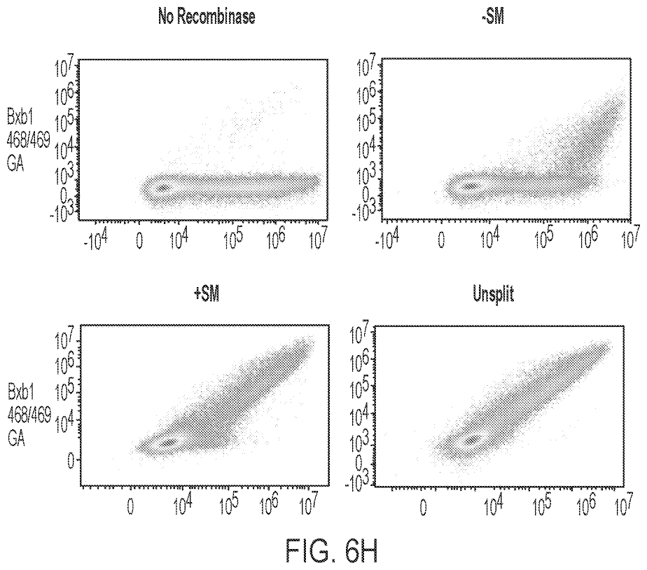

The activities (basal and induced) of additional split recombinases were tested (FIGS. 6A-6H).

TABLE 1
List of Split Recombinases Studied in Examples 1-4

| Recom- | Split | Recognition | Dimerization | |
|---|---|---|---|---|
| binase | Site | Sites | domains | Inducer |
| Flp | 27/28 | FRT/FRT | GID1, GAI | Gibberellic Acid (GA) |
| Flp | 396/397 | FRT/FRT | ABI, PYL | Abscisic Acid (ABA) |
| Bxb1 | 37/38 | attB/attP | GID1, GAI | Gibberellic Acid (GA) |
| Bxb1 | 169/170 | attB/attP | GID1, GAI | Gibberellic Acid (GA) |
| Bxb1 | 208/209 | attB/attP | GID1, GAI | Gibberellic Acid (GA) |
| Bxb1 | 222/223 | attB/attP | GID1, GAI | Gibberellic Acid (GA) |
| Bxb1 | 259/260 | attB/attP | GID1, GAI | Gibberellic Acid (GA) |
| Bxb1 | 262/263 | attB/attP | GID1, GAI | Gibberellic Acid (GA) |
| Bxb1 | 363/364 | attB/attP | GID1, GAI | Gibberellic Acid (GA) |
| Bxb1 | 370/371 | attB/attP | GID1, GAI | Gibberellic Acid (GA) |
| Bxb1 | 399/400 | attB/attP | GID1, GAI | Gibberellic Acid (GA) |
| Bxb1 | 440/441 | attB/attP | GID1, GAI | Gibberellic Acid (GA) |
| Bxb1 | 468/469 | attB/attP | GID1, GAI | Gibberellic Acid (GA) |
| PhiC31 | 233/234 | attB/attP | GID1, GAI | Gibberellic Acid (GA) |
| PhiC31 | 571/572 | attB/attP | FRB, FKBP | Rapalog (Rap) |
| TP901 | 326/327 | attB/attP | GID1, GAI | Gibberellic Acid (GA) |
| Cre | 229/230 | loxP/loxP | ABI, PYL | Abscisic Acid (ABA) |
| Cre | 269/270 | loxP/loxP | ABI, PYL | Abscisic Acid (ABA) |
| Vcre | 269/270 | VloxP/VloxP | GID1, GAI | Gibberellic Acid (GA) |

TABLE 2
Exemplary Recombinase Amino Acid Sequences

| SEQ ID | | |
|---|---|---|
| NO: | Description. | Sequence |
| 1 | FLP | MSQFDILCKTPPKVLVRQFVERFERPSGEKIASCAAELTYLC |
| | | WMITHNGTAIKRATFMSYNTIISNSLSFDIVNKSLQFKYKTQK |
| | | ATILEASLKKLIPAWEFTIIPYNGQKHQSDITDIVSSLQLQFESS |
| | | EEADKGNSHSKKMLKALLSEGESIWEITEKILNSFEYTSRFTK |
| | | TKTLYQFLFLATFINCGRFSDIKNVDPKSFKLVQNKYLGVIIQ |
| | | CLVTETKTSVSRHIYFFSARGRIDPLVYLDEFLRNSEPVLKRV |
| | | NRTGNSSSNKQEYQLLKDNLVRSYNKALKKNAPYPIFAIKNG |
| | | PKSHIGRHLMTSFLSMKGLTELTNVVGNWSDKRASAVARTT |
| | | YTHQITAIPDHYFALVSRYYAYDPISKEMIALKDETNPIEEWQ |
| | | HIEQLKGSAEGSIRYPAWNGIISQEVLDYLSSYINRRI |
| 2 | Bxb1 | MRALVVIRLSRVTDATTSPERQLESCQQLCAQRGWDVVGVA |
| | | EDLDVSGAVDPFDRKRRPNLARWLAFEEQPFDVIVAYRVDR |
| | | LTRSIRHLQQLVHWAEDHKKLVVSATEAHFDTTTPFAAVVIA |
| | | LMGTVAQMELEAIKERNRSAAHFNIRAGKYRGSLPPWGYLP |
| | | TRVDGEWRLVPDPVQRERILEVYHRVVDNHEPLHLVAHDLN |
| | | RRGVLSPKDYFAQLQGREPQGREWSATALKRSMISEAMLGY |
| | | ATLNGKTVRDDDGAPLVRAEPILTREQLEALRAELVKTSRAK |
| | | PAVSTPSLLLRVLFCAVCGEPAYKFAGGGRKHPRYRCRSMG |
| | | FPKHCGNGTVAMAEWDAFCEEQVLDLLGDAERLEKVWVA |
| | | GSDSAVELAEVNAELVDLTSLIGSPAYRAGSPQREALDARIA |
| | | ALAARQEELEGLEARPSGWEWRETGQRFGDWWREQDTAA |
| | | KNTWLRSMNVRLTFDVRGGLTRTIDFGDLQEYEQHLRLGSV |
| | | VERLHTGMS |
| 3 | PhiC31 | MDTYAGAYDRQSRERENSSAASPATQRSANEDKAADLQRE |
| | | VERDGGRFRFVGHFSEAPGTSAFGTAERPEFERILNECRAGRL |
| | | NMIIVYDVSRFSRLKVMDAIPIVSELLALGVTIVSTQEGVFRQ |
| | | GNVMDLIHLIMRLDASHKESSLKSAKILDTKNLQRELGGYVG |
| | | GKAPYGFELVSETKEITRNGRMVNVVINKLAHSTTPLTGPFE |
| | | FEPDVIRWWWREIKTHKHLPFKPGSQAAIHPGSITGLCKRMD |
| | | ADAVPTRGETIGKKTASSAWDPATVMRILRDPRIAGFAAEVI |
| | | YKKKPDGTPTTKIEGYRIQRDPITLRPVELDCGPIIEPAEWYEL |
| | | QAWLDGRGRGKGLSRGQAILSAMDKLYCECGAVMTSKRGE |
| | | ESIKDSYRCRRRKVVDPSAPGQHEGTCNVSMAALDKFVAERI |
| | | FNKIRHAEGDEETLALLWEAARRFGKLTEAPEKSGERANLV |
| | | AERADALNALEELYEDRAAGAYDGPVGRKHFRKQQAALTL |
| | | RQQGAEERLAELEAAEAPKLPLDQWFPEDADADPTGPKSW |
| | | WGRASVDDKRVFVGLFVDKIVVTKSTTGRGQGTPIEKRASIT |
| | | WAKPPTDDDEDDAQDGTEDVAA |
| 4 | TP901 | MTKKVAIYTRVSTTNQAEEGFSIDEQIDRLTKYAEAMGWQV |
| | | SDTYTDAGFSGAKLERPAMQRLINDIENKAFDTVLVYKLDRL |
| | | SRSVRDTLYLVKDVFTKNKIDFISLNESIDTSSAMGSLFLTILS |
| | | AINEFERENIKERMTMGKLGRAKSGKSMMWTKTAFGYYHN |
| | | RKTGILEIVPLQATIVEQIFTDYLSGISLTKLRDKLNESGHIGK |
| | | DIPWSYRTLRQTLDNPVYCGYIKFKDSLFEGMHKPIIPYETYL |
| | | KVQKELEERQQQTYERNNNPRPFQAKYMLSGMARCGYCGA |
| | | PLKIVLGHKRKDGSRTMKYHCANRFPRKTKGITVYNDNKKC |
| | | DSGTYDLSNLENTVIDNLIGFQENNDSLLKIINGNNQPILDTSS |
| | | FKKQISQIDKKIQKNSDLYLNDFITMDELKDRTDSLQAEKKL |
| | | LKAKISENKFNDSTDVFELVKTQLGSIPINELSYDNKKKIVNN |
| | | LVSKVDVTADNVDIIFKFQLA |
| 5 | Cre | MSNLLTVHQNLPALPVDATSDEVRKNLMDMFRDRQAFSEH |
| | | TWKMLLSVCRSWAAWCKLNNRKWFPAEPEDVRDYLLYLQ |
| | | ARGLAVKTIQQHLGQLNMLHRRSGLPRPSDSNAVSLVMRRI |
| | | RKENVDAGERAKQALAFERTDFDQVRSLMENSDRCQDIRNL |
| | | AFLGIAYNTLLRIAEIARIRVKDISRTDGGRMLIHIGRTKTLVS |
| | | TAGVEKALSLGVTKLVERWISVSGVADDPNNYLFCRVRKNG |
| | | VAAPSATSQLSTRALEGIFEATHRLIYGAKDDSGQRYLAWSG |
| | | HSARVGAARDMARAGVSIPEIMQAGGWTNVNIVMNYIRNL |
| | | DSETGAMVRLLEDGD |
| 6 | Vcre | MIENQLSLLGDFSGVRPDDVKTAIQAAQKKGINVAENEQFK |
| | | AAFEHLLNEFKKREERYSPNTLRRLESAWTCFVDWCLANHR |
| | | HSLPATPDTVEAFFIERAEELHRNTLSVYRWAISRVHRVAGC |
| | | PDPCLDIYVEDRLKAIARKKVREGEAVKQASPFNEQHLLKLT |
| | | SLWYRSDKLLLRRNLALLAVAYESMLRASELANIRVSDMEL |
| | | AGDGTAILTIPITKTNHSGEPDTCILSQDVVSLLMDYTEAGKL |
| | | DMSSDGFLFVGVSKHNTCIKPKKDKQTGEVLHKPITTKTVEG |
| | | VFYSAWETLDLGRQGVKPFTAHSARVGAAQDLLKKGYNTL |
| | | QIQQSGRWSSGAMVARYGRAILARDGAMAHSRVKTRSAPM |
| | | QWGKDEKD |
| 7 | Int1 | MTNPASRPKAYSYIRMSSAIQIKGDSFRRQAEASAKYAAEHD |
| | | LDLIDDYKLADLGVSAFKSDNLTTGALGRFVAECEAGEIEAG |
| | | SFLLIESLDRLSRDKILDAFSLFARILKTGVKIVTLSDGQVYDG |
| | | SSDQVGSIYYAISVMIRSNDESKIKSTRGLANWSQKRKLAAE |
| | | HGVKMSSQCPAWLKLSVDRKSYLIDKERAKIVQRIFEASASG |
| | | KGANLITKELNRDKVPTFGRGALWAEAFVSKTLRNRAVLGE |
| | | FQPGQYVSGKRQPAGDPIPGYFPPVIEEELFDIVQASLRGRLL |
| | | AGGRRGEGQSNIFTHVAFCGYCGSKMRHRSKGSRVKGNPPH |
| | | RYLTCFNRFNGPGCDCKPLPYAAFERSFLTFVRDVDLRGLLE |
| | | GAKRKSEAKTIADRITVNEEKVRKADERIRDYLIKIEGAPDLA |
| | | EIFMERIRELKAEKDDLVRSIEESNDALSKIKSDNVTDEELAS |
| | | LISTFQNPCGENRIRLADRIKSIIERIDVYPNGEIRKDDPAIDLV |
| | | RASGDPDAEKIIAAMNAGSRLKDDPYFIVTFRNGAVQTVVPN |
| | | PSNPDDIRVSVYAGEKTRRVEGSAYEYESD |
| 8 | Int2 | MPIAPEFLSLAYPGQEFPAYLYGRASRDPKRKGRSVQSQLDE |
| | | GRATCLDAGWPIAGEFKDVDRSASAYARRTRDEFEEMIAGIQ |
| | | AGECRILVAFEASRYYRDLEAYVRLRRVCREAGVLLCYNGQ |
| | | VYDLSKSADRKATAQDAVNAEGEADDIRERNLRTTRLNAKR |
| | | GGAHGPVPDGYKRRYDPDSGDLVDQIPHPDRAGLITEIFRRA |
| | | AAAEPLAAICRDLNERGETTHRGKAWQRHHLHAILRNPAYI |
| | | GHRRHLGVDTGKGMWAPICDDEDFAETFQAVQEILSLPGRQ |
| | | LSPGPEAQHLQTGIALCGEHPDEPPLRSVTVRGRTNYNCSTR |
| | | YDVAMREDRMDAFVEESVITWLASDEAVAAFEDNTDDERT |
| | | RKARIRLKVLEEQLEAAQKQARTLRPDGMGMLLSIDSLAGL |
| | | EAELTPQIDKARQESRSLHVPALLRDLLGKPRADVDRAWNE |
| | | ALTLPQRRMILRMVVTIRLFKAGSRGVRAIEPGRITLSYVGEP |
| | | GFKPVGGNRAKQ |
| 9 | Int3 | MRKVAIYSRVSTINQAEEGYSIQGQIEALTKYCEAMEWKIYK |
| | | NYSDAGFSGGKLERPAITELIEDGKNNKFDTILVYKLDRLSRN |
| | | VKDTLYLVKDVFTANNIHFVSLKENIDTSSAMGNLFLTLLSAI |
| | | AEFEREQIKERMQFGVMNRAKSGKTTAWKTPPYGYRYNKD |
| | | EKTLSVNELEAANVRQMFDMIISGCSIMSITNYARDNFVGNT |
| | | WTHVKVKRILENETYKGLVKYREQTFSGDHQAIIDEKTYNK |
| | | AQIALAHRTDTKTNTRPFQGKYMLSHIAKCGYCGAPLKVCT |
| | | GRAKNDGTRRQTYVCVNKTESLARRSVNNYNNQKICNTGR |
| | | YEKKHIEKYVIDVLYKLQHDKEYLKKIKKDDNIIDITPLKKEI |
| | | EIIDKKINRLNDLYINDLIDLPKLKKDIEELNHLKDDYNKAIKL |
| | | NYLDKKNEDSLGMLMDNLDIRKSSYDVQSRIVKQLIDRVEV |
| | | TMDNIDIIFKF |
| 10 | Int4 | MITTRKVAIYVRVSTTNQAEEGYSIQGQIDSLIKYCEAMGWII |
| | | YEEYTDAGFSGGKIDRPAMSKLITDAKHKRFDTILVYKLDRL |
| | | SRSVRDTLYLVKDVFNQNNIHFVSLQENIDTSSAMGNLFLTL |
| | | LSAIAEFEREQITERMTMGKIGRAKSGKTMAWTYTPFGYDY |
| | | NKEKGELILDPAKAPIVKMIYTDYLKGMSIQKIVDKLNKMDY |
| | | NGKDCTWFPHGVKHLLDNPVYYGMTRYNNKLFPGNHQPIIT |
| | | KELFDKTQRERQRRRLGIEENHYTIPFQAKYMLSKFLRCRQC |
| | | GSRMGLELGRPRKKEGKRSKKYYCLNSRPKRTASCDTPLYD |
| | | AETLEDYVLHEIAKIQKDPSIASRQKHIEDHELKYKRERIEANI |
| | | NKTVNQLSKLNNLYLNDLITLEDLKTQTNTLIAKKRLLENEL |
| | | DKTCDNDDELDRQETIADFLALPDVWTMDYEGQKYAVELL |
| | | VQRVKVDRDNIDIHWTF |
| 11 | Int5 | MPGMTTETGPDPAGLIDLFCRKSKAVKSRANGAGQRRKQEI |
| | | SIAAQETLGRKVAALLGMQVRHVWKEVGSASRFRKGKARD |
| | | DQSKALKALESGEVGALWCYRLDRWDRGGAGAILKIIEPED |
| | | GMPRRLLFGWDEDTGRPVLDSTNKRDRGELIRRAEEAREEA |
| | | EKLSERVRDTKAHQRENGEWVNARAPYGLRVVLVTVSDEE |
| | | GDEYDERKLAADDEDAGGPDGLTKAEAARLVFTLPVTDRLS |
| | | YAGTAHAMNTREIPSPTGGPWIAVTVRDMIQNPAYAGWQTT |
| | | GRQDGKQRRLTFYNGEGKRVSVMHGPPLVTDEEQEAAKAA |
| | | VKGEDGVGVPLDGSDHDTRRKHLLSGRMRCPGCGGSCSYS |
| | | GNGYRCWRSSVKGGCPAPTYVARKSVEEYVAFRWAAKLAA |
| | | SEPDDPFVIAVADRWAALTHPQASEDEKYAKAAVREAEKNL |
| | | GRLLRDRQNGVYDGPAEQFFAPAYQEALSTLQAAKDAVSES |
| | | SASAAVDVSWIVDSSDYEELWLRATPTMRNAIIDTCIDEIWV |
| | | AKGQRGRPFDGDERVKIKWAART |
| 12 | Int6 | MQLDATLTLRDEGLSAFHQRHIKQGALGVFLRAIEDGRIQPG |
| | | SVLIVEGLDRLSRAEPIQAQAQLAQIINAGITVVTASDGREYN |
| | | RERLKAQPMDLVYSLLVMIRAHEESDTKSKRVKAAIRRQCE |
| | | GWVAGTWRGIIRNGKDPHWVRLGEHGKFEHVPERVLAVRT |
| | | MIDLFLEGHGAIEITRRLTEQNLYVSNAGNYSVHMYRIVRNQ |
| | | ALIGEKRISVDGEEFRLDGYYPPILTREEFAELQQTMSERGRR |
| | | KGKGEIPNIITGLSITVCGYCGRAMTTQNSKARAPKGKSVVR |
| | | RLSCPMNSFNEGCPIGGSCESEIVERALMRYCSDQFNLSRLLE |
| | | GDDGTARRTAQLAVARQRASDIEAQIQRVTDALLSDDGKAP |
| | | AAFTRRARELETQLEEQRREIEALEHQIAASSAHGIPAAAEA |
| | | WAQLVDGVLALDYDARMKARQLVADTFRKIVVYQRGFAPI |
| | | DDAAADRWKRSGTIGLMLVTKRGGMRLLNVDRRTGCWQA |
| | | EDDLDPSLIPSDGLPMLPLDA |
| 13 | Int7 | MKVAIYVRVSTDEQAKEGFSIPAQRERLRAFCASQGWEIVQE |
| | | YIEEGWSAKDLDRPQMQRLLKDIKKGNIDIVLVYRLDRLTRS |
| | | VLDLYLLLQTFEKYNVAFRSATEVYDTSTAMGRLFITLVAAL |
| | | AQWERENLAERVKFGIEQMIDEGKKPGGHSPYGYKFDKDFN |
| | | CTIIEEEADVVRMIYRMYCDGYGYRSIADRLNELMVKPRIAK |
| | | EWNHNSVRDILTNDIYIGTYRWGDKVVPNNHPPIISETLFKK |
| | | AQKEKEKRGVDRKRVGKFLFTGLLQCGNCGGHKMQGHFDK |
| | | REQKTYYRCTKCHRITNEKNILEPLLDEIQLLITSKEYFMSKFS |
| | | DRYDQQEVVDVSALTKELEKIKRQKEKWYDLYMDDRNPIP |
| | | KEELFAKINELNKKEEEIYSKLSEVEEDKEPVEEKYNRLSKMI |
| | | DFKQQFEQANDFTKKELLFSIFEKIVIYREKGKLKKITLDYTL |
| | | K |
| 14 | Int8 | MKVAVYCRVSTLEQKEHGHSIEEQERKLKSFCDINDWTVYD |
| | | TYIDAGYSGAKRDRPELQRLMNDINKFDLVLVYKLDRLTRN |
| | | VRDLLDLLEIFEKNDVSFRSATEVYDTTTAMGRLFVTLVGA |
| | | MAEWERETIRERTQMGKLAALRKGIMLTTPPFYYDRVDNKF |
| | | VPNKYKDVILWAYDEAMKGQSAKAIARKLNNSDIPPPNNTQ |
| | | WQGRTITHALRNPFTRGHFDWGGVHIENNHEPIITDEMYEKV |
| | | KDRLNERVNTKKVRHTSIFRGKLVCPVCNARLTLNSHKKKS |
| | | NSGYIFVKQYYCNNCKVTPNLKPVYIKEKEVIKVFYNYLKRF |
| | | DLEKYEVTQKQNEPEITIDINKVMEQRKRYHKLYASGLMQE |
| | | DELFDLIKETDQTIAEYEKQNENREVKQYDIEDIKQYKDLLLE |
| | | MWDISSDEDKEDFIKMAIKNIYFEYIIGTGNTSRKRNSLKITSI |
| | | EFY |
| 15 | Int9 | MKVAIYTRVSTLEQKEKGHSIEEQERKLRAYSDINDWKIHKV |
| | | YTDAGYSGAKKDRPALQEMLNEIDNFDLVLVYKLDRLTRSV |
| | | KDLLEILELFENKNVLFRSATEVYDTTSAMGRLFVTLVGAM |
| | | AEWERTTIQERTAMGRRASARKGLAKTVPPFYYDRVNDKFV |
| | | PNEYKKVLRFAVEEAKKGTSLREITIKLNNSKYKAPLGKNW |
| | | HRSVIGNALTSPVARGHLVFGDIFVENTHEAIISEEEYEEIKLR |
| | | ISEKTNSTIVKHNAIFRSKLLCPNCNQKLTLNTVKHTPKNKEV |
| | | WYSKLYFCSNCKNTKNKNACNIDEGEVLKQFYNYLKQFDLT |
| | | SYKIENQPKEIEDVGIDIEKLRKERARCQTLFIEGMMDKDEAF |
| | | PIISRIDKEIHEYEKRKDNDKGKTFNYEKIKNFKYSLLNGWEL |
| | | MEDELKTEFIKMAIKNIHFEYVKGIKGKRQNSLKITGIEFY |
| 16 | Int10 | MITTNKVAIYVRVSTTNQVEEGYSIDEQKDKLSSYCDIKDWN |
| | | VYKVYTDGGFSGSNTDRPALESLIKDAKKRKFDTVLVYKLD |
| | | RLSRSQKDTLHLIEDVFIKNGIEFLSLQENFDTSTPFGKAMIGL |
| | | LSVFAQLEREQIKERMQLGKLGRAKSGKSMMWAKTSYGYD |
| | | YHKETGTVTINPAQALTIKFIFESYLRGRSITKLRDDLNEKYP |
| | | KHVPWSYRAVRTILDNPVYCGFNQYKGEIYPGNHEPIISKEE |
| | | YDKTQSELKIRQRTAAENVNPRPFQAKYILSGIAQCGYCGAP |
| | | LKIMLGVKRKDGSRLKKYECHQRHPRTLRGVTTYNDNKKC |
| | | DSGFYYKDKLEAYVLKEISKLQDDADYLDKIFSGDNAETIDR |
| | | ESYKKQIEELSKKLSRLNDLYIDDRITLEELQSKSAEFISMRGT |
| | | LETELENDPALRKNKRKADMRKLLNAEKVFSMDYESQKVL |
| | | VRRLINKVKVTAEDIVINWKI |
| 17 | Int11 | MLRCAIYIRVSTEEQAMHGLSMDAQKADLTDYAKKHNYEII |
| | | DYYVDSGKTARKRLSKRKDLQRMIEDVKLNKIDIIIFTKLDR |
| | | WFRNVRDYYKIQEVLEDHNVDWKTIFENYDTSTANGRLHIN |
| | | IMLSVAQDEADRTSERIKRVFENKLKNNEPTSGSLPIGYKIKE |
| | | KSIIIDEEKAPIAKDVFDFYYYHQSQTKVFKEILNKYNLSLCE |
| | | KTIRRMLENKLYIGIYREHENFCPPLIDKNKFDEVQLILKRRNI |
| | | KYIPTKRIFLFTSLLICKECRHKMIGNAQIRNTKAGKIEYILYR |
| | | CNQSYARHTCNHRKVIYENKIETYLLNNIESELKKFIYDYELE |
| | | DIPKVKNKVNKTNIKRKLEKLKELYINDLIDIDMYKEDYKKY |
| | | TEILNTKEEKIEQRNLQPLKDFLNSDFKSLYSSISREEKRLLWR |
| | | GIISEIQIDCNNDITIIPHP |
| 18 | Int12 | MKVAIYTRVSSAEQANEGYSIHEQKKKLISYCEIHDWNEYKV |
| | | FTDAGISGGSMKRPALQKLMKHLSSFDLVLVYKLDRLTRNV |
| | | RDLLDMLEEFEQYNVSFKSATEVFDTTSAIGKLFITMVGAMA |
| | | EWERETIRERSLFGSRAAVREGNYIREAPFCYDNIEGKLHPNE |
| | | YAKVIDLIVSMFKKGISANEIARRLNSSKVHVPNKKSWNRNS |
| | | LIRLMRSPVLRGHTKYGDMLIENTHEPVLSEHDYNAINNAISS |
| | | KTHKSKVKHHAIFRGALVCPQCNRRLHLYAGTVKDRKGYK |
| | | YDVRRYKCETCSKNKDVKNVSFNESEVENKFVNLLKSYELN |
| | | KFHIRKVEPVKKIEYDIDKINKQKINYTRSWSLGYIEDDEYFE |
| | | LMEEINATKKMIEEQTTENKQSVSKEQIQSINNFILKGWEELT |
| | | IKDKEELILSTVDKIEFNFIPKDKKHKTNTLDINNIHFKF |
| 19 | Int13 | MAVGIYIRVSTQEQASEGHSIESQKKKLASYCEIQGWDDYRF |
| | | YIEEGISGKNTNRPKLKLLMEHIEKGKINILLVYRLDRLTRSVI |
| | | DLHKLLNFLQEHGCAFKSATETYDTTTANGRMSMGIVSLLA |
| | | QWETENMSERIKLNLEHKVLVEGERVGAIPYGFDLSDDEKL |
| | | VKNEKSAILLDMVERVENGWSVNRIVNYLNLTNNDRNWSP |
| | | NGVLRLLRNPALYGATRWNDKIAENTHEGIISKERFNRLQQI |
| | | LADRSIHHRRDVKGTYIFQGVLRCPVCDQTLSVNRFIKKRKD |
| | | GTEYCGVLYRCQPCIKQNKYNLAIGEARFLKALNEYMSTVE |
| | | FQTVEDEVIPKKSEREMLESQLQQIARKREKYQKAWASDLM |
| | | SDDEFEKLMVETRETYDECKQKLESCEDPIKIDETYLKEIVY |
| | | MFHQTFNDLESEKQKEFISKFIRTIRYTVKEQQPIRPDKSKTG |
| | | KGKQKVIITEVEFYQ |
| 20 | Int 14 | MTVGIYIRVSTEEQVKEGFSISAQKEKLKAYCTAQGWEDFKF |
| | | YVDEGKSAKDMHRPLLQEMISHIKKGLIDTVLVYKLDRLTRS |
| | | VVDLHNLLSIFDEFNCAFKSATEVYDTSSAMGRFFITIISSVAQ |
| | | FERENTSERVSFGMAEKVRQGEYIPLAPFGYTKGTDGKLIVN |
| | | KIEKEIFLQVVEMVSTGYSLRQTCEYLTNIGLKTRRSNDVWK |
| | | VSTLIWMLKNPAVYGAIKWNNEIYENTHEPLIDKATFNKVA |
| | | KILSIRSKSTTSRRGHVHHIFKNRLICPACGKRLSGLRTKYINK |
| | | NKETFYNNNYRCATCKEHRRPAVQISEQKIEKAFIDYISNYTL |
| | | NKANISSKKLDNNLRKQEMIQKEIISLQRKREKFQKAWAADL |
| | | MNDDEFSKLMIDTKMEIDAAEDRKKEYDVSLFVSPEDIAKR |
| | | NNILRELKINWTSLSPTEKTDFISMFIEGIEYVKDDENKAVITK |
| | | ISFL |
| 21 | Int15 | MKAAIYIRVSTQEQIENYSIQAQTEKLTALCRSKDWDVYDIFI |
| | | DGGYSGSNMNRPALNEMLSKLHEIDAVVVYRLDRLSRSQRD |
| | | TITLIEEYFLKNNVEFVSLSETLDTSSPFGRAMIGILSVFAQLE |
| | | RETIRDRMVMGKIKRIEAGLPLTTAKGRTFGYDVIDTKLYINE |
| | | EEAKQLQMIYDIFEEEKSITTLQKRLKKLGFKVKSYSSYNNW |
| | | LTNDLYCGYVSYADKVHTKGVHEPIISEEQFYRVQEIFSRMG |
| | | KNPNMNRDSASLLNNLVVCGKCGLGFVHRRKDTISRGKKY |
| | | HYRYYSCKTYKHTHELEKCGNKIWRADKLEELIIDRVNNYSF |
| | | ASRNVDKEDELDNLNEKLKTEHKKKKRLFDLYISGSYEVSEL |
| | | DAMMADIDAQINYYEAQIEANEELKKNKKIQENLADLATVD |
| | | FDSLEFREKQLYLKSLINKIYIDGEQVTIEWL |
| 22 | Int16 | MKGESELDKKAAIYIRVSTQEQATEGYSIQAQTDRLIKYVEA |
| | | KDFILYKKYIDAGYSASKLERPAMQDLIQDVQSKKVDVVIVY |
| | | KLDRLSRSQKDTMYLIEDIFRPNDVELISMQESFDTSTAFGSA |
| | | TVGMLSVFAQLERKSISERMITGRVERAKKGFYHTGGQDRPP |
| | | AGYQFNSDNQLIINEYEAAAIKDLFRLYNDGLGKSSISEYLKK |
| | | NYPGKNKWLPSSIDRMLKNSLYIGKVKFSGAEYDGIHEPIIDE |
| | | VTFYKTQKEIARRKQTNTKRYNYVALLGGLCECGICGAKMA |
| | | NRRAVGRKGKVYRYYRCYSKKGSPKHMMKTDGCSSKAQQ |
| | | QFIIDEAVINNLKNIDVEAELKRRSAPQTNTSLISSQIESIDKQI |
| | | NKLIDLFQVDSMPLDVISEKIDKLNKEKQSMEKLLERKNKLD |
| | | KTELQHRFDVLKSFDWDNSSIESKRVVIEMLVQKVIIHDNSIE |
| | | IILVE |
| 23 | Int 17 | MRTNEHNFHNIEEEIKHVAVYLRLSRGEDESELDNHKTRLLN |
| | | RCELNNWSYELYKEIGSGSTIDDRPVMQKLLTDVEKNLYDA |
| | | VLVVDLDRLSRGNGTDNDRILYSMKVSETLIVVESPYQVLDA |
| | | NNESDEEIILFKGFFARFEFKQINKRMREGKKLAQSRGQWVN |
| | | SVTPYGYIVNKTTKKLTPSEEEAKVVIMIKDFFFEGKSTSDIA |
| | | WELNKRKIKPRRATEWRSSSIANILQNEVYVGNIVYNKSVGN |
| | | KKPSKSKTRVTTPYRRLPEEEWRRVYNAHQPLYSKEEFDRIK |
| | | QYFECNVKSHKGSEVRTYALTGLCKTPDGKTMRVTQGKKG |
| | | TDDDLYLFPKKNKHGDSSIYKGISYNVVYETLKEVILQVKDY |
| | | LDSVLDQNENKDLVEELKEELMKKEDELETIQKAKNRIVQG |
| | | FLIGLYDEQDSIELKVEKEKEIDEKEKEIEAIKMKIDNAKTVN |
| | | NSIKKTKIERLLSDVQSAESEKEINRFYKTLIKEIIVDRTDENE |
| | | AKIKVNFL |
| 24 | Int18 | MITTNKVAIYVRVSTTNQVEEGYSIDEQKDKLEAYCKIKDW |
| | | KIYDVYVDGGFSGANTQRPELERLISDVKRKKVDIVLVYKLD |
| | | RLSRSQKDTLFLIEDVFAKNDVAFISLQENFDTSTPFGKASIG |
| | | MLSVFAQLEREQIKERMMLGKEGRAKNGKSMSWTTIAFGY |
| | | DYSKETGVLSVNPTQALIVNRIFTEYLNGKPVVKIIRDLNAEG |
| | | HVGRKRPWGETITKYLLKNETYLGKVKYKDKVYEGQHEPII |
| | | TQELFDLVQLEVERRQISAYEKYNNPRPFRAKYMLSGLMKC |
| | | GYCGASLGLRYTRKDKNGISHHKYQCRNRHSKDLEKRCESG |
| | | WYSKEELERGVIKELERIKFDPKYKNETLAKKEETIKVEEIKK |
| | | QLERINNQVSKLTELYLDEIITRKELDEKNDKIKTERQFLEEQ |
| | | LENQKSNVLSIRKRKLTRLLKDFDVEKLSYEDASKIVKNIIKEI |
| | | IVTKDGMSITLDF |
| 25 | Int 19 | MGKSITVIPAKKVQTSVLHQDRKKIKVAAYCRVSTDQEEQLS |
| | | SYENQVNYYREFISKHEDYELVDIYADEGISATNTKKRDAFN |
| | | RLIQDCRAGKVDRILVKSISRFARNTLDCIKYVRELKELGVG |
| | | VTFEKENIDSLDSKGEVLLTILSSLAQDESRSISENATWGIRKK |
| | | FERGEVRVNTTKFMGYDKDENGRLIINPQQAETVKFIYEKFL |
| | | EGYSPESIAKYLNDNEIPGWTGKANWYPSAIQKMLQNEKYK |
| | | GDALLQKTFTVDFLTKKRVQNDGQVNQYYVENSHEAIIDEE |
| | | TWETVQLEMARRKTYRDEHQLKSYIMQSEDNPFTTKVFCGA |
| | | CGSAFGRKNWATSRGKRKVWQCNNRYRIKGVEGCYSSHLD |
| | | EATLEQIFLKALELLSENIDLLDGKWEKILAENRLLDKHYSM |
| | | ALSDLLRQEQIDFNPSDMCRVLDHIRIGLDGEITVCLLEGTEV |
| | | DL |
| 26 | Int20 | MRTVRRIQPIKSPCKPRFKVAAYARVSDSRLHHSLSTQISYYN |
| | | RLIQAHPDWELVGIYYDEGISGKEQSNRQGFLNLIKDCEDGKI |
| | | DRIITKSIARFGRNTVELLTTVRQLRLKNIGVTFEKENIDSLSS |
| | | EGELMLTLLASVAQEESQNLSENIRWRIQKKFEKGIPHTPQD |
| | | MYGYRWDGEQYQIEPNEAKVIRKVFKWYLDGDSVQQIVDK |
| | | LNQEQVLTRLGNPFTVASIREFFKQEAYFGRLVLQKTYREAF |
| | | SRNPKRNKGQRNKYIIENAHEPIVTKEYFDLVLHEKERRNQL |
| | | MHQESHLNKGIFRDKISCSECGCLMIVKVDSKQVNKTVRYY |
| | | CRTRNRFGASSCSCRTLGEKRLLASFKSKLGIVPDKEWVENN |
| | | IKHIEYDFGYRILRVTPVKGRKYLIEIREGRY |
| 27 | Int21 | MRNKVAIYVRVSTASQADEGYSIDEQKSKLEAYCEIKDWKI |
| | | YDTYIDGGFSGANTQRPELERLISDAKRKKIDIVLVYKLDRLS |
| | | RSQKDTLFLIEDVFAKNDVAFISLQENFDTSTPFGKASIGMLS |
| | | VFAQLEREQIKERMMLGKEGRAKNGKSMSWTTIPFGYDYSK |
| | | ETGILSVNPTQALIVKRIFTEYLNGKSVVKIIRDLNAEGHVGR |
| | | KRPWGETITKYLLKNETYLGKSKYKGKVFEGQHDAIISQELF |
| | | DLVQLEVEKRQISAFEKYNNPRPFRAKYMLSGLMKCGYCGA |
| | | SLGLYVAPKNKNGVSKYKYQCRHRYHKDKAIRCNSGWYSK |
| | | DELEKRVIKELERLKFDPKYKKETLAKKDETIKVEDIKKQLE |
| | | RINKQVSKLTELYLDEVITRKDLDEKNAKIKTERQYLEEQLE |
| | | NQKSNVMSIRKRKLSRLLKDFDIEKLSYEEASKIVKSVIKEIV |
| | | VTKDDMTITLDF |
| 28 | Int22 | MKVATYVRVSTDEQAKEGFSIPAQRERLRAFCESQGWEIVEE |
| | | YIEEGWSAKDLDRPQMQRLLKDIKKGNIDIVLVYRLDRLTRS |
| | | VLDLYLLLQTFEKYNVAFRSATEVYDTSTAMGRLFITLVAAL |
| | | AQWERENLAERVKFGIEQMIDEGKKPGGHSPYGYKFDKDFN |
| | | CTIIEDEANTVRMIYRMYCDGYGYHSIAKRLNELGIKPRIAKE |
| | | WNHNSVRDILTNDIYIGTYRWGNKVVLNNHPPIISETLFRKV |
| | | QKEKEKRRVDRTRVGKFLLTGLLYCGNCNGHKMQGTFDKR |
| | | EQKTYYRCLKCNRITNEKNILEPLLDEIQLLITSKEYFMSKFSD |
| | | QYDQKEEVDVSALKKELEKIKRQKEKWYDLYMDDRNPIPKE |
| | | DLFAKINELNKKEEEIYNKLNEVEPEDKEPVEEKYNRLSKMI |
| | | DFKQQFEQANDFTKKELLFSIFEKIVIYREKGKLKKITLDYTL |
| | | K |
| 29 | Int23 | MLRVALYIRVSTEEQALNGDSIRTQIEALEQYSKENDFNIVGK |
| | | YIDEGCSATNLKRPNLQRLLRDVEKDKVDLVLMTKIDRLSR |
| | | GVKNYYKIMETLEKHKCDWKTILENYDSSTAAGRLHINIMLS |
| | | VAENEAAQTSERIKFVFQDKLRRKEVISGTIPIGYKIENKHLVI |
| | | DKEKKYIVKAIFDEYEKSGSVRTLIETINNLHGELYSYNKIKNI |
| | | LRNELYIGIYNKRGFYVEDYCEPIISKKQFKQIQRILEKNKKTT |
| | | PNKNIHYHIFSGLLKCKECGYTLKGNSSNVGEKLYLSYRCST |
| | | FYLNKNCVHNVTHNEKHIENYLLTNLKPQLHKHMVKLEAQ |
| | | NEKIRRNKKSNKKDEKKKIMKKLDKIKDLYLEDLIDKETYRK |
| | | DYEKLQSQLDNITEEQESQIIDTSHIKKFLDIDINEMYSDLSRV |
| | | ERRRFWLSIIDYIEIDNNKNITINFI |
| 30 | Int24 | MKITLLYYIKKFNIYCNRYLSQQINISVDIIGFYQFKNVTNSVT |
| | | DVLKRGDNLDRICIYLRKSRADEELEKTIGVGETLSKHRKAL |
| | | LKFAKEKKLNIMEIKEEIVSADSIFFRPKMIELLKEVENNQYT |
| | | GVLVMDIQRLGRGDTEDQGIIARIFKESHTKIITPMKTYDLDD |
| | | DLDEDYFEFESFMGRKEYKMIKKRMQGGRVRSVEDGNYIAT |
| | | NPPFGYDIHWINKSRTLKFNSKESEIVKLIFKLYTEGNGAGTIS |
| | | NYLNSLGYKTKFGNNFSNSSIIFILKNPVYIGKITWKKKDIRKS |
| | | KDPHKVKDTRTRDKSEWIIADGKHEPIIDEKIWNKAQEILNN |
| | | KYHIPYKIANGPANPLAGVVICSKCNSKMVMRKYGKKLPHLI |
| | | CNNKECNNKSARFDYIEKAVLEGLDEYLKNYKVNVKANNK |
| | | TSDIEPYEQQSNALNKELILLNEQKLKLFDFLEREIYTEEIFLE |
| | | RSKNLDERINTTTLAINKIKKILDNEKKKNNKNDIVKFEKILE |
| | | GYKKTNDIQKKNELMKSLVFKIEYKKEQHQRNDGLLYIYFLS |
| | | FCVRCISYLTQFISFFVYPYRILEIYLTFSFFIISYEH |
| 31 | Int25 | MRICMYLRKSRADEELEKTLGEGETLSKHRKALLKFAKEKN |
| | | LNIVEIKEEIVSGESLFFRPKMLELLKEIENKQYSGVLVMDMQ |
| | | RLGRGNMQDQGIILETFKKSNTKIITPMKTYDLSNDFDEEYSE |
| | | FEAFMSRKELKMINRRMQGGRVRSVEDGNYIATNAPYGYDI |
| | | HWINKARTLKPNQKESEIVKLIFKLYIEGNGAGTIAKHLNSLG |
| | | YKTKFGNSFNNSSIIFILKNPVYIGKITWKKKDIRKSKDPNKV |
| | | KDTRTRDKSEWIIVDGKHDPIIDQITWKQAQEILNNRYHVPY |
| | | KLVNGPANPLAGLIICTTCKSKMVMRKLRGTDRILCKNNKC |
| | | NNISNRFDAVEKSVVESLENYLKAYKVNLPELNKTSNLKLYE |
| | | QQISTLKKELKILNEQKLKLFDFLERGIYDEDTFLKRSKNLDE |
| | | RIEITNESLSNLNQIIAKENKAIKKEDIIKFEKVLDSYKSTADIR |
| | | LKNELMKTLIFKIEYTKNKKGNDFKIKVFPKLKPLNI |
| 32 | Int26 | MIAAIYSRKSKFTGKGESVENQIEMCKEYLKRNFNNIDDIEIY |
| | | EDEGFSGKDTNRPKFKKMIKAAKNKKFNILICYRLDRISRNV |
| | | ADFSNTIEELQKYNIDFISIKEQFDTSTPMGRAMMNIAAVFAQ |
| | | LERETIAERIKDNMVELAKTGRWLGGTSPLGYKSEPIEYSNE |
| | | DGKSKKMYKLTEVENEMNIVKLIYKLYLEKRGFSSVATYLC |
| | | KNKYKGKNGGEFSRETARQIVINPVYCISDKTIFKWFKSKGA |
| | | TTYGTPDGIHGLMVYNKREGGKKDKPINEWIIAVGKHRGVIS |
| | | SDIWLKCQNLIQQNNAKSSPRSGTGEKFLLSGMVVCKECGSG |
| | | MSSWSHFNKKTNFMERYYRCNLRNRASNRCSTKMLNAYKA |
| | | EEYVANYLKELDINAIKKMYHSNKKNIIDYDAKYEVNKLNK |
| | | SIEENKKIIQGIIKKIALFDDLDILGMLKNELERLKKENDEMKI |
| | | KLKELKSILELEDEEEIFLSTMEENISNFKKFYDFVNITQKRILI |
| | | KGLVESIVWDTGGEEKILEINLIGSNTKLPSGKVKRRE |
| 33 | Int27 | MSKKVAIYTRVSTTNQAEEGYSIDEQIDKLKMYCEAMDWK |
| | | VSEIYTDAGFTGSKLTRPAMEKMITDIGLKKFDTVIVYKLDR |
| | | LSRSVRDTLYLVKDVFTKNEIDFISLSESIDTSSAMGSLFLTILS |
| | | AINEFERENIKERMTMGKIGRAKSGKSMMWAKTAFGYSHN |
| | | QETGILEINPLEASIVEQIFNEYLKGTSITKLRDKLNEDGHIAK |
| | | ELPWSYRTIRQTLDNPVYCGYIKYKNNTFEGLHKPIISHETYL |
| | | SVQKELEARQQQTYEKNNNPRPFQAKYLLSGIARCGYCGAP |
| | | LRIVLGHRRKDGSRTMKYQCVNRFPRKTKGVTTYNDNKKC |
| | | DSGAYDMQWIEDIVLKTLNGFQKSDKKLRKILNIKEESKVDT |
| | | SGFQKQLKSINNKIQKNSDLYLNDFITMDDLKKRTEMLQGEK |
| | | KLIQARINEVDKPSTSEIFDLVKSELGETTISKISYEDKKKIVN |
| | | NLISKVDVTADNIDIIFKFQLA |
| 34 | Int28 | MNEQKDKLKKYCEIKDWTIVKEYVDPGRSGSNINRPSMQQL |
| | | IKDADTGLYDAVLVYKLDRLSRSQKDTLYLIEDVFQKNNIHF |
| | | ISLSENFDTSTAFGKAMIGILSVFAQLEREQIKERMSMGRVGR |
| | | AKSGKIMEFNNPAFGYEVDGDNYKVDPLRAEIVKRIYKMYL |
| | | SGTSINKIKETLNLEGHIGNKKNWSDTRIRYILSNPTYLGKIRY |
| | | DGKTYDGKFSPIIDEETFNKTQNELKERQTATYKRFNMKLRP |
| | | FQSKYMLSGLLRCGYCGATLFVNSYVYNGKRKLRYNCPSTY |
| | | KSKQKTRTYKIMDPNCPFKLVYAKDLEPAVINEIKNLALNPQ |
| | | SIQKPVKKKPDIDVEAIQKELAKVRKQQQRLIDLYVISDDVNI |
| | | DNISKKSADLKLQEETLKKQLAPLEEPNDDDKIVAFNEILAQI |
| | | KDIDSLDYDKQKFIVKKLIKKIDVWNDNKIKIHWNI |
| 35 | Int29 | MKTAIYLRKSRADLEAEARGEGETLAKHRSTLLKIAKEMNL |
| | | NVLSVREEIVSGESLVKRPEMLALLEEIEDNKYDAVLCMDM |
| | | DRLGRGGMKEQGIILETFKRSNTKIMTPRKTYDLNDEWDEE |
| | | YSEFEAFMARKELKIITRRMQRGRIASVEAGNYLGTHAPFGY |
| | | DIHRLNKRERTLTINSEEASVVRMIFDWYANEDMGASAIRNK |
| | | LNDLGYKSKLGNDWNPYSILDILKNNIYIGKVTWQKRKEVK |
| | | RPDAVKRSCARQDKSDWIIADGKHEPIIPESLFEQAQEKLNSR |
| | | YHVPYNTNGIKNPLAGIIKCSKCGYSMVQRYPKNRKETMDC |
| | | KHRGCENKSSYTELIEKRLLEALKEWYINYKADFEAHKQGD |
| | | KLKETQVIQMNEAALRKLEKELVDVQKQKNNLHDLLERGV |
| | | YTVDMFLERSQVISDRINEITSTMENLKKEIKTEIKKEKVKKD |
| | | TIPQVEHVLDLYFKTDDPKKKNSLLKSVLEKAVYKKEKWQR |
| | | LDDFELVLYPKLPQDGDI |
| 36 | Int30 | MYRPESLDVCIYLRKSRKDVEEERRAIEEGSSYNALERHRKR |
| | | LFAIAKAENHNIIDIFEEVASGESIQERPQMQQLLRKLEGNEID |
| | | GVLVIDLDRLGRGDMLDAGMIDRAFRYSSTKIITPTDVYDPD |
| | | DESWELVFGIKSLISRQELKSITKRLQNGRIDSVKEGKHIGKK |
| | | PPYGYLKDENLRLYPDPEKAWIVKKIFELMCDGKGRQMIAA |
| | | ELDRLGIDPPVTKRGAWDSSTITSIIKNEVYTGVIVWGKFKHK |
| | | KRNGKYTRHKNPQEKWIMYENAHEPIISKELFDAANEAHSSR |
| | | HKPAVITSKKLTNPLAGILKCKLCGYTMLIQTRKDRPHNYLR |
| | | CNNPACKGKQKQSVFNLVEEKLLYSLQQIVDEYQAQKVEEV |
| | | EIDDSKLISFKEKAIISKEKELKELQAQKGNLHDLLEQGIYTVE |
| | | IFLERQKNLVERITSIENDIEVLQKEIETEQIKEHNKTEFIPALK |
| | | TVIESYHKTTNIELKNQLLKTILSTVTYYRHPDWKTNEFEIQV |
| | | YFKI |
| 37 | Int31 | MKYLALHENSRIAVYSRKSREDRDSEDTLAKHRNELEYLIKR |
| | | ENFKNVQWFEKVVSGETIDERPMFSLLLPRIENGEFDAVCAV |
| | | AMDRLSRGSQIDSGRILEAFKQSGTLFITPKKTYDLSIEGDEM |
| | | LSEFESIIARSEYRAIKRRTINGKKNATREGRLHSGSVPYGYK |
| | | WDKNLKAAVVVEEKKKIYRMMIKWFLEEEYSCTVIAEMLN |
| | | ELKVPSPSGRSIWYGEVVSEILSNDFHRGYVWFGKYKKSKSN |
| | | NSIVQNKNLDEVLIAKGHHETMKTDEEHALILNRIEKLRTYK |
| | | VAGRRLNMNTHRLSGIVRCPYCHKAQAIEQPKGRRKHVRKC |
| | | LRKSAERTKECEETKGIHEEVLFQSIMKEIKKYNESLFSPTEQ |
| | | DVNDDSYTAQLIGLREKAVKKAKGRIERIKEMYLDGDISKTE |
| | | YKEKLKISQETLQKAENELAELIASTEFQNALSAETKKEKWS |
| | | HHKVQEMIESTDGMSNSEINLILKMLISHVTYTVEDLGDGTK |
| | | NLNIKVYYN |
| 38 | Int32 | MDPQHKPTRALIVIRLSRLTDETTSPERQLEACERFCAARGW |
| | | EVVGVAEDLDVSAGTTSPFERPSLSQWIGDGKDNPGRIGEFD |
| | | TVVFYRVDRLVRRVRHLHDVIAWSERFDVNMVSATESHFDL |
| | | STTIGALIAQLVASFAEMELEGISQRATSAHRHNVQLGKFVG |
| | | GSPPFGYMPEETPDGWRLVHDPDVVPIILEVVDRVLEGEPLR |
| | | RITDDLNARGATTARDLVKQRKGKETEGHKWHSNVLKRRL |
| | | MSPAMLGYALRREPLTDSKGKPKLSAKGAKLYGPEEIVRGP |
| | | DGLPVQRAEPILPKPLFDRVVAELEARELQKEPTKRINSMLLR |
| | | VLYCGVCGQPVYRAKGQGGRSDRYRCRSIQDGANCGNPSV |
| | | LTYELDDLVEESILVLMGDSERLAHVWNPGEDNASELAEVE |
| | | ARLADRTGLIGVGAYKAGTPQRATLDTLIEADAKLYERLKA |
| | | ATPRPAGWTWEPTGETFAEWWAALDTGARNVYLRNMGVR |
| | | VTYDKRPVPEQVSAGEKPRVHLELGEVRKMAEQVAVIGTIG |
| | | TLTRNYTRLGEIGITHVDIDAGSGKAVFVTKSGERFELPLNIPE |
| | | E |
| 39 | Int33 | MKAIAIYARKSLFTGKGDSIGAQVDTCKRFIDYKFANEDYEI |
| | | RTFKDEGWSGKTTDRPDFTNMVNLIKSKKIDYVITYKLDRIG |
| | | RTARDLHNFLYELDNLGIVYLSATEPYDTTTSAGRFMISILAA |
| | | MAQMERERLAERVKSGMIQIAKKGRWLGGQCPLGFDSKREI |
| | | YIDDMGKERQMMRLTPNKEEIKIVKLIYDKYLEMGSMSQVR |
| | | KYCLENSIRGKNGGDFSTNTLKQLLTSPIYVKSSDNIFKYLES |
| | | QNINVFGTPNGNGMLTFNKTKEIRIERDKSEWIAAVGKHKGII |
| | | DDNKWLQIQQQLQQQSEKQIKSSGRQGTTSTGLLSGIIKCSK |
| | | CGNNLLIKTGHKSKKNPGTTYSYYVCGKKDNSYGHKCDNK |
| | | NVRTDEADSAVITQLKLYNKELLIKNLKEALIQNEKTDTDNIE |
| | | ILESKLKEKEKAVSNLVKKLSLIDDESISNIILNEVTNINKEIND |
| | | IKLQLSNETLKINEVTKATLDTEIYIKILENFNKKIDDITDPIEK |
| | | MNLLKSALESVEWNGDSGEFKINLIGSKKK |
| 40 | Int34 | MKVAIYTRVSTLEQREKGHSIDEQERKLRSFCDINDWTVKDV |
| | | YVDAGFSGAKRDRPELTRLLDDISEFDLVLVYKLDRLTRSVR |
| | | DLLDLLEVFENNNVAFRSATEVYDTTTAIGRLFVTLVGAMA |
| | | EWERETIRERSLMGKRAAIKKGMILTAPPFYYDRVNNTYIPN |
| | | QYKDVVLDVYNKVKKGYSIAHIARLYNNSDVKPPNGNEEW |
| | | TTRMLMHALRNPVTRGHYQWGEIYIEDSHEPIITDEMYNTIID |
| | | RLDKHTNTKVVAHTSVFRGKLICPNCGYALTLNSQKRKRKN |
| | | DTIVYKTYYCNNCKITKGMKPHHITETETLRVFKDHLSKIDL |
| | | KQYETQEKEKQSHVTIDLSKVMEQRKRYHKLYASGMMQEN |
| | | ELFELIKETDEMIEEYEKQRKQVDVKEFDICKIKEIKDVLLKS |
| | | WDIFTLEDKADFIQMSIKAINIEYTKLKRGKSSNSMKIKDIEFY |
| 238 | R4 | MNRGGPTVRADIYVRISLDRTGEELGVERQEESCRELCKSLG |
| | | MEVGQVWVDNDLSATKKNVVRPDFEAMIASNPQAIVCWHT |
| | | DRLIRVTRDLERVIDLGVNVHAVMAGHLDLSTPAGRAVART |
| | | VTAWATYEGEQKAERQKLANIQNARAGKPYTPGIRPFGYGD |
| | | DHMTIVTAEADAIRDGAKMILDGWSLSAVARYWEELKLQSP |
| | | RSMAAGGKGWSLRGVKKVLTSPRYVGRSSYLGEVVGDAQ |
| | | WPPILDPDVYYGVVAILNNPDRFSGGPRTGRTPGTLLAGIAL |
| | | CGECGKTVSGRGYRGVLVYGCKDTHTRTPRSIADGRASSSTL |
| | | ARLMFPDFLPGLLASGQAEDGQSAASKHSEAQTLRERLDGL |
| | | ATAYAEGAISLSQMTAGSEALRKKLEVIEADLVGSAGIPPFDP |
| | | VAGVAGLISGWPTTPLPTRRAWVDFCLVVTLNTQKGRHASS |
| | | MTVDDHVTIEWRDVAE |
| 239 | Dre | MPKKKRKVGSSELIISGSSGGFLRNIGKEYQEAAENFMRFMN |
| | | DQGAYAPNTLRDLRLVFHSWARWCHARQLAWFPISPEMAR |
| | | EYFLQLHDADLASTTIDKHYAMLNMLLSHCGLPPLSDDKSV |
| | | SLAMRRIRREAATEKGERTGQAIPLRWDDLKLLDVLLSRSER |
| | | LVDLRNRAFLFVAYNTLMRMSEISRIRVGDLDQTGDTVTLHI |
| | | SHTKTITTAAGLDKVLSRRTTAVLNDWLDVSGLREHPDAVL |
| | | FPPIHRSNKARITTTPLTAPAMEKIFSDAWVLLNKRDATPNKG |
| | | RYRTWTGHSARVGAAIDMAEKQVSMVEIMQEGTWKKPETL |
| | | MRYLRRGGVSVGANSRLMDS |

TABLE 3
Exemplary Split Positions for Split Recombinases

| | | SEQ |
|---|---|---|
| | N-terminal Portion AA | ID |
| Descr. | C-terminal Portion AA | NO: |
| Flp | MSQFDILCKTPPKVLVRQFVERFERPS | 41 |
| 27/28 | GEKIASCAAELTYLCWMITHNGTAIKRATFMSYNTIISNSLSFDIVNKSLQ | 42 |
| | FKYKTQKATILEASLKKLIPAWEFTIIPYNGQKHQSDITDIVSSLQLQFESS | |
| | EEADKGNSHSKKMLKALLSEGESIWEITEKILNSFEYTSRFTKTKTLYQFL | |
| | FLATFINCGRFSDIKNVDPKSFKLVQNKYLGVIIQCLVTETKTSVSRHIYFF | |
| | SARGRIDPLVYLDEFLRNSEPVLKRVNRTGNSSSNKQEYQLLKDNLVRS | |
| | YNKALKKNAPYPIFAIKNGPKSHIGRHLMTSFLSMKGLTELTNVVGNWS | |
| | DKRASAVARTTYTHQITAIPDHYFALVSRYYAYDPISKEMIALKDETNPI | |
| | EEWQHIEQLKGSAEGSIRYPAWNGIISQEVLDYLSSYINRRI | |
| Flp | MSQFDILCKTPPKVLVRQFVERFERPSGEKIASCAAELTYLCWMITHNGT | 43 |
| 396/397 | AIKRATFMSYNTIISNSLSFDIVNKSLQFKYKTQKATILEASLKKLIPAWEF | |
| | TIIPYNGQKHQSDITDIVSSLQLQFESSEEADKGNSHSKKMLKALLSEGES | |
| | IWEITEKILNSFEYTSRFTKTKTLYQFLFLATFINCGRFSDIKNVDPKSFKL | |
| | VQNKYLGVIIQCLVTETKTSVSRHIYFFSARGRIDPLVYLDEFLRNSEPVL | |
| | KRVNRTGNSSSNKQEYQLLKDNLVRSYNKALKKNAPYPIFAIKNGPKSH | |
| | IGRHLMTSFLSMKGLTELTNVVGNWSDKRASAVARTTYTHQITAIPDHY | |
| | FALVSRYYAYDPISKEMIALKDETNPIEEWQHIEQLKGSAEG | |
| | SIRYPAWNGIISQEVLDYLSSYINRRI | 44 |
| Bxb1 | MRALVVIRLSRVTDATTSPERQLESCQQLCAQRGWDV | 45 |
| 37/38 | VGVAEDLDVSGAVDPFDRKRRPNLARWLAFEEQPFDVIVAYRVDRLTR | 46 |
| | SIRHLQQLVHWAEDHKKLVVSATEAHFDTTTPFAAVVIALMGTVAQME | |
| | LEAIKERNRSAAHFNIRAGKYRGSLPPWGYLPTRVDGEWRLVPDPVQRE | |
| | RILEVYHRVVDNHEPLHLVAHDLNRRGVLSPKDYFAQLQGREPQGREW | |
| | SATALKRSMISEAMLGYATLNGKTVRDDDGAPLVRAEPILTREQLEALR | |
| | AELVKTSRAKPAVSTPSLLLRVLFCAVCGEPAYKFAGGGRKHPRYRCRS | |
| | MGFPKHCGNGTVAMAEWDAFCEEQVLDLLGDAERLEKVWVAGSDSA | |
| | VELAEVNAELVDLTSLIGSPAYRAGSPQREALDARIAALAARQEELEGLE | |
| | ARPSGWEWRETGQRFGDWWREQDTAAKNTWLRSMNVRLTFDVRGGL | |
| | TRTIDFGDLQEYEQHLRLGSVVERLHTGMS | |
| Bxb1 | MRALVVIRLSRVTDATTSPERQLESCQQLCAQRGWDVVGVAEDLDVSG | 47 |
| 169/170 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVD | |
| | GEWRLVPDPVQRERILEVYHRVVDNHEPLHLVAHDLNRRGVLSPKDYF | 48 |
| | AQLQGREPQGREWSATALKRSMISEAMLGYATLNGKTVRDDDGAPLVR | |
| | AEPILTREQLEALRAELVKTSRAKPAVSTPSLLLRVLFCAVCGEPAYKFA | |
| | GGGRKHPRYRCRSMGFPKHCGNGTVAMAEWDAFCEEQVLDLLGDAER | |
| | LEKVWVAGSDSAVELAEVNAELVDLTSLIGSPAYRAGSPQREALDARIA | |
| | ALAARQEELEGLEARPSGWEWRETGQRFGDWWREQDTAAKNTWLRS | |
| | MNVRLTFDVRGGLTRTIDFGDLQEYEQHLRLGSVVERLHTGMS | |
| Bxb1 | MRALVVIRLSRVTDATTSPERQLESCQQLCAQRGWDVVGVAEDLDVSG | 49 |
| 208/209 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRR | |
| | GVLSPKDYFAQLQGREPQGREWSATALKRSMISEAMLGYATLNGKTVR | 50 |
| | DDDGAPLVRAEPILTREQLEALRAELVKTSRAKPAVSTPSLLLRVLFCAV | |
| | CGEPAYKFAGGGRKHPRYRCRSMGFPKHCGNGTVAMAEWDAFCEEQV | |
| | LDLLGDAERLEKVWVAGSDSAVELAEVNAELVDLTSLIGSPAYRAGSPQ | |
| | REALDARIAALAARQEELEGLEARPSGWEWRETGQRFGDWWREQDTA | |
| | AKNTWLRSMNVRLTFDVRGGLTRTIDFGDLQEYEQHLRLGSVVERLHT | |
| | GMS | |
| Bxb1 | MRALVVIRLSRVTDATTSPERQLESCQQLCAQRGWDVVGVAEDLDVSG | 51 |
| 222/223 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQG | |
| | REPQGREWSATALKRSMISEAMLGYATLNGKTVRDDDGAPLVRAEPILT | 52 |
| | REQLEALRAELVKTSRAKPAVSTPSLLLRVLFCAVCGEPAYKFAGGGRK | |
| | HPRYRCRSMGFPKHCGNGTVAMAEWDAFCEEQVLDLLGDAERLEKVW | |
| | VAGSDSAVELAEVNAELVDLTSLIGSPAYRAGSPQREALDARIAALAAR | |
| | QEELEGLEARPSGWEWRETGQRFGDWWREQDTAAKNTWLRSMNVRLT | |
| | FDVRGGLTRTIDFGDLQEYEQHLRLGSVVERLHTGMS | |
| Bxb1 | MRALVVIRLSRVTDATTSPERQLESCQQLCAQRGWDVVGVAEDLDVSG | 53 |
| 259/260 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDD | |
| | GAPLVRAEPILTREQLEALRAELVKTSRAKPAVSTPSLLLRVLFCAVCGE | 54 |
| | PAYKFAGGGRKHPRYRCRSMGFPKHCGNGTVAMAEWDAFCEEQVLDL | |
| | LGDAERLEKVWVAGSDSAVELAEVNAELVDLTSLIGSPAYRAGSPQREA | |
| | LDARIAALAARQEELEGLEARPSGWEWRETGQRFGDWWREQDTAAKN | |
| | TWLRSMNVRLTFDVRGGLTRTIDFGDLQEYEQHLRLGSVVERLHTGMS | |
| Bxb1 | MRALVVIRLSRVTDATTSPERQLESCQQLCAQRGWDVVGVAEDLDVSG | 55 |
| 262/263 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAP | |
| | LVRAEPILTREQLEALRAELVKTSRAKPAVSTPSLLLRVLFCAVCGEPAY | 56 |
| | KFAGGGRKHPRYRCRSMGFPKHCGNGTVAMAEWDAFCEEQVLDLLGD | |
| | AERLEKVWVAGSDSAVELAEVNAELVDLTSLIGSPAYRAGSPQREALDA | |
| | RIAALAARQEELEGLEARPSGWEWRETGQRFGDWWREQDTAAKNTWL | |
| | RSMNVRLTFDVRGGLTRTIDFGDLQEYEQHLRLGSVVERLHTGMS | |
| Bxb1 | MRALVVIRLSRVTDATTSPERQLESCQQLCAQRGWDVVGVAEDLDVSG | 57 |
| 363/364 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAPLVRAEPILTREQLEALRAELVKTSRAKPAV | |
| | STPSLLLRVLFCAVCGEPAYKFAGGGRKHPRYRCRSMGFPKHCGNGTV | |
| | AMAEWDAFCEEQVLDLLGDAERL | |
| | EKVWVAGSDSAVELAEVNAELVDLTSLIGSPAYRAGSPQREALDARIAA | 58 |
| | LAARQEELEGLEARPSGWEWRETGQRFGDWWREQDTAAKNTWLRSM | |
| | NVRLTFDVRGGLTRTIDFGDLQEYEQHLRLGSVVERLHTGMS | |
| Bxb1 | MRALVVIRLSRVTDATTSPERQLESCQQLCAQRGWDVVGVAEDLDVSG | 59 |
| 370/371 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAPLVRAEPILTREQLEALRAELVKTSRAKPAV | |
| | STPSLLLRVLFCAVCGEPAYKFAGGGRKHPRYRCRSMGFPKHCGNGTV | |
| | AMAEWDAFCEEQVLDLLGDAERLEKVWVAG | |
| | SDSAVELAEVNAELVDLTSLIGSPAYRAGSPQREALDARIAALAARQEEL | 60 |
| | EGLEARPSGWEWRETGQRFGDWWREQDTAAKNTWLRSMNVRLTFDV | |
| | RGGLTRTIDFGDLQEYEQHLRLGSVVERLHTGMS | |
| Bxb1 | MRALVVIRLSRVTDATTSPERQLESCQQLCAQRGWDVVGVAEDLDVSG | 61 |
| 399/400 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAPLVRAEPILTREQLEALRAELVKTSRAKPAV | |
| | STPSLLLRVLFCAVCGEPAYKFAGGGRKHPRYRCRSMGFPKHCGNGTV | |
| | AMAEWDAFCEEQVLDLLGDAERLEKVWVAGSDSAVELAEVNAELVDL | |
| | TSLIGSPAYRAG | |
| | SPQREALDARIAALAARQEELEGLEARPSGWEWRETGQRFGDWWREQD | 62 |
| | TAAKNTWLRSMNVRLTFDVRGGLTRTIDFGDLQEYEQHLRLGSVVERL | |
| | HTGMS | |
| Bxb1 | MRALVVIRLSRVTDATTSPERQLESCQQLCAQRGWDVVGVAEDLDVSG | 63 |
| 400/401 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAPLVRAEPILTREQLEALRAELVKTSRAKPAV | |
| | STPSLLLRVLFCAVCGEPAYKFAGGGRKHPRYRCRSMGFPKHCGNGTV | |
| | AMAEWDAFCEEQVLDLLGDAERLEKVWVAGSDSAVELAEVNAELVDL | |
| | TSLIGSPAYRAGS | |
| | PQREALDARIAALAARQEELEGLEARPSGWEWRETGQRFGDWWREQDT | 64 |
| | AAKNTWLRSMNVRLTFDVRGGLTRTIDFGDLQEYEQHLRLGSVVERLH | |
| | TGMS | |
| Bxb1 | MRALVVIRLSRVTDATTSPERQLESCQQLCAQRGWDVVGVAEDLDVSG | 65 |
| 468/469 | AVDPFDRKRRPNLARWLAFEEQPFDVIVAYRVDRLTRSIRHLQQLVHWA | |
| | EDHKKLVVSATEAHFDTTTPFAAVVIALMGTVAQMELEAIKERNRSAAH | |
| | FNIRAGKYRGSLPPWGYLPTRVDGEWRLVPDPVQRERILEVYHRVVDN | |
| | HEPLHLVAHDLNRRGVLSPKDYFAQLQGREPQGREWSATALKRSMISEA | |
| | MLGYATLNGKTVRDDDGAPLVRAEPILTREQLEALRAELVKTSRAKPAV | |
| | STPSLLLRVLFCAVCGEPAYKFAGGGRKHPRYRCRSMGFPKHCGNGTV | |
| | AMAEWDAFCEEQVLDLLGDAERLEKVWVAGSDSAVELAEVNAELVDL | |
| | TSLIGSPAYRAGSPQREALDARIAALAARQEELEGLEARPSGWEWRETG | |
| | QRFGDWWREQDTAAKNTWLRSMNVRLTFDVRG | |
| | GLTRTIDFGDLQEYEQHLRLGSVVERLHTGMS | 66 |
| PhiC31 | MDTYAGAYDRQSRERENSSAASPATQRSANEDKAADLQREVERDGGRF | 67 |
| 233/234 | RFVGHFSEAPGTSAFGTAERPEFERILNECRAGRLNMIIVYDVSRFSRLKV | |
| | MDAIPIVSELLALGVTIVSTQEGVFRQGNVMDLIHLIMRLDASHKESSLK | |
| | SAKILDTKNLQRELGGYVGGKAPYGFELVSETKEITRNGRMVNVVINKL | |
| | AHSTTPLTGPFEFEPDVIRWWWREIKTHKHLPFKP | |
| | GSQAAIHPGSITGLCKRMDADAVPTRGETIGKKTASSAWDPATVMRILR | 68 |
| | DPRIAGFAAEVIYKKKPDGTPTTKIEGYRIQRDPITLRPVELDCGPIIEPAE | |
| | WYELQAWLDGRGRGKGLSRGQAILSAMDKLYCECGAVMTSKRGEESIK | |
| | DSYRCRRRKVVDPSAPGQHEGTCNVSMAALDKFVAERIFNKIRHAEGDE | |
| | ETLALLWEAARRFGKLTEAPEKSGERANLVAERADALNALEELYEDRA | |
| | AGAYDGPVGRKHFRKQQAALTLRQQGAEERLAELEAAEAPKLPLDQWF | |
| | PEDADADPTGPKSWWGRASVDDKRVFVGLFVDKIVVTKSTTGRGQGTP | |
| | IEKRASITWAKPPTDDDEDDAQDGTEDVAA | |
| PhiC31 | MDTYAGAYDRQSRERENSSAASPATQRSANEDKAADLQREVERDGGRF | 69 |
| 571/572 | RFVGHFSEAPGTSAFGTAERPEFERILNECRAGRLNMIIVYDVSRFSRLKV | |
| | MDAIPIVSELLALGVTIVSTQEGVFRQGNVMDLIHLIMRLDASHKESSLK | |
| | SAKILDTKNLQRELGGYVGGKAPYGFELVSETKEITRNGRMVNVVINKL | |
| | AHSTTPLTGPFEFEPDVIRWWWREIKTHKHLPFKPGSQAAIHPGSITGLCK | |
| | RMDADAVPTRGETIGKKTASSAWDPATVMRILRDPRIAGFAAEVIYKKK | |
| | PDGTPTTKIEGYRIQRDPITLRPVELDCGPIIEPAEWYELQAWLDGRGRGK | |
| | GLSRGQAILSAMDKLYCECGAVMTSKRGEESIKDSYRCRRRKVVDPSAP | |
| | GQHEGTCNVSMAALDKFVAERIFNKIRHAEGDEETLALLWEAARRFGK | |
| | LTEAPEKSGERANLVAERADALNALEELYEDRAAGAYDGPVGRKHFRK | |
| | QQAALTLRQQGAEERLAELEAAEAPKLPLDQWFPEDADADPTGPKSWW | |
| | GRASVDDKRVFVGLFVDKIVVTKSTTGRG | |
| | QGTPIEKRASITWAKPPTDDDEDDAQDGTEDVAA | 70 |
| TP901 | MTKKVAIYTRVSTTNQAEEGFSIDEQIDRLTKYAEAMGWQVSDTYTDA | 71 |
| 326/327 | GFSGAKLERPAMQRLINDIENKAFDTVLVYKLDRLSRSVRDTLYLVKDV | |
| | FTKNKIDFISLNESIDTSSAMGSLFLTILSAINEFERENIKERMTMGKLGRA | |
| | KSGKSMMWTKTAFGYYHNRKTGILEIVPLQATIVEQIFTDYLSGISLTKL | |
| | RDKLNESGHIGKDIPWSYRTLRQTLDNPVYCGYIKFKDSLFEGMHKPIIP | |
| | YETYLKVQKELEERQQQTYERNNNPRPFQAKYMLSGMARCGYCGAPL | |
| | KIVLGHKRKDGSRTMKYHCANRFPRKTKGI | |
| | TVYNDNKKCDSGTYDLSNLENTVIDNLIGFQENNDSLLKIINGNNQPILD | 72 |
| | TSSFKKQISQIDKKIQKNSDLYLNDFITMDELKDRTDSLQAEKKLLKAKIS | |
| | ENKFNDSTDVFELVKTQLGSIPINELSYDNKKKIVNNLVSKVDVTADNV | |
| | DIIFKFQLA | |
| Cre | MSNLLTVHQNLPALPVDATSDEVRKNLMDMFRDRQAFSEHTWKMLLS | 73 |
| 229/230 | VCRSWAAWCKLNNRKWFPAEPEDVRDYLLYLQARGLAVKTIQQHLGQ | |
| | LNMLHRRSGLPRPSDSNAVSLVMRRIRKENVDAGERAKQALAFERTDFD | |
| | QVRSLMENSDRCQDIRNLAFLGIAYNTLLRIAEIARIRVKDISRTDGGRML | |
| | IHIGRTKTLVSTAGVEKALSLGVTKLVERWISVSG | |
| | VADDPNNYLFCRVRKNGVAAPSATSQLSTRALEGIFEATHRLIYGAKDD | 74 |
| | SGQRYLAWSGHSARVGAARDMARAGVSIPEIMQAGGWTNVNIVMNYI | |
| | RNLDSETGAMVRLLEDGD | |
| Cre | MSNLLTVHQNLPALPVDATSDEVRKNLMDMFRDRQAFSEHTWKMLLS | 75 |
| 269/270 | VCRSWAAWCKLNNRKWFPAEPEDVRDYLLYLQARGLAVKTIQQHLGQ | |
| | LNMLHRRSGLPRPSDSNAVSLVMRRIRKENVDAGERAKQALAFERTDFD | |
| | QVRSLMENSDRCQDIRNLAFLGIAYNTLLRIAEIARIRVKDISRTDGGRML | |
| | IHIGRTKTLVSTAGVEKALSLGVTKLVERWISVSGVADDPNNYLFCRVR | |
| | KNGVAAPSATSQLSTRALEGIFEATH | |
| | RLIYGAKDDSGQRYLAWSGHSARVGAARDMARAGVSIPEIMQAGGWT | 76 |
| | NVNIVMNYIRNLDSETGAMVRLLEDGD | |
| Vcre | MIENQLSLLGDFSGVRPDDVKTAIQAAQKKGINVAENEQFKAAFEHLLN | 77 |
| 269/270 | EFKKREERYSPNTLRRLESAWTCFVDWCLANHRHSLPATPDTVEAFFIER | |
| | AEELHRNTLSVYRWAISRVHRVAGCPDPCLDIYVEDRLKAIARKKVREG | |
| | EAVKQASPFNEQHLLKLTSLWYRSDKLLLRRNLALLAVAYESMLRASEL | |
| | ANIRVSDMELAGDGTAILTIPITKTNHSGEPDTCILSQDVVSLLMDYTEAG | |
| | KLDMSSDGFLFVGVSKHNTCI | |
| | KPKKDKQTGEVLHKPITTKTVEGVFYSAWETLDLGRQGVKPFTAHSAR | 78 |
| | VGAAQDLLKKGYNTLQIQQSGRWSSGAMVARYGRAILARDGAMAHSR | |
| | VKTRSAPMQWGKDEKD | |

TABLE 4
Exemplary Dimerization Domain Pair Amino Acid Sequences

| | | | SEQ |
|---|---|---|---|
| | | Portion 1 AA | ID |
| Description | | Portion 2 AA | NO: |
| Gibberellic | GID1 | MAASDEVNLIESRTVVPLNTWVLISNFKVAYNILRRPDGTFN | 79 |
| Acid (GA) | | RHLAEYLDRKVTANANPVDGVFSFDVLIDRRINLLSRVYRPA | |
| Inducible | | YADQEQPPSILDLEKPVDGDIVPVILFFHGGSFAHSSANSAIY | |
| | | DTLCRRLVGLCKCVVVSVNYRRAPENPYPCAYDDGWIALN | |
| | | WVNSRSWLKSKKDSKVHIFLAGDSSGGNIAHNVALRAGESG | |
| | | IDVLGNILLNPMFGGNERTESEKSLDGKYFVTVRDRDWYWK | |
| | | AFLPEGEDREHPACNPFSPRGKSLEGVSFPKSLVVVAGLDLIR | |
| | | DWQLAYAEGLKKAGQEVKLMHLEKATVGFYLLPNNNHFH | |
| | | NVMDEISAFVNAEC | |
| | GA1 | QDKKTMMMNEEDDGNGMDELLAVLGYKVRSSEMADVAQ | 80 |
| | | KLEQLEVMMSNVQEDDLSQLATETVHYNPAELYTWLDSML | |
| | | TDLN | |
| Abscisic | ABI | PLYGFTSICGRRPEMEDAVSTIPRFLQSSSGSMLDGRFDPQSA | 81 |
| Acid | | AHFFGVYDGHGGSQVANYCRERMHLALAEEIAKEKPMLCD | |
| (ABA) | | GDTWLEKWKKALFNSFLRVDSEIGSVAPETVGSTSVVAVVF | |
| Inducible | | PSHIFVANCGDSRAVLCRGKTALPLSVDHKPDREDEAARIEA | |
| | | AGGKVIQWNGARVFGVLAMSRSIGDRYLKPSIIPDPEVTAVK | |
| | | RVKEDDCLILASDGVWDVMTDEEACEMARKRILLWHKKNA | |
| | | VAGDASLLADERRKEGKDPAAMSAAEYLSKLAIQRGSKDNI | |
| | | SVVVVDLK | |
| | PYL | TQDEFTQLSQSIAEFHTYQLGNGRCSSLLAQRIHAPPETVWSV | 82 |
| | | VRRFDRPQIYKHFIKSCNVSEDFEMRVGCTRDVNVISGLPAN | |
| | | TSRERLDLLDDDRRVTGFSITGGEHRLRNYKSVTTVHRFEKE | |
| | | EEEERIWTVVLESYVVDVPEGNSEEDTRLFADTVIRLNLQKL | |
| | | ASITEAMN | |
| Rapalog | FRB | ILWHEMWHEGLEEASRLYFGERNVKGMFEVLEPLHAMMER | 83 |
| (Rap) | | GPQTLKETSFNQAYGRDLMEAQEWCRKYMKSGNVKDLLQA | |
| Inducible | | WDLYYHVFRRIS | |
| | FKBP | GVQVETISPGDGRTFPKRGQTCVVHYTGMLEDGKKFDSSRD | 84 |
| | | RNKPFKFMLGKQEVIRGWEEGVAQMSVGQRAKLTISPDYAY | |
| | | GATGHPGIIPPHATLVFDVELLKLE | |

TABLE 5
Exemplary IRES Polycistronic Expression Element Nucleic Acid sequences

| SEQ ID | | |
|---|---|---|
| NO: | Description | Sequence |
| 85 | EMCV | CCCTAACGTTACTGGCCGAAGCCGCTTGGAATAAGGCCGGTGT |
| | IRES | GCGTTTGTCTATATGTTATTTTCCACCATATTGCCGTCTTTTGGC |
| | | AATGTGAGGGCCCGGAAACCTGGCCCTGTCTTCTTGACGAGCAT |
| | | TCCTAGGGGTCTTTCCCCTCTCGCCAAAGGAATGCAAGGTCTGT |
| | | TGAATGTCGTGAAGGAAGCAGTTCCTCTGGAAGCTTCTTGAAGA |
| | | CAAACAACGTCTGTAGCGACCCTTTGCAGGCAGCGGAACCCCC |
| | | CACCTGGCGACAGGTGCCTCTGCGGCCAAAAGCCACGTGTATA |
| | | AGATACACCTGCAAAGGCGGCACAACCCCAGTGCCACGTTGTG |
| | | AGTTGGATAGTTGTGGAAAGAGTCAAATGGCTCTCCTCAAGCGT |
| | | ATTCAACAAGGGGCTGAAGGATGCCCAGAAGGTACCCCATTGT |
| | | ATGGGATCTGATCTGGGGCCTCGGTGCACATGCTTTACATGTGT |
| | | TTAGTCGAGGTTAAAAAAACGTCTAGGCCCCCCGAACCACGGG |
| | | GACGTGGTTTTCCTTTGAAAAACACGATGATAAT |
| 86 | PV IRES | AGTTCAATAGAAGGGGGTACAAACCAGTACCACCACGAACAAG |
| | | CACTTCTGTTTCCCCGGTGATGTCGTATAGACTGCTTGCGTGGTT |
| | | GAAAGCGACGGATCCGTTATCCGCTTATGTACTTCGAGAAGCCC |
| | | AGTACCACCTCGGAATCTTCGATGCGTTGCGCTCAGCACTCAAC |
| | | CCCAGAGTGTAGCTTAGGCTGATGAGTCTGGACATCCCTCACCG |
| | | GTGACGGTGGTCCAGGCTGCGTTGGCGGCCTACCTATGGCTAAC |
| | | GCCATGGGACGCTAGTTGTGAACAAGGTGTGAAGAGCCTATTG |
| | | AGCTACATAAGAATCCTCCGGCCCCTGAATGCGGCTAATCCCAA |
| | | CCTCGGAGCAGGTGGTCACAAACCAGTGATTGGCCTGTCGTAA |
| | | CGCGCAAGTCCGTGGCGGAACCGACTACTTTGGGTGTCCGTGTT |
| | | TCCTTTTATTTTATTGTGGCTGCTTATGGTGACAATCACAGATTG |
| | | TTATCATAAAGCGA |
| 87 | FMDV | AAGCAGGTTTCCCCAACTGACACAAACCGTGCAATTTGGAACTC |
| | IRES | CGCCTGGTCTTTCCAGGTCTAGAGGGGTGACACTTTGTACTGTG |
| | | TTTGGCTCCACGCTCGGTCCACTGGCGAGTGTTAGTAACAGCAC |
| | | CGTTGCTTCGTAGCGGAGCATGATGGCCGTGGGAACTCCTCCTT |
| | | GGTAACAAGGACCCACGGGGCCGAAAGCCACGTCCAATCGGAC |
| | | CCATCATGTGTGCAACCCCAGCACAGCAACTTTTCTGCGAAACT |
| | | CACTTCAAGGTGACACTGATACTGGTACTCAAACACTGGTGACA |
| | | GGCTAAGGATGCCCTTCAGGTACCCCGAGGTAACACGCGTCAC |
| | | TCGGGATCTGAGAAGGGGACTGGGGCTTCTATAAAAGCGTCCA |
| | | GTTTAAAAAGCTTCTATGCCTGAATAGGTGACCGGAGGCCGGC |
| | | ACCTTTTCTTTACAGCCACTGACTTT |

TABLE 6
Exemplary 2A Polycistronic Expression Element
Amino Acid Sequences

| SEQ ID | | |
|---|---|---|
| NO: | Description | Sequence |
| 88 | P2A | ATNFSLLKQAGDVEENPGP |
| 89 | T2A | EGRGSLLTCGDVEENPGP |
| 236 | E2A | QCTNYALLKLAGDVESNPGP |
| 237 | F2A | VKQTLNFDLLKLAGDVESNPGP |

TABLE 7
Exemplary Split Recombinases Amino Acid Sequences

| SEQ | | |
|---|---|---|
| ID | | |
| NO: | Descr. | Sequence |
| 90 | GA_Flp | MSQFDILCKTPPKVLVRQFVERFERPSSGGSGSGSSGGSGTMAASDEVN |
| | (27/28) | LIESRTVVPLNTWVLISNFKVAYNILRRPDGTFNRHLAEYLDRKVTANA |
| | | NPVDGVFSFDVLIDRRINLLSRVYRPAYADQEQPPSILDLEKPVDGDIVP |
| | | VILFFHGGSFAHSSANSAIYDTLCRRLVGLCKCVVVSVNYRRAPENPYP |
| | | CAYDDGWIALNWVNSRSWLKSKKDSKVHIFLAGDSSGGNIAHNVALR |
| | | AGESGIDVLGNILLNPMFGGNERTESEKSLDGKYFVTVRDRDWYWKAF |
| | | LPEGEDREHPACNPFSPRGKSLEGVSFPKSLVVVAGLDLIRDWQLAYAE |
| | | GLKKAGQEVKLMHLEKATVGFYLLPNNNHFHNVMDEISAFVNAECPK |
| | | KKRKVATNFSLLKQAGDVEENPGPMKRDHHHHHHQDKKTMMMNEE |
| | | DDGNGMDELLAVLGYKVRSSEMADVAQKLEQLEVMMSNVQEDDLSQ |
| | | LATETVHYNPAELYTWLDSMLTDLNSGGSGSGSSGGSGTGEKIASCAA |
| | | ELTYLCWMITHNGTAIKRATFMSYNTIISNSLSFDIVNKSLQFKYKTQKA |
| | | TILEASLKKLIPAWEFTIIPYNGQKHQSDITDIVSSLQLQFESSEEADKGN |
| | | SHSKKMLKALLSEGESIWEITEKILNSFEYTSRFTKTKTLYQFLFLATFIN |
| | | CGRFSDIKNVDPKSFKLVQNKYLGVIIQCLVTETKTSVSRHIYFFSARGRI |
| | | DPLVYLDEFLRNSEPVLKRVNRTGNSSSNKQEYQLLKDNLVRSYNKAL |
| | | KKNAPYPIFAIKNGPKSHIGRHLMTSFLSMKGLTELTNVVGNWSDKRAS |
| | | AVARTTYTHQITAIPDHYFALVSRYYAYDPISKEMIALKDETNPIEEWQ |
| | | HIEQLKGSAEGSIRYPAWNGIISQEVLDYLSSYINRRIPKKKRKV |
| 91 | ABA_Flp | MSQFDILCKTPPKVLVRQFVERFERPSGEKIASCAAELTYLCWMITHNG |
| | (396/397) | TAIKRATFMSYNTIISNSLSFDIVNKSLQFKYKTQKATILEASLKKLIPAW |
| | | EFTIIPYNGQKHQSDITDIVSSLQLQFESSEEADKGNSHSKKMLKALLSE |
| | | GESIWEITEKILNSFEYTSRFTKTKTLYQFLFLATFINCGRFSDIKNVDPKS |
| | | FKLVQNKYLGVIIQCLVTETKTSVSRHIYFFSARGRIDPLVYLDEFLRNS |
| | | EPVLKRVNRTGNSSSNKQEYQLLKDNLVRSYNKALKKNAPYPIFAIKN |
| | | GPKSHIGRHLMTSFLSMKGLTELTNVVGNWSDKRASAVARTTYTHQIT |
| | | AIPDHYFALVSRYYAYDPISKEMIALKDETNPIEEWQHIEQLKGSAEGSG |
| | | GSGSGSSGGSGTPLYGFTSICGRRPEMEDAVSTIPRFLQSSSGSMLDGRF |
| | | DPQSAAHFFGVYDGHGGSQVANYCRERMHLALAEEIAKEKPMLCDGD |
| | | TWLEKWKKALFNSFLRVDSEIGSVAPETVGSTSVVAVVFPSHIFVANCG |
| | | DSRAVLCRGKTALPLSVDHKPDREDEAARIEAAGGKVIQWNGARVFG |
| | | VLAMSRSIGDRYLKPSIIPDPEVTAVKRVKEDDCLILASDGVWDVMTDE |
| | | EACEMARKRILLWHKKNAVAGDASLLADERRKEGKDPAAMSAAEYLS |
| | | KLAIQRGSKDNISVVVVDLKDYKDDDDKPKKKRKVATNFSLLKQAGD |
| | | VEENPGPMAPTQDEFTQLSQSIAEFHTYQLGNGRCSSLLAQRIHAPPETV |
| | | WSVVRRFDRPQIYKHFIKSCNVSEDFEMRVGCTRDVNVISGLPANTSRE |
| | | RLDLLDDDRRVTGFSITGGEHRLRNYKSVTTVHRFEKEEEEERIWTVVL |
| | | ESYVVDVPEGNSEEDTRLFADTVIRLNLQKLASITEAMNYPYDVPDYAS |
| | | GGSGSGSSGGSGTSIRYPAWNGIISQEVLDYLSSYINRRIPKKKRKV |
| 92 | GA_Bxb1 | MRALVVIRLSRVTDATTSPERQLESCQQLCAQRGWDVSGGSGSGSSGG |
| | (37/38) | SGTMAASDEVNLIESRTVVPLNTWVLISNFKVAYNILRRPDGTFNRHLA |
| | | EYLDRKVTANANPVDGVFSFDVLIDRRINLLSRVYRPAYADQEQPPSIL |
| | | DLEKPVDGDIVPVILFFHGGSFAHSSANSAIYDTLCRRLVGLCKCVVVS |
| | | VNYRRAPENPYPCAYDDGWIALNWVNSRSWLKSKKDSKVHIFLAGDS |
| | | SGGNIAHNVALRAGESGIDVLGNILLNPMFGGNERTESEKSLDGKYFVT |
| | | VRDRDWYWKAFLPEGEDREHPACNPFSPRGKSLEGVSFPKSLVVVAGL |
| | | DLIRDWQLAYAEGLKKAGQEVKLMHLEKATVGFYLLPNNNHFHNVM |
| | | DEISAFVNAECPKKKRKVATNFSLLKQAGDVEENPGPMKRDHHHHHH |
| | | QDKKTMMMNEEDDGNGMDELLAVLGYKVRSSEMADVAQKLEQLEV |
| | | MMSNVQEDDLSQLATETVHYNPAELYTWLDSMLTDLNSGGSGSGSSG |
| | | GSGTVGVAEDLDVSGAVDPFDRKRRPNLARWLAFEEQPFDVIVAYRVD |
| | | RLTRSIRHLQQLVHWAEDHKKLVVSATEAHFDTTTPFAAVVIALMGTV |
| | | AQMELEAIKERNRSAAHFNIRAGKYRGSLPPWGYLPTRVDGEWRLVPD |
| | | PVQRERILEVYHRVVDNHEPLHLVAHDLNRRGVLSPKDYFAQLQGREP |
| | | QGREWSATALKRSMISEAMLGYATLNGKTVRDDDGAPLVRAEPILTRE |
| | | QLEALRAELVKTSRAKPAVSTPSLLLRVLFCAVCGEPAYKFAGGGRKH |
| | | PRYRCRSMGFPKHCGNGTVAMAEWDAFCEEQVLDLLGDAERLEKVW |
| | | VAGSDSAVELAEVNAELVDLTSLIGSPAYRAGSPQREALDARIAALAAR |
| | | QEELEGLEARPSGWEWRETGQRFGDWWREQDTAAKNTWLRSMNVRL |
| | | TFDVRGGLTRTIDFGDLQEYEQHLRLGSVVERLHTGMSGSPKKKRKV |
| 93 | GA_Bxb1 | MRALVVIRLSRVTDATTSPERQLESCQQLCAQRGWDVVGVAEDLDVS |
| | (169/170) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDSGGSGSGSSGGSGTMAASDEVN |
| | | LIESRTVVPLNTWVLISNFKVAYNILRRPDGTFNRHLAEYLDRKVTANA |
| | | NPVDGVFSFDVLIDRRINLLSRVYRPAYADQEQPPSILDLEKPVDGDIVP |
| | | VILFFHGGSFAHSSANSAIYDTLCRRLVGLCKCVVVSVNYRRAPENPYP |
| | | CAYDDGWIALNWVNSRSWLKSKKDSKVHIFLAGDSSGGNIAHNVALR |
| | | AGESGIDVLGNILLNPMFGGNERTESEKSLDGKYFVTVRDRDWYWKAF |
| | | LPEGEDREHPACNPFSPRGKSLEGVSFPKSLVVVAGLDLIRDWQLAYAE |
| | | GLKKAGQEVKLMHLEKATVGFYLLPNNNHFHNVMDEISAFVNAECPK |
| | | KKRKVATNFSLLKQAGDVEENPGPMKRDHHHHHHQDKKTMMMNEE |
| | | DDGNGMDELLAVLGYKVRSSEMADVAQKLEQLEVMMSNVQEDDLSQ |
| | | LATETVHYNPAELYTWLDSMLTDLNSGGSGSGSSGGSGTGEWRLVPDP |
| | | VQRERILEVYHRVVDNHEPLHLVAHDLNRRGVLSPKDYFAQLQGREPQ |
| | | GREWSATALKRSMISEAMLGYATLNGKTVRDDDGAPLVRAEPILTREQ |
| | | LEALRAELVKTSRAKPAVSTPSLLLRVLFCAVCGEPAYKFAGGGRKHP |
| | | RYRCRSMGFPKHCGNGTVAMAEWDAFCEEQVLDLLGDAERLEKVWV |
| | | AGSDSAVELAEVNAELVDLTSLIGSPAYRAGSPQREALDARIAALAARQ |
| | | EELEGLEARPSGWEWRETGQRFGDWWREQDTAAKNTWLRSMNVRLT |
| | | FDVRGGLTRTIDFGDLQEYEQHLRLGSVVERLHTGMSGSPKKKRKV |
| 94 | GA_Bxb1 | MRALVVIRLSRVTDATTSPERQLESCQQLCAQRGWDVVGVAEDLDVS |
| | (195/196) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHSGGSGSGSSGGSGTMAASDEVNLIESRTVVPLNTWVLISNFKVA |
| | | YNILRRPDGTFNRHLAEYLDRKVTANANPVDGVFSFDVLIDRRINLLSR |
| | | VYRPAYADQEQPPSILDLEKPVDGDIVPVILFFHGGSFAHSSANSAIYDT |
| | | LCRRLVGLCKCVVVSVNYRRAPENPYPCAYDDGWIALNWVNSRSWLK |
| | | SKKDSKVHIFLAGDSSGGNIAHNVALRAGESGIDVLGNILLNPMFGGNE |
| | | RTESEKSLDGKYFVTVRDRDWYWKAFLPEGEDREHPACNPFSPRGKSL |
| | | EGVSFPKSLVVVAGLDLIRDWQLAYAEGLKKAGQEVKLMHLEKATVG |
| | | FYLLPNNNHFHNVMDEISAFVNAECPKKKRKVATNFSLLKQAGDVEEN |
| | | PGPMKRDHHHHHHQDKKTMMMNEEDDGNGMDELLAVLGYKVRSSE |
| | | MADVAQKLEQLEVMMSNVQEDDLSQLATETVHYNPAELYTWLDSML |
| | | TDLNSGGSGSGSSGGSGTEPLHLVAHDLNRRGVLSPKDYFAQLQGREP |
| | | QGREWSATALKRSMISEAMLGYATLNGKTVRDDDGAPLVRAEPILTRE |
| | | QLEALRAELVKTSRAKPAVSTPSLLLRVLFCAVCGEPAYKFAGGGRKH |
| | | PRYRCRSMGFPKHCGNGTVAMAEWDAFCEEQVLDLLGDAERLEKVW |
| | | VAGSDSAVELAEVNAELVDLTSLIGSPAYRAGSPQREALDARIAALAAR |
| | | QEELEGLEARPSGWEWRETGQRFGDWWREQDTAAKNTWLRSMNVRL |
| | | TFDVRGGLTRTIDFGDLQEYEQHLRLGSVVERLHTGMSGSPKKKRKV |
| 95 | GA_Bxb1 | MRALVVIRLSRVTDATTSPERQLESCQQLCAQRGWDVVGVAEDLDVS |
| | (208/209) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRSGGSGSGSSGGSGTMAASDEVNLIESRTVVP |
| | | LNTWVLISNFKVAYNILRRPDGTFNRHLAEYLDRKVTANANPVDGVFS |
| | | FDVLIDRRINLLSRVYRPAYADQEQPPSILDLEKPVDGDIVPVILFFHGGS |
| | | FAHSSANSAIYDTLCRRLVGLCKCVVVSVNYRRAPENPYPCAYDDGWI |
| | | ALNWVNSRSWLKSKKDSKVHIFLAGDSSGGNIAHNVALRAGESGIDVL |
| | | GNILLNPMFGGNERTESEKSLDGKYFVTVRDRDWYWKAFLPEGEDRE |
| | | HPACNPFSPRGKSLEGVSFPKSLVVVAGLDLIRDWQLAYAEGLKKAGQ |
| | | EVKLMHLEKATVGFYLLPNNNHFHNVMDEISAFVNAECPKKKRKVAT |
| | | NFSLLKQAGDVEENPGPMKRDHHHHHHQDKKTMMMNEEDDGNGMD |
| | | ELLAVLGYKVRSSEMADVAQKLEQLEVMMSNVQEDDLSQLATETVHY |
| | | NPAELYTWLDSMLTDLNSGGSGSGSSGGSGTGVLSPKDYFAQLQGREP |
| | | QGREWSATALKRSMISEAMLGYATLNGKTVRDDDGAPLVRAEPILTRE |
| | | QLEALRAELVKTSRAKPAVSTPSLLLRVLFCAVCGEPAYKFAGGGRKH |
| | | PRYRCRSMGFPKHCGNGTVAMAEWDAFCEEQVLDLLGDAERLEKVW |
| | | VAGSDSAVELAEVNAELVDLTSLIGSPAYRAGSPQREALDARIAALAAR |
| | | QEELEGLEARPSGWEWRETGQRFGDWWREQDTAAKNTWLRSMNVRL |
| | | TFDVRGGLTRTIDFGDLQEYEQHLRLGSVVERLHTGMSGSPKKKRKV |
| 96 | GA_Bxb1 | MRALVVIRLSRVTDATTSPERQLESCQQLCAQRGWDVVGVAEDLDVS |
| | (222/223) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGSGGSGSGSSGGSGTMA |
| | | ASDEVNLIESRTVVPLNTWVLISNFKVAYNILRRPDGTFNRHLAEYLDR |
| | | KVTANANPVDGVFSFDVLIDRRINLLSRVYRPAYADQEQPPSILDLEKP |
| | | VDGDIVPVILFFHGGSFAHSSANSAIYDTLCRRLVGLCKCVVVSVNYRR |
| | | APENPYPCAYDDGWIALNWVNSRSWLKSKKDSKVHIFLAGDSSGGNIA |
| | | HNVALRAGESGIDVLGNILLNPMFGGNERTESEKSLDGKYFVTVRDRD |
| | | WYWKAFLPEGEDREHPACNPFSPRGKSLEGVSFPKSLVVVAGLDLIRD |
| | | WQLAYAEGLKKAGQEVKLMHLEKATVGFYLLPNNNHFHNVMDEISAF |
| | | VNAECPKKKRKVATNFSLLKQAGDVEENPGPMKRDHHHHHHQDKKT |
| | | MMMNEEDDGNGMDELLAVLGYKVRSSEMADVAQKLEQLEVMMSNV |
| | | QEDDLSQLATETVHYNPAELYTWLDSMLTDLNSGGSGSGSSGGSGTRE |
| | | PQGREWSATALKRSMISEAMLGYATLNGKTVRDDDGAPLVRAEPILTR |
| | | EQLEALRAELVKTSRAKPAVSTPSLLLRVLFCAVCGEPAYKFAGGGRK |
| | | HPRYRCRSMGFPKHCGNGTVAMAEWDAFCEEQVLDLLGDAERLEKV |
| | | WVAGSDSAVELAEVNAELVDLTSLIGSPAYRAGSPQREALDARIAALA |
| | | ARQEELEGLEARPSGWEWRETGQRFGDWWREQDTAAKNTWLRSMNV |
| | | RLTFDVRGGLTRTIDFGDLQEYEQHLRLGSVVERLHTGMSGSPKKKRK |
| | | V |
| 97 | GA_Bxb1 | MRALVVIRLSRVTDATTSPERQLESCQQLCAQRGWDVVGVAEDLDVS |
| | (259/260) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDSGGSGSGSSGGSGTMAASDEVNLIESR |
| | | TVVPLNTWVLISNFKVAYNILRRPDGTFNRHLAEYLDRKVTANANPVD |
| | | GVFSFDVLIDRRINLLSRVYRPAYADQEQPPSILDLEKPVDGDIVPVILFF |
| | | HGGSFAHSSANSAIYDTLCRRLVGLCKCVVVSVNYRRAPENPYPCAYD |
| | | DGWIALNWVNSRSWLKSKKDSKVHIFLAGDSSGGNIAHNVALRAGES |
| | | GIDVLGNILLNPMFGGNERTESEKSLDGKYFVTVRDRDWYWKAFLPEG |
| | | EDREHPACNPFSPRGKSLEGVSFPKSLVVVAGLDLIRDWQLAYAEGLK |
| | | KAGQEVKLMHLEKATVGFYLLPNNNHFHNVMDEISAFVNAECPKKKR |
| | | KVATNFSLLKQAGDVEENPGPMKRDHHHHHHQDKKTMMMNEEDDG |
| | | NGMDELLAVLGYKVRSSEMADVAQKLEQLEVMMSNVQEDDLSQLAT |
| | | ETVHYNPAELYTWLDSMLTDLNSGGSGSGSSGGSGTGAPLVRAEPILTR |
| | | EQLEALRAELVKTSRAKPAVSTPSLLLRVLFCAVCGEPAYKFAGGGRK |
| | | HPRYRCRSMGFPKHCGNGTVAMAEWDAFCEEQVLDLLGDAERLEKV |
| | | WVAGSDSAVELAEVNAELVDLTSLIGSPAYRAGSPQREALDARIAALA |
| | | ARQEELEGLEARPSGWEWRETGQRFGDWWREQDTAAKNTWLRSMNV |
| | | RLTFDVRGGLTRTIDFGDLQEYEQHLRLGSVVERLHTGMSGSPKKKRK |
| | | V |
| 98 | GA_Bxb1 | MRALVVIRLSRVTDATTSPERQLESCQQLCAQRGWDVVGVAEDLDVS |
| | (262/263) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPSGGSGSGSSGGSGTMAASDEVNLI |
| | | ESRTVVPLNTWVLISNFKVAYNILRRPDGTFNRHLAEYLDRKVTANAN |
| | | PVDGVFSFDVLIDRRINLLSRVYRPAYADQEQPPSILDLEKPVDGDIVPVI |
| | | LFFHGGSFAHSSANSAIYDTLCRRLVGLCKCVVVSVNYRRAPENPYPCA |
| | | YDDGWIALNWVNSRSWLKSKKDSKVHIFLAGDSSGGNIAHNVALRAG |
| | | ESGIDVLGNILLNPMFGGNERTESEKSLDGKYFVTVRDRDWYWKAFLP |
| | | EGEDREHPACNPFSPRGKSLEGVSFPKSLVVVAGLDLIRDWQLAYAEGL |
| | | KKAGQEVKLMHLEKATVGFYLLPNNNHFHNVMDEISAFVNAECPKKK |
| | | RKVATNFSLLKQAGDVEENPGPMKRDHHHHHHQDKKTMMMNEEDD |
| | | GNGMDELLAVLGYKVRSSEMADVAQKLEQLEVMMSNVQEDDLSQLA |
| | | TETVHYNPAELYTWLDSMLTDLNSGGSGSGSSGGSGTLVRAEPILTREQ |
| | | LEALRAELVKTSRAKPAVSTPSLLLRVLFCAVCGEPAYKFAGGGRKHP |
| | | RYRCRSMGFPKHCGNGTVAMAEWDAFCEEQVLDLLGDAERLEKVWV |
| | | AGSDSAVELAEVNAELVDLTSLIGSPAYRAGSPQREALDARIAALAARQ |
| | | EELEGLEARPSGWEWRETGQRFGDWWREQDTAAKNTWLRSMNVRLT |
| | | FDVRGGLTRTIDFGDLQEYEQHLRLGSVVERLHTGMSGSPKKKRKV |
| 99 | GA_Bxbl | MRALVVIRLSRVTDATTSPERQLESCQQLCAQRGWDVVGVAEDLDVS |
| | (363/364) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPLVRAEPILTREQLEALRAELVKTS |
| | | RAKPAVSTPSLLLRVLFCAVCGEPAYKFAGGGRKHPRYRCRSMGFPKH |
| | | CGNGTVAMAEWDAFCEEQVLDLLGDAERLSGGSGSGSSGGSGTMAAS |
| | | DEVNLIESRTVVPLNTWVLISNFKVAYNILRRPDGTFNRHLAEYLDRKV |
| | | TANANPVDGVFSFDVLIDRRINLLSRVYRPAYADQEQPPSILDLEKPVD |
| | | GDIVPVILFFHGGSFAHSSANSAIYDTLCRRLVGLCKCVVVSVNYRRAP |
| | | ENPYPCAYDDGWIALNWVNSRSWLKSKKDSKVHIFLAGDSSGGNIAHN |
| | | VALRAGESGIDVLGNILLNPMFGGNERTESEKSLDGKYFVTVRDRDWY |
| | | WKAFLPEGEDREHPACNPFSPRGKSLEGVSFPKSLVVVAGLDLIRDWQL |
| | | AYAEGLKKAGQEVKLMHLEKATVGFYLLPNNNHFHNVMDEISAFVNA |
| | | ECPKKKRKVATNFSLLKQAGDVEENPGPMKRDHHHHHHQDKKTMMM |
| | | NEEDDGNGMDELLAVLGYKVRSSEMADVAQKLEQLEVMMSNVQEDD |
| | | LSQLATETVHYNPAELYTWLDSMLTDLNSGGSGSGSSGGSGTEKVWV |
| | | AGSDSAVELAEVNAELVDLTSLIGSPAYRAGSPQREALDARIAALAARQ |
| | | EELEGLEARPSGWEWRETGQRFGDWWREQDTAAKNTWLRSMNVRLT |
| | | FDVRGGLTRTIDFGDLQEYEQHLRLGSVVERLHTGMSGSPKKKRKV |
| 100 | GA_Bxb1 | MRALVVIRLSRVTDATTSPERQLESCQQLCAQRGWDVVGVAEDLDVS |
| | (370/371) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPLVRAEPILTREQLEALRAELVKTS |
| | | RAKPAVSTPSLLLRVLFCAVCGEPAYKFAGGGRKHPRYRCRSMGFPKH |
| | | CGNGTVAMAEWDAFCEEQVLDLLGDAERLEKVWVAGSGGSGSGSSG |
| | | GSGTMAASDEVNLIESRTVVPLNTWVLISNFKVAYNILRRPDGTFNRHL |
| | | AEYLDRKVTANANPVDGVFSFDVLIDRRINLLSRVYRPAYADQEQPPSI |
| | | LDLEKPVDGDIVPVILFFHGGSFAHSSANSAIYDTLCRRLVGLCKCVVVS |
| | | VNYRRAPENPYPCAYDDGWIALNWVNSRSWLKSKKDSKVHIFLAGDS |
| | | SGGNIAHNVALRAGESGIDVLGNILLNPMFGGNERTESEKSLDGKYFVT |
| | | VRDRDWYWKAFLPEGEDREHPACNPFSPRGKSLEGVSFPKSLVVVAGL |
| | | DLIRDWQLAYAEGLKKAGQEVKLMHLEKATVGFYLLPNNNHFHNVM |
| | | DEISAFVNAECPKKKRKVATNFSLLKQAGDVEENPGPMKRDHHHHHH |
| | | QDKKTMMMNEEDDGNGMDELLAVLGYKVRSSEMADVAQKLEQLEV |
| | | MMSNVQEDDLSQLATETVHYNPAELYTWLDSMLTDLNSGGSGSGSSG |
| | | GSGTSDSAVELAEVNAELVDLTSLIGSPAYRAGSPQREALDARIAALAA |
| | | RQEELEGLEARPSGWEWRETGQRFGDWWREQDTAAKNTWLRSMNVR |
| | | LTFDVRGGLTRTIDFGDLQEYEQHLRLGSVVERLHTGMSGSPKKKRKV |
| 101 | GA_Bxb1 | MRALVVIRLSRVTDATTSPERQLESCQQLCAQRGWDVVGVAEDLDVS |
| | (399/400) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPLVRAEPILTREQLEALRAELVKTS |
| | | RAKPAVSTPSLLLRVLFCAVCGEPAYKFAGGGRKHPRYRCRSMGFPKH |
| | | CGNGTVAMAEWDAFCEEQVLDLLGDAERLEKVWVAGSDSAVELAEV |
| | | NAELVDLTSLIGSPAYRAGSGGSGSGSSGGSGTMAASDEVNLIESRTVV |
| | | PLNTWVLISNFKVAYNILRRPDGTFNRHLAEYLDRKVTANANPVDGVF |
| | | SFDVLIDRRINLLSRVYRPAYADQEQPPSILDLEKPVDGDIVPVILFFHGG |
| | | SFAHSSANSAIYDTLCRRLVGLCKCVVVSVNYRRAPENPYPCAYDDGW |
| | | IALNWVNSRSWLKSKKDSKVHIFLAGDSSGGNIAHNVALRAGESGIDV |
| | | LGNILLNPMFGGNERTESEKSLDGKYFVTVRDRDWYWKAFLPEGEDRE |
| | | HPACNPFSPRGKSLEGVSFPKSLVVVAGLDLIRDWQLAYAEGLKKAGQ |
| | | EVKLMHLEKATVGFYLLPNNNHFHNVMDEISAFVNAECPKKKRKVAT |
| | | NFSLLKQAGDVEENPGPMKRDHHHHHHQDKKTMMMNEEDDGNGMD |
| | | ELLAVLGYKVRSSEMADVAQKLEQLEVMMSNVQEDDLSQLATETVHY |
| | | NPAELYTWLDSMLTDLNSGGSGSGSSGGSGTSPQREALDARIAALAAR |
| | | QEELEGLEARPSGWEWRETGQRFGDWWREQDTAAKNTWLRSMNVRL |
| | | TFDVRGGLTRTIDFGDLQEYEQHLRLGSVVERLHTGMSGSPKKKRKV |
| 102 | GA_Bxb1 | MRALVVIRLSRVTDATTSPERQLESCQQLCAQRGWDVVGVAEDLDVS |
| | (440/441) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPLVRAEPILTREQLEALRAELVKTS |
| | | RAKPAVSTPSLLLRVLFCAVCGEPAYKFAGGGRKHPRYRCRSMGFPKH |
| | | CGNGTVAMAEWDAFCEEQVLDLLGDAERLEKVWVAGSDSAVELAEV |
| | | NAELVDLTSLIGSPAYRAGSPQREALDARIAALAARQEELEGLEARPSG |
| | | WEWRETGQRFGSGGSGSGSSGGSGTMAASDEVNLIESRTVVPLNTWVL |
| | | ISNFKVAYNILRRPDGTFNRHLAEYLDRKVTANANPVDGVFSFDVLIDR |
| | | RINLLSRVYRPAYADQEQPPSILDLEKPVDGDIVPVILFFHGGSFAHSSA |
| | | NSAIYDTLCRRLVGLCKCVVVSVNYRRAPENPYPCAYDDGWIALNWV |
| | | NSRSWLKSKKDSKVHIFLAGDSSGGNIAHNVALRAGESGIDVLGNILLN |
| | | PMFGGNERTESEKSLDGKYFVTVRDRDWYWKAFLPEGEDREHPACNP |
| | | FSPRGKSLEGVSFPKSLVVVAGLDLIRDWQLAYAEGLKKAGQEVKLMH |
| | | LEKATVGFYLLPNNNHFHNVMDEISAFVNAECPKKKRKVATNFSLLKQ |
| | | AGDVEENPGPMKRDHHHHHHQDKKTMMMNEEDDGNGMDELLAVLG |
| | | YKVRSSEMADVAQKLEQLEVMMSNVQEDDLSQLATETVHYNPAELYT |
| | | WLDSMLTDLNSGGSGSGSSGGSGTDWWREQDTAAKNTWLRSMNVRL |
| | | TFDVRGGLTRTIDFGDLQEYEQHLRLGSVVERLHTGMSGSPKKKRKV |
| 103 | GA_Bxb1 | MRALVVIRLSRVTDATTSPERQLESCQQLCAQRGWDVVGVAEDLDVS |
| | (468/469) | GAVDPFDRKRRPNLARWLAFEEQPFDVIVAYRVDRLTRSIRHLQQLVH |
| | | WAEDHKKLVVSATEAHFDTTTPFAAVVIALMGTVAQMELEAIKERNRS |
| | | AAHFNIRAGKYRGSLPPWGYLPTRVDGEWRLVPDPVQRERILEVYHRV |
| | | VDNHEPLHLVAHDLNRRGVLSPKDYFAQLQGREPQGREWSATALKRS |
| | | MISEAMLGYATLNGKTVRDDDGAPLVRAEPILTREQLEALRAELVKTS |
| | | RAKPAVSTPSLLLRVLFCAVCGEPAYKFAGGGRKHPRYRCRSMGFPKH |
| | | CGNGTVAMAEWDAFCEEQVLDLLGDAERLEKVWVAGSDSAVELAEV |
| | | NAELVDLTSLIGSPAYRAGSPQREALDARIAALAARQEELEGLEARPSG |
| | | WEWRETGQRFGDWWREQDTAAKNTWLRSMNVRLTFDVRGSGGSGS |
| | | GSSGGSGTMAASDEVNLIESRTVVPLNTWVLISNFKVAYNILRRPDGTF |
| | | NRHLAEYLDRKVTANANPVDGVFSFDVLIDRRINLLSRVYRPAYADQE |
| | | QPPSILDLEKPVDGDIVPVILFFHGGSFAHSSANSAIYDTLCRRLVGLCKC |
| | | VVVSVNYRRAPENPYPCAYDDGWIALNWVNSRSWLKSKKDSKVHIFL |
| | | AGDSSGGNIAHNVALRAGESGIDVLGNILLNPMFGGNERTESEKSLDGK |
| | | YFVTVRDRDWYWKAFLPEGEDREHPACNPFSPRGKSLEGVSFPKSLVV |
| | | VAGLDLIRDWQLAYAEGLKKAGQEVKLMHLEKATVGFYLLPNNNHFH |
| | | NVMDEISAFVNAECPKKKRKVATNFSLLKQAGDVEENPGPMKRDHHH |
| | | HHHQDKKTMMMNEEDDGNGMDELLAVLGYKVRSSEMADVAQKLEQ |
| | | LEVMMSNVQEDDLSQLATETVHYNPAELYTWLDSMLTDLNSGGSGSG |
| | | SSGGSGTGLTRTIDFGDLQEYEQHLRLGSVVERLHTGMSPKKKRKV |
| 104 | GA_PhiC31 | MDTYAGAYDRQSRERENSSAASPATQRSANEDKAADLQREVERDGGR |
| | (233/234) | FRFVGHFSEAPGTSAFGTAERPEFERILNECRAGRLNMIIVYDVSRFSRL |
| | | KVMDAIPIVSELLALGVTIVSTQEGVFRQGNVMDLIHLIMRLDASHKES |
| | | SLKSAKILDTKNLQRELGGYVGGKAPYGFELVSETKEITRNGRMVNVVI |
| | | NKLAHSTTPLTGPFEFEPDVIRWWWREIKTHKHLPFKPSGGSGSGSSGG |
| | | SGTMAASDEVNLIESRTVVPLNTWVLISNFKVAYNILRRPDGTFNRHLA |
| | | EYLDRKVTANANPVDGVFSFDVLIDRRINLLSRVYRPAYADQEQPPSIL |
| | | DLEKPVDGDIVPVILFFHGGSFAHSSANSAIYDTLCRRLVGLCKCVVVS |
| | | VNYRRAPENPYPCAYDDGWIALNWVNSRSWLKSKKDSKVHIFLAGDS |
| | | SGGNIAHNVALRAGESGIDVLGNILLNPMFGGNERTESEKSLDGKYFVT |
| | | VRDRDWYWKAFLPEGEDREHPACNPFSPRGKSLEGVSFPKSLVVVAGL |
| | | DLIRDWQLAYAEGLKKAGQEVKLMHLEKATVGFYLLPNNNHFHNVM |
| | | DEISAFVNAECPKKKRKVATNFSLLKQAGDVEENPGPMKRDHHHHHH |
| | | QDKKTMMMNEEDDGNGMDELLAVLGYKVRSSEMADVAQKLEQLEV |
| | | MMSNVQEDDLSQLATETVHYNPAELYTWLDSMLTDLNSGGSGSGSSG |
| | | GSGTGSQAAIHPGSITGLCKRMDADAVPTRGETIGKKTASSAWDPATV |
| | | MRILRDPRIAGFAAEVIYKKKPDGTPTTKIEGYRIQRDPITLRPVELDCGP |
| | | IIEPAEWYELQAWLDGRGRGKGLSRGQAILSAMDKLYCECGAVMTSK |
| | | RGEESIKDSYRCRRRKVVDPSAPGQHEGTCNVSMAALDKFVAERIFNKI |
| | | RHAEGDEETLALLWEAARRFGKLTEAPEKSGERANLVAERADALNALE |
| | | ELYEDRAAGAYDGPVGRKHFRKQQAALTLRQQGAEERLAELEAAEAP |
| | | KLPLDQWFPEDADADPTGPKSWWGRASVDDKRVFVGLFVDKIVVTKS |
| | | TTGRGQGTPIEKRASITWAKPPTDDDEDDAQDGTEDVAAPKKKRKV |
| 105 | RAP_PhiC31 | MDTYAGAYDRQSRERENSSAASPATQRSANEDKAADLQREVERDGGR |
| | (571/572) | FRFVGHFSEAPGTSAFGTAERPEFERILNECRAGRLNMIIVYDVSRFSRL |
| | | KVMDAIPIVSELLALGVTIVSTQEGVFRQGNVMDLIHLIMRLDASHKES |
| | | SLKSAKILDTKNLQRELGGYVGGKAPYGFELVSETKEITRNGRMVNVVI |
| | | NKLAHSTTPLTGPFEFEPDVIRWWWREIKTHKHLPFKPGSQAAIHPGSIT |
| | | GLCKRMDADAVPTRGETIGKKTASSAWDPATVMRILRDPRIAGFAAEV |
| | | IYKKKPDGTPTTKIEGYRIQRDPITLRPVELDCGPIIEPAEWYELQAWLD |
| | | GRGRGKGLSRGQAILSAMDKLYCECGAVMTSKRGEESIKDSYRCRRRK |
| | | VVDPSAPGQHEGTCNVSMAALDKFVAERIFNKIRHAEGDEETLALLWE |
| | | AARRFGKLTEAPEKSGERANLVAERADALNALEELYEDRAAGAYDGP |
| | | VGRKHFRKQQAALTLRQQGAEERLAELEAAEAPKLPLDQWFPEDADA |
| | | DPTGPKSWWGRASVDDKRVFVGLFVDKIVVTKSTTGRGSGGSGSGSSG |
| | | GSGTILWHEMWHEGLEEASRLYFGERNVKGMFEVLEPLHAMMERGPQ |
| | | TLKETSFNQAYGRDLMEAQEWCRKYMKSGNVKDLLQAWDLYYHVFR |
| | | RISPKKKRKVATNFSLLKQAGDVEENPGPMSRGVQVETISPGDGRTFPK |
| | | RGQTCVVHYTGMLEDGKKFDSSRDRNKPFKFMLGKQEVIRGWEEGVA |
| | | QMSVGQRAKLTISPDYAYGATGHPGIIPPHATLVFDVELLKLESGGSGS |
| | | GSSGGSGTQGTPIEKRASITWAKPPTDDDEDDAQDGTEDVAAPKKKRK |
| | | V |
| 106 | GA_TP901 | MTKKVAIYTRVSTTNQAEEGFSIDEQIDRLTKYAEAMGWQVSDTYTDA |
| | (326/327) | GFSGAKLERPAMQRLINDIENKAFDTVLVYKLDRLSRSVRDTLYLVKD |
| | | VFTKNKIDFISLNESIDTSSAMGSLFLTILSAINEFERENIKERMTMGKLG |
| | | RAKSGKSMMWTKTAFGYYHNRKTGILEIVPLQATIVEQIFTDYLSGISLT |
| | | KLRDKLNESGHIGKDIPWSYRTLRQTLDNPVYCGYIKFKDSLFEGMHKP |
| | | IIPYETYLKVQKELEERQQQTYERNNNPRPFQAKYMLSGMARCGYCGA |
| | | PLKIVLGHKRKDGSRTMKYHCANRFPRKTKGISGGSGSGSSGGSGTMA |
| | | ASDEVNLIESRTVVPLNTWVLISNFKVAYNILRRPDGTFNRHLAEYLDR |
| | | KVTANANPVDGVFSFDVLIDRRINLLSRVYRPAYADQEQPPSILDLEKP |
| | | VDGDIVPVILFFHGGSFAHSSANSAIYDTLCRRLVGLCKCVVVSVNYRR |
| | | APENPYPCAYDDGWIALNWVNSRSWLKSKKDSKVHIFLAGDSSGGNIA |
| | | HNVALRAGESGIDVLGNILLNPMFGGNERTESEKSLDGKYFVTVRDRD |
| | | WYWKAFLPEGEDREHPACNPFSPRGKSLEGVSFPKSLVVVAGLDLIRD |
| | | WQLAYAEGLKKAGQEVKLMHLEKATVGFYLLPNNNHFHNVMDEISAF |
| | | VNAECPKKKRKVATNFSLLKQAGDVEENPGPMKRDHHHHHHQDKKT |
| | | MMMNEEDDGNGMDELLAVLGYKVRSSEMADVAQKLEQLEVMMSNV |
| | | QEDDLSQLATETVHYNPAELYTWLDSMLTDLNSGGSGSGSSGGSGTTV |
| | | YNDNKKCDSGTYDLSNLENTVIDNLIGFQENNDSLLKIINGNNQPILDTS |
| | | SFKKQISQIDKKIQKNSDLYLNDFITMDELKDRTDSLQAEKKLLKAKISE |
| | | NKFNDSTDVFELVKTQLGSIPINELSYDNKKKIVNNLVSKVDVTADNVD |
| | | IIFKFQLA |
| 107 | GA_Cre | MSNLLTVHQNLPALPVDATSDEVRKNLMDMFRDRQAFSEHTWKMLLS |
| | (229/230) | VCRSWAAWCKLNNRKWFPAEPEDVRDYLLYLQARGLAVKTIQQHLG |
| | | QLNMLHRRSGLPRPSDSNAVSLVMRRIRKENVDAGERAKQALAFERTD |
| | | FDQVRSLMENSDRCQDIRNLAFLGIAYNTLLRIAEIARIRVKDISRTDGG |
| | | RMLIHIGRTKTLVSTAGVEKALSLGVTKLVERWISVSGSGGSGSGSSGG |
| | | SGTMAASDEVNLIESRTVVPLNTWVLISNFKVAYNILRRPDGTFNRHLA |
| | | EYLDRKVTANANPVDGVFSFDVLIDRRINLLSRVYRPAYADQEQPPSIL |
| | | DLEKPVDGDIVPVILFFHGGSFAHSSANSAIYDTLCRRLVGLCKCVVVS |
| | | VNYRRAPENPYPCAYDDGWIALNWVNSRSWLKSKKDSKVHIFLAGDS |
| | | SGGNIAHNVALRAGESGIDVLGNILLNPMFGGNERTESEKSLDGKYFVT |
| | | VRDRDWYWKAFLPEGEDREHPACNPFSPRGKSLEGVSFPKSLVVVAGL |
| | | DLIRDWQLAYAEGLKKAGQEVKLMHLEKATVGFYLLPNNNHFHNVM |
| | | DEISAFVNAECPKKKRKVATNFSLLKQAGDVEENPGPATMKRDHHHH |
| | | HHQDKKTMMMNEEDDGNGMDELLAVLGYKVRSSEMADVAQKLEQL |
| | | EVMMSNVQEDDLSQLATETVHYNPAELYTWLDSMLTDLNSGGSGSGS |
| | | SGGSGTVADDPNNYLFCRVRKNGVAAPSATSQLSTRALEGIFEATHRLI |
| | | YGAKDDSGQRYLAWSGHSARVGAARDMARAGVSIPEIMQAGGWTNV |
| | | NIVMNYIRNLDSETGAMVRLLEDGDPKKKRKV |
| 108 | PYL1- | MAPTQDEFTQLSQSIAEFHTYQLGNGRCSSLLAQRIHAPPETVWSVVRR |
| | CreC(271)- | FDRPQIYKHFIKSCNVSEDFEMRVGCTRDVNVISGLPANTSRERLDLLD |
| | 2A- | DDRRVTGFSITGGEHRLRNYKSVTTVHRFEKEEEEERIWTVVLESYVVD |
| | CreN(270)- | VPEGNSEEDTRLFADTVIRLNLQKLASITEAMNYPYDVPDYASGGSGSG |
| | ABI | SSGGSGTLIYGAKDDSGQRYLAWSGHSARVGAARDMARAGVSIPEIMQ |
| | | AGGWTNVNIVMNYIRNLDSETGAMVRLLEDGDPKKKRKVATNFSLLK |
| | | QAGDVEENPGPMSNLLTVHQNLPALPVDATSDEVRKNLMDMFRDRQA |
| | | FSEHTWKMLLSVCRSWAAWCKLNNRKWFPAEPEDVRDYLLYLQARG |
| | | LAVKTIQQHLGQLNMLHRRSGLPRPSDSNAVSLVMRRIRKENVDAGER |
| | | AKQALAFERTDFDQVRSLMENSDRCQDIRNLAFLGIAYNTLLRIAEIARI |
| | | RVKDISRTDGGRMLIHIGRTKTLVSTAGVEKALSLGVTKLVERWISVSG |
| | | VADDPNNYLFCRVRKNGVAAPSATSQLSTRALEGIFEATHRSGGSGSGS |
| | | SGGSGTPLYGFTSICGRRPEMEAAVSTIPRFLQSSSGSMLDGRFDPQSAA |
| | | HFFGVYDGHGGSQVANYCRERMHLALAEEIAKEKPMLCDGDTWLEK |
| | | WKKALFNSFLRVDSEIESVAPETVGSTSVVAVVFPSHIFVANCGDSRAV |
| | | LCRGKTALPLSVDHKPDREDEAARIEAAGGKVIQWNGARVFGVLAMS |
| | | RSIGDRYLKPSIIPDPEVTAVKRVKEDDCLILASDGVWDVMTDEEACEM |
| | | ARKRILLWHKKNAVAGDASLLADERRKEGKDPAAMSAAEYLSKLAIQ |
| | | RGSKDNISVVVVDLKDYKDDDDKPKKKRKV |
| 109 | CreN(270)- | MSNLLTVHQNLPALPVDATSDEVRKNLMDMFRDRQAFSEHTWKMLLS |
| | ABI-2A- | VCRSWAAWCKLNNRKWFPAEPEDVRDYLLYLQARGLAVKTIQQHLG |
| | PYL1- | QLNMLHRRSGLPRPSDSNAVSLVMRRIRKENVDAGERAKQALAFERTD |
| | CreC(271) | FDQVRSLMENSDRCQDIRNLAFLGIAYNTLLRIAEIARIRVKDISRTDGG |
| | | RMLIHIGRTKTLVSTAGVEKALSLGVTKLVERWISVSGVADDPNNYLFC |
| | | RVRKNGVAAPSATSQLSTRALEGIFEATHRSGGSGSGSSGGSGTPLYGF |
| | | TSICGRRPEMEAAVSTIPRFLQSSSGSMLDGRFDPQSAAHFFGVYDGHG |
| | | GSQVANYCRERMHLALAEEIAKEKPMLCDGDTWLEKWKKALFNSFLR |
| | | VDSEIESVAPETVGSTSVVAVVFPSHIFVANCGDSRAVLCRGKTALPLSV |
| | | DHKPDREDEAARIEAAGGKVIQWNGARVFGVLAMSRSIGDRYLKPSIIP |
| | | DPEVTAVKRVKEDDCLILASDGVWDVMTDEEACEMARKRILLWHKKN |
| | | AVAGDASLLADERRKEGKDPAAMSAAEYLSKLAIQRGSKDNISVVVVD |
| | | LKDYKDDDDKPKKKRKVATNFSLLKQAGDVEENPGPMAPTQDEFTQL |
| | | SQSIAEFHTYQLGNGRCSSLLAQRIHAPPETVWSVVRRFDRPQIYKHFIK |
| | | SCNVSEDFEMRVGCTRDVNVISGLPANTSRERLDLLDDDRRVTGFSITG |
| | | GEHRLRNYKSVTTVHRFEKEEEEERIWTVVLESYVVDVPEGNSEEDTRL |
| | | FADTVIRLNLQKLASITEAMNYPYDVPDYASGGSGSGSSGGSGTLIYGA |
| | | KDDSGQRYLAWSGHSARVGAARDMARAGVSIPEIMQAGGWTNVNIV |
| | | MNYIRNLDSETGAMVRLLEDGDPKKKRKV |
| 110 | GA_Vcre | MIENQLSLLGDFSGVRPDDVKTAIQAAQKKGINVAENEQFKAAFEHLL |
| | (269/270) | NEFKKREERYSPNTLRRLESAWTCFVDWCLANHRHSLPATPDTVEAFFI |
| | | ERAEELHRNTLSVYRWAISRVHRVAGCPDPCLDIYVEDRLKAIARKKV |
| | | REGEAVKQASPFNEQHLLKLTSLWYRSDKLLLRRNLALLAVAYESMLR |
| | | ASELANIRVSDMELAGDGTAILTIPITKTNHSGEPDTCILSQDVVSLLMD |
| | | YTEAGKLDMSSDGFLFVGVSKHNTCISGGSGSGSSGGSGTMAASDEVN |
| | | LIESRTVVPLNTWVLISNFKVAYNILRRPDGTFNRHLAEYLDRKVTANA |
| | | NPVDGVFSFDVLIDRRINLLSRVYRPAYADQEQPPSILDLEKPVDGDIVP |
| | | VILFFHGGSFAHSSANSAIYDTLCRRLVGLCKCVVVSVNYRRAPENPYP |
| | | CAYDDGWIALNWVNSRSWLKSKKDSKVHIFLAGDSSGGNIAHNVALR |
| | | AGESGIDVLGNILLNPMFGGNERTESEKSLDGKYFVTVRDRDWYWKAF |
| | | LPEGEDREHPACNPFSPRGKSLEGVSFPKSLVVVAGLDLIRDWQLAYAE |
| | | GLKKAGQEVKLMHLEKATVGFYLLPNNNHFHNVMDEISAFVNAECPK |
| | | KKRKVATNFSLLKQAGDVEENPGPMKRDHHHHHHQDKKTMMMNEE |
| | | DDGNGMDELLAVLGYKVRSSEMADVAQKLEQLEVMMSNVQEDDLSQ |
| | | LATETVHYNPAELYTWLDSMLTDLNSGGSGSGSSGGSGTKPKKDKQTG |
| | | EVLHKPITTKTVEGVFYSAWETLDLGRQGVKPFTAHSARVGAAQDLLK |
| | | KGYNTLQIQQSGRWSSGAMVARYGRAILARDGAMAHSRVKTRSAPMQ |
| | | WGKDEKDPKKKRKV |

TABLE 8
Exemplary Split Recombinases Nucleic Acid Sequences

| SEQ | | |
|---|---|---|
| ID | | |
| NO: | Descr. | Sequence |
| 111 | GA_Flp | atgagccagttcgacatcctgtgcaagaccccccccaaggtgctggtgcggcagttcgtggagagattcgagag |
| | (27/28) | gcccagctccggagggtctggctccggatcaagtggtggcagcggtaccatggccgccagcgacgaagtgaa |
| | | cctgatcgagagcagaaccgtggtgcccctgaacacctgggtgctgatctccaacttcaaggtggcctacaacat |
| | | cctgcggaggcccgacggcaccttcaacagacacctggccgagtacctggaccggaaagtgaccgccaacgc |
| | | caaccctgtggacggcgtgttcagcttcgacgtgctgatcgaccggcggatcaacctgctgagccgggtgtaca |
| | | gacccgcctacgccgatcaggaacagcccccctctatcctggatctggaaaagcccgtggatggcgacatcgtg |
| | | cccgtgatcctgttcttccacggcggcagctttgcccacagcagcgccaatagcgccatctacgacaccctgtgc |
| | | agacggctcgtgggcctgtgcaaatgcgtggtggtgtccgtgaactaccgcagagcccccgagaacccttaccc |
| | | ctgcgcctacgatgatggctggatcgccctgaactgggtcaacagcagaagctggctgaagtccaagaaagaca |
| | | gcaaggtgcacatctttctggccggcgatagcagcggcggcaatatcgcccataacgtggccctgagagccgg |
| | | cgagtctggcatcgatgtgctgggcaatatcctgctgaaccccatgttcggcggcaacgagcggaccgagagcg |
| | | agaagtctctggacggcaagtacttcgtgaccgtgcgggaccgggactggtactggaaggcctttctgcccgag |
| | | ggcgaggacagagagcaccccgcctgcaatcccttcagccccagaggcaaaagcctggaaggcgtgtccttcc |
| | | caaagtccctggtggtggtggccggcctggacctgatcagagattggcagctggcctatgccgagggcctgaag |
| | | aaagccggccaggaagtgaagctgatgcacctggaaaaggccaccgtgggcttctacctgctgcccaacaaca |
| | | accacttccacaacgtgatggacgagatcagcgccttcgtgaacgccgagtgccccaagaaaaagcggaaggt |
| | | gGCCACCAACTTTAGCCTGCTGAAACAGGCTGGCGACGTGGAAGAG |
| | | AACCCTGGACCTatgaaggggaccaccaccatcaccatcatcaggacaagaaaaccatgatgatga |
| | | acgaagaggacgacggcaacggcatggacgagctgctggctgtgctgggctacaaagtgcggagcagcgag |
| | | atggccgacgtggcccagaaactggaacagctggaagtgatgatgagcaacgtgcaggaagatgacctgtccc |
| | | agctggccaccgagacagtgcactacaaccccgccgagctgtacacctggctggactccatgctgaccgacctg |
| | | aactccggagggtctggctccggatcaagtggtggcagcggtaccggcgagaagatcgccagctgtgccgccg |
| | | agctgacctacctgtgctggatgatcacccacaacggcaccgccatcaagagggccaccttcatgagctacaac |
| | | accatcatcagcaacagcctgagcttcgacatcgtgaacaagagcctgcagttcaagtacaagacccagaaggc |
| | | caccatcctggaggccagcctgaagaagctgatccccgcctgggagttcaccatcatcccttacaacggccaga |
| | | agcaccagagcgacatcaccgacatcgtgtccagcctgcagctgcagttcgagagcagcgaggaggccgaca |
| | | agggcaacagccacagcaagaagatgctgaaggccctgctgtccgagggcgagagcatctgggagatcaccg |
| | | agaagatcctgaacagcttcgagtacaccagcaggttcaccaagaccaagaccctgtaccagttcctgttcctggc |
| | | cacattcatcaactgcggcaggttcagcgacatcaagaacgtggaccccaagagcttcaagctggtgcagaaca |
| | | agtacctgggcgtgatcattcagtgcctggtgaccgaAaccaagacaagcgtgtccaggcacatctactttttcag |
| | | cgccagaggcaggatcgaccccctggtgtacctggacgagttcctgaggaacagcgagcccgtgctgaagaga |
| | | gtgaacaggaccggcaacagcagcagcaacaagcaggagtaccagctgctgaaggacaacctggtgcgcag |
| | | ctacaacaaggccctgaagaagaacgccccctaccccatcttcgctatcaagaacggccctaagagccacatcg |
| | | gcaggcacctgatgaccagctttctgagcatgaagggcctgaccgagctgacaaacgtggtgggcaactggag |
| | | cgacaagagggcctccgccgtggccaggaccacctacacccaccagatcaccgccatccccgaccactacttc |
| | | gccctggtgtccaggtactacgcctacgaccccatcagcaaggagatgatcgccctgaaggacgaAaccaacc |
| | | ccatcgaggagtggcagcacatcgagcagctgaagggcagcgccgagggcagcatcagataccccgcctgg |
| | | aacggcatcatcagccaggaggtgctggactacctgagcagctacatcaacaggcggatccccaagaaaaagc |
| | | ggaaggtgtga |
| 112 | ABA_Flp | atgagccagttcgacatcctgtgcaagaccccccccaaggtgctggtgcggcagttcgtggagagattcgagag |
| | (396/397) | gcccagcggcgagaagatcgccagctgtgccgccgagctgacctacctgtgctggatgatcacccacaacggc |
| | | accgccatcaagagggccaccttcatgagctacaacaccatcatcagcaacagcctgagcttcgacatcgtgaac |
| | | aagagcctgcagttcaagtacaagacccagaaggccaccatcctggaggccagcctgaagaagctgatccccg |
| | | cctgggagttcaccatcatcccttacaacggccagaagcaccagagcgacatcaccgacatcgtgtccagcctg |
| | | cagctgcagttcgagagcagcgaggaggccgacaagggcaacagccacagcaagaagatgctgaaggccct |
| | | gctgtccgagggcgagagcatctgggagatcaccgagaagatcctgaacagcttcgagtacaccagcaggttca |
| | | ccaagaccaagaccctgtaccagttcctgttcctggccacattcatcaactgcggcaggttcagcgacatcaagaa |
| | | cgtggaccccaagagcttcaagctggtgcagaacaagtacctgggcgtgatcattcagtgcctggtgaccgaAa |
| | | ccaagacaagcgtgtccaggcacatctactttttcagcgccagaggcaggatcgaccccctggtgtacctggacg |
| | | agttcctgaggaacagcgagcccgtgctgaagagagtgaacaggaccggcaacagcagcagcaacaagcag |
| | | gagtaccagctgctgaaggacaacctggtgcgcagctacaacaaggccctgaagaagaacgccccctacccca |
| | | tcttcgctatcaagaacggccctaagagccacatcggcaggcacctgatgaccagctttctgagcatgaagggcc |
| | | tgaccgagctgacaaacgtggtgggcaactggagcgacaagagggcctccgccgtggccaggaccacctaca |
| | | cccaccagatcaccgccatccccgaccactacttcgccctggtgtccaggtactacgcctacgaccccatcagca |
| | | aggagatgatcgccctgaaggacgaAaccaaccccatcgaggagtggcagcacatcgagcagctgaagggc |
| | | agcgccgagggctccggagggtctggctccggatcaagtggtggcagcggtacccctttgtatggttttacttcga |
| | | tttgtggaagaagGcctgagatggaagatgctgtttcgactataccaagattccttcaatcttcctctggttcgatgtt |
| | | agatggtcggtttgatcctcaatccgccgctcatttcttcggtgtttacgacggccatggggttctcaggtagcgaa |
| | | ctattgtagagagaggatgcatttggctttggcggaggagatagctaaggagaaaccgatgctctgcgatggtgat |
| | | acgtggctggagaagtggaagaaagctcttttcaactcgttcctgagagttgactcggagattgggtcagttgcgc |
| | | cggaAacggttgggtcaacgtcggtggttgccgttgttttcccAtctcacatcttcgtcgctaactgcggtgactct |
| | | agagccgttctttgccgcggcaaaactgcacttccattatccgttgaccataaaccggatagagaagatgaagctg |
| | | cgaggattgaagccgcaggagggaaagtgattcagtggaatggagctcgtgttttcggtgttctcgccatgtcgag |
| | | atccattggcgatagatacttgaaaccatccatcattcctgatccggaagtgacggctgtgaagagagtaaaagaa |
| | | gatgattgtctgattttggcgagtgacggggtttgggatgtaatgacggatgaagaagcgtgtgagatggcaagga |
| | | agcggattctcttgtggcacaagaaaaacgcggtggctggggatgcatcgttgctcgcggatgagcggagaaag |
| | | gaagggaaagatcctgcggcgatgtccgcggctgagtatttgtcaaagctggcgatacagagaggaagcaaag |
| | | acaacataagtgtggtggtggttgatttgaaggattacaaggacgatgacgataagcccaagaaaaagcggaag |
| | | gtgGCCACCAACTTTAGCCTGCTGAAACAGGCTGGCGACGTGGAAGA |
| | | GAACCCTGGACCTatggcgccaactcaagacgagttcacccaactctcccaatcaatcgccgagttcc |
| | | acacgtaccaactcggtaacggccgttgctcatctctcctagctcagcgaatccacgcgccgccggaaacagtat |
| | | ggtccgtggtgagGcgtttcgataggccacagatttacaaacacttcatcaaaagctgtaacgtgagtgaagattt |
| | | cgagatgcgagtgggatgcacgcgcgacgtgaacgtgataagtggattaccggcgaatacCtctcgagagaga |
| | | ttagatctgttggacgatgatcggagagtgactgggtttagtataaccggtggtgaacataggctgaggaattataa |
| | | atcggttacgacggttcatagatttgagaaagaagaagaagaagaaaggatctggaccgttgttttggaatcttatg |
| | | ttgttgatgtaccggaaggtaattcggaggaagatacgagattgtttgctgatacggttattagattgaatcttcagaa |
| | | acttgcttcgatcactgaagctatgaactacccatacgatgttccagattacgcttccggagggtctggctccggatc |
| | | aagtggtggcagcggtaccagcatcagataccccgcctggaacggcatcatcagccaggaggtgctggactac |
| | | ctgagcagctacatcaacaggcggatccccaagaaaaagcggaaggtgtga |
| 113 | GA_Bxb1 | ATGAGAGCCCTGGTGGTCATCCGGCTGTCTAGAGTGACCGACGCTAC |
| | (37/38) | CACCTCTCCTGAGCGGCAGCTGGAATCTTGCCAGCAGCTGTGTGCTC |
| | | AGCGCGGATGGGATGTTtccggagggtctggctccggatcaagtggtggcagcggtaccatgg |
| | | ccgccagcgacgaagtgaacctgatcgagagcagaaccgtggtgcccctgaacacctgggtgctgatctccaa |
| | | cttcaaggtggcctacaacatcctgcggaggcccgacggcaccttcaacagacacctggccgagtacctggacc |
| | | ggaaagtgaccgccaacgccaaccctgtggacggcgtgttcagcttcgacgtgctgatcgaccggcggatcaac |
| | | ctgctgagccgggtgtacagacccgcctacgccgatcaggaacagcccccctctatcctggatctggaaaagcc |
| | | cgtggatggcgacatcgtgcccgtgatcctgttcttccacggcggcagctttgcccacagcagcgccaatagcgc |
| | | catctacgacaccctgtgcagacggctcgtgggcctgtgcaaatgcgtggtggtgtccgtgaactaccgcagagc |
| | | ccccgagaacccttacccctgcgcctacgatgatggctggatcgccctgaactgggtcaacagcagaagctggc |
| | | tgaagtccaagaaagacagcaaggtgcacatctttctggccggcgatagcagcggcggcaatatcgcccataac |
| | | gtggccctgagagccggcgagtctggcatcgatgtgctgggcaatatcctgctgaaccccatgttcggcggcaa |
| | | cgagcggaccgagagcgagaagtctctggacggcaagtacttcgtgaccgtgcgggaccgggactggtactg |
| | | gaaggcctttctgcccgagggcgaggacagagagcaccccgcctgcaatcccttcagccccagaggcaaaag |
| | | cctggaaggcgtgtccttcccaaagtccctggtggtggtggccggcctggacctgatcagagattggcagctggc |
| | | ctatgccgagggcctgaagaaagccggccaggaagtgaagctgatgcacctggaaaaggccaccgtgggcttc |
| | | tacctgctgcccaacaacaaccacttccacaacgtgatggacgagatcagcgccttcgtgaacgccgagtgccc |
| | | caagaaaaagcggaaggtgGCCACCAACTTTAGCCTGCTGAAACAGGCTGGCG |
| | | ACGTGGAAGAGAACCCTGGACCTatgaagcgggaccaccaccatcaccatcatcaggaca |
| | | agaaaaccatgatgatgaacgaagaggacgacggcaacggcatggacgagctgctggctgtgctgggctacaa |
| | | agtgcggagcagcgagatggccgacgtggcccagaaactggaacagctggaagtgatgatgagcaacgtgca |
| | | ggaagatgacctgtcccagctggccaccgagacagtgcactacaaccccgccgagctgtacacctggctggact |
| | | ccatgctgaccgacctgaactccggagggtctggctccggatcaagtggtggcagcggtaccGTGGGAG |
| | | TCGCTGAGGACCTGGATGTGTCTGGTGCCGTGGATCCTTTCGACCGG |
| | | AAGCGGAGGCCTAACCTGGCTAGATGGCTGGCCTTTGAGGAACAGC |
| | | CCTTCGACGTGATCGTGGCCTACAGAGTGGACCGGCTGACCCGGTCT |
| | | ATCAGACATCTGCAGCAGCTGGTCCACTGGGCCGAAGATCACAAGA |
| | | AACTGGTGGTGTCCGCCACCGAGGCTCACTTCGATACCACCACACCT |
| | | TTTGCCGCCGTCGTGATCGCTCTGATGGGAACCGTTGCTCAGATGGA |
| | | ACTGGAAGCCATCAAAGAGCGGAACAGATCCGCCGCTCACTTCAAC |
| | | ATCAGAGCCGGCAAGTACCGGGGCTCTTTGCCTCCTTGGGGCTACCT |
| | | GCCAACAAGAGTGGATGGCGAATGGCGGCTGGTGCCTGATCCTGTG |
| | | CAGCGGGAAAGAATCCTGGAAGTGTACCACAGAGTGGTGGACAACC |
| | | ACGAGCCTCTGCACCTGGTGGCCCACGACTTGAATAGAAGAGGCGT |
| | | GCTGTCCCCTAAGGACTACTTCGCCCAGCTGCAGGGCAGAGAGCCTC |
| | | AGGGAAGAGAGTGGAGCGCTACCGCTCTGAAGCGGTCCATGATCTC |
| | | TGAGGCCATGCTGGGCTACGCTACCCTGAATGGAAAGACCGTGCGG |
| | | GACGATGATGGCGCCCCTCTTGTTAGAGCCGAGCCTATCCTGACCAG |
| | | AGAGCAGCTCGAAGCCCTGAGAGCTGAGCTGGTCAAGACCTCCAGA |
| | | GCCAAGCCTGCTGTGTCTACCCCTAGCCTGCTGCTGAGAGTGCTGTT |
| | | CTGTGCTGTGTGTGGCGAGCCCGCCTACAAGTTTGCTGGCGGCGGAA |
| | | GAAAGCACCCCAGATACCGGTGTCGGTCCATGGGCTTCCCTAAGCA |
| | | CTGTGGCAATGGCACCGTGGCCATGGCTGAGTGGGATGCCTTCTGCG |
| | | AAGAACAGGTGCTGGATCTGCTGGGCGACGCCGAGAGACTGGAAAA |
| | | AGTGTGGGTGGCCGGCTCCGACTCTGCTGTGGAACTGGCTGAAGTG |
| | | AACGCCGAGCTGGTGGACCTGACCTCTCTGATCGGCTCTCCCGCTTA |
| | | TAGAGCTGGCTCCCCTCAGAGAGAAGCCCTGGACGCTAGAATCGCT |
| | | GCCCTGGCTGCTAGACAAGAGGAACTCGAAGGCCTGGAAGCTCGGC |
| | | CTTCAGGATGGGAGTGGCGAGAGACAGGCCAGAGATTTGGCGACTG |
| | | GTGGCGCGAGCAAGATACCGCCGCTAAGAACACCTGGCTGCGGTCT |
| | | ATGAATGTGCGGCTGACCTTCGATGTGCGCGGAGGACTGACCAGAA |
| | | CCATCGACTTCGGCGACCTGCAAGAGTACGAGCAGCATCTGAGACT |
| | | GGGCTCCGTGGTGGAAAGACTGCACACCGGCATGTCCggttcaCCAAAG |
| | | AAAAAGCGGAAAGTGTGA |
| 114 | GA_Bxb1 | ATGAGAGCCCTGGTGGTCATCCGGCTGTCTAGAGTGACCGACGCTAC |
| | (169/170) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATtccggagg |
| | | gtctggctccggatcaagtggtggcagcggtaccatggccgccagcgacgaagtgaacctgatcgagagcaga |
| | | accgtggtgcccctgaacacctgggtgctgatctccaacttcaaggtggcctacaacatcctgcggaggcccgac |
| | | ggcaccttcaacagacacctggccgagtacctggaccggaaagtgaccgccaacgccaaccctgtggacggc |
| | | gtgttcagcttcgacgtgctgatcgaccggcggatcaacctgctgagccgggtgtacagacccgcctacgccgat |
| | | caggaacagcccccctctatcctggatctggaaaagcccgtggatggcgacatcgtgcccgtgatcctgttcttcc |
| | | acggcggcagctttgcccacagcagcgccaatagcgccatctacgacaccctgtgcagacggctcgtgggcct |
| | | gtgcaaatgcgtggtggtgtccgtgaactaccgcagagcccccgagaacccttacccctgcgcctacgatgatg |
| | | gctggatcgccctgaactgggtcaacagcagaagctggctgaagtccaagaaagacagcaaggtgcacatcttt |
| | | ctggccggcgatagcagcggcggcaatatcgcccataacgtggccctgagagccggcgagtctggcatcgatg |
| | | tgctgggcaatatcctgctgaaccccatgttcggcggcaacgagcggaccgagagcgagaagtctctggacgg |
| | | caagtacttcgtgaccgtgcgggaccgggactggtactggaaggcctttctgcccgagggcgaggacagagag |
| | | caccccgcctgcaatcccttcagccccagaggcaaaagcctggaaggcgtgtccttcccaaagtccctggtggt |
| | | ggtggccggcctggacctgatcagagattggcagctggcctatgccgagggcctgaagaaagccggccagga |
| | | agtgaagctgatgcacctggaaaaggccaccgtgggcttctacctgctgcccaacaacaaccacttccacaacgt |
| | | gatggacgagatcagcgccttcgtgaacgccgagtgccccaagaaaaagcggaaggtgGCCACCAAC |
| | | TTTAGCCTGCTGAAACAGGCTGGCGACGTGGAAGAGAACCCTGGAC |
| | | CTatgaagcgggaccaccaccatcaccatcatcaggacaagaaaaccatgatgatgaacgaagaggacgacg |
| | | gcaacggcatggacgagctgctggctgtgctgggctacaaagtgcggagcagcgagatggccgacgtggccc |
| | | agaaactggaacagctggaagtgatgatgagcaacgtgcaggaagatgacctgtcccagctggccaccgagac |
| | | agtgcactacaaccccgccgagctgtacacctggctggactccatgctgaccgacctgaactccggagggtctg |
| | | gctccggatcaagtggtggcagcggtaccGGCGAATGGCGGCTGGTGCCTGATCCTG |
| | | TGCAGCGGGAAAGAATCCTGGAAGTGTACCACAGAGTGGTGGACAA |
| | | CCACGAGCCTCTGCACCTGGTGGCCCACGACTTGAATAGAAGAGGC |
| | | GTGCTGTCCCCTAAGGACTACTTCGCCCAGCTGCAGGGCAGAGAGC |
| | | CTCAGGGAAGAGAGTGGAGCGCTACCGCTCTGAAGCGGTCCATGAT |
| | | CTCTGAGGCCATGCTGGGCTACGCTACCCTGAATGGAAAGACCGTG |
| | | CGGGACGATGATGGCGCCCCTCTTGTTAGAGCCGAGCCTATCCTGAC |
| | | CAGAGAGCAGCTCGAAGCCCTGAGAGCTGAGCTGGTCAAGACCTCC |
| | | AGAGCCAAGCCTGCTGTGTCTACCCCTAGCCTGCTGCTGAGAGTGCT |
| | | GTTCTGTGCTGTGTGTGGCGAGCCCGCCTACAAGTTTGCTGGCGGCG |
| | | GAAGAAAGCACCCCAGATACCGGTGTCGGTCCATGGGCTTCCCTAA |
| | | GCACTGTGGCAATGGCACCGTGGCCATGGCTGAGTGGGATGCCTTCT |
| | | GCGAAGAACAGGTGCTGGATCTGCTGGGCGACGCCGAGAGACTGGA |
| | | AAAAGTGTGGGTGGCCGGCTCCGACTCTGCTGTGGAACTGGCTGAA |
| | | GTGAACGCCGAGCTGGTGGACCTGACCTCTCTGATCGGCTCTCCCGC |
| | | TTATAGAGCTGGCTCCCCTCAGAGAGAAGCCCTGGACGCTAGAATC |
| | | GCTGCCCTGGCTGCTAGACAAGAGGAACTCGAAGGCCTGGAAGCTC |
| | | GGCCTTCAGGATGGGAGTGGCGAGAGACAGGCCAGAGATTTGGCGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 115 | GA_Bxb1 | ATGAGAGCCCTGGTGGTCATCCGGCTGTCTAGAGTGACCGACGCTAC |
| | (195/196) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACtccggagggtctggctccggatcaagtggtg |
| | | gcagcggtaccatggccgccagcgacgaagtgaacctgatcgagagcagaaccgtggtgcccctgaacacct |
| | | gggtgctgatctccaacttcaaggtggcctacaacatcctgcggaggcccgacggcaccttcaacagacacctg |
| | | gccgagtacctggaccggaaagtgaccgccaacgccaaccctgtggacggcgtgttcagcttcgacgtgctgat |
| | | cgaccggcggatcaacctgctgagccgggtgtacagacccgcctacgccgatcaggaacagcccccctctatc |
| | | ctggatctggaaaagcccgtggatggcgacatcgtgcccgtgatcctgttcttccacggcggcagctttgcccaca |
| | | gcagcgccaatagcgccatctacgacaccctgtgcagacggctcgtgggcctgtgcaaatgcgtggtggtgtcc |
| | | gtgaactaccgcagagcccccgagaacccttacccctgcgcctacgatgatggctggatcgccctgaactgggt |
| | | caacagcagaagctggctgaagtccaagaaagacagcaaggtgcacatctttctggccggcgatagcagcggc |
| | | ggcaatatcgcccataacgtggccctgagagccggcgagtctggcatcgatgtgctgggcaatatcctgctgaac |
| | | cccatgttcggcggcaacgagcggaccgagagcgagaagtctctggacggcaagtacttcgtgaccgtgcggg |
| | | accgggactggtactggaaggcctttctgcccgagggcgaggacagagagcaccccgcctgcaatcccttcag |
| | | ccccagaggcaaaagcctggaaggcgtgtccttcccaaagtccctggtggtggtggccggcctggacctgatca |
| | | gagattggcagctggcctatgccgagggcctgaagaaagccggccaggaagtgaagctgatgcacctggaaa |
| | | aggccaccgtgggcttctacctgctgcccaacaacaaccacttccacaacgtgatggacgagatcagcgccttcg |
| | | tgaacgccgagtgccccaagaaaaagcggaaggtgGCCACCAACTTTAGCCTGCTGAAA |
| | | CAGGCTGGCGACGTGGAAGAGAACCCTGGACCTatgaaggggaccaccacca |
| | | tcaccatcatcaggacaagaaaaccatgatgatgaacgaagaggacgacggcaacggcatggacgagctgctg |
| | | gctgtgctgggctacaaagtgcggagcagcgagatggccgacgtggcccagaaactggaacagctggaagtg |
| | | atgatgagcaacgtgcaggaagatgacctgtcccagctggccaccgagacagtgcactacaaccccgccgagc |
| | | tgtacacctggctggactccatgctgaccgacctgaactccggagggtctggctccggatcaagtggtggcagc |
| | | ggtaccGAGCCTCTGCACCTGGTGGCCCACGACTTGAATAGAAGAGGC |
| | | GTGCTGTCCCCTAAGGACTACTTCGCCCAGCTGCAGGGCAGAGAGC |
| | | CTCAGGGAAGAGAGTGGAGCGCTACCGCTCTGAAGCGGTCCATGAT |
| | | CTCTGAGGCCATGCTGGGCTACGCTACCCTGAATGGAAAGACCGTG |
| | | CGGGACGATGATGGCGCCCCTCTTGTTAGAGCCGAGCCTATCCTGAC |
| | | CAGAGAGCAGCTCGAAGCCCTGAGAGCTGAGCTGGTCAAGACCTCC |
| | | AGAGCCAAGCCTGCTGTGTCTACCCCTAGCCTGCTGCTGAGAGTGCT |
| | | GTTCTGTGCTGTGTGTGGCGAGCCCGCCTACAAGTTTGCTGGCGGCG |
| | | GAAGAAAGCACCCCAGATACCGGTGTCGGTCCATGGGCTTCCCTAA |
| | | GCACTGTGGCAATGGCACCGTGGCCATGGCTGAGTGGGATGCCTTCT |
| | | GCGAAGAACAGGTGCTGGATCTGCTGGGCGACGCCGAGAGACTGGA |
| | | AAAAGTGTGGGTGGCCGGCTCCGACTCTGCTGTGGAACTGGCTGAA |
| | | GTGAACGCCGAGCTGGTGGACCTGACCTCTCTGATCGGCTCTCCCGC |
| | | TTATAGAGCTGGCTCCCCTCAGAGAGAAGCCCTGGACGCTAGAATC |
| | | GCTGCCCTGGCTGCTAGACAAGAGGAACTCGAAGGCCTGGAAGCTC |
| | | GGCCTTCAGGATGGGAGTGGCGAGAGACAGGCCAGAGATTTGGCGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 116 | GA_Bxb1 | ATGAGAGCCCTGGTGGTCATCCGGCTGTCTAGAGTGACCGACGCTAC |
| | (208/209) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAtccggagggtctggctccggatcaagtggtggcagcggtacc |
| | | atggccgccagcgacgaagtgaacctgatcgagagcagaaccgtggtgcccctgaacacctgggtgctgatctc |
| | | caacttcaaggtggcctacaacatcctgcggaggcccgacggcaccttcaacagacacctggccgagtacctgg |
| | | accggaaagtgaccgccaacgccaaccctgtggacggcgtgttcagcttcgacgtgctgatcgaccggggatc |
| | | aacctgctgagccgggtgtacagacccgcctacgccgatcaggaacagcccccctctatcctggatctggaaaa |
| | | gcccgtggatggcgacatcgtgcccgtgatcctgttcttccacggcggcagctttgcccacagcagcgccaatag |
| | | cgccatctacgacaccctgtgcagacggctcgtgggcctgtgcaaatgcgtggtggtgtccgtgaactaccgcag |
| | | agcccccgagaacccttacccctgcgcctacgatgatggctggatcgccctgaactgggtcaacagcagaagct |
| | | ggctgaagtccaagaaagacagcaaggtgcacatctttctggccggcgatagcagcggcggcaatatcgcccat |
| | | aacgtggccctgagagccggcgagtctggcatcgatgtgctgggcaatatcctgctgaaccccatgttcggcgg |
| | | caacgagcggaccgagagcgagaagtctctggacggcaagtacttcgtgaccgtgcgggaccgggactggta |
| | | ctggaaggcctttctgcccgagggcgaggacagagagcaccccgcctgcaatcccttcagccccagaggcaaa |
| | | agcctggaaggcgtgtccttcccaaagtccctggtggtggtggccggcctggacctgatcagagattggcagctg |
| | | gcctatgccgagggcctgaagaaagccggccaggaagtgaagctgatgcacctggaaaaggccaccgtgggc |
| | | ttctacctgctgcccaacaacaaccacttccacaacgtgatggacgagatcagcgccttcgtgaacgccgagtgc |
| | | cccaagaaaaagcggaaggtgGCCACCAACTTTAGCCTGCTGAAACAGGCTGGC |
| | | GACGTGGAAGAGAACCCTGGACCTatgaaggggaccaccaccatcaccatcatcagga |
| | | caagaaaaccatgatgatgaacgaagaggacgacggcaacggcatggacgagctgctggctgtgctgggctac |
| | | aaagtgcggagcagcgagatggccgacgtggcccagaaactggaacagctggaagtgatgatgagcaacgtg |
| | | caggaagatgacctgtcccagctggccaccgagacagtgcactacaaccccgccgagctgtacacctggctgg |
| | | actccatgctgaccgacctgaactccggagggtctggctccggatcaagtggtggcagcggtaccGGCGT |
| | | GCTGTCCCCTAAGGACTACTTCGCCCAGCTGCAGGGCAGAGAGCCTC |
| | | AGGGAAGAGAGTGGAGCGCTACCGCTCTGAAGCGGTCCATGATCTC |
| | | TGAGGCCATGCTGGGCTACGCTACCCTGAATGGAAAGACCGTGCGG |
| | | GACGATGATGGCGCCCCTCTTGTTAGAGCCGAGCCTATCCTGACCAG |
| | | AGAGCAGCTCGAAGCCCTGAGAGCTGAGCTGGTCAAGACCTCCAGA |
| | | GCCAAGCCTGCTGTGTCTACCCCTAGCCTGCTGCTGAGAGTGCTGTT |
| | | CTGTGCTGTGTGTGGCGAGCCCGCCTACAAGTTTGCTGGCGGCGGAA |
| | | GAAAGCACCCCAGATACCGGTGTCGGTCCATGGGCTTCCCTAAGCA |
| | | CTGTGGCAATGGCACCGTGGCCATGGCTGAGTGGGATGCCTTCTGCG |
| | | AAGAACAGGTGCTGGATCTGCTGGGCGACGCCGAGAGACTGGAAAA |
| | | AGTGTGGGTGGCCGGCTCCGACTCTGCTGTGGAACTGGCTGAAGTG |
| | | AACGCCGAGCTGGTGGACCTGACCTCTCTGATCGGCTCTCCCGCTTA |
| | | TAGAGCTGGCTCCCCTCAGAGAGAAGCCCTGGACGCTAGAATCGCT |
| | | GCCCTGGCTGCTAGACAAGAGGAACTCGAAGGCCTGGAAGCTCGGC |
| | | CTTCAGGATGGGAGTGGCGAGAGACAGGCCAGAGATTTGGCGACTG |
| | | GTGGCGCGAGCAAGATACCGCCGCTAAGAACACCTGGCTGCGGTCT |
| | | ATGAATGTGCGGCTGACCTTCGATGTGCGCGGAGGACTGACCAGAA |
| | | CCATCGACTTCGGCGACCTGCAAGAGTACGAGCAGCATCTGAGACT |
| | | GGGCTCCGTGGTGGAAAGACTGCACACCGGCATGTCCggttcaCCAAAG |
| | | AAAAAGCGGAAAGTGTGA |
| 117 | GA_Bxb1 | ATGAGAGCCCTGGTGGTCATCCGGCTGTCTAGAGTGACCGACGCTAC |
| | (222/223) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCtccggagggtctggctccggatcaagtggtggcagcggtaccatggccg |
| | | ccagcgacgaagtgaacctgatcgagagcagaaccgtggtgcccctgaacacctgggtgctgatctccaacttc |
| | | aaggtggcctacaacatcctgcggaggcccgacggcaccttcaacagacacctggccgagtacctggaccgga |
| | | aagtgaccgccaacgccaaccctgtggacggcgtgttcagcttcgacgtgctgatcgaccggcggatcaacctg |
| | | ctgagccgggtgtacagacccgcctacgccgatcaggaacagcccccctctatcctggatctggaaaagcccgt |
| | | ggatggcgacatcgtgcccgtgatcctgttcttccacggcggcagctttgcccacagcagcgccaatagcgccat |
| | | ctacgacaccctgtgcagacggctcgtgggcctgtgcaaatgcgtggtggtgtccgtgaactaccgcagagccc |
| | | ccgagaacccttacccctgcgcctacgatgatggctggatcgccctgaactgggtcaacagcagaagctggctg |
| | | aagtccaagaaagacagcaaggtgcacatctttctggccggcgatagcagcggcggcaatatcgcccataacgt |
| | | ggccctgagagccggcgagtctggcatcgatgtgctgggcaatatcctgctgaaccccatgttcggcggcaacg |
| | | agcggaccgagagcgagaagtctctggacggcaagtacttcgtgaccgtgcgggaccgggactggtactggaa |
| | | ggcctttctgcccgagggcgaggacagagagcaccccgcctgcaatcccttcagccccagaggcaaaagcctg |
| | | gaaggcgtgtccttcccaaagtccctggtggtggtggccggcctggacctgatcagagattggcagctggcctat |
| | | gccgagggcctgaagaaagccggccaggaagtgaagctgatgcacctggaaaaggccaccgtgggcttctac |
| | | ctgctgcccaacaacaaccacttccacaacgtgatggacgagatcagcgccttcgtgaacgccgagtgccccaa |
| | | gaaaaagcggaaggtgGCCACCAACTTTAGCCTGCTGAAACAGGCTGGCGAC |
| | | GTGGAAGAGAACCCTGGACCTatgaagcgggaccaccaccatcaccatcatcaggacaaga |
| | | aaaccatgatgatgaacgaagaggacgacggcaacggcatggacgagctgctggctgtgctgggctacaaagt |
| | | gcggagcagcgagatggccgacgtggcccagaaactggaacagctggaagtgatgatgagcaacgtgcagg |
| | | aagatgacctgtcccagctggccaccgagacagtgcactacaaccccgccgagctgtacacctggctggactcc |
| | | atgctgaccgacctgaactccggagggtctggctccggatcaagtggtggcagcggtaccAGAGAGCC |
| | | TCAGGGAAGAGAGTGGAGCGCTACCGCTCTGAAGCGGTCCATGATC |
| | | TCTGAGGCCATGCTGGGCTACGCTACCCTGAATGGAAAGACCGTGC |
| | | GGGACGATGATGGCGCCCCTCTTGTTAGAGCCGAGCCTATCCTGACC |
| | | AGAGAGCAGCTCGAAGCCCTGAGAGCTGAGCTGGTCAAGACCTCCA |
| | | GAGCCAAGCCTGCTGTGTCTACCCCTAGCCTGCTGCTGAGAGTGCTG |
| | | TTCTGTGCTGTGTGTGGCGAGCCCGCCTACAAGTTTGCTGGCGGCGG |
| | | AAGAAAGCACCCCAGATACCGGTGTCGGTCCATGGGCTTCCCTAAG |
| | | CACTGTGGCAATGGCACCGTGGCCATGGCTGAGTGGGATGCCTTCTG |
| | | CGAAGAACAGGTGCTGGATCTGCTGGGCGACGCCGAGAGACTGGAA |
| | | AAAGTGTGGGTGGCCGGCTCCGACTCTGCTGTGGAACTGGCTGAAG |
| | | TGAACGCCGAGCTGGTGGACCTGACCTCTCTGATCGGCTCTCCCGCT |
| | | TATAGAGCTGGCTCCCCTCAGAGAGAAGCCCTGGACGCTAGAATCG |
| | | CTGCCCTGGCTGCTAGACAAGAGGAACTCGAAGGCCTGGAAGCTCG |
| | | GCCTTCAGGATGGGAGTGGCGAGAGACAGGCCAGAGATTTGGCGAC |
| | | TGGTGGCGCGAGCAAGATACCGCCGCTAAGAACACCTGGCTGCGGT |
| | | CTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 118 | GA_Bxb1 | ATGAGAGCCCTGGTGGTCATCCGGCTGTCTAGAGTGACCGACGCTAC |
| | (259/260) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATtccggagggtctggctccg |
| | | gatcaagtggtggcagcggtaccatggccgccagcgacgaagtgaacctgatcgagagcagaaccgtggtgc |
| | | ccctgaacacctgggtgctgatctccaacttcaaggtggcctacaacatcctgcggaggcccgacggcaccttca |
| | | acagacacctggccgagtacctggaccggaaagtgaccgccaacgccaaccctgtggacggcgtgttcagctt |
| | | cgacgtgctgatcgaccggcggatcaacctgctgagccgggtgtacagacccgcctacgccgatcaggaacag |
| | | cccccctctatcctggatctggaaaagcccgtggatggcgacatcgtgcccgtgatcctgttcttccacggcggca |
| | | gctttgcccacagcagcgccaatagcgccatctacgacaccctgtgcagacggctcgtgggcctgtgcaaatgc |
| | | gtggtggtgtccgtgaactaccgcagagcccccgagaacccttacccctgcgcctacgatgatggctggatcgc |
| | | cctgaactgggtcaacagcagaagctggctgaagtccaagaaagacagcaaggtgcacatctttctggccggcg |
| | | atagcagcggcggcaatatcgcccataacgtggccctgagagccggcgagtctggcatcgatgtgctgggcaat |
| | | atcctgctgaaccccatgttcggcggcaacgagcggaccgagagcgagaagtctctggacggcaagtacttcgt |
| | | gaccgtgcgggaccgggactggtactggaaggcctttctgcccgagggcgaggacagagagcaccccgcctg |
| | | caatcccttcagccccagaggcaaaagcctggaaggcgtgtccttcccaaagtccctggtggtggtggccggcc |
| | | tggacctgatcagagattggcagctggcctatgccgagggcctgaagaaagccggccaggaagtgaagctgat |
| | | gcacctggaaaaggccaccgtgggcttctacctgctgcccaacaacaaccacttccacaacgtgatggacgaga |
| | | tcagcgccttcgtgaacgccgagtgccccaagaaaaagcggaaggtgGCCACCAACTTTAGCCT |
| | | GCTGAAACAGGCTGGCGACGTGGAAGAGAACCCTGGACCTatgaagcgg |
| | | gaccaccaccatcaccatcatcaggacaagaaaaccatgatgatgaacgaagaggacgacggcaacggcatg |
| | | gacgagctgctggctgtgctgggctacaaagtgcggagcagcgagatggccgacgtggcccagaaactggaa |
| | | cagctggaagtgatgatgagcaacgtgcaggaagatgacctgtcccagctggccaccgagacagtgcactaca |
| | | accccgccgagctgtacacctggctggactccatgctgaccgacctgaactccggagggtctggctccggatca |
| | | agtggtggcagcggtaccGGCGCCCCTCTTGTTAGAGCCGAGCCTATCCTGAC |
| | | CAGAGAGCAGCTCGAAGCCCTGAGAGCTGAGCTGGTCAAGACCTCC |
| | | AGAGCCAAGCCTGCTGTGTCTACCCCTAGCCTGCTGCTGAGAGTGCT |
| | | GTTCTGTGCTGTGTGTGGCGAGCCCGCCTACAAGTTTGCTGGCGGCG |
| | | GAAGAAAGCACCCCAGATACCGGTGTCGGTCCATGGGCTTCCCTAA |
| | | GCACTGTGGCAATGGCACCGTGGCCATGGCTGAGTGGGATGCCTTCT |
| | | GCGAAGAACAGGTGCTGGATCTGCTGGGCGACGCCGAGAGACTGGA |
| | | AAAAGTGTGGGTGGCCGGCTCCGACTCTGCTGTGGAACTGGCTGAA |
| | | GTGAACGCCGAGCTGGTGGACCTGACCTCTCTGATCGGCTCTCCCGC |
| | | TTATAGAGCTGGCTCCCCTCAGAGAGAAGCCCTGGACGCTAGAATC |
| | | GCTGCCCTGGCTGCTAGACAAGAGGAACTCGAAGGCCTGGAAGCTa |
| | | GGCCTTCAGGATGGGAGTGGCGAGAGACAGGCCAGAGATTTGGCGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 119 | GA_Bxb1 | ATGAGAGCCCTGGTGGTCATCCGGCTGTCTAGAGTGACCGACGCTAC |
| | (262/263) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATGGCGCCCCTtccgg |
| | | agggtctggctccggatcaagtggtggcagcggtaccatggccgccagcgacgaagtgaacctgatcgagagc |
| | | agaaccgtggtgcccctgaacacctgggtgctgatctccaacttcaaggtggcctacaacatcctgcggaggccc |
| | | gacggcaccttcaacagacacctggccgagtacctggaccggaaagtgaccgccaacgccaaccctgtggac |
| | | ggcgtgttcagcttcgacgtgctgatcgaccggcggatcaacctgctgagccgggtgtacagacccgcctacgc |
| | | cgatcaggaacagcccccctctatcctggatctggaaaagcccgtggatggcgacatcgtgcccgtgatcctgtt |
| | | cttccacggcggcagctttgcccacagcagcgccaatagcgccatctacgacaccctgtgcagacggctcgtgg |
| | | gcctgtgcaaatgcgtggtggtgtccgtgaactaccgcagagcccccgagaacccttacccctgcgcctacgatg |
| | | atggctggatcgccctgaactgggtcaacagcagaagctggctgaagtccaagaaagacagcaaggtgcacat |
| | | ctttctggccggcgatagcagcggcggcaatatcgcccataacgtggccctgagagccggcgagtctggcatcg |
| | | atgtgctgggcaatatcctgctgaaccccatgttcggcggcaacgagcggaccgagagcgagaagtctctggac |
| | | ggcaagtacttcgtgaccgtgcgggaccgggactggtactggaaggcctttctgcccgagggcgaggacagag |
| | | agcaccccgcctgcaatcccttcagccccagaggcaaaagcctggaaggcgtgtccttcccaaagtccctggtg |
| | | gtggtggccggcctggacctgatcagagattggcagctggcctatgccgagggcctgaagaaagccggccag |
| | | gaagtgaagctgatgcacctggaaaaggccaccgtgggcttctacctgctgcccaacaacaaccacttccacaa |
| | | cgtgatggacgagatcagcgccttcgtgaacgccgagtgccccaagaaaaagcggaaggtgGCCACCA |
| | | ACTTTAGCCTGCTGAAACAGGCTGGCGACGTGGAAGAGAACCCTGG |
| | | ACCTatgaagcgggaccaccaccatcaccatcatcaggacaagaaaaccatgatgatgaacgaagaggacg |
| | | acggcaacggcatggacgagctgctggctgtgctgggctacaaagtgcggagcagcgagatggccgacgtgg |
| | | cccagaaactggaacagctggaagtgatgatgagcaacgtgcaggaagatgacctgtcccagctggccaccga |
| | | gacagtgcactacaaccccgccgagctgtacacctggctggactccatgctgaccgacctgaactccggagggt |
| | | ctggctccggatcaagtggtggcagcggtaccCTTGTTAGAGCCGAGCCTATCCTGACC |
| | | AGAGAGCAGCTCGAAGCCCTGAGAGCTGAGCTGGTCAAGACCTCCA |
| | | GAGCCAAGCCTGCTGTGTCTACCCCTAGCCTGCTGCTGAGAGTGCTG |
| | | TTCTGTGCTGTGTGTGGCGAGCCCGCCTACAAGTTTGCTGGCGGCGG |
| | | AAGAAAGCACCCCAGATACCGGTGTCGGTCCATGGGCTTCCCTAAG |
| | | CACTGTGGCAATGGCACCGTGGCCATGGCTGAGTGGGATGCCTTCTG |
| | | CGAAGAACAGGTGCTGGATCTGCTGGGCGACGCCGAGAGACTGGAA |
| | | AAAGTGTGGGTGGCCGGCTCCGACTCTGCTGTGGAACTGGCTGAAG |
| | | TGAACGCCGAGCTGGTGGACCTGACCTCTCTGATCGGCTCTCCCGCT |
| | | TATAGAGCTGGCTCCCCTCAGAGAGAAGCCCTGGACGCTAGAATCG |
| | | CTGCCCTGGCTGCTAGACAAGAGGAACTCGAAGGCCTGGAAGCTCG |
| | | GCCTTCAGGATGGGAGTGGCGAGAGACAGGCCAGAGATTTGGCGAC |
| | | TGGTGGCGCGAGCAAGATACCGCCGCTAAGAACACCTGGCTGCGGT |
| | | CTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 120 | GA_Bxb1 | ATGAGAGCCCTGGTGGTCATCCGGCTGTCTAGAGTGACCGACGCTAC |
| | (363/364) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATGGCGCCCCTCTT |
| | | GTTAGAGCCGAGCCTATCCTGACCAGAGAGCAGCTCGAAGCCCTGA |
| | | GAGCTGAGCTGGTCAAGACCTCCAGAGCCAAGCCTGCTGTGTCTACC |
| | | CCTAGCCTGCTGCTGAGAGTGCTGTTCTGTGCTGTGTGTGGCGAGCC |
| | | CGCCTACAAGTTTGCTGGCGGCGGAAGAAAGCACCCCAGATACCGG |
| | | TGTCGGTCCATGGGCTTCCCTAAGCACTGTGGCAATGGCACCGTGGC |
| | | CATGGCTGAGTGGGATGCCTTCTGCGAAGAACAGGTGCTGGATCTG |
| | | CTGGGCGACGCCGAGAGACTGtccggagggtctggctccggatcaagtggtggcagcggta |
| | | ccatggccgccagcgacgaagtgaacctgatcgagagcagaaccgtggtgcccctgaacacctgggtgctgat |
| | | ctccaacttcaaggtggcctacaacatcctgcggaggcccgacggcaccttcaacagacacctggccgagtacc |
| | | tggaccggaaagtgaccgccaacgccaaccctgtggacggcgtgttcagcttcgacgtgctgatcgaccggcg |
| | | gatcaacctgctgagccgggtgtacagacccgcctacgccgatcaggaacagcccccctctatcctggatctgg |
| | | aaaagcccgtggatggcgacatcgtgcccgtgatcctgttcttccacggcggcagctttgcccacagcagcgcca |
| | | atagcgccatctacgacaccctgtgcagacggctcgtgggcctgtgcaaatgcgtggtggtgtccgtgaactacc |
| | | gcagagcccccgagaacccttacccctgcgcctacgatgatggctggatcgccctgaactgggtcaacagcaga |
| | | agctggctgaagtccaagaaagacagcaaggtgcacatctttctggccggcgatagcagcggcggcaatatcgc |
| | | ccataacgtggccctgagagccggcgagtctggcatcgatgtgctgggcaatatcctgctgaaccccatgttcgg |
| | | cggcaacgagcggaccgagagcgagaagtctctggacggcaagtacttcgtgaccgtgcgggaccgggactg |
| | | gtactggaaggcctttctgcccgagggcgaggacagagagcaccccgcctgcaatcccttcagccccagaggc |
| | | aaaagcctggaaggcgtgtccttcccaaagtccctggtggtggtggccggcctggacctgatcagagattggca |
| | | gctggcctatgccgagggcctgaagaaagccggccaggaagtgaagctgatgcacctggaaaaggccaccgt |
| | | gggcttctacctgctgcccaacaacaaccacttccacaacgtgatggacgagatcagcgccttcgtgaacgccga |
| | | gtgccccaagaaaaagcggaaggtgGCCACCAACTTTAGCCTGCTGAAACAGGCT |
| | | GGCGACGTGGAAGAGAACCCTGGACCTatgaagcgggaccaccaccatcaccatcatc |
| | | aggacaagaaaaccatgatgatgaacgaagaggacgacggcaacggcatggacgagctgctggctgtgctgg |
| | | gctacaaagtgcggagcagcgagatggccgacgtggcccagaaactggaacagctggaagtgatgatgagca |
| | | acgtgcaggaagatgacctgtcccagctggccaccgagacagtgcactacaaccccgccgagctgtacacctg |
| | | gctggactccatgctgaccgacctgaactccggagggtctggctccggatcaagtggtggcagcggtaccGA |
| | | AAAAGTGTGGGTGGCCGGCTCCGACTCTGCTGTGGAACTGGCTGAA |
| | | GTGAACGCCGAGCTGGTGGACCTGACCTCTCTGATCGGCTCTCCCGC |
| | | TTATAGAGCTGGCTCCCCTCAGAGAGAAGCCCTGGACGCTAGAATC |
| | | GCTGCCCTGGCTGCTAGACAAGAGGAACTCGAAGGCCTGGAAGCTC |
| | | GGCCTTCAGGATGGGAGTGGCGAGAGACAGGCCAGAGATTTGGCGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 121 | GA_Bxb1 | ATGAGAGCCCTGGTGGTCATCCGGCTGTCTAGAGTGACCGACGCTAC |
| | (370/371) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATGGCGCCCCTCTT |
| | | GTTAGAGCCGAGCCTATCCTGACCAGAGAGCAGCTCGAAGCCCTGA |
| | | GAGCTGAGCTGGTCAAGACCTCCAGAGCCAAGCCTGCTGTGTCTACC |
| | | CCTAGCCTGCTGCTGAGAGTGCTGTTCTGTGCTGTGTGTGGCGAGCC |
| | | CGCCTACAAGTTTGCTGGCGGCGGAAGAAAGCACCCCAGATACCGG |
| | | TGTCGGTCCATGGGCTTCCCTAAGCACTGTGGCAATGGCACCGTGGC |
| | | CATGGCTGAGTGGGATGCCTTCTGCGAAGAACAGGTGCTGGATCTG |
| | | CTGGGCGACGCCGAGAGACTGGAAAAAGTGTGGGTGGCCGGCtccgga |
| | | gggtctggctccggatcaagtggtggcagcggtaccatggccgccagcgacgaagtgaacctgatcgagagca |
| | | gaaccgtggtgcccctgaacacctgggtgctgatctccaacttcaaggtggcctacaacatcctgcggaggcccg |
| | | acggcaccttcaacagacacctggccgagtacctggaccggaaagtgaccgccaacgccaaccctgtggacg |
| | | gcgtgttcagcttcgacgtgctgatcgaccggcggatcaacctgctgagccgggtgtacagacccgcctacgcc |
| | | gatcaggaacagcccccctctatcctggatctggaaaagcccgtggatggcgacatcgtgcccgtgatcctgttct |
| | | tccacggcggcagctttgcccacagcagcgccaatagcgccatctacgacaccctgtgcagacggctcgtggg |
| | | cctgtgcaaatgcgtggtggtgtccgtgaactaccgcagagcccccgagaacccttacccctgcgcctacgatga |
| | | tggctggatcgccctgaactgggtcaacagcagaagctggctgaagtccaagaaagacagcaaggtgcacatct |
| | | ttctggccggcgatagcagcggcggcaatatcgcccataacgtggccctgagagccggcgagtctggcatcgat |
| | | gtgctgggcaatatcctgctgaaccccatgttcggcggcaacgagcggaccgagagcgagaagtctctggacg |
| | | gcaagtacttcgtgaccgtgcgggaccgggactggtactggaaggcctttctgcccgagggcgaggacagaga |
| | | gcaccccgcctgcaatcccttcagccccagaggcaaaagcctggaaggcgtgtccttcccaaagtccctggtgg |
| | | tggtggccggcctggacctgatcagagattggcagctggcctatgccgagggcctgaagaaagccggccagga |
| | | agtgaagctgatgcacctggaaaaggccaccgtgggcttctacctgctgcccaacaacaaccacttccacaacgt |
| | | gatggacgagatcagcgccttcgtgaacgccgagtgccccaagaaaaagcggaaggtgGCCACCAAC |
| | | TTTAGCCTGCTGAAACAGGCTGGCGACGTGGAAGAGAACCCTGGAC |
| | | CTatgaagcgggaccaccaccatcaccatcatcaggacaagaaaaccatgatgatgaacgaagaggacgacg |
| | | gcaacggcatggacgagctgctggctgtgctgggctacaaagtgcggagcagcgagatggccgacgtggccc |
| | | agaaactggaacagctggaagtgatgatgagcaacgtgcaggaagatgacctgtcccagctggccaccgagac |
| | | agtgcactacaaccccgccgagctgtacacctggctggactccatgctgaccgacctgaactccggagggtctg |
| | | gctccggatcaagtggtggcagcggtaccTCCGACTCTGCTGTGGAACTGGCTGAAG |
| | | TGAACGCCGAGCTGGTGGACCTGACCTCTCTGATCGGCTCTCCCGCT |
| | | TATAGAGCTGGCTCCCCTCAGAGAGAAGCCCTGGACGCTAGAATCG |
| | | CTGCCCTGGCTGCTAGACAAGAGGAACTCGAAGGCCTGGAAGCTCG |
| | | GCCTTCAGGATGGGAGTGGCGAGAGACAGGCCAGAGATTTGGCGAC |
| | | TGGTGGCGCGAGCAAGATACCGCCGCTAAGAACACCTGGCTGCGGT |
| | | CTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 122 | GA_Bxb1 | ATGAGAGCCCTGGTGGTCATCCGGCTGTCTAGAGTGACCGACGCTAC |
| | (399/400) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATGGCGCCCCTCTT |
| | | GTTAGAGCCGAGCCTATCCTGACCAGAGAGCAGCTCGAAGCCCTGA |
| | | GAGCTGAGCTGGTCAAGACCTCCAGAGCCAAGCCTGCTGTGTCTACC |
| | | CCTAGCCTGCTGCTGAGAGTGCTGTTCTGTGCTGTGTGTGGCGAGCC |
| | | CGCCTACAAGTTTGCTGGCGGCGGAAGAAAGCACCCCAGATACCGG |
| | | TGTCGGTCCATGGGCTTCCCTAAGCACTGTGGCAATGGCACCGTGGC |
| | | CATGGCTGAGTGGGATGCCTTCTGCGAAGAACAGGTGCTGGATCTG |
| | | CTGGGCGACGCCGAGAGACTGGAAAAAGTGTGGGTGGCCGGCTCCG |
| | | ACTCTGCTGTGGAACTGGCTGAAGTGAACGCCGAGCTGGTGGACCT |
| | | GACCTCTCTGATCGGCTCTCCCGCTTATAGAGCTGGCtccggagggtctggct |
| | | ccggatcaagtggtggcagcggtaccatggccgccagcgacgaagtgaacctgatcgagagcagaaccgtgg |
| | | tgcccctgaacacctgggtgctgatctccaacttcaaggtggcctacaacatcctgcggaggcccgacggcacct |
| | | tcaacagacacctggccgagtacctggaccggaaagtgaccgccaacgccaaccctgtggacggcgtgttcag |
| | | cttcgacgtgctgatcgaccggcggatcaacctgctgagccgggtgtacagacccgcctacgccgatcaggaac |
| | | agcccccctctatcctggatctggaaaagcccgtggatggcgacatcgtgcccgtgatcctgttcttccacggcgg |
| | | cagctttgcccacagcagcgccaatagcgccatctacgacaccctgtgcagacggctcgtgggcctgtgcaaat |
| | | gcgtggtggtgtccgtgaactaccgcagagcccccgagaacccttacccctgcgcctacgatgatggctggatc |
| | | gccctgaactgggtcaacagcagaagctggctgaagtccaagaaagacagcaaggtgcacatctttctggccgg |
| | | cgatagcagcggcggcaatatcgcccataacgtggccctgagagccggcgagtctggcatcgatgtgctgggc |
| | | aatatcctgctgaaccccatgttcggcggcaacgagcggaccgagagcgagaagtctctggacggcaagtactt |
| | | cgtgaccgtgcgggaccgggactggtactggaaggcctttctgcccgagggcgaggacagagagcaccccgc |
| | | ctgcaatcccttcagccccagaggcaaaagcctggaaggcgtgtccttcccaaagtccctggtggtggtggccg |
| | | gcctggacctgatcagagattggcagctggcctatgccgagggcctgaagaaagccggccaggaagtgaagct |
| | | gatgcacctggaaaaggccaccgtgggcttctacctgctgcccaacaacaaccacttccacaacgtgatggacg |
| | | agatcagcgccttcgtgaacgccgagtgccccaagaaaaagcggaaggtgGCCACCAACTTTAG |
| | | CCTGCTGAAACAGGCTGGCGACGTGGAAGAGAACCCTGGACCTatgaa |
| | | gcgggaccaccaccatcaccatcatcaggacaagaaaaccatgatgatgaacgaagaggacgacggcaacgg |
| | | catggacgagctgctggctgtgctgggctacaaagtgcggagcagcgagatggccgacgtggcccagaaactg |
| | | gaacagctggaagtgatgatgagcaacgtgcaggaagatgacctgtcccagctggccaccgagacagtgcact |
| | | acaaccccgccgagctgtacacctggctggactccatgctgaccgacctgaactccggagggtctggctccgga |
| | | tcaagtggtggcagcggtaccTCCCCTCAGAGAGAAGCCCTGGACGCTAGAATC |
| | | GCTGCCCTGGCTGCTAGACAAGAGGAACTCGAAGGCCTGGAAGCTC |
| | | GGCCTTCAGGATGGGAGTGGCGAGAGACAGGCCAGAGATTTGGCGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 123 | GA_Bxb1 | ATGAGAGCCCTGGTGGTCATCCGGCTGTCTAGAGTGACCGACGCTAC |
| | (440/441) | CACCTCTCCTGAGCGGCAGCTGGAATCTTGCCAGCAGCTGTGTGCTC |
| | | AGCGCGGATGGGATGTTGTGGGAGTCGCTGAGGACCTGGATGTGTC |
| | | TGGTGCCGTGGATCCTTTCGACCGGAAGCGGAGGCCTAACCTGGCTA |
| | | GATGGCTGGCCTTTGAGGAACAGCCCTTCGACGTGATCGTGGCCTAC |
| | | AGAGTGGACCGGCTGACCCGGTCTATCAGACATCTGCAGCAGCTGG |
| | | TCCACTGGGCCGAAGATCACAAGAAACTGGTGGTGTCCGCCACCGA |
| | | GGCTCACTTCGATACCACCACACCTTTTGCCGCCGTCGTGATCGCTC |
| | | TGATGGGAACCGTTGCTCAGATGGAACTGGAAGCCATCAAAGAGCG |
| | | GAACAGATCCGCCGCTCACTTCAACATCAGAGCCGGCAAGTACCGG |
| | | GGCTCTTTGCCTCCTTGGGGCTACCTGCCAACAAGAGTGGATGGCGA |
| | | ATGGCGGCTGGTGCCTGATCCTGTGCAGCGGGAAAGAATCCTGGAA |
| | | GTGTACCACAGAGTGGTGGACAACCACGAGCCTCTGCACCTGGTGG |
| | | CCCACGACTTGAATAGAAGAGGCGTGCTGTCCCCTAAGGACTACTTC |
| | | GCCCAGCTGCAGGGCAGAGAGCCTCAGGGAAGAGAGTGGAGCGCT |
| | | ACCGCTCTGAAGCGGTCCATGATCTCTGAGGCCATGCTGGGCTACGC |
| | | TACCCTGAATGGAAAGACCGTGCGGGACGATGATGGCGCCCCTCTT |
| | | GTTAGAGCCGAGCCTATCCTGACCAGAGAGCAGCTCGAAGCCCTGA |
| | | GAGCTGAGCTGGTCAAGACCTCCAGAGCCAAGCCTGCTGTGTCTACC |
| | | CCTAGCCTGCTGCTGAGAGTGCTGTTCTGTGCTGTGTGTGGCGAGCC |
| | | CGCCTACAAGTTTGCTGGCGGCGGAAGAAAGCACCCCAGATACCGG |
| | | TGTCGGTCCATGGGCTTCCCTAAGCACTGTGGCAATGGCACCGTGGC |
| | | CATGGCTGAGTGGGATGCCTTCTGCGAAGAACAGGTGCTGGATCTG |
| | | CTGGGCGACGCCGAGAGACTGGAAAAAGTGTGGGTGGCCGGCTCCG |
| | | ACTCTGCTGTGGAACTGGCTGAAGTGAACGCCGAGCTGGTGGACCT |
| | | GACCTCTCTGATCGGCTCTCCCGCTTATAGAGCTGGCTCCCCTCAGA |
| | | GAGAAGCCCTGGACGCTAGAATCGCTGCCCTGGCTGCTAGACAAGA |
| | | GGAACTCGAAGGCCTGGAAGCTCGGCCTTCAGGATGGGAGTGGCGA |
| | | GAGACAGGCCAGAGATTTGGCtccggagggtctggctccggatcaagtggtggcagcggta |
| | | ccatggccgccagcgacgaagtgaacctgatcgagagcagaaccgtggtgcccctgaacacctgggtgctgat |
| | | ctccaacttcaaggtggcctacaacatcctgcggaggcccgacggcaccttcaacagacacctggccgagtacc |
| | | tggaccggaaagtgaccgccaacgccaaccctgtggacggcgtgttcagcttcgacgtgctgatcgaccggcg |
| | | gatcaacctgctgagccgggtgtacagacccgcctacgccgatcaggaacagcccccctctatcctggatctgg |
| | | aaaagcccgtggatggcgacatcgtgcccgtgatcctgttcttccacggcggcagctttgcccacagcagcgcca |
| | | atagcgccatctacgacaccctgtgcagacggctcgtgggcctgtgcaaatgcgtggtggtgtccgtgaactacc |
| | | gcagagcccccgagaacccttacccctgcgcctacgatgatggctggatcgccctgaactgggtcaacagcaga |
| | | agctggctgaagtccaagaaagacagcaaggtgcacatctttctggccggcgatagcagcggcggcaatatcgc |
| | | ccataacgtggccctgagagccggcgagtctggcatcgatgtgctgggcaatatcctgctgaaccccatgttcgg |
| | | cggcaacgagcggaccgagagcgagaagtctctggacggcaagtacttcgtgaccgtgcgggaccgggactg |
| | | gtactggaaggcctttctgcccgagggcgaggacagagagcaccccgcctgcaatcccttcagccccagaggc |
| | | aaaagcctggaaggcgtgtccttcccaaagtccctggtggtggtggccggcctggacctgatcagagattggca |
| | | gctggcctatgccgagggcctgaagaaagccggccaggaagtgaagctgatgcacctggaaaaggccaccgt |
| | | gggcttctacctgctgcccaacaacaaccacttccacaacgtgatggacgagatcagcgccttcgtgaacgccga |
| | | gtgccccaagaaaaagcggaaggtgGCCACCAACTTTAGCCTGCTGAAACAGGCT |
| | | GGCGACGTGGAAGAGAACCCTGGACCTatgaaggggaccaccaccatcaccatcatc |
| | | aggacaagaaaaccatgatgatgaacgaagaggacgacggcaacggcatggacgagctgctggctgtgctgg |
| | | gctacaaagtgcggagcagcgagatggccgacgtggcccagaaactggaacagctggaagtgatgatgagca |
| | | acgtgcaggaagatgacctgtcccagctggccaccgagacagtgcactacaaccccgccgagctgtacacctg |
| | | gctggactccatgctgaccgacctgaactccggagggtctggctccggatcaagtggtggcagcggtaccGA |
| | | CTGGTGGCGCGAGCAAGATACCGCCGCTAAGAACACCTGGCTGCGG |
| | | TCTATGAATGTGCGGCTGACCTTCGATGTGCGCGGAGGACTGACCAG |
| | | AACCATCGACTTCGGCGACCTGCAAGAGTACGAGCAGCATCTGAGA |
| | | CTGGGCTCCGTGGTGGAAAGACTGCACACCGGCATGTCCggttcaCCAA |
| | | AGAAAAAGCGGAAAGTGTGA |
| 124 | GA_Bxb1 | atgcgagccctggtggtcattcgcctgagcagagtcacagacgctactacaagccctgagcggcagctggagtc |
| | (468/469) | ctgtcagcagctgtgcgcacagcgaggatgggatgtggtcggagtggcagaggatctggacgtgagcggggct |
| | | gtcgatccattcgaccgaaagcggagGcccaacctggcacgatggctggctttcgaggaacagccctttgatgt |
| | | gatcgtcgcctacagagtggacaggctgacacgctcaattcgacatctgcagcagctggtgcattgggccgagg |
| | | atcacaagaaactggtggtcagcgcaactgaagcccacttcgacaccacaactccttttgccgctgtggtcatcgc |
| | | actgatgggcaccgtggcccagatggagctggaagctatcaaggagcgaaaccggagcgcagcccatttcaat |
| | | attcgggccgggaaatacagaggcagcctgcccccttggggctatctgcctacccgggtggatggggagtgga |
| | | gactggtgccagaccccgtccagagagagaggattctggaagtgtaccacagagtggtggacaaccacgaacc |
| | | actgcatctggtggcccacgatctgaataggcgcggagtcctgtctccaaaggactattttgctcagctgcaggga |
| | | agggagccacagggacgagaatggagtgctaccgcactgaagcggtctatgatcagtgaggctatgctgggct |
| | | atgcaactctgaatgggaaaaccgtgagagaTgatgacggagcaccactggtgcgggctgagcctattctgaca |
| | | agagagcagctggaagctctgagggcagaactggtgaaaaccagtagggccaagcctgctgtgtcaacaccaa |
| | | gcctgctgctgcgagtgctgttctgcgcagtctgtggcgagccagcatacaaatttgccggcgggggaaggaag |
| | | catccccgctatcgatgccggagcatggggttccctaagcactgtggaaacggcactgtggctatggccgaatgg |
| | | gacgccttttgtgaggaacaggtgctggatctgctgggggacgcagagcgcctggaaaaagtgtgggtcgctgg |
| | | aagcgattccgctgtggagctggcagaagtcaatgccgagctggtggacctgacctccctgatcggatctcctgc |
| | | atacagggcaggctccccacagcgagaagctctggatgcacgaattgctgcactggcagctcgacaggaggaa |
| | | ctggaggggctggaagccagaccctctggatgggagtggcgagaaacaggccagcggtttggggattggtgg |
| | | agggagcaggacacagcagccaagaacacttggctgagatccatgaatgtcaggctgactttcgacgtgcgag |
| | | gatccggagggtctggctccggatcaagtggtggcagcggtaccatggccgccagcgacgaagtgaacctgat |
| | | cgagagcagaaccgtggtgcccctgaacacctgggtgctgatctccaacttcaaggtggcctacaacatcctgcg |
| | | gaggcccgacggcaccttcaacagacacctggccgagtacctggaccggaaagtgaccgccaacgccaaccc |
| | | tgtggacggcgtgttcagcttcgacgtgctgatcgaccggcggatcaacctgctgagccgggtgtacagacccg |
| | | cctacgccgatcaggaacagcccccctctatcctggatctggaaaagcccgtggatggcgacatcgtgcccgtg |
| | | atcctgttcttccacggcggcagctttgcccacagcagcgccaatagcgccatctacgacaccctgtgcagacgg |
| | | ctcgtgggcctgtgcaaatgcgtggtggtgtccgtgaactaccgcagagcccccgagaacccttacccctgcgc |
| | | ctacgatgatggctggatcgccctgaactgggtcaacagcagaagctggctgaagtccaagaaagacagcaag |
| | | gtgcacatctttctggccggcgatagcagcggcggcaatatcgcccataacgtggccctgagagccggcgagtc |
| | | tggcatcgatgtgctgggcaatatcctgctgaaccccatgttcggcggcaacgagcggaccgagagcgagaagt |
| | | ctctggacggcaagtacttcgtgaccgtgcgggaccgggactggtactggaaggcctttctgcccgagggcgag |
| | | gacagagagcaccccgcctgcaatcccttcagccccagaggcaaaagcctggaaggcgtgtccttcccaaagt |
| | | ccctggtggtggtggccggcctggacctgatcagagattggcagctggcctatgccgagggcctgaagaaagc |
| | | cggccaggaagtgaagctgatgcacctggaaaaggccaccgtgggcttctacctgctgcccaacaacaaccact |
| | | tccacaacgtgatggacgagatcagcgccttcgtgaacgccgagtgccccaagaaaaagcggaaggtgGCC |
| | | ACCAACTTTAGCCTGCTGAAACAGGCTGGCGACGTGGAAGAGAACC |
| | | CTGGACCTatgaagcgggaccaccaccatcaccatcatcaggacaagaaaaccatgatgatgaacgaag |
| | | aggacgacggcaacggcatggacgagctgctggctgtgctgggctacaaagtgcggagcagcgagatggccg |
| | | acgtggcccagaaactggaacagctggaagtgatgatgagcaacgtgcaggaagatgacctgtcccagctggc |
| | | caccgagacagtgcactacaaccccgccgagctgtacacctggctggactccatgctgaccgacctgaactccg |
| | | gagggtctggctccggatcaagtggtggcagcggtaccggactgacccgaacaatcgattttggcgacctgcag |
| | | gagtatgaacagcatctgcgcctgggaagtgtggtcgagcgactgcacaccggcatgtcacccaagaaaaagc |
| | | ggaaggtgtga |
| 125 | GA_PhiC31 | atggatacctacgccggagcctacgacagacagagccgggagagagagaacagcagcgccgccagccccgc |
| | (233/234) | cacccagagaagcgccaacgaggataaggccgccgatctgcagagagaggtggagagggacggcggcaga |
| | | ttcagatttgtgggccacttcagcgaggcccctggcaccagcgccttcggcaccgccgagagGcccgagttcg |
| | | agagaatcctgaacgagtgtagggccggcaggctgaacatgatcatcgtgtacgacgtgtcccggttcagcagg |
| | | ctgaaggtgatggacgccatccctatcgtgtccgagctgctggccctgggcgtgaccatcgtgtccacccaggaa |
| | | ggcgtctttagacagggcaacgtgatggacctgatccacctgatcatgaggctggacgccagccacaaggaga |
| | | gcagcctgaaAagcgccaagatcctggacaccaagaacctgcagagggagctgggcggctatgtgggcggc |
| | | aaggccccctacggcttcgagctggtgtccgaAaccaaggagatcacccggaacggcaggatggtgaacgtg |
| | | gtgatcaacaagctggcccacagcaccacccccctgaccggccccttcgagtttgagcccgacgtgatcaggtg |
| | | gtggtggcgggagatcaagacccacaagcacctgcctttcaagccctccggagggtctggctccggatcaagtg |
| | | gtggcagcggtaccatggccgccagcgacgaagtgaacctgatcgagagcagaaccgtggtgcccctgaaca |
| | | cctgggtgctgatctccaacttcaaggtggcctacaacatcctgcggaggcccgacggcaccttcaacagacacc |
| | | tggccgagtacctggaccggaaagtgaccgccaacgccaaccctgtggacggcgtgttcagcttcgacgtgctg |
| | | atcgaccggcggatcaacctgctgagccgggtgtacagacccgcctacgccgatcaggaacagcccccctctat |
| | | cctggatctggaaaagcccgtggatggcgacatcgtgcccgtgatcctgttcttccacggcggcagctttgcccac |
| | | agcagcgccaatagcgccatctacgacaccctgtgcagacggctcgtgggcctgtgcaaatgcgtggtggtgtc |
| | | cgtgaactaccgcagagcccccgagaacccttacccctgcgcctacgatgatggctggatcgccctgaactggg |
| | | tcaacagcagaagctggctgaagtccaagaaagacagcaaggtgcacatctttctggccggcgatagcagcgg |
| | | cggcaatatcgcccataacgtggccctgagagccggcgagtctggcatcgatgtgctgggcaatatcctgctgaa |
| | | ccccatgttcggcggcaacgagcggaccgagagcgagaagtctctggacggcaagtacttcgtgaccgtgcgg |
| | | gaccgggactggtactggaaggcctttctgcccgagggcgaggacagagagcaccccgcctgcaatcccttca |
| | | gccccagaggcaaaagcctggaaggcgtgtccttcccaaagtccctggtggtggtggccggcctggacctgatc |
| | | agagattggcagctggcctatgccgagggcctgaagaaagccggccaggaagtgaagctgatgcacctggaa |
| | | aaggccaccgtgggcttctacctgctgcccaacaacaaccacttccacaacgtgatggacgagatcagcgccttc |
| | | gtgaacgccgagtgccccaagaaaaagcggaaggtgGCCACCAACTTTAGCCTGCTGAA |
| | | ACAGGCTGGCGACGTGGAAGAGAACCCTGGACCTatgaagcgggaccaccac |
| | | catcaccatcatcaggacaagaaaaccatgatgatgaacgaagaggacgacggcaacggcatggacgagctg |
| | | ctggctgtgctgggctacaaagtgcggagcagcgagatggccgacgtggcccagaaactggaacagctggaa |
| | | gtgatgatgagcaacgtgcaggaagatgacctgtcccagctggccaccgagacagtgcactacaaccccgccg |
| | | agctgtacacctggctggactccatgctgaccgacctgaactccggagggtctggctccggatcaagtggtggca |
| | | gcggtaccggcagccaggccgccatccaccccggcagcatcaccggcctgtgtaagagaatggacgccgacg |
| | | ccgtgcccaccagaggcgaAaccatcggcaagaaaaccgccagcagcgcctgggaccccgccaccgtgatg |
| | | agaatcctgagggaccctaggatcgccggcttcgccgccgaggtgatctacaagaagaagcccgacggcaccc |
| | | ccaccaccaagatcgagggctacagaatccagagGgaccccatcaccctgagGcctgtggagctggactgtg |
| | | gccctatcatcgagcctgccgagtggtacgagctgcaggcctggctggacggcagaggcagaggcaagggcc |
| | | tgagcagaggccaggccatcctgagcgccatggacaagctgtactgtgagtgtggcgccgtgatgaccagcaa |
| | | gagaggcgaggagagcatcaaggacagctaccggtgccggagaagaaaggtggtggaccccagcgcccctg |
| | | gccagcacgagggcacctgtaatgtgagcatggccgccctggacaagttcgtggccgagcggatcttcaacaa |
| | | gatccggcacgccgagggcgacgaggaAaccctggccctgctgtgggaggccgccagaagattcggcaagc |
| | | tgaccgaggcccccgaAaagagcggcgagagggccaacctggtggccgagagagccgacgccctgaacgc |
| | | cctggaggagctgtacgaggacagagccgccggagcctatgacggccctgtgggcaggaagcacttcagaaa |
| | | gcagcaggccgccctgaccctgagacagcagggcgccgaggaaagactggccgagctggaggccgccgag |
| | | gcccctaagctgcccctggatcagtggttccccgaggatgccgacgccgaccccaccggccccaagtcctggt |
| | | ggggcagagccagcgtggacgacaagagggtgttcgtgggcctgttcgtggataagatcgtggtgaccaagag |
| | | caccaccggcaggggccagggcacccccatcgagaagagagccagcatcacctgggccaagcctcccaccg |
| | | acgacgacgaggatgacgcccaggacggcaccgaggacgtggccgcccccaagaaaaagcggaaggtgtg |
| | | a |
| 126 | RAP_PhiC31 | atggatacctacgccggagcctacgacagacagagccgggagagagagaacagcagcgccgccagccccgc |
| | (571/572) | cacccagagaagcgccaacgaggataaggccgccgatctgcagagagaggtggagagggacggcggcaga |
| | | ttcagatttgtgggccacttcagcgaggcccctggcaccagcgccttcggcaccgccgagagGcccgagttcg |
| | | agagaatcctgaacgagtgtagggccggcaggctgaacatgatcatcgtgtacgacgtgtcccggttcagcagg |
| | | ctgaaggtgatggacgccatccctatcgtgtccgagctgctggccctgggcgtgaccatcgtgtccacccaggaa |
| | | ggcgtctttagacagggcaacgtgatggacctgatccacctgatcatgaggctggacgccagccacaaggaga |
| | | gcagcctgaaAagcgccaagatcctggacaccaagaacctgcagagggagctgggcggctatgtgggcggc |
| | | aaggccccctacggcttcgagctggtgtccgaAaccaaggagatcacccggaacggcaggatggtgaacgtg |
| | | gtgatcaacaagctggcccacagcaccacccccctgaccggccccttcgagtttgagcccgacgtgatcaggtg |
| | | gtggtggcgggagatcaagacccacaagcacctgcctttcaagcccggcagccaggccgccatccaccccgg |
| | | cagcatcaccggcctgtgtaagagaatggacgccgacgccgtgcccaccagaggcgaAaccatcggcaaga |
| | | aaaccgccagcagcgcctgggaccccgccaccgtgatgagaatcctgagggaccctaggatcgccggcttcgc |
| | | cgccgaggtgatctacaagaagaagcccgacggcacccccaccaccaagatcgagggctacagaatccagag |
| | | GgaccccatcaccctgagGcctgtggagctggactgtggccctatcatcgagcctgccgagtggtacgagctg |
| | | caggcctggctggacggcagaggcagaggcaagggcctgagcagaggccaggccatcctgagcgccatgga |
| | | caagctgtactgtgagtgtggcgccgtgatgaccagcaagagaggcgaggagagcatcaaggacagctaccg |
| | | gtgccggagaagaaaggtggtggaccccagcgcccctggccagcacgagggcacctgtaatgtgagcatggc |
| | | cgccctggacaagttcgtggccgagcggatcttcaacaagatccggcacgccgagggcgacgaggaAaccct |
| | | ggccctgctgtgggaggccgccagaagattcggcaagctgaccgaggcccccgaAaagagcggcgagagg |
| | | gccaacctggtggccgagagagccgacgccctgaacgccctggaggagctgtacgaggacagagccgccgg |
| | | agcctatgacggccctgtgggcaggaagcacttcagaaagcagcaggccgccctgaccctgagacagcaggg |
| | | cgccgaggaaagactggccgagctggaggccgccgaggcccctaagctgcccctggatcagtggttccccga |
| | | ggatgccgacgccgaccccaccggccccaagtcctggggggcagagccagcgtggacgacaagagggtgtt |
| | | cgtgggcctgttcgtggataagatcgtggtgaccaagagcaccaccggcaggggctccggagggtctggctcc |
| | | ggatcaagtggtggcagcggtaccatcctctggcatgagatgtggcatgaaggcctggaagaggcatctcgtttg |
| | | tactttggggaaaggaacgtgaaaggcatgtttgaggtgctggagcccttgcatgctatgatggaacggggcccc |
| | | cagactctgaaggaaacatcctttaatcaggcctatggtcgagatttaatggaggcccaagagtggtgcaggaagt |
| | | acatgaaatcagggaatgtcaaggacctcctccaagcctgggacctctattatcatgtgttccgacgaatctcaccc |
| | | aagaaaaagcggaaggtgGCCACCAACTTTAGCCTGCTGAAACAGGCTGGCG |
| | | ACGTGGAAGAGAACCCTGGACCTatgtctagaggagtgcaggtggaaaccatctccccag |
| | | gGgacggAcgcaccttccccaagcgcggccagacctgcgtggtgcactacaccgggatgcttgaagatggaa |
| | | agaaatttgattcctcccgggacagaaacaagccctttaagtttatgctaggcaagcaggaggtgatccgaggctg |
| | | ggaagaaggggttgcccagatgagtgtgggtcagagagccaaactgactatatctccagattatgcctatggtgc |
| | | cactgggcacccaggcatcatcccaccacatgccactctcgtGttcgatgtggagcttctaaaactggaatccgg |
| | | agggtctggctccggatcaagtggtggcagcggtacccagggcacccccatcgagaagagagccagcatcac |
| | | ctgggccaagcctcccaccgacgacgacgaggatgacgcccaggacggcaccgaggacgtggccgccccca |
| | | agaaaaagcggaaggtgtga |
| 127 | GA_TP901 | atgaccaagaaggtggccatctacaccagagtgtccaccaccaaccaggccgaggaaggcttcagcatcgacg |
| | (326/327) | agcagatcgaccggctgaccaaatacgccgaggccatgggatggcaggtgtccgatacctacaccgacgccg |
| | | gctttagcggcgccaagctggaaagacccgccatgcagcggctgatcaacgacatcgagaacaaggccttcga |
| | | caccgtgctggtgtacaagctggacaggctgagcagaagcgtgcgggacaccctgtacctcgtgaaggacgtgt |
| | | tcaccaagaacaagatcgacttcatcagcctgaacgagagcatcgacaccagcagcgctatgggcagcctgttc |
| | | ctgaccatcctgagcgccatcaacgagttcgagcgcgagaacatcaaagaacggatgaccatgggcaagctgg |
| | | gcagagccaagagcggcaagagcatgatgtggaccaagaccgccttcggctactaccacaacagaaagaccg |
| | | gcatcctggaaatagtgccactgcaggccaccatcgtggaacagatcttcaccgactacctgagcggcatctccc |
| | | tgaccaagctgagagacaagctgaacgagtccggccacatcggcaaggacatcccttggagctaccggaccct |
| | | gcggcagaccctggacaaccctgtgtactgcggctacatcaagttcaaggactccctgttcgagggcatgcacaa |
| | | gcccatcatcccttacgagacatacctgaaggtgcagaaagagctggaagagagacagcagcagacctacgag |
| | | cggaacaacaaccccagacccttccaggccaagtacatgctgtccggcatggccagatgcggctactgtggcgc |
| | | ccctctgaagatcgtgctgggccacaagagaaaggacggcagccggaccatgaagtaccactgcgccaaccg |
| | | gttccctagaaagaccaagggcatctccggagggtctggctccggatcaagtggtggcagcggtaccatggccg |
| | | ccagcgacgaagtgaacctgatcgagagcagaaccgtggtgcccctgaacacctgggtgctgatctccaacttc |
| | | aaggtggcctacaacatcctgcggaggcccgacggcaccttcaacagacacctggccgagtacctggaccgga |
| | | aagtgaccgccaacgccaaccctgtggacggcgtgttcagcttcgacgtgctgatcgaccggcggatcaacctg |
| | | ctgagccgggtgtacagacccgcctacgccgatcaggaacagcccccctctatcctggatctggaaaagcccgt |
| | | ggatggcgacatcgtgcccgtgatcctgttcttccacggcggcagctttgcccacagcagcgccaatagcgccat |
| | | ctacgacaccctgtgcagacggctcgtgggcctgtgcaaatgcgtggtggtgtccgtgaactaccgcagagccc |
| | | ccgagaacccttacccctgcgcctacgatgatggctggatcgccctgaactgggtcaacagcagaagctggctg |
| | | aagtccaagaaagacagcaaggtgcacatctttctggccggcgatagcagcggcggcaatatcgcccataacgt |
| | | ggccctgagagccggcgagtctggcatcgatgtgctgggcaatatcctgctgaaccccatgttcggcggcaacg |
| | | agcggaccgagagcgagaagtctctggacggcaagtacttcgtgaccgtgcgggaccgggactggtactggaa |
| | | ggcctttctgcccgagggcgaggacagagagcaccccgcctgcaatcccttcagccccagaggcaaaagcctg |
| | | gaaggcgtgtccttcccaaagtccctggtggtggtggccggcctggacctgatcagagattggcagctggcctat |
| | | gccgagggcctgaagaaagccggccaggaagtgaagctgatgcacctggaaaaggccaccgtgggcttctac |
| | | ctgctgcccaacaacaaccacttccacaacgtgatggacgagatcagcgccttcgtgaacgccgagtgccccaa |
| | | gaaaaagcggaaggtgGCCACCAACTTTAGCCTGCTGAAACAGGCTGGCGAC |
| | | GTGGAAGAGAACCCTGGACCTatgaagcgggaccaccaccatcaccatcatcaggacaaga |
| | | aaaccatgatgatgaacgaagaggacgacggcaacggcatggacgagctgctggctgtgctgggctacaaagt |
| | | gcggagcagcgagatggccgacgtggcccagaaactggaacagctggaagtgatgatgagcaacgtgcagg |
| | | aagatgacctgtcccagctggccaccgagacagtgcactacaaccccgccgagctgtacacctggctggactcc |
| | | atgctgaccgacctgaactccggagggtctggctccggatcaagtggtggcagcggtaccaccgtgtacaacga |
| | | caacaagaagtgcgacagcggcacctacgacctgagcaacctggaaaacaccgtgatcgacaacctgatcggc |
| | | ttccaggaaaacaacgacagcctgctgaagatcatcaacggcaacaaccagcccatcctggacacctccagctt |
| | | caagaagcagatcagccagatcgacaagaagatccagaagaacagcgacctgtacctgaacgatttcatcacca |
| | | tggacgagctgaaggaccggaccgactctctgcaggccgagaagaagctgctgaaggccaagatctctgagaa |
| | | caagttcaacgatagcaccgacgtgttcgagctcgtgaaaacacagctgggctccatccccatcaatgagctgag |
| | | ctacgataacaagaaaaagattgtgaacaacctggtgtctaaggtggacgtgaccgccgacaacgtggacatcat |
| | | cttcaagttccagctggcctga |
| 128 | GA_Cre | atgtccaacctgctgactgtgcaccaaaacctgcctgccctccctgtggatgccacctctgatgaagtcaggaaga |
| | (229/230) | acctgatggacatgttcagggacaggcaggccttctctgaacacacctggaagatgctcctgtctgtgtgcagatc |
| | | ctgggctgcctggtgcaagctgaacaacaggaaatggttccctgctgaacctgaggatgtgagggactacctcct |
| | | gtacctgcaagccagaggcctggctgtgaagaccatccaacagcacctgggccagctcaacatgctgcacagg |
| | | agatctggcctgcctcgcccttctgactccaatgctgtgtccctggtgatgaggagaatcagaaaggagaatgtgg |
| | | atgctggggagagagccaagcaggccctggcctttgaacgcactgactttgaccaagtcagatccctgatggag |
| | | aactctgacagatgccaggacatcaggaacctggccttcctgggcattgcctacaacaccctgctgcgcattgcc |
| | | gaaattgccagaatcagagtgaaggacatctcccgcaccgatggtgggagaatgctgatccacattggcaggac |
| | | caagaccctggtgtccacagctggtgtggagaaggccctgtccctgggggttaccaagctggtggagagatgga |
| | | tctctgtgtctggttccggagggtctggctccggatcaagtggtggcagcggtaccatggccgccagcgacgaa |
| | | gtgaacctgatcgagagcagaaccgtggtgcccctgaacacctgggtgctgatctccaacttcaaggtggcctac |
| | | aacatcctgcggaggcccgacggcaccttcaacagacacctggccgagtacctggaccggaaagtgaccgcc |
| | | aacgccaaccctgtggacggcgtgttcagcttcgacgtgctgatcgaccggcggatcaacctgctgagccgggt |
| | | gtacagacccgcctacgccgatcaggaacagcccccctctatcctggatctggaaaagcccgtggatggcgaca |
| | | tcgtgcccgtgatcctgttcttccacggcggcagctttgcccacagcagcgccaatagcgccatctacgacaccct |
| | | gtgcagacggctcgtgggcctgtgcaaatgcgtggtggtgtccgtgaactaccgcagagcccccgagaaccctt |
| | | acccctgcgcctacgatgatggctggatcgccctgaactgggtcaacagcagaagctggctgaagtccaagaaa |
| | | gacagcaaggtgcacatctttctggccggcgatagcagcggcggcaatatcgcccataacgtggccctgagagc |
| | | cggcgagtctggcatcgatgtgctgggcaatatcctgctgaaccccatgttcggcggcaacgagcggaccgaga |
| | | gcgagaagtctctggacggcaagtacttcgtgaccgtgcgggaccgggactggtactggaaggcctttctgccc |
| | | gagggcgaggacagagagcaccccgcctgcaatcccttcagccccagaggcaaaagcctggaaggcgtgtcc |
| | | ttcccaaagtccctggtggtggtggccggcctggacctgatcagagattggcagctggcctatgccgagggcctg |
| | | aagaaagccggccaggaagtgaagctgatgcacctggaaaaggccaccgtgggcttctacctgctgcccaaca |
| | | acaaccacttccacaacgtgatggacgagatcagcgccttcgtgaacgccgagtgccccaagaaaaagcggaa |
| | | ggtgtgaGCCACCAACTTTAGCCTGCTGAAACAGGCTGGCGACGTGGAA |
| | | GAGAACCCTGGACCTgccaccatgaaggggaccaccaccatcaccatcatcaggacaagaaaa |
| | | ccatgatgatgaacgaagaggacgacggcaacggcatggacgagctgctggctgtgctgggctacaaagtgcg |
| | | gagcagcgagatggccgacgtggcccagaaactggaacagctggaagtgatgatgagcaacgtgcaggaaga |
| | | tgacctgtcccagctggccaccgagacagtgcactacaaccccgccgagctgtacacctggctggactccatgct |
| | | gaccgacctgaactccggagggtctggctccggatcaagtggtggcagcggtaccgtggctgatgaccccaac |
| | | aactacctgttctgccgggtcagaaagaatggtgtggctgccccttctgccacctcccaactgtccacccgggccc |
| | | tggaagggatctttgaggccacccaccgcctgatctatggtgccaaggatgactctgggcagagatacctggcct |
| | | ggtctggccactctgccagagtgggtgctgccagggacatggccagggctggtgtgtccatccctgaaatcatgc |
| | | aggctggtggctggaccaatgtgaacattgtgatgaactacatcagaaacctggactctgagactggggccatgg |
| | | tgaggctgctcgaggatggggaccccaagaaaaagcggaaggtgtga |
| 129 | PYL1- | atggcgccaactcaagacgaattcacccaactctcccaatcaatcgccgagttccacacgtaccaactcggtaac |
| | CreC(271)- | ggccgttgctcatctctcctagctcagcgaatccacgcgccgccggaaacagtatggtccgtggtgagacgtttcg |
| | 2A- | ataggccacagatttacaaacacttcatcaaaagctgtaacgtgagtgaagatttcgagatgcgagtgggatgcac |
| | CreN(270)- | gcgcgacgtgaacgtgataagtggattaccggcgaatacgtctcgagagagattagatctgttggacgatgatcg |
| | ABI | gagagtgactgggtttagtataaccggtggtgaacataggctgaggaattataaatcggttacgacggttcatagat |
| | | ttgagaaagaagaagaagaagaaaggatctggaccgttgttttggaatcttatgttgttgatgtaccggaaggtaatt |
| | | cggaggaagatacgagattgtttgctgatacggttattagattgaatcttcagaaacttgcttcgatcactgaagctat |
| | | gaactacccatacgatgttccagattacgcttccggagggtctggctccggatcaagtggtggcagcggtaccC |
| | | TGATCTACGGCGCCAAGGACGATAGCGGCCAGAGATATTTGGCTTG |
| | | GAGCGGCCACTCCGCTAGAGTGGGAGCTGCTAGAGATATGGCTAGA |
| | | GCCGGCGTGTCCATTCCTGAGATCATGCAAGCTGGCGGCTGGACCA |
| | | ACGTGAACATCGTGATGAACTACATCCGCAACCTGGACTCCGAGAC |
| | | AGGCGCTATGGTTCGACTGCTGGAAGATGGCGACcccaagaaaaagcggaag |
| | | gtgGCCACCAACTTTAGCCTGCTGAAACAGGCTGGCGACGTGGAAGA |
| | | GAACCCTGGACCTATGTCCAATCTGCTGACCGTGCACCAGAACCTGC |
| | | CTGCTCTGCCCGTGGACGCCACCAGCGACGAGGTGCGCAAGAACCT |
| | | GATGGACATGTTCCGCGACCGCCAGGCCTTCAGCGAGCACACCTGG |
| | | AAGATGCTGCTGAGCGTGTGCCGCAGCTGGGCCGCCTGGTGCAAGC |
| | | TGAACAACCGCAAGTGGTTCCCCGCCGAGCCCGAGGACGTGCGCGA |
| | | CTACCTGCTGTACCTGCAGGCCCGCGGCCTGGCCGTGAAAACCATCC |
| | | AGCAGCACCTGGGCCAGCTGAACATGCTGCACCGCCGCAGCGGCCT |
| | | GcctAGGCCATCTGACTCTAATGCCGTGTCTCTGGTCATGCGGCGGAT |
| | | CCGGAAAGAAAACGTGGACGCCGGCGAGAGAGCTAAGCAGGCTCT |
| | | GGCTTTCGAGAGAACCGACTTCGACCAAGTGCGGTCCCTGATGGAA |
| | | AACTCCGACCGGTGCCAGGATATCCGGAACCTGGCTTTTCTGGGAAT |
| | | CGCCTACAACACCCTGCTGCGGATCGCTGAGATCGCCCGGATCAGA |
| | | GTGAAGGACATCTCTAGAACCGACGGCGGCAGAATGCTGATCCACA |
| | | TCGGCAGAACAAAGACCCTGGTGTCCACAGCTGGCGTGGAAAAGGC |
| | | TCTGTCTCTGGGCGTGACCAAGCTGGTGGAACGGTGGATTTCTGTGT |
| | | CCGGCGTGGCCGACGATCCCAACAACTACCTGTTCTGCAGAGTCCGG |
| | | AAGAACGGCGTGGCAGCCCCTTCTGCTACATCCCAGCTGTCTACAAG |
| | | AGCCCTGGAAGGCATCTTCGAGGCTACCCACAGAtccggagggtctggctccgg |
| | | atcaagtggtggcagcggtacccctttgtatggttttacttcgatttgtggaagaagGcctgagatggaagctgctg |
| | | tttcgactataccaagattccttcaatcttcctctggttcgatgttagatggtcggtttgatcctcaatccgccgctcattt |
| | | cttcggtgtttacgacggccatggcggttctcaggtagcgaactattgtagagagaggatgcatttggctttggcgg |
| | | aggagatagctaaggagaaaccgatgctctgcgatggtgatacgtggctggagaagtggaagaaagctcttttca |
| | | actcgttcctgagagttgactcggagattgagtcagttgcgccggagacggttgggtcaacgtcggtggttgccgt |
| | | tgttttcccgtctcacatcttcgtcgctaactgcggtgactctagagccgttctttgccgcggcaaaactgcacttcca |
| | | ttatccgttgaccataaaccggatagagaagatgaagctgcgaggattgaagccgcaggagggaaagtgattca |
| | | gtggaatggagctcgtgttttcggtgttctcgccatgtcgagatccattggcgatagatacttgaaaccatccatcatt |
| | | cctgatccggaagtgacggctgtgaagagagtaaaagaagatgattgtctgattttggcgagtgacggggtttgg |
| | | gatgtaatgacggatgaagaagcgtgtgagatggcaaggaagcggattctcttgtggcacaagaaaaacgcggt |
| | | ggctggggatgcatcgttgctcgcggatgagcggagaaaggaagggaaagatcctgcggcgatgtccgcggct |
| | | gagtatttgtcaaagctggcgatacagagaggaagcaaagacaacataagtgtggtggtggttgatttgaaggatt |
| | | acaaggacgatgacgataagcccaagaaaaagcggaaggtgtga |
| 130 | CreN(270)- | ATGTCCAATCTGCTGACCGTGCACCAGAACCTGCCTGCTCTGCCCGT |
| | ABI-2A- | GGACGCCACCAGCGACGAGGTGCGCAAGAACCTGATGGACATGTTC |
| | PYL1- | CGCGACCGCCAGGCCTTCAGCGAGCACACCTGGAAGATGCTGCTGA |
| | CreC(271) | GCGTGTGCCGCAGCTGGGCCGCCTGGTGCAAGCTGAACAACCGCAA |
| | | GTGGTTCCCCGCCGAGCCCGAGGACGTGCGCGACTACCTGCTGTACC |
| | | TGCAGGCCCGCGGCCTGGCCGTGAAAACCATCCAGCAGCACCTGGG |
| | | CCAGCTGAACATGCTGCACCGCCGCAGCGGCCTGcctAGGCCATCTGA |
| | | CTCTAATGCCGTGTCTCTGGTCATGCGGCGGATCCGGAAAGAAAAC |
| | | GTGGACGCCGGCGAGAGAGCTAAGCAGGCTCTGGCTTTCGAGAGAA |
| | | CCGACTTCGACCAAGTGCGGTCCCTGATGGAAAACTCCGACCGGTG |
| | | CCAGGATATCCGGAACCTGGCTTTTCTGGGAATCGCCTACAACACCC |
| | | TGCTGCGGATCGCTGAGATCGCCCGGATCAGAGTGAAGGACATCTC |
| | | TAGAACCGACGGCGGCAGAATGCTGATCCACATCGGCAGAACAAAG |
| | | ACCCTGGTGTCCACAGCTGGCGTGGAAAAGGCTCTGTCTCTGGGCGT |
| | | GACCAAGCTGGTGGAACGGTGGATTTCTGTGTCCGGCGTGGCCGAC |
| | | GATCCCAACAACTACCTGTTCTGCAGAGTCCGGAAGAACGGCGTGG |
| | | CAGCCCCTTCTGCTACATCCCAGCTGTCTACAAGAGCCCTGGAAGGC |
| | | ATCTTCGAGGCTACCCACAGAtccggagggtctggctccggatcaagtggtggcagcggta |
| | | cccctttgtatggttttacttcgatttgtggaagaagGcctgagatggaagctgctgtttcgactataccaagattcctt |
| | | caatcttcctctggttcgatgttagatggtcggtttgatcctcaatccgccgctcatttcttcggtgtttacgacggccat |
| | | ggcggttctcaggtagcgaactattgtagagagaggatgcatttggctttggcggaggagatagctaaggagaaa |
| | | ccgatgctctgcgatggtgatacgtggctggagaagtggaagaaagctcttttcaactcgttcctgagagttgactc |
| | | ggagattgagtcagttgcgccggagacggttgggtcaacgtcggtggttgccgttgttttcccgtctcacatcttcgt |
| | | cgctaactgcggtgactctagagccgttctttgccgcggcaaaactgcacttccattatccgttgaccataaaccgg |
| | | atagagaagatgaagctgcgaggattgaagccgcaggagggaaagtgattcagtggaatggagctcgtgttttc |
| | | ggtgttctcgccatgtcgagatccattggcgatagatacttgaaaccatccatcattcctgatccggaagtgacggc |
| | | tgtgaagagagtaaaagaagatgattgtctgattttggcgagtgacggggtttgggatgtaatgacggatgaagaa |
| | | gcgtgtgagatggcaaggaagcggattctcttgtggcacaagaaaaacgcggtggctggggatgcatcgttgct |
| | | cgcggatgagcggagaaaggaagggaaagatcctgcggcgatgtccgcggctgagtatttgtcaaagctggcg |
| | | atacagagaggaagcaaagacaacataagtgtggtggtggttgatttgaaggattacaaggacgatgacgataag |
| | | cccaagaaaaagcggaaggtgGCCACCAACTTTAGCCTGCTGAAACAGGCTGGC |
| | | GACGTGGAAGAGAACCCTGGACCTatggcgccaactcaagacgaattcacccaactctcc |
| | | caatcaatcgccgagttccacacgtaccaactcggtaacggccgttgctcatctctcctagctcagcgaatccacg |
| | | cgccgccggaaacagtatggtccgtggtgagacgtttcgataggccacagatttacaaacacttcatcaaaagctg |
| | | taacgtgagtgaagatttcgagatgcgagtgggatgcacgcgcgacgtgaacgtgataagtggattaccggcga |
| | | atacgtctcgagagagattagatctgttggacgatgatcggagagtgactgggtttagtataaccggtggtgaacat |
| | | aggctgaggaattataaatcggttacgacggttcatagatttgagaaagaagaagaagaagaaaggatctggacc |
| | | gttgttttggaatcttatgttgttgatgtaccggaaggtaattcggaggaagatacgagattgtttgctgatacggttatt |
| | | agattgaatcttcagaaacttgcttcgatcactgaagctatgaactacccatacgatgttccagattacgcttccgga |
| | | gggtctggctccggatcaagtggtggcagcggtaccCTGATCTACGGCGCCAAGGACGA |
| | | TAGCGGCCAGAGATATTTGGCTTGGAGCGGCCACTCCGCTAGAGTG |
| | | GGAGCTGCTAGAGATATGGCTAGAGCCGGCGTGTCCATTCCTGAGA |
| | | TCATGCAAGCTGGCGGCTGGACCAACGTGAACATCGTGATGAACTA |
| | | CATCCGCAACCTGGACTCCGAGACAGGCGCTATGGTTCGACTGCTGG |
| | | AAGATGGCGACcccaagaaaaagcggaaggtgtga |
| 131 | GA_Vcre | atgatcgagaaccagctgagcctgctgggcgacttttctggcgtgcggcccgacgatgtgaaaaccgccattcag |
| | (269/270) | gccgcccagaaaaagggcatcaacgtggccgagaacgagcagttcaaggccgccttcgagcatctgctgaacg |
| | | agttcaagaagcgggaagagagatacagccccaacaccctgcggcggctggaaagcgcctggacctgcttcgt |
| | | ggattggtgcctggccaaccacagacacagcctgcctgccacccccgataccgtggaagccttcttcatcgagc |
| | | gggccgaggaactgcaccggaacaccctgagcgtgtacagatgggccatcagccgggtgcacagagtggccg |
| | | gatgccctgatccctgcctggacatctacgtggaagatcggctgaaggccattgcccggaagaaagtgcgggaa |
| | | ggcgaggccgtgaagcaggccagccctttcaacgagcagcatctgctgaagctgaccagcctgtggtacagaa |
| | | gcgacaagctgctgctgcggcggaacctggctctgctggctgtggcctacgagagcatgctgagagccagcga |
| | | gctggccaacatccgggtgtccgatatggaactggccggcgacggaaccgccatcctgaccatccctatcacca |
| | | agaccaaccactccggcgagcccgatacctgcatcctgtcccaggatgtggtgtccctgctgatggactacaccg |
| | | aggccggcaagctggatatgagcagcgacggcttcctgttcgtgggcgtgtccaagcacaacacctgtatctccg |
| | | gagggtctggctccggatcaagtggtggcagcggtaccatggccgccagcgacgaagtgaacctgatcgagag |
| | | cagaaccgtggtgcccctgaacacctgggtgctgatctccaacttcaaggtggcctacaacatcctgcggaggcc |
| | | cgacggcaccttcaacagacacctggccgagtacctggaccggaaagtgaccgccaacgccaaccctgtggac |
| | | ggcgtgttcagcttcgacgtgctgatcgaccggcggatcaacctgctgagccgggtgtacagacccgcctacgc |
| | | cgatcaggaacagcccccctctatcctggatctggaaaagcccgtggatggcgacatcgtgcccgtgatcctgtt |
| | | cttccacggcggcagctttgcccacagcagcgccaatagcgccatctacgacaccctgtgcagacggctcgtgg |
| | | gcctgtgcaaatgcgtggtggtgtccgtgaactaccgcagagcccccgagaacccttacccctgcgcctacgatg |
| | | atggctggatcgccctgaactgggtcaacagcagaagctggctgaagtccaagaaagacagcaaggtgcacat |
| | | ctttctggccggcgatagcagcggcggcaatatcgcccataacgtggccctgagagccggcgagtctggcatcg |
| | | atgtgctgggcaatatcctgctgaaccccatgttcggcggcaacgagcggaccgagagcgagaagtctctggac |
| | | ggcaagtacttcgtgaccgtgcgggaccgggactggtactggaaggcctttctgcccgagggcgaggacagag |
| | | agcaccccgcctgcaatcccttcagccccagaggcaaaagcctggaaggcgtgtccttcccaaagtccctggtg |
| | | gtggtggccggcctggacctgatcagagattggcagctggcctatgccgagggcctgaagaaagccggccag |
| | | gaagtgaagctgatgcacctggaaaaggccaccgtgggcttctacctgctgcccaacaacaaccacttccacaa |
| | | cgtgatggacgagatcagcgccttcgtgaacgccgagtgccccaagaaaaagcggaaggtgGCCACCA |
| | | ACTTTAGCCTGCTGAAACAGGCTGGCGACGTGGAAGAGAACCCTGG |
| | | ACCTatgaagcgggaccaccaccatcaccatcatcaggacaagaaaaccatgatgatgaacgaagaggacg |
| | | acggcaacggcatggacgagctgctggctgtgctgggctacaaagtgcggagcagcgagatggccgacgtgg |
| | | cccagaaactggaacagctggaagtgatgatgagcaacgtgcaggaagatgacctgtcccagctggccaccga |
| | | gacagtgcactacaaccccgccgagctgtacacctggctggactccatgctgaccgacctgaactccggagggt |
| | | ctggctccggatcaagtggtggcagcggtaccaagcccaagaaggacaagcagaccggcgaggtgctgcaca |
| | | agcccatcaccaccaagacagtggaaggcgtgttctacagcgcctgggagacactggacctgggcagacagg |
| | | gcgtgaagcctttcacagcccacagcgccagagtgggagccgctcaggacctgctgaagaagggctacaatac |
| | | cctgcagatccagcagtccggccggtggtctagcggagccatggtggccagatacggcagagccatcctggct |
| | | agggatggcgctatggcccacagcagagtgaaaaccagatccgcccccatgcagtggggcaaggacgagaa |
| | | ggaccccaagaaaaagcggaaggtgtga |

TABLE 9
Exemplary Expression Cassette Nucleic Acid Sequences

| SEQ | | |
|---|---|---|
| ID NO: | Descr. | Sequence |
| 132 | pAI-2469 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatgagccagttcgacatcctgtgcaagaccccccccaaggtgctggtgcggcagttcgtggagagattcga |
| | | gaggcccagcggcgagaagatcgccagctgtgccgccgagctgacctacctgtgctggatgatcacccacaac |
| | | ggcaccgccatcaagagggccaccttcatgagctacaacaccatcatcagcaacagcctgagcttcgacatcgt |
| | | gaacaagagcctgcagttcaagtacaagacccagaaggccaccatcctggaggccagcctgaagaagctgatc |
| | | cccgcctgggagttcaccatcatcccttacaacggccagaagcaccagagcgacatcaccgacatcgtgtccag |
| | | cctgcagctgcagttcgagagcagcgaggaggccgacaagggcaacagccacagcaagaagatgctgaagg |
| | | ccctgctgtccgagggcgagagcatctgggagatcaccgagaagatcctgaacagcttcgagtacaccagcag |
| | | gttcaccaagaccaagaccctgtaccagttcctgttcctggccacattcatcaactgcggcaggttcagcgacatc |
| | | aagaacgtggaccccaagagcttcaagctggtgcagaacaagtacctgggcgtgatcattcagtgcctggtgac |
| | | cgaAaccaagacaagcgtgtccaggcacatctactttttcagcgccagaggcaggatcgaccccctggtgtacc |
| | | tggacgagttcctgaggaacagcgagcccgtgctgaagagagtgaacaggaccggcaacagcagcagcaaca |
| | | agcaggagtaccagctgctgaaggacaacctggtgcgcagctacaacaaggccctgaagaagaacgccccct |
| | | accccatcttcgctatcaagaacggccctaagagccacatcggcaggcacctgatgaccagctttctgagcatga |
| | | agggcctgaccgagctgacaaacgtggtgggcaactggagcgacaagagggcctccgccgtggccaggacc |
| | | acctacacccaccagatcaccgccatccccgaccactacttcgccctggtgtccaggtactacgcctacgacccc |
| | | atcagcaaggagatgatcgccctgaaggacgaAaccaaccccatcgaggagtggcagcacatcgagcagctg |
| | | aagggcagcgccgagggctccggagggtctggctccggatcaagtggtggcagcggtacccctttgtatggtttt |
| | | acttcgatttgtggaagaagGcctgagatggaagatgctgtttcgactataccaagattccttcaatcttcctctggtt |
| | | cgatgttagatggtcggtttgatcctcaatccgccgctcatttcttcggtgtttacgacggccatggcggttctcaggt |
| | | agcgaactattgtagagagaggatgcatttggctttggcggaggagatagctaaggagaaaccgatgctctgcga |
| | | tggtgatacgtggctggagaagtggaagaaagctcttttcaactcgttcctgagagttgactcggagattgggtcag |
| | | ttgcgccggaAacggttgggtcaacgtcggtggttgccgttgttttcccAtctcacatcttcgtcgctaactgcggt |
| | | gactctagagccgttctttgccgcggcaaaactgcacttccattatccgttgaccataaaccggatagagaagatga |
| | | agctgcgaggattgaagccgcaggagggaaagtgattcagtggaatggagctcgtgttttcggtgttctcgccatg |
| | | tcgagatccattggcgatagatacttgaaaccatccatcattcctgatccggaagtgacggctgtgaagagagtaa |
| | | aagaagatgattgtctgattttggcgagtgacggggtttgggatgtaatgacggatgaagaagcgtgtgagatggc |
| | | aaggaagcggattctcttgtggcacaagaaaaacgcggtggctggggatgcatcgttgctcgcggatgagcgga |
| | | gaaaggaagggaaagatcctgcggcgatgtccgcggctgagtatttgtcaaagctggcgatacagagaggaag |
| | | caaagacaacataagtgtggtggtggttgatttgaaggattacaaggacgatgacgataagcccaagaaaaagc |
| | | ggaaggtgGCCACCAACTTTAGCCTGCTGAAACAGGCTGGCGACGTGGA |
| | | AGAGAACCCTGGACCTatggcgccaactcaagacgagttcacccaactctcccaatcaatcgccg |
| | | agttccacacgtaccaactcggtaacggccgttgctcatctctcctagctcagcgaatccacgcgccgccggaaa |
| | | cagtatggtccgtggtgagGcgtttcgataggccacagatttacaaacacttcatcaaaagctgtaacgtgagtga |
| | | agatttcgagatgcgagtgggatgcacgcgcgacgtgaacgtgataagtggattaccggcgaatacCtctcgag |
| | | agagattagatctgttggacgatgatcggagagtgactgggtttagtataaccggtggtgaacataggctgaggaa |
| | | ttataaatcggttacgacggttcatagatttgagaaagaagaagaagaagaaaggatctggaccgttgttttggaat |
| | | cttatgttgttgatgtaccggaaggtaattcggaggaagatacgagattgtttgctgatacggttattagattgaatctt |
| | | cagaaacttgcttcgatcactgaagctatgaactacccatacgatgttccagattacgcttccggagggtctggctc |
| | | cggatcaagtggtggcagcggtaccagcatcagataccccgcctggaacggcatcatcagccaggaggtgctg |
| | | gactacctgagcagctacatcaacaggcggatccccaagaaaaagcggaaggtgtgatgaCCCAccactgg |
| | | attgtacaattacGAGTCTAAGTAAGGATCCCTGTGCCTTCTAGTTGCCAGCC |
| | | ATCTGTTGTTTGCCCCTCCCCCGTGCCTTCCTTGACCCTGGAAGGTGC |
| | | CACTCCCACTGTCCTTTCCTAATAAAATGAGGAAATTGCATCGCATT |
| | | GTCTGAGTAGGTGTCATTCTATTCTGGGGGGTGGGGTGGGGCAGGA |
| | | CAGCAAGGGGGAGGATTGGGATGACAATAGCAGGCATGCTGGGGAT |
| | | GCGGTGGGCTCTATGG |
| 133 | pAI-2468 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatgagccagttcgacatcctgtgcaagaccccccccaaggtgctggtgcggcagttcgtggagagattcga |
| | | gaggcccagctccggagggtctggctccggatcaagtggtggcagcggtaccatggccgccagcgacgaagt |
| | | gaacctgatcgagagcagaaccgtggtgcccctgaacacctgggtgctgatctccaacttcaaggtggcctacaa |
| | | catcctgcggaggcccgacggcaccttcaacagacacctggccgagtacctggaccggaaagtgaccgccaa |
| | | cgccaaccctgtggacggcgtgttcagcttcgacgtgctgatcgaccggcggatcaacctgctgagccgggtgt |
| | | acagacccgcctacgccgatcaggaacagcccccctctatcctggatctggaaaagcccgtggatggcgacatc |
| | | gtgcccgtgatcctgttcttccacggcggcagctttgcccacagcagcgccaatagcgccatctacgacaccctgt |
| | | gcagacggctcgtgggcctgtgcaaatgcgtggtggtgtccgtgaactaccgcagagcccccgagaacccttac |
| | | ccctgcgcctacgatgatggctggatcgccctgaactgggtcaacagcagaagctggctgaagtccaagaaaga |
| | | cagcaaggtgcacatctttctggccggcgatagcagcggcggcaatatcgcccataacgtggccctgagagccg |
| | | gcgagtctggcatcgatgtgctgggcaatatcctgctgaaccccatgttcggcggcaacgagcggaccgagagc |
| | | gagaagtctctggacggcaagtacttcgtgaccgtgcgggaccgggactggtactggaaggcctttctgcccga |
| | | gggcgaggacagagagcaccccgcctgcaatcccttcagccccagaggcaaaagcctggaaggcgtgtccttc |
| | | ccaaagtccctggtggtggtggccggcctggacctgatcagagattggcagctggcctatgccgagggcctgaa |
| | | gaaagccggccaggaagtgaagctgatgcacctggaaaaggccaccgtgggcttctacctgctgcccaacaac |
| | | aaccacttccacaacgtgatggacgagatcagcgccttcgtgaacgccgagtgccccaagaaaaagcggaagg |
| | | tgGCCACCAACTTTAGCCTGCTGAAACAGGCTGGCGACGTGGAAGAG |
| | | AACCCTGGACCTatgaagcgggaccaccaccatcaccatcatcaggacaagaaaaccatgatgatga |
| | | acgaagaggacgacggcaacggcatggacgagctgctggctgtgctgggctacaaagtgcggagcagcgag |
| | | atggccgacgtggcccagaaactggaacagctggaagtgatgatgagcaacgtgcaggaagatgacctgtccc |
| | | agctggccaccgagacagtgcactacaaccccgccgagctgtacacctggctggactccatgctgaccgacctg |
| | | aactccggagggtctggctccggatcaagtggtggcagcggtaccggcgagaagatcgccagctgtgccgccg |
| | | agctgacctacctgtgctggatgatcacccacaacggcaccgccatcaagagggccaccttcatgagctacaac |
| | | accatcatcagcaacagcctgagcttcgacatcgtgaacaagagcctgcagttcaagtacaagacccagaaggc |
| | | caccatcctggaggccagcctgaagaagctgatccccgcctgggagttcaccatcatcccttacaacggccaga |
| | | agcaccagagcgacatcaccgacatcgtgtccagcctgcagctgcagttcgagagcagcgaggaggccgaca |
| | | agggcaacagccacagcaagaagatgctgaaggccctgctgtccgagggcgagagcatctgggagatcaccg |
| | | agaagatcctgaacagcttcgagtacaccagcaggttcaccaagaccaagaccctgtaccagttcctgttcctggc |
| | | cacattcatcaactgcggcaggttcagcgacatcaagaacgtggaccccaagagcttcaagctggtgcagaaca |
| | | agtacctgggcgtgatcattcagtgcctggtgaccgaAaccaagacaagcgtgtccaggcacatctactttttcag |
| | | cgccagaggcaggatcgaccccctggtgtacctggacgagttcctgaggaacagcgagcccgtgctgaagaga |
| | | gtgaacaggaccggcaacagcagcagcaacaagcaggagtaccagctgctgaaggacaacctggtgcgcag |
| | | ctacaacaaggccctgaagaagaacgccccctaccccatcttcgctatcaagaacggccctaagagccacatcg |
| | | gcaggcacctgatgaccagctttctgagcatgaagggcctgaccgagctgacaaacgtggtgggcaactggag |
| | | cgacaagagggcctccgccgtggccaggaccacctacacccaccagatcaccgccatccccgaccactacttc |
| | | gccctggtgtccaggtactacgcctacgaccccatcagcaaggagatgatcgccctgaaggacgaAaccaacc |
| | | ccatcgaggagtggcagcacatcgagcagctgaagggcagcgccgagggcagcatcagataccccgcctgg |
| | | aacggcatcatcagccaggaggtgctggactacctgagcagctacatcaacaggcggatccccaagaaaaagc |
| | | ggaaggtgtgatgaCCCAccactggattgtacaattacGAGTCTAAGTAAGGATCCCTGT |
| | | GCCTTCTAGTTGCCAGCCATCTGTTGTTTGCCCCTCCCCCGTGCCTTC |
| | | CTTGACCCTGGAAGGTGCCACTCCCACTGTCCTTTCCTAATAAAATG |
| | | AGGAAATTGCATCGCATTGTCTGAGTAGGTGTCATTCTATTCTGGGG |
| | | GGTGGGGTGGGGCAGGACAGCAAGGGGGAGGATTGGGATGACAAT |
| | | AGCAGGCATGCTGGGGATGCGGTGGGCTCTATGG |
| 134 | pAI-2474 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatgcgagccctggtggtcattcgcctgagcagagtcacagacgctactacaagccctgagcggcagctgga |
| | | gtcctgtcagcagctgtgcgcacagcgaggatgggatgtggtcggagtggcagaggatctggacgtgagcggg |
| | | gctgtcgatccattcgaccgaaagcggagGcccaacctggcacgatggctggctttcgaggaacagccctttga |
| | | tgtgatcgtcgcctacagagtggacaggctgacacgctcaattcgacatctgcagcagctggtgcattgggccga |
| | | ggatcacaagaaactggtggtcagcgcaactgaagcccacttcgacaccacaactccttttgccgctgtggtcatc |
| | | gcactgatgggcaccgtggcccagatggagctggaagctatcaaggagcgaaaccggagcgcagcccatttca |
| | | atattcgggccgggaaatacagaggcagcctgcccccttggggctatctgcctacccgggtggatggggagtgg |
| | | agactggtgccagaccccgtccagagagagaggattctggaagtgtaccacagagtggtggacaaccacgaac |
| | | cactgcatctggtggcccacgatctgaataggcgcggagtcctgtctccaaaggactattttgctcagctgcaggg |
| | | aagggagccacagggacgagaatggagtgctaccgcactgaagcggtctatgatcagtgaggctatgctgggc |
| | | tatgcaactctgaatgggaaaaccgtgagagaTgatgacggagcaccactggtgcgggctgagcctattctgac |
| | | aagagagcagctggaagctctgagggcagaactggtgaaaaccagtagggccaagcctgctgtgtcaacacca |
| | | agcctgctgctgcgagtgctgttctgcgcagtctgtggcgagccagcatacaaatttgccggcgggggaaggaa |
| | | gcatccccgctatcgatgccggagcatggggttccctaagcactgtggaaacggcactgtggctatggccgaatg |
| | | ggacgccttttgtgaggaacaggtgctggatctgctgggggacgcagagcgcctggaaaaagtgtgggtcgctg |
| | | gaagcgattccgctgtggagctggcagaagtcaatgccgagctggtggacctgacctccctgatcggatctcctg |
| | | catacagggcaggctccccacagcgagaagctctggatgcacgaattgctgcactggcagctcgacaggagga |
| | | actggaggggctggaagccagaccctctggatgggagtggcgagaaacaggccagcggtttggggattggtg |
| | | gagggagcaggacacagcagccaagaacacttggctgagatccatgaatgtcaggctgactttcgacgtgcga |
| | | ggatccggagggtctggctccggatcaagtggtggcagcggtaccatggccgccagcgacgaagtgaacctga |
| | | tcgagagcagaaccgtggtgcccctgaacacctgggtgctgatctccaacttcaaggtggcctacaacatcctgc |
| | | ggaggcccgacggcaccttcaacagacacctggccgagtacctggaccggaaagtgaccgccaacgccaacc |
| | | ctgtggacggcgtgttcagcttcgacgtgctgatcgaccggcggatcaacctgctgagccgggtgtacagaccc |
| | | gcctacgccgatcaggaacagcccccctctatcctggatctggaaaagcccgtggatggcgacatcgtgcccgt |
| | | gatcctgttcttccacggcggcagctttgcccacagcagcgccaatagcgccatctacgacaccctgtgcagacg |
| | | gctcgtgggcctgtgcaaatgcgtggtggtgtccgtgaactaccgcagagcccccgagaacccttacccctgcg |
| | | cctacgatgatggctggatcgccctgaactgggtcaacagcagaagctggctgaagtccaagaaagacagcaa |
| | | ggtgcacatctttctggccggcgatagcagcggcggcaatatcgcccataacgtggccctgagagccggcgagt |
| | | ctggcatcgatgtgctgggcaatatcctgctgaaccccatgttcggcggcaacgagcggaccgagagcgagaa |
| | | gtctctggacggcaagtacttcgtgaccgtgcgggaccgggactggtactggaaggcctttctgcccgagggcg |
| | | aggacagagagcaccccgcctgcaatcccttcagccccagaggcaaaagcctggaaggcgtgtccttcccaaa |
| | | gtccctggtggtggtggccggcctggacctgatcagagattggcagctggcctatgccgagggcctgaagaaag |
| | | ccggccaggaagtgaagctgatgcacctggaaaaggccaccgtgggcttctacctgctgcccaacaacaacca |
| | | cttccacaacgtgatggacgagatcagcgccttcgtgaacgccgagtgccccaagaaaaagcggaaggtgGC |
| | | CACCAACTTTAGCCTGCTGAAACAGGCTGGCGACGTGGAAGAGAAC |
| | | CCTGGACCTatgaagcgggaccaccaccatcaccatcatcaggacaagaaaaccatgatgatgaacga |
| | | agaggacgacggcaacggcatggacgagctgctggctgtgctgggctacaaagtgcggagcagcgagatggc |
| | | cgacgtggcccagaaactggaacagctggaagtgatgatgagcaacgtgcaggaagatgacctgtcccagctg |
| | | gccaccgagacagtgcactacaaccccgccgagctgtacacctggctggactccatgctgaccgacctgaactc |
| | | cggagggtctggctccggatcaagtggtggcagcggtaccggactgacccgaacaatcgattttggcgacctgc |
| | | aggagtatgaacagcatctgcgcctgggaagtgtggtcgagcgactgcacaccggcatgtcacccaagaaaaa |
| | | gcggaaggtgtgatgaCCCAccactggattgtacaattacGAGTCTAAGTAAGGATCCCT |
| | | GTGCCTTCTAGTTGCCAGCCATCTGTTGTTTGCCCCTCCCCCGTGCCT |
| | | TCCTTGACCCTGGAAGGTGCCACTCCCACTGTCCTTTCCTAATAAAA |
| | | TGAGGAAATTGCATCGCATTGTCTGAGTAGGTGTCATTCTATTCTGG |
| | | GGGGTGGGGTGGGGCAGGACAGCAAGGGGGAGGATTGGGATGACA |
| | | ATAGCAGGCATGCTGGGGATGCGGTGGGCTCTATGG |
| 135 | pAI-7049 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCtCTGGGCGGCAAGGCTGGCCCGGTCGGCACCAGTTGCGTGAGCGG |
| | | AAAGATGGCCGCTTCCCGGCCCTGCTGCAGGGAGCaCAAAATGGAG |
| | | GACGCGGCGCTCGGGAGAGCGGGCGGGTGAGTCACCCACACAAAG |
| | | GAAAAGGGCCTTTCCGTCCTCAGCCGTCGCTTCATGTGACTCCACGG |
| | | AGTACCGGGCGCCGTCCAGGCACCTCGATTAGTTCTCcAGCTTTTGG |
| | | AGTACGTCGTCTTTAGGTTGGGGGGAGGGGTTTTATGCGATGGAGTT |
| | | TCCCCACACTGAGTGGGTGGAGACTGAAGTTAGGCCAGCTTGGCAC |
| | | TTGATGTAATTCTCCTTGGAATTTGCCCTTTTTGAGTTTGGATCTTGG |
| | | TTCATTCTCAAGCCTCAGACAGTGGTTCAAAGTTTTTTTCTTCCATTT |
| | | CAGGTGTCGTgaCAACTAAGTATGTACTATACAGAGGCAAgccaccATG |
| | | AGAGCCCTGGTGGTCATCCGGCTGTCTAGAGTGACCGACGCTACCAC |
| | | CTCTCCTGAGCGGCAGCTGGAATCTTGCCAGCAGCTGTGTGCTCAGC |
| | | GCGGATGGGATGTTGTGGGAGTCGCTGAGGACCTGGATGTGTCTGG |
| | | TGCCGTGGATCCTTTCGACCGGAAGCGGAGGCCTAACCTGGCTAGAT |
| | | GGCTGGCCTTTGAGGAACAGCCCTTCGACGTGATCGTGGCCTACAGA |
| | | GTGGACCGGCTGACCCGGTCTATCAGACATCTGCAGCAGCTGGTCCA |
| | | CTGGGCCGAAGATCACAAGAAACTGGTGGTGTCCGCCACCGAGGCT |
| | | CACTTCGATACCACCACACCTTTTGCCGCCGTCGTGATCGCTCTGAT |
| | | GGGAACCGTTGCTCAGATGGAACTGGAAGCCATCAAAGAGCGGAAC |
| | | AGATCCGCCGCTCACTTCAACATCAGAGCCGGCAAGTACCGGGGCT |
| | | CTTTGCCTCCTTGGGGCTACCTGCCAACAAGAGTGGATtccggagggtctgg |
| | | ctccggatcaagtggtggcagcggtaccatggccgccagcgacgaagtgaacctgatcgagagcagaaccgtg |
| | | gtgcccctgaacacctgggtgctgatctccaacttcaaggtggcctacaacatcctgcggaggcccgacggcac |
| | | cttcaacagacacctggccgagtacctggaccggaaagtgaccgccaacgccaaccctgtggacggcgtgttca |
| | | gcttcgacgtgctgatcgaccggcggatcaacctgctgagccgggtgtacagacccgcctacgccgatcaggaa |
| | | cagcccccctctatcctggatctggaaaagcccgtggatggcgacatcgtgcccgtgatcctgttcttccacggcg |
| | | gcagctttgcccacagcagcgccaatagcgccatctacgacaccctgtgcagacggctcgtgggcctgtgcaaa |
| | | tgcgtggtggtgtccgtgaactaccgcagagcccccgagaacccttacccctgcgcctacgatgatggctggatc |
| | | gccctgaactgggtcaacagcagaagctggctgaagtccaagaaagacagcaaggtgcacatctttctggccgg |
| | | cgatagcagcggcggcaatatcgcccataacgtggccctgagagccggcgagtctggcatcgatgtgctgggc |
| | | aatatcctgctgaaccccatgttcggcggcaacgagcggaccgagagcgagaagtctctggacggcaagtactt |
| | | cgtgaccgtgcgggaccgggactggtactggaaggcctttctgcccgagggcgaggacagagagcaccccgc |
| | | ctgcaatcccttcagccccagaggcaaaagcctggaaggcgtgtccttcccaaagtccctggtggtggtggccg |
| | | gcctggacctgatcagagattggcagctggcctatgccgagggcctgaagaaagccggccaggaagtgaagct |
| | | gatgcacctggaaaaggccaccgtgggcttctacctgctgcccaacaacaaccacttccacaacgtgatggacg |
| | | agatcagcgccttcgtgaacgccgagtgccccaagaaaaagcggaaggtgGCCACCAACTTTAG |
| | | CCTGCTGAAACAGGCTGGCGACGTGGAAGAGAACCCTGGACCTatgaa |
| | | gcgggaccaccaccatcaccatcatcaggacaagaaaaccatgatgatgaacgaagaggacgacggcaacgg |
| | | catggacgagctgctggctgtgctgggctacaaagtgcggagcagcgagatggccgacgtggcccagaaactg |
| | | gaacagctggaagtgatgatgagcaacgtgcaggaagatgacctgtcccagctggccaccgagacagtgcact |
| | | acaaccccgccgagctgtacacctggctggactccatgctgaccgacctgaactccggagggtctggctccgga |
| | | tcaagtggtggcagcggtaccGGCGAATGGCGGCTGGTGCCTGATCCTGTGCAG |
| | | CGGGAAAGAATCCTGGAAGTGTACCACAGAGTGGTGGACAACCACG |
| | | AGCCTCTGCACCTGGTGGCCCACGACTTGAATAGAAGAGGCGTGCT |
| | | GTCCCCTAAGGACTACTTCGCCCAGCTGCAGGGCAGAGAGCCTCAG |
| | | GGAAGAGAGTGGAGCGCTACCGCTCTGAAGCGGTCCATGATCTCTG |
| | | AGGCCATGCTGGGCTACGCTACCCTGAATGGAAAGACCGTGCGGGA |
| | | CGATGATGGCGCCCCTCTTGTTAGAGCCGAGCCTATCCTGACCAGAG |
| | | AGCAGCTCGAAGCCCTGAGAGCTGAGCTGGTCAAGACCTCCAGAGC |
| | | CAAGCCTGCTGTGTCTACCCCTAGCCTGCTGCTGAGAGTGCTGTTCT |
| | | GTGCTGTGTGTGGCGAGCCCGCCTACAAGTTTGCTGGCGGCGGAAG |
| | | AAAGCACCCCAGATACCGGTGTCGGTCCATGGGCTTCCCTAAGCACT |
| | | GTGGCAATGGCACCGTGGCCATGGCTGAGTGGGATGCCTTCTGCGA |
| | | AGAACAGGTGCTGGATCTGCTGGGCGACGCCGAGAGACTGGAAAAA |
| | | GTGTGGGTGGCCGGCTCCGACTCTGCTGTGGAACTGGCTGAAGTGA |
| | | ACGCCGAGCTGGTGGACCTGACCTCTCTGATCGGCTCTCCCGCTTAT |
| | | AGAGCTGGCTCCCCTCAGAGAGAAGCCCTGGACGCTAGAATCGCTG |
| | | CCCTGGCTGCTAGACAAGAGGAACTCGAAGGCCTGGAAGCTCGGCC |
| | | TTCAGGATGGGAGTGGCGAGAGACAGGCCAGAGATTTGGCGACTGG |
| | | TGGCGCGAGCAAGATACCGCCGCTAAGAACACCTGGCTGCGGTCTA |
| | | TGAATGTGCGGCTGACCTTCGATGTGCGCGGAGGACTGACCAGAAC |
| | | CATCGACTTCGGCGACCTGCAAGAGTACGAGCAGCATCTGAGACTG |
| | | GGCTCCGTGGTGGAAAGACTGCACACCGGCATGTCCggttcaCCAAAGA |
| | | AAAAGCGGAAAGTGTGACCCACCACTGGTTTCTACATTTACACCCatg |
| | | ctagcgcggccgcatcgataagcttgtcgacgatatcTTCACTCCTCAGGTGCAGGCTGCC |
| | | TATCAGAAGGTGGTGGCTGGTGTGGCCAATGCCCTGGCTCACAAAT |
| | | ACCACTGAGATCTTTTTCCCTCTGCCAAAAATTATGGGGACATCATG |
| | | AAGCCCCTTGAGCATCTGACTTCTGGCTAATAAAGGAAATTTATTTT |
| | | CATTGCAATAGTGTGTTGGAATTTTTTGTGTCTCTCACTCGGAAGGA |
| | | CATATGGGAGGGCAAATCATTTAAAACATCAGAATGAGTATTTGGTT |
| | | TAGAGTTTGGCAACATATGCCCATATGCTGGCTGCCATGAACAAAG |
| | | GTTGGCTATAAAGAGGTCATCAGTATATGAAACAGCCCCCTGCTGTC |
| | | CATTCCTTATTCCATAGAAAAGCCTTGACTTGAGGTTAGATTTTTTTT |
| | | ATATTTTGTTTTGTGTTATTTTTTTCTTTAACATCCCTAAAATTTTCCT |
| | | TACATGTTTTACTAGCCAGATTTTTCCTCCTCTCCTGACTACTCCCAG |
| | | TCATAGCTGTCCCTCTTCTCTTATGGAGAT |
| 136 | pAI-2471 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatggatacctacgccggagcctacgacagacagagccgggagagagagaacagcagcgccgccagccc |
| | | cgccacccagagaagcgccaacgaggataaggccgccgatctgcagagagaggtggagagggacggcggc |
| | | agattcagatttgtgggccacttcagcgaggcccctggcaccagcgccttcggcaccgccgagagGcccgagt |
| | | tcgagagaatcctgaacgagtgtagggccggcaggctgaacatgatcatcgtgtacgacgtgtcccggttcagca |
| | | ggctgaaggtgatggacgccatccctatcgtgtccgagctgctggccctgggcgtgaccatcgtgtccacccag |
| | | gaaggcgtctttagacagggcaacgtgatggacctgatccacctgatcatgaggctggacgccagccacaagga |
| | | gagcagcctgaaAagcgccaagatcctggacaccaagaacctgcagagggagctgggcggctatgtgggcg |
| | | gcaaggccccctacggcttcgagctggtgtccgaAaccaaggagatcacccggaacggcaggatggtgaacg |
| | | tggtgatcaacaagctggcccacagcaccacccccctgaccggccccttcgagtttgagcccgacgtgatcagg |
| | | tggtggtggcgggagatcaagacccacaagcacctgcctttcaagccctccggagggtctggctccggatcaag |
| | | tggtggcagcggtaccatggccgccagcgacgaagtgaacctgatcgagagcagaaccgtggtgcccctgaac |
| | | acctgggtgctgatctccaacttcaaggtggcctacaacatcctgcggaggcccgacggcaccttcaacagacac |
| | | ctggccgagtacctggaccggaaagtgaccgccaacgccaaccctgtggacggcgtgttcagcttcgacgtgct |
| | | gatcgaccggcggatcaacctgctgagccgggtgtacagacccgcctacgccgatcaggaacagcccccctct |
| | | atcctggatctggaaaagcccgtggatggcgacatcgtgcccgtgatcctgttcttccacggcggcagctttgccc |
| | | acagcagcgccaatagcgccatctacgacaccctgtgcagacggctcgtgggcctgtgcaaatgcgtggtggtg |
| | | tccgtgaactaccgcagagcccccgagaacccttacccctgcgcctacgatgatggctggatcgccctgaactg |
| | | ggtcaacagcagaagctggctgaagtccaagaaagacagcaaggtgcacatctttctggccggcgatagcagc |
| | | ggcggcaatatcgcccataacgtggccctgagagccggcgagtctggcatcgatgtgctgggcaatatcctgct |
| | | gaaccccatgttcggcggcaacgagcggaccgagagcgagaagtctctggacggcaagtacttcgtgaccgtg |
| | | cgggaccgggactggtactggaaggcctttctgcccgagggcgaggacagagagcaccccgcctgcaatccct |
| | | tcagccccagaggcaaaagcctggaaggcgtgtccttcccaaagtccctggtggtggtggccggcctggacctg |
| | | atcagagattggcagctggcctatgccgagggcctgaagaaagccggccaggaagtgaagctgatgcacctgg |
| | | aaaaggccaccgtgggcttctacctgctgcccaacaacaaccacttccacaacgtgatggacgagatcagcgcc |
| | | ttcgtgaacgccgagtgccccaagaaaaagcggaaggtgGCCACCAACTTTAGCCTGCTGA |
| | | AACAGGCTGGCGACGTGGAAGAGAACCCTGGACCTatgaagcgggaccacca |
| | | ccatcaccatcatcaggacaagaaaaccatgatgatgaacgaagaggacgacggcaacggcatggacgagct |
| | | gctggctgtgctgggctacaaagtgcggagcagcgagatggccgacgtggcccagaaactggaacagctgga |
| | | agtgatgatgagcaacgtgcaggaagatgacctgtcccagctggccaccgagacagtgcactacaaccccgcc |
| | | gagctgtacacctggctggactccatgctgaccgacctgaactccggagggtctggctccggatcaagtggtgg |
| | | cagcggtaccggcagccaggccgccatccaccccggcagcatcaccggcctgtgtaagagaatggacgccga |
| | | cgccgtgcccaccagaggcgaAaccatcggcaagaaaaccgccagcagcgcctgggaccccgccaccgtg |
| | | atgagaatcctgagggaccctaggatcgccggcttcgccgccgaggtgatctacaagaagaagcccgacggca |
| | | cccccaccaccaagatcgagggctacagaatccagagGgaccccatcaccctgagGcctgtggagctggact |
| | | gtggccctatcatcgagcctgccgagtggtacgagctgcaggcctggctggacggcagaggcagaggcaagg |
| | | gcctgagcagaggccaggccatcctgagcgccatggacaagctgtactgtgagtgtggcgccgtgatgaccag |
| | | caagagaggcgaggagagcatcaaggacagctaccggtgccggagaagaaaggtggtggaccccagcgcc |
| | | cctggccagcacgagggcacctgtaatgtgagcatggccgccctggacaagttcgtggccgagcggatcttcaa |
| | | caagatccggcacgccgagggcgacgaggaAaccctggccctgctgtgggaggccgccagaagattcggca |
| | | agctgaccgaggcccccgaAaagagcggcgagagggccaacctggtggccgagagagccgacgccctgaa |
| | | cgccctggaggagctgtacgaggacagagccgccggagcctatgacggccctgtgggcaggaagcacttcag |
| | | aaagcagcaggccgccctgaccctgagacagcagggcgccgaggaaagactggccgagctggaggccgcc |
| | | gaggcccctaagctgcccctggatcagtggttccccgaggatgccgacgccgaccccaccggccccaagtcct |
| | | ggtggggcagagccagcgtggacgacaagagggtgttcgtgggcctgttcgtggataagatcgtggtgaccaa |
| | | gagcaccaccggcaggggccagggcacccccatcgagaagagagccagcatcacctgggccaagcctccca |
| | | ccgacgacgacgaggatgacgcccaggacggcaccgaggacgtggccgcccccaagaaaaagcggaaggt |
| | | gtgatgaCCCAccactggattgtacaattacGAGTCTAAGTAAGGATCCCTGTGCCTT |
| | | CTAGTTGCCAGCCATCTGTTGTTTGCCCCTCCCCCGTGCCTTCCTTGA |
| | | CCCTGGAAGGTGCCACTCCCACTGTCCTTTCCTAATAAAATGAGGAA |
| | | ATTGCATCGCATTGTCTGAGTAGGTGTCATTCTATTCTGGGGGGTGG |
| | | GGTGGGGCAGGACAGCAAGGGGGAGGATTGGGATGACAATAGCAG |
| | | GCATGCTGGGGATGCGGTGGGCTCTATGG |
| 137 | pAI-2472 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatggatacctacgccggagcctacgacagacagagccgggagagagagaacagcagcgccgccagccc |
| | | cgccacccagagaagcgccaacgaggataaggccgccgatctgcagagagaggtggagagggacggcggc |
| | | agattcagatttgtgggccacttcagcgaggcccctggcaccagcgccttcggcaccgccgagagGcccgagt |
| | | tcgagagaatcctgaacgagtgtagggccggcaggctgaacatgatcatcgtgtacgacgtgtcccggttcagca |
| | | ggctgaaggtgatggacgccatccctatcgtgtccgagctgctggccctgggcgtgaccatcgtgtccacccag |
| | | gaaggcgtctttagacagggcaacgtgatggacctgatccacctgatcatgaggctggacgccagccacaagga |
| | | gagcagcctgaaAagcgccaagatcctggacaccaagaacctgcagagggagctgggcggctatgtgggcg |
| | | gcaaggccccctacggcttcgagctggtgtccgaAaccaaggagatcacccggaacggcaggatggtgaacg |
| | | tggtgatcaacaagctggcccacagcaccacccccctgaccggccccttcgagtttgagcccgacgtgatcagg |
| | | tggtggtggcgggagatcaagacccacaagcacctgcctttcaagcccggcagccaggccgccatccaccccg |
| | | gcagcatcaccggcctgtgtaagagaatggacgccgacgccgtgcccaccagaggcgaAaccatcggcaag |
| | | aaaaccgccagcagcgcctgggaccccgccaccgtgatgagaatcctgagggaccctaggatcgccggcttcg |
| | | ccgccgaggtgatctacaagaagaagcccgacggcacccccaccaccaagatcgagggctacagaatccaga |
| | | gGgaccccatcaccctgagGcctgtggagctggactgtggccctatcatcgagcctgccgagtggtacgagct |
| | | gcaggcctggctggacggcagaggcagaggcaagggcctgagcagaggccaggccatcctgagcgccatg |
| | | gacaagctgtactgtgagtgtggcgccgtgatgaccagcaagagaggcgaggagagcatcaaggacagctac |
| | | cggtgccggagaagaaaggtggtggaccccagcgcccctggccagcacgagggcacctgtaatgtgagcatg |
| | | gccgccctggacaagttcgtggccgagcggatcttcaacaagatccggcacgccgagggcgacgaggaAac |
| | | cctggccctgctgtgggaggccgccagaagattcggcaagctgaccgaggcccccgaAaagagcggcgaga |
| | | gggccaacctggtggccgagagagccgacgccctgaacgccctggaggagctgtacgaggacagagccgcc |
| | | ggagcctatgacggccctgtgggcaggaagcacttcagaaagcagcaggccgccctgaccctgagacagcag |
| | | ggcgccgaggaaagactggccgagctggaggccgccgaggcccctaagctgcccctggatcagtggttcccc |
| | | gaggatgccgacgccgaccccaccggccccaagtcctggtggggcagagccagcgtggacgacaagagggt |
| | | gttcgtgggcctgttcgtggataagatcgtggtgaccaagagcaccaccggcaggggctccggagggtctggct |
| | | ccggatcaagtggtggcagcggtaccatcctctggcatgagatgtggcatgaaggcctggaagaggcatctcgtt |
| | | tgtactttggggaaaggaacgtgaaaggcatgtttgaggtgctggagcccttgcatgctatgatggaacggggcc |
| | | cccagactctgaaggaaacatcctttaatcaggcctatggtcgagatttaatggaggcccaagagtggtgcagga |
| | | agtacatgaaatcagggaatgtcaaggacctcctccaagcctgggacctctattatcatgtgttccgacgaatctca |
| | | cccaagaaaaagcggaaggtgGCCACCAACTTTAGCCTGCTGAAACAGGCTGGC |
| | | GACGTGGAAGAGAACCCTGGACCTatgtctagaggagtgcaggtggaaaccatctcccca |
| | | ggGgacggAcgcaccttccccaagcgcggccagacctgcgtggtgcactacaccgggatgcttgaagatgg |
| | | aaagaaatttgattcctcccgggacagaaacaagccctttaagtttatgctaggcaagcaggaggtgatccgagg |
| | | ctgggaagaaggggttgcccagatgagtgtgggtcagagagccaaactgactatatctccagattatgcctatggt |
| | | gccactgggcacccaggcatcatcccaccacatgccactctcgtGttcgatgtggagcttctaaaactggaatcc |
| | | ggagggtctggctccggatcaagtggtggcagcggtacccagggcacccccatcgagaagagagccagcatc |
| | | acctgggccaagcctcccaccgacgacgacgaggatgacgcccaggacggcaccgaggacgtggccgcccc |
| | | caagaaaaagcggaaggtgtgatgaCCCAccactggattgtacaattacGAGTCTAAGTAAGG |
| | | ATCCCTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAA |
| | | TAAAATGAGGAAATTGCATCGCATTGTCTGAGTAGGTGTCATTCTAT |
| | | TCTGGGGGGTGGGGTGGGGCAGGACAGCAAGGGGGAGGATTGGGA |
| | | TGACAATAGCAGGCATGCTGGGGATGCGGTGGGCTCTATGG |
| 138 | pAI-2473 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatgaccaagaaggtggccatctacaccagagtgtccaccaccaaccaggccgaggaaggcttcagcatcg |
| | | acgagcagatcgaccggctgaccaaatacgccgaggccatgggatggcaggtgtccgatacctacaccgacgc |
| | | cggctttagcggcgccaagctggaaagacccgccatgcagcggctgatcaacgacatcgagaacaaggccttc |
| | | gacaccgtgctggtgtacaagctggacaggctgagcagaagcgtgcgggacaccctgtacctcgtgaaggacg |
| | | tgttcaccaagaacaagatcgacttcatcagcctgaacgagagcatcgacaccagcagcgctatgggcagcctgt |
| | | tcctgaccatcctgagcgccatcaacgagttcgagcgcgagaacatcaaagaacggatgaccatgggcaagctg |
| | | ggcagagccaagagcggcaagagcatgatgtggaccaagaccgccttcggctactaccacaacagaaagacc |
| | | ggcatcctggaaatagtgccactgcaggccaccatcgtggaacagatcttcaccgactacctgagcggcatctcc |
| | | ctgaccaagctgagagacaagctgaacgagtccggccacatcggcaaggacatcccttggagctaccggaccc |
| | | tgcggcagaccctggacaaccctgtgtactgcggctacatcaagttcaaggactccctgttcgagggcatgcaca |
| | | agcccatcatcccttacgagacatacctgaaggtgcagaaagagctggaagagagacagcagcagacctacga |
| | | gcggaacaacaaccccagacccttccaggccaagtacatgctgtccggcatggccagatgcggctactgtggc |
| | | gcccctctgaagatcgtgctgggccacaagagaaaggacggcagccggaccatgaagtaccactgcgccaac |
| | | cggttccctagaaagaccaagggcatctccggagggtctggctccggatcaagtggtggcagcggtaccatggc |
| | | cgccagcgacgaagtgaacctgatcgagagcagaaccgtggtgcccctgaacacctgggtgctgatctccaact |
| | | tcaaggtggcctacaacatcctgcggaggcccgacggcaccttcaacagacacctggccgagtacctggaccg |
| | | gaaagtgaccgccaacgccaaccctgtggacggcgtgttcagcttcgacgtgctgatcgaccggcggatcaacc |
| | | tgctgagccgggtgtacagacccgcctacgccgatcaggaacagcccccctctatcctggatctggaaaagccc |
| | | gtggatggcgacatcgtgcccgtgatcctgttcttccacggcggcagctttgcccacagcagcgccaatagcgcc |
| | | atctacgacaccctgtgcagacggctcgtgggcctgtgcaaatgcgtggtggtgtccgtgaactaccgcagagcc |
| | | cccgagaacccttacccctgcgcctacgatgatggctggatcgccctgaactgggtcaacagcagaagctggct |
| | | gaagtccaagaaagacagcaaggtgcacatctttctggccggcgatagcagcggcggcaatatcgcccataac |
| | | gtggccctgagagccggcgagtctggcatcgatgtgctgggcaatatcctgctgaaccccatgttcggcggcaa |
| | | cgagcggaccgagagcgagaagtctctggacggcaagtacttcgtgaccgtgcgggaccgggactggtactg |
| | | gaaggcctttctgcccgagggcgaggacagagagcaccccgcctgcaatcccttcagccccagaggcaaaag |
| | | cctggaaggcgtgtccttcccaaagtccctggtggtggtggccggcctggacctgatcagagattggcagctggc |
| | | ctatgccgagggcctgaagaaagccggccaggaagtgaagctgatgcacctggaaaaggccaccgtgggcttc |
| | | tacctgctgcccaacaacaaccacttccacaacgtgatggacgagatcagcgccttcgtgaacgccgagtgccc |
| | | caagaaaaagcggaaggtgGCCACCAACTTTAGCCTGCTGAAACAGGCTGGCG |
| | | ACGTGGAAGAGAACCCTGGACCTatgaagcgggaccaccaccatcaccatcatcaggaca |
| | | agaaaaccatgatgatgaacgaagaggacgacggcaacggcatggacgagctgctggctgtgctgggctacaa |
| | | agtgcggagcagcgagatggccgacgtggcccagaaactggaacagctggaagtgatgatgagcaacgtgca |
| | | ggaagatgacctgtcccagctggccaccgagacagtgcactacaaccccgccgagctgtacacctggctggact |
| | | ccatgctgaccgacctgaactccggagggtctggctccggatcaagtggtggcagcggtaccaccgtgtacaac |
| | | gacaacaagaagtgcgacagcggcacctacgacctgagcaacctggaaaacaccgtgatcgacaacctgatcg |
| | | gcttccaggaaaacaacgacagcctgctgaagatcatcaacggcaacaaccagcccatcctggacacctccagc |
| | | ttcaagaagcagatcagccagatcgacaagaagatccagaagaacagcgacctgtacctgaacgatttcatcacc |
| | | atggacgagctgaaggaccggaccgactctctgcaggccgagaagaagctgctgaaggccaagatctctgaga |
| | | acaagttcaacgatagcaccgacgtgttcgagctcgtgaaaacacagctgggctccatccccatcaatgagctga |
| | | gctacgataacaagaaaaagattgtgaacaacctggtgtctaaggtggacgtgaccgccgacaacgtggacatc |
| | | atcttcaagttccagctggcctgatgaCCCAccactggattgtacaattacGAGTCTAAGTAAGG |
| | | ATCCCTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAA |
| | | TAAAATGAGGAAATTGCATCGCATTGTCTGAGTAGGTGTCATTCTAT |
| | | TCTGGGGGGTGGGGTGGGGCAGGACAGCAAGGGGGAGGATTGGGA |
| | | TGACAATAGCAGGCATGCTGGGGATGCGGTGGGCTCTATGG |
| 139 | pAI-1504 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAgccacc |
| | | ATGTCCAATCTGCTGACCGTGCACCAGAACCTGCCTGCTCTGCCCGT |
| | | GGACGCCACCAGCGACGAGGTGCGCAAGAACCTGATGGACATGTTC |
| | | CGCGACCGCCAGGCCTTCAGCGAGCACACCTGGAAGATGCTGCTGA |
| | | GCGTGTGCCGCAGCTGGGCCGCCTGGTGCAAGCTGAACAACCGCAA |
| | | GTGGTTCCCCGCCGAGCCCGAGGACGTGCGCGACTACCTGCTGTACC |
| | | TGCAGGCCCGCGGCCTGGCCGTGAAAACCATCCAGCAGCACCTGGG |
| | | CCAGCTGAACATGCTGCACCGCCGCAGCGGCCTGcctAGGCCATCTGA |
| | | CTCTAATGCCGTGTCTCTGGTCATGCGGCGGATCCGGAAAGAAAAC |
| | | GTGGACGCCGGCGAGAGAGCTAAGCAGGCTCTGGCTTTCGAGAGAA |
| | | CCGACTTCGACCAAGTGCGGTCCCTGATGGAAAACTCCGACCGGTG |
| | | CCAGGATATCCGGAACCTGGCTTTTCTGGGAATCGCCTACAACACCC |
| | | TGCTGCGGATCGCTGAGATCGCCCGGATCAGAGTGAAGGACATCTC |
| | | TAGAACCGACGGCGGCAGAATGCTGATCCACATCGGCAGAACAAAG |
| | | ACCCTGGTGTCCACAGCTGGCGTGGAAAAGGCTCTGTCTCTGGGCGT |
| | | GACCAAGCTGGTGGAACGGTGGATTTCTGTGTCCGGCGTGGCCGAC |
| | | GATCCCAACAACTACCTGTTCTGCAGAGTCCGGAAGAACGGCGTGG |
| | | CAGCCCCTTCTGCTACATCCCAGCTGTCTACAAGAGCCCTGGAAGGC |
| | | ATCTTCGAGGCTACCCACAGAtccggagggtctggctccggatcaagtggtggcagcggta |
| | | cccctttgtatggttttacttcgatttgtggaagaagGcctgagatggaagctgctgtttcgactataccaagattcctt |
| | | caatcttcctctggttcgatgttagatggtcggtttgatcctcaatccgccgctcatttcttcggtgtttacgacggccat |
| | | ggcggttctcaggtagcgaactattgtagagagaggatgcatttggctttggcggaggagatagctaaggagaaa |
| | | ccgatgctctgcgatggtgatacgtggctggagaagtggaagaaagctcttttcaactcgttcctgagagttgactc |
| | | ggagattgagtcagttgcgccggagacggttgggtcaacgtcggtggttgccgttgttttcccgtctcacatcttcgt |
| | | cgctaactgcggtgactctagagccgttctttgccgcggcaaaactgcacttccattatccgttgaccataaaccgg |
| | | atagagaagatgaagctgcgaggattgaagccgcaggagggaaagtgattcagtggaatggagctcgtgttttc |
| | | ggtgttctcgccatgtcgagatccattggcgatagatacttgaaaccatccatcattcctgatccggaagtgacggc |
| | | tgtgaagagagtaaaagaagatgattgtctgattttggcgagtgacggggtttgggatgtaatgacggatgaagaa |
| | | gcgtgtgagatggcaaggaagcggattctcttgtggcacaagaaaaacgcggtggctggggatgcatcgttgct |
| | | cgcggatgagcggagaaaggaagggaaagatcctgcggcgatgtccgcggctgagtatttgtcaaagctggcg |
| | | atacagagaggaagcaaagacaacataagtgtggtggtggttgatttgaaggattacaaggacgatgacgataag |
| | | cccaagaaaaagcggaaggtgGCCACCAACTTTAGCCTGCTGAAACAGGCTGGC |
| | | GACGTGGAAGAGAACCCTGGACCTatggcgccaactcaagacgaattcacccaactctcc |
| | | caatcaatcgccgagttccacacgtaccaactcggtaacggccgttgctcatctctcctagctcagcgaatccacg |
| | | cgccgccggaaacagtatggtccgtggtgagacgtttcgataggccacagatttacaaacacttcatcaaaagctg |
| | | taacgtgagtgaagatttcgagatgcgagtgggatgcacgcgcgacgtgaacgtgataagtggattaccggcga |
| | | atacgtctcgagagagattagatctgttggacgatgatcggagagtgactgggtttagtataaccggtggtgaacat |
| | | aggctgaggaattataaatcggttacgacggttcatagatttgagaaagaagaagaagaagaaaggatctggacc |
| | | gttgttttggaatcttatgttgttgatgtaccggaaggtaattcggaggaagatacgagattgtttgctgatacggttatt |
| | | agattgaatcttcagaaacttgcttcgatcactgaagctatgaactacccatacgatgttccagattacgcttccgga |
| | | gggtctggctccggatcaagtggtggcagcggtaccCTGATCTACGGCGCCAAGGACGA |
| | | TAGCGGCCAGAGATATTTGGCTTGGAGCGGCCACTCCGCTAGAGTG |
| | | GGAGCTGCTAGAGATATGGCTAGAGCCGGCGTGTCCATTCCTGAGA |
| | | TCATGCAAGCTGGCGGCTGGACCAACGTGAACATCGTGATGAACTA |
| | | CATCCGCAACCTGGACTCCGAGACAGGCGCTATGGTTCGACTGCTGG |
| | | AAGATGGCGACcccaagaaaaagcggaaggtgtgatgaCCCACCACTGGTTTCTAC |
| | | ATTTACGAGTatgctagcgcggccgcatcgataagcttgtcgacgatatcTTCACTCCTCAG |
| | | GTGCAGGCTGCCTATCAGAAGGTGGTGGCTGGTGTGGCCAATGCCCT |
| | | GGCTCACAAATACCACTGAGATCTTTTTCCCTCTGCCAAAAATTATG |
| | | GGGACATCATGAAGCCCCTTGAGCATCTGACTTCTGGCTAATAAAGG |
| | | AAATTTATTTTCATTGCAATAGTGTGTTGGAATTTTTTGTGTCTCTCA |
| | | CTCGGAAGGACATATGGGAGGGCAAATCATTTAAAACATCAGAATG |
| | | AGTATTTGGTTTAGAGTTTGGCAACATATGCCCATATGCTGGCTGCC |
| | | ATGAACAAAGGTTGGCTATAAAGAGGTCATCAGTATATGAAACAGC |
| | | CCCCTGCTGTCCATTCCTTATTCCATAGAAAAGCCTTGACTTGAGGTT |
| | | AGATTTTTTTTATATTTTGTTTTGTGTTATTTTTTTCTTTAACATCCCT |
| | | AAAATTTTCCTTACATGTTTTACTAGCCAGATTTTTCCTCCTCTCCTG |
| | | ACTACTCCCAGTCATAGCTGTCCCTCTTCTCTTATGGAGAT |
| 140 | pAI-1505 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAgccacc |
| | | atggcgccaactcaagacgaattcacccaactctcccaatcaatcgccgagttccacacgtaccaactcggtaac |
| | | ggccgttgctcatctctcctagctcagcgaatccacgcgccgccggaaacagtatggtccgtggtgagacgtttcg |
| | | ataggccacagatttacaaacacttcatcaaaagctgtaacgtgagtgaagatttcgagatgcgagtgggatgcac |
| | | gcgcgacgtgaacgtgataagtggattaccggcgaatacgtctcgagagagattagatctgttggacgatgatcg |
| | | gagagtgactgggtttagtataaccggtggtgaacataggctgaggaattataaatcggttacgacggttcatagat |
| | | ttgagaaagaagaagaagaagaaaggatctggaccgttgttttggaatcttatgttgttgatgtaccggaaggtaatt |
| | | cggaggaagatacgagattgtttgctgatacggttattagattgaatcttcagaaacttgcttcgatcactgaagctat |
| | | gaactacccatacgatgttccagattacgcttccggagggtctggctccggatcaagtggtggcagcggtaccC |
| | | TGATCTACGGCGCCAAGGACGATAGCGGCCAGAGATATTTGGCTTG |
| | | GAGCGGCCACTCCGCTAGAGTGGGAGCTGCTAGAGATATGGCTAGA |
| | | GCCGGCGTGTCCATTCCTGAGATCATGCAAGCTGGCGGCTGGACCA |
| | | ACGTGAACATCGTGATGAACTACATCCGCAACCTGGACTCCGAGAC |
| | | AGGCGCTATGGTTCGACTGCTGGAAGATGGCGACcccaagaaaaagcggaag |
| | | gtgGCCACCAACTTTAGCCTGCTGAAACAGGCTGGCGACGTGGAAGA |
| | | GAACCCTGGACCTATGTCCAATCTGCTGACCGTGCACCAGAACCTGC |
| | | CTGCTCTGCCCGTGGACGCCACCAGCGACGAGGTGCGCAAGAACCT |
| | | GATGGACATGTTCCGCGACCGCCAGGCCTTCAGCGAGCACACCTGG |
| | | AAGATGCTGCTGAGCGTGTGCCGCAGCTGGGCCGCCTGGTGCAAGC |
| | | TGAACAACCGCAAGTGGTTCCCCGCCGAGCCCGAGGACGTGCGCGA |
| | | CTACCTGCTGTACCTGCAGGCCCGCGGCCTGGCCGTGAAAACCATCC |
| | | AGCAGCACCTGGGCCAGCTGAACATGCTGCACCGCCGCAGCGGCCT |
| | | GcctAGGCCATCTGACTCTAATGCCGTGTCTCTGGTCATGCGGCGGAT |
| | | CCGGAAAGAAAACGTGGACGCCGGCGAGAGAGCTAAGCAGGCTCT |
| | | GGCTTTCGAGAGAACCGACTTCGACCAAGTGCGGTCCCTGATGGAA |
| | | AACTCCGACCGGTGCCAGGATATCCGGAACCTGGCTTTTCTGGGAAT |
| | | CGCCTACAACACCCTGCTGCGGATCGCTGAGATCGCCCGGATCAGA |
| | | GTGAAGGACATCTCTAGAACCGACGGCGGCAGAATGCTGATCCACA |
| | | TCGGCAGAACAAAGACCCTGGTGTCCACAGCTGGCGTGGAAAAGGC |
| | | TCTGTCTCTGGGCGTGACCAAGCTGGTGGAACGGTGGATTTCTGTGT |
| | | CCGGCGTGGCCGACGATCCCAACAACTACCTGTTCTGCAGAGTCCGG |
| | | AAGAACGGCGTGGCAGCCCCTTCTGCTACATCCCAGCTGTCTACAAG |
| | | AGCCCTGGAAGGCATCTTCGAGGCTACCCACAGAtccggagggtctggctccgg |
| | | atcaagtggtggcagcggtacccctttgtatggttttacttcgatttgtggaagaagGcctgagatggaagctgctg |
| | | tttcgactataccaagattccttcaatcttcctctggttcgatgttagatggtcggtttgatcctcaatccgccgctcattt |
| | | cttcggtgtttacgacggccatggcggttctcaggtagcgaactattgtagagagaggatgcatttggctttggcgg |
| | | aggagatagctaaggagaaaccgatgctctgcgatggtgatacgtggctggagaagtggaagaaagctcttttca |
| | | actcgttcctgagagttgactcggagattgagtcagttgcgccggagacggttgggtcaacgtcggtggttgccgt |
| | | tgttttcccgtctcacatcttcgtcgctaactgcggtgactctagagccgttctttgccgcggcaaaactgcacttcca |
| | | ttatccgttgaccataaaccggatagagaagatgaagctgcgaggattgaagccgcaggagggaaagtgattca |
| | | gtggaatggagctcgtgttttcggtgttctcgccatgtcgagatccattggcgatagatacttgaaaccatccatcatt |
| | | cctgatccggaagtgacggctgtgaagagagtaaaagaagatgattgtctgattttggcgagtgacggggtttgg |
| | | gatgtaatgacggatgaagaagcgtgtgagatggcaaggaagcggattctcttgtggcacaagaaaaacgcggt |
| | | ggctggggatgcatcgttgctcgcggatgagcggagaaaggaagggaaagatcctgcggcgatgtccgcggct |
| | | gagtatttgtcaaagctggcgatacagagaggaagcaaagacaacataagtgtggtggtggttgatttgaaggatt |
| | | acaaggacgatgacgataagcccaagaaaaagcggaaggtgtgaCCCACCACTGGTTTCTAC |
| | | ATTTACGAGTatgctagcgcggccgcatcgataagcttgtcgacgatatcTTCACTCCTCAG |
| | | GTGCAGGCTGCCTATCAGAAGGTGGTGGCTGGTGTGGCCAATGCCCT |
| | | GGCTCACAAATACCACTGAGATCTTTTTCCCTCTGCCAAAAATTATG |
| | | GGGACATCATGAAGCCCCTTGAGCATCTGACTTCTGGCTAATAAAGG |
| | | AAATTTATTTTCATTGCAATAGTGTGTTGGAATTTTTTGTGTCTCTCA |
| | | CTCGGAAGGACATATGGGAGGGCAAATCATTTAAAACATCAGAATG |
| | | AGTATTTGGTTTAGAGTTTGGCAACATATGCCCATATGCTGGCTGCC |
| | | ATGAACAAAGGTTGGCTATAAAGAGGTCATCAGTATATGAAACAGC |
| | | CCCCTGCTGTCCATTCCTTATTCCATAGAAAAGCCTTGACTTGAGGTT |
| | | AGATTTTTTTTATATTTTGTTTTGTGTTATTTTTTTCTTTAACATCCCT |
| | | AAAATTTTCCTTACATGTTTTACTAGCCAGATTTTTCCTCCTCTCCTG |
| | | ACTACTCCCAGTCATAGCTGTCCCTCTTCTCTTATGGAGAT |
| 141 | pAI-1506 | TCCCTATCAGTGATAGAGATCCATGTGCAGTCTACTCCCTATCAGTG |
| | TU | ATAGAGAAGCTATGTCCAGCTTACTCCCTATCAGTGATAGAgaTGGT |
| | | ATGTCCAGTACTCTCCCTATCAGTGATAGAgaACATATGTGGAGTGTA |
| | | TCCCTATCAGTGATAGAgaAACTATCTGCAGATTACTCCCTATCAGTG |
| | | ATAGAgtATGTATGTCGAGGTAGGCGTGTACGGTGGGAGGCCTATAT |
| | | AAGCAGAGCTCGTTTAGTGAACCGTCAGATCGCtggagaattcgagctcggtacc |
| | | cggggaCAACATCATAAGGTTACGATAGGAGAGCAAgccaccATGTCCAA |
| | | TCTGCTGACCGTGCACCAGAACCTGCCTGCTCTGCCCGTGGACGCCA |
| | | CCAGCGACGAGGTGCGCAAGAACCTGATGGACATGTTCCGCGACCG |
| | | CCAGGCCTTCAGCGAGCACACCTGGAAGATGCTGCTGAGCGTGTGC |
| | | CGCAGCTGGGCCGCCTGGTGCAAGCTGAACAACCGCAAGTGGTTCC |
| | | CCGCCGAGCCCGAGGACGTGCGCGACTACCTGCTGTACCTGCAGGC |
| | | CCGCGGCCTGGCCGTGAAAACCATCCAGCAGCACCTGGGCCAGCTG |
| | | AACATGCTGCACCGCCGCAGCGGCCTGcctAGGCCATCTGACTCTAAT |
| | | GCCGTGTCTCTGGTCATGCGGCGGATCCGGAAAGAAAACGTGGACG |
| | | CCGGCGAGAGAGCTAAGCAGGCTCTGGCTTTCGAGAGAACCGACTT |
| | | CGACCAAGTGCGGTCCCTGATGGAAAACTCCGACCGGTGCCAGGAT |
| | | ATCCGGAACCTGGCTTTTCTGGGAATCGCCTACAACACCCTGCTGCG |
| | | GATCGCTGAGATCGCCCGGATCAGAGTGAAGGACATCTCTAGAACC |
| | | GACGGCGGCAGAATGCTGATCCACATCGGCAGAACAAAGACCCTGG |
| | | TGTCCACAGCTGGCGTGGAAAAGGCTCTGTCTCTGGGCGTGACCAA |
| | | GCTGGTGGAACGGTGGATTTCTGTGTCCGGCGTGGCCGACGATCCCA |
| | | ACAACTACCTGTTCTGCAGAGTCCGGAAGAACGGCGTGGCAGCCCC |
| | | TTCTGCTACATCCCAGCTGTCTACAAGAGCCCTGGAAGGCATCTTCG |
| | | AGGCTACCCACAGAtccggagggtctggctccggatcaagtggtggcagcggtacccctttgtatg |
| | | gttttacttcgatttgtggaagaagGcctgagatggaagctgctgtttcgactataccaagattccttcaatcttcctct |
| | | ggttcgatgttagatggtcggtttgatcctcaatccgccgctcatttcttcggtgtttacgacggccatggcggttctc |
| | | aggtagcgaactattgtagagagaggatgcatttggctttggcggaggagatagctaaggagaaaccgatgctct |
| | | gcgatggtgatacgtggctggagaagtggaagaaagctcttttcaactcgttcctgagagttgactcggagattga |
| | | gtcagttgcgccggagacggttgggtcaacgtcggtggttgccgttgttttcccgtctcacatcttcgtcgctaactg |
| | | cggtgactctagagccgttctttgccgcggcaaaactgcacttccattatccgttgaccataaaccggatagagaa |
| | | gatgaagctgcgaggattgaagccgcaggagggaaagtgattcagtggaatggagctcgtgttttcggtgttctc |
| | | gccatgtcgagatccattggcgatagatacttgaaaccatccatcattcctgatccggaagtgacggctgtgaaga |
| | | gagtaaaagaagatgattgtctgattttggcgagtgacggggtttgggatgtaatgacggatgaagaagcgtgtga |
| | | gatggcaaggaagcggattctcttgtggcacaagaaaaacgcggtggctggggatgcatcgttgctcgcggatg |
| | | agcggagaaaggaagggaaagatcctgcggcgatgtccgcggctgagtatttgtcaaagctggcgatacagag |
| | | aggaagcaaagacaacataagtgtggtggtggttgatttgaaggattacaaggacgatgacgataagcccaaga |
| | | aaaagcggaaggtgGCCACCAACTTTAGCCTGCTGAAACAGGCTGGCGACG |
| | | TGGAAGAGAACCCTGGACCTatggcgccaactcaagacgaattcacccaactctcccaatcaa |
| | | tcgccgagttccacacgtaccaactcggtaacggccgttgctcatctctcctagctcagcgaatccacgcgccgcc |
| | | ggaaacagtatggtccgtggtgagacgtttcgataggccacagatttacaaacacttcatcaaaagctgtaacgtg |
| | | agtgaagatttcgagatgcgagtgggatgcacgcgcgacgtgaacgtgataagtggattaccggcgaatacgtct |
| | | cgagagagattagatctgttggacgatgatcggagagtgactgggtttagtataaccggtggtgaacataggctga |
| | | ggaattataaatcggttacgacggttcatagatttgagaaagaagaagaagaagaaaggatctggaccgttgttttg |
| | | gaatcttatgttgttgatgtaccggaaggtaattcggaggaagatacgagattgtttgctgatacggttattagattga |
| | | atcttcagaaacttgcttcgatcactgaagctatgaactacccatacgatgttccagattacgcttccggagggtctg |
| | | gctccggatcaagtggtggcagcggtaccCTGATCTACGGCGCCAAGGACGATAGCG |
| | | GCCAGAGATATTTGGCTTGGAGCGGCCACTCCGCTAGAGTGGGAGC |
| | | TGCTAGAGATATGGCTAGAGCCGGCGTGTCCATTCCTGAGATCATGC |
| | | AAGCTGGCGGCTGGACCAACGTGAACATCGTGATGAACTACATCCG |
| | | CAACCTGGACTCCGAGACAGGCGCTATGGTTCGACTGCTGGAAGAT |
| | | GGCGACcccaagaaaaagcggaaggtgtgaCCCACCACTGGTTTCTACATTTACG |
| | | AGTatgctagcgcggccgcatcgataagcttgtcgacgatatcTTCACTCCTCAGGTGCAGG |
| | | CTGCCTATCAGAAGGTGGTGGCTGGTGTGGCCAATGCCCTGGCTCAC |
| | | AAATACCACTGAGATCTTTTTCCCTCTGCCAAAAATTATGGGGACAT |
| | | CATGAAGCCCCTTGAGCATCTGACTTCTGGCTAATAAAGGAAATTTA |
| | | TTTTCATTGCAATAGTGTGTTGGAATTTTTTGTGTCTCTCACTCGGAA |
| | | GGACATATGGGAGGGCAAATCATTTAAAACATCAGAATGAGTATTT |
| | | GGTTTAGAGTTTGGCAACATATGCCCATATGCTGGCTGCCATGAACA |
| | | AAGGTTGGCTATAAAGAGGTCATCAGTATATGAAACAGCCCCCTGC |
| | | TGTCCATTCCTTATTCCATAGAAAAGCCTTGACTTGAGGTTAGATTTT |
| | | TTTTATATTTTGTTTTGTGTTATTTTTTTCTTTAACATCCCTAAAATTT |
| | | TCCTTACATGTTTTACTAGCCAGATTTTTCCTCCTCTCCTGACTACTC |
| | | CCAGTCATAGCTGTCCCTCTTCTCTTATGGAGAT |
| 142 | pAI-5761 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAgccacc |
| | | atgtccaacctgctgactgtgcaccaaaacctgcctgccctccctgtggatgccacctctgatgaagtcaggaaga |
| | | acctgatggacatgttcagggacaggcaggccttctctgaacacacctggaagatgctcctgtctgtgtgcagatc |
| | | ctgggctgcctggtgcaagctgaacaacaggaaatggttccctgctgaacctgaggatgtgagggactacctcct |
| | | gtacctgcaagccagaggcctggctgtgaagaccatccaacagcacctgggccagctcaacatgctgcacagg |
| | | agatctggcctgcctcgcccttctgactccaatgctgtgtccctggtgatgaggagaatcagaaaggagaatgtgg |
| | | atgctggggagagagccaagcaggccctggcctttgaacgcactgactttgaccaagtcagatccctgatggag |
| | | aactctgacagatgccaggacatcaggaacctggccttcctgggcattgcctacaacaccctgctgcgcattgcc |
| | | gaaattgccagaatcagagtgaaggacatctcccgcaccgatggtgggagaatgctgatccacattggcaggac |
| | | caagaccctggtgtccacagctggtgtggagaaggccctgtccctgggggttaccaagctggtggagagatgga |
| | | tctctgtgtctggttccggagggtctggctccggatcaagtggtggcagcggtaccatggccgccagcgacgaa |
| | | gtgaacctgatcgagagcagaaccgtggtgcccctgaacacctgggtgctgatctccaacttcaaggtggcctac |
| | | aacatcctgcggaggcccgacggcaccttcaacagacacctggccgagtacctggaccggaaagtgaccgcc |
| | | aacgccaaccctgtggacggcgtgttcagcttcgacgtgctgatcgaccggcggatcaacctgctgagccgggt |
| | | gtacagacccgcctacgccgatcaggaacagcccccctctatcctggatctggaaaagcccgtggatggcgaca |
| | | tcgtgcccgtgatcctgttcttccacggcggcagctttgcccacagcagcgccaatagcgccatctacgacaccct |
| | | gtgcagacggctcgtgggcctgtgcaaatgcgtggtggtgtccgtgaactaccgcagagcccccgagaaccctt |
| | | acccctgcgcctacgatgatggctggatcgccctgaactgggtcaacagcagaagctggctgaagtccaagaaa |
| | | gacagcaaggtgcacatctttctggccggcgatagcagcggcggcaatatcgcccataacgtggccctgagagc |
| | | cggcgagtctggcatcgatgtgctgggcaatatcctgctgaaccccatgttcggcggcaacgagcggaccgaga |
| | | gcgagaagtctctggacggcaagtacttcgtgaccgtgcgggaccgggactggtactggaaggcctttctgccc |
| | | gagggcgaggacagagagcaccccgcctgcaatcccttcagccccagaggcaaaagcctggaaggcgtgtcc |
| | | ttcccaaagtccctggtggtggtggccggcctggacctgatcagagattggcagctggcctatgccgagggcctg |
| | | aagaaagccggccaggaagtgaagctgatgcacctggaaaaggccaccgtgggcttctacctgctgcccaaca |
| | | acaaccacttccacaacgtgatggacgagatcagcgccttcgtgaacgccgagtgccccaagaaaaagcggaa |
| | | ggtgtgaGCCACCAACTTTAGCCTGCTGAAACAGGCTGGCGACGTGGAA |
| | | GAGAACCCTGGACCTgccaccatgaaggggaccaccaccatcaccatcatcaggacaagaaaa |
| | | ccatgatgatgaacgaagaggacgacggcaacggcatggacgagctgctggctgtgctgggctacaaagtgcg |
| | | gagcagcgagatggccgacgtggcccagaaactggaacagctggaagtgatgatgagcaacgtgcaggaaga |
| | | tgacctgtcccagctggccaccgagacagtgcactacaaccccgccgagctgtacacctggctggactccatgct |
| | | gaccgacctgaactccggagggtctggctccggatcaagtggtggcagcggtaccgtggctgatgaccccaac |
| | | aactacctgttctgccgggtcagaaagaatggtgtggctgccccttctgccacctcccaactgtccacccgggccc |
| | | tggaagggatctttgaggccacccaccgcctgatctatggtgccaaggatgactctgggcagagatacctggcct |
| | | ggtctggccactctgccagagtgggtgctgccagggacatggccagggctggtgtgtccatccctgaaatcatgc |
| | | aggctggtggctggaccaatgtgaacattgtgatgaactacatcagaaacctggactctgagactggggccatgg |
| | | tgaggctgctcgaggatggggaccccaagaaaaagcggaaggtgtgaCCCACCACTGGTTTCT |
| | | ACATTTACGAGTatgctagcgcggccgcatcgataagcttgtcgacgatatcTTCACTCCTC |
| | | AGGTGCAGGCTGCCTATCAGAAGGTGGTGGCTGGTGTGGCCAATGC |
| | | CCTGGCTCACAAATACCACTGAGATCTTTTTCCCTCTGCCAAAAATT |
| | | ATGGGGACATCATGAAGCCCCTTGAGCATCTGACTTCTGGCTAATAA |
| | | AGGAAATTTATTTTCATTGCAATAGTGTGTTGGAATTTTTTGTGTCTC |
| | | TCACTCGGAAGGACATATGGGAGGGCAAATCATTTAAAACATCAGA |
| | | ATGAGTATTTGGTTTAGAGTTTGGCAACATATGCCCATATGCTGGCT |
| | | GCCATGAACAAAGGTTGGCTATAAAGAGGTCATCAGTATATGAAAC |
| | | AGCCCCCTGCTGTCCATTCCTTATTCCATAGAAAAGCCTTGACTTGA |
| | | GGTTAGATTTTTTTTATATTTTGTTTTGTGTTATTTTTTTCTTTAACAT |
| | | CCCTAAAATTTTCCTTACATGTTTTACTAGCCAGATTTTTCCTCCTCT |
| | | CCTGACTACTCCCAGTCATAGCTGTCCCTCTTCTCTTATGGAGAT |
| 143 | pAI-2470 | TTGGCTCCGGTGCCCGTCAGTGGGCAGAGCGCACATCGCCCACAGTC |
| | TU | CCCGAGAAGTTGgGGGGAGGGGTCGGCAATTGAACCGGTGCCTAGA |
| | | GAAGGTGGCGCGGGGTAAACTGGGAAAGTGATGTCGTGTACTGGCT |
| | | CCGCCTTTTTCCCGAGGGTGGGGGAGAACCGTATATAAGTGCAGTA |
| | | GTCGCCGTGAACGTTCTTTTTCGCAACGGGTTTGCCGCCAGAACACA |
| | | GGTAAGTGCCGTGTGTGGTTCCCGCGGGCCTGGCCTCTTTACGGGTT |
| | | ATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGtG |
| | | GCCTTGCGCTTAAGGAGCCCCTTCGCCTCGTGCTTGAGTTGtGGCCTG |
| | | GCCTGGGCGCTGGGGCCGCCGCGTGCGAATCTGGTGGCACCTTCGC |
| | | GCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGC |
| | | GGGCCAAGATCaGCACACTGGTATTTCGGTTTTTGGGGCCGCGGGCG |
| | | GCGACGGGGCCCGTGCGTCCCAGCGCACATGTTCGGCGAGGCGGGG |
| | | CCTGCGAGCGCGGCCACCGAGAATCGGACGGGGGTAGTCTCAAGCT |
| | | GcCCGGCCTGCTCTGGTGCCTGGCCTCGCGCCGCCGTGTATCGCCCC |
| | | GCCCTGGGCGGCAAGGCTGGCCCGGTCGGCACCAGTTGCGTGAGCG |
| | | GAAAGATGGCCGCTTCCCGGCCCTGCTGCAGGGAGCaCAAAATGGA |
| | | GGACGCGGCGCTCGGGAGAGCGGGCGGGTGAGTCACCCACACAAA |
| | | GGAAAAGGGCCTTTCCGTCCTCAGCCGTCGCTTCATGTGACTCCACG |
| | | GAGTACCGGGCGCCGTCCAGGCACCTCGATTAGTTCTCcAGCTTTTG |
| | | GAGTACGTCGTCTTTAGGTTGGGGGGAGGGGTTTTATGCGATGGAGT |
| | | TTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTgaCAACATCATAAGGTTACGATAGGAGAGCAAGCCA |
| | | CCatgatcgagaaccagctgagcctgctgggcgacttttctggcgtgcggcccgacgatgtgaaaaccgccatt |
| | | caggccgcccagaaaaagggcatcaacgtggccgagaacgagcagttcaaggccgccttcgagcatctgctga |
| | | acgagttcaagaagcgggaagagagatacagccccaacaccctgcggcggctggaaagcgcctggacctgct |
| | | tcgtggattggtgcctggccaaccacagacacagcctgcctgccacccccgataccgtggaagccttcttcatcg |
| | | agcgggccgaggaactgcaccggaacaccctgagcgtgtacagatgggccatcagccgggtgcacagagtgg |
| | | ccggatgccctgatccctgcctggacatctacgtggaagatcggctgaaggccattgcccggaagaaagtgcgg |
| | | gaaggcgaggccgtgaagcaggccagccctttcaacgagcagcatctgctgaagctgaccagcctgtggtaca |
| | | gaagcgacaagctgctgctgcggcggaacctggctctgctggctgtggcctacgagagcatgctgagagccag |
| | | cgagctggccaacatccgggtgtccgatatggaactggccggcgacggaaccgccatcctgaccatccctatca |
| | | ccaagaccaaccactccggcgagcccgatacctgcatcctgtcccaggatgtggtgtccctgctgatggactaca |
| | | ccgaggccggcaagctggatatgagcagcgacggcttcctgttcgtgggcgtgtccaagcacaacacctgtatct |
| | | ccggagggtctggctccggatcaagtggtggcagcggtaccatggccgccagcgacgaagtgaacctgatcga |
| | | gagcagaaccgtggtgcccctgaacacctgggtgctgatctccaacttcaaggtggcctacaacatcctgcggag |
| | | gcccgacggcaccttcaacagacacctggccgagtacctggaccggaaagtgaccgccaacgccaaccctgtg |
| | | gacggcgtgttcagcttcgacgtgctgatcgaccggcggatcaacctgctgagccgggtgtacagacccgccta |
| | | cgccgatcaggaacagcccccctctatcctggatctggaaaagcccgtggatggcgacatcgtgcccgtgatcct |
| | | gttcttccacggcggcagctttgcccacagcagcgccaatagcgccatctacgacaccctgtgcagacggctcgt |
| | | gggcctgtgcaaatgcgtggtggtgtccgtgaactaccgcagagcccccgagaacccttacccctgcgcctacg |
| | | atgatggctggatcgccctgaactgggtcaacagcagaagctggctgaagtccaagaaagacagcaaggtgca |
| | | catctttctggccggcgatagcagcggcggcaatatcgcccataacgtggccctgagagccggcgagtctggca |
| | | tcgatgtgctgggcaatatcctgctgaaccccatgttcggcggcaacgagcggaccgagagcgagaagtctctg |
| | | gacggcaagtacttcgtgaccgtgcgggaccgggactggtactggaaggcctttctgcccgagggcgaggaca |
| | | gagagcaccccgcctgcaatcccttcagccccagaggcaaaagcctggaaggcgtgtccttcccaaagtccctg |
| | | gtggtggtggccggcctggacctgatcagagattggcagctggcctatgccgagggcctgaagaaagccggcc |
| | | aggaagtgaagctgatgcacctggaaaaggccaccgtgggcttctacctgctgcccaacaacaaccacttccac |
| | | aacgtgatggacgagatcagcgccttcgtgaacgccgagtgccccaagaaaaagcggaaggtgGCCACC |
| | | AACTTTAGCCTGCTGAAACAGGCTGGCGACGTGGAAGAGAACCCTG |
| | | GACCTatgaagcgggaccaccaccatcaccatcatcaggacaagaaaaccatgatgatgaacgaagagga |
| | | cgacggcaacggcatggacgagctgctggctgtgctgggctacaaagtgcggagcagcgagatggccgacgt |
| | | ggcccagaaactggaacagctggaagtgatgatgagcaacgtgcaggaagatgacctgtcccagctggccacc |
| | | gagacagtgcactacaaccccgccgagctgtacacctggctggactccatgctgaccgacctgaactccggagg |
| | | gtctggctccggatcaagtggtggcagcggtaccaagcccaagaaggacaagcagaccggcgaggtgctgca |
| | | caagcccatcaccaccaagacagtggaaggcgtgttctacagcgcctgggagacactggacctgggcagacag |
| | | ggcgtgaagcctttcacagcccacagcgccagagtgggagccgctcaggacctgctgaagaagggctacaata |
| | | ccctgcagatccagcagtccggccggtggtctagcggagccatggtggccagatacggcagagccatcctggc |
| | | tagggatggcgctatggcccacagcagagtgaaaaccagatccgcccccatgcagtggggcaaggacgagaa |
| | | ggaccccaagaaaaagcggaaggtgtgatgaCCCAccactggattgtacaattacGAGTCTAAGT |
| | | AAGGATCCCTGTGCCTTCTAGTTGCCAGCCATCTGTTGTTTGCCCCTC |
| | | CCCCGTGCCTTCCTTGACCCTGGAAGGTGCCACTCCCACTGTCCTTTC |
| | | CTAATAAAATGAGGAAATTGCATCGCATTGTCTGAGTAGGTGTCATT |
| | | CTATTCTGGGGGGTGGGGTGGGGCAGGACAGCAAGGGGGAGGATTG |
| | | GGATGACAATAGCAGGCATGCTGGGGATGCGGTGGGCTCTATGG |

TABLE 10
Exemplary Recombinase Attachment Sites

| SEQ ID NO: | Description. | Sequence |
|---|---|---|
| 144 | FRT | GAAGTTCCTATTCTCTAGAAAGTATAGGAACTTC |
| 145 | FRT F1 | GAAGTTCCTATTCTCTAGATAGTATAGGAACTTC |
| 146 | FRT F2 | GAAGTTCCTATTCTCTACTTAGTATAGGAACTTC |
| 147 | FRT F3 | GAAGTTCCTATTCTTCAAATAGTATAGGAACTTC |
| 148 | FRT F4 | GAAGTTCCTATTCTCTAGAAGGTATAGGAACTTC |
| 149 | FRT F5 | GAAGTTCCTATTCTTCAAAAGGTATAGGAACTTC |
| 150 | FRTF10 | GAAGTTCCTATTCACTAGAATGTATAGGAACTTC |
| 151 | FRT F11 | GAAGTTCCTATTCTGAACTAAGTATAGGAACTTC |
| 152 | FRT F12 | GAAGTTCCTATTCTTTCTGAAGTATAGGAACTTC |
| 153 | FRT F13 | GAAGTTCCTATTCTCATATAAGTATAGGAACTTC |
| 154 | FRT F14 | GAAGTTCCTATTCTATCAGAAGTATAGGAACTTC |
| 155 | FRT F15 | GAAGTTCCTATTCTTATAGGAGTATAGGAACTTC |
| 156 | FRT F16 | GAAGTTCCTATTCTCCGGGCAGTATAGGAACTTC |
| 157 | Bxb1 attB [AA] | GGCTTGTCGACGACGGCGAACTCCGTCGTCAGGATCAT |
| 158 | Bxb1 attB [AC] | GGCTTGTCGACGACGGCGACCTCCGTCGTCAGGATCAT |
| 159 | Bxb1 attB [AG] | GGCTTGTCGACGACGGCGAGCTCCGTCGTCAGGATCAT |
| 160 | Bxb1 attB [AT] | GGCTTGTCGACGACGGCGATCTCCGTCGTCAGGATCAT |
| 161 | Bxb1 attB [CA] | GGCTTGTCGACGACGGCGCACTCCGTCGTCAGGATCAT |
| 162 | Bxb1 attB [CC] | GGCTTGTCGACGACGGCGCCCTCCGTCGTCAGGATCAT |
| 163 | Bxb1 attB [CG] | GGCTTGTCGACGACGGCGCGCTCCGTCGTCAGGATCAT |
| 164 | Bxb1 attB [CT] | GGCTTGTCGACGACGGCGCTCTCCGTCGTCAGGATCAT |
| 165 | Bxb1 attB [GA] | GGCTTGTCGACGACGGCGGACTCCGTCGTCAGGATCAT |
| 166 | Bxb1 attB [GC] | GGCTTGTCGACGACGGCGGCCTCCGTCGTCAGGATCAT |
| 167 | Bxb1 attB [GG] | GGCTTGTCGACGACGGCGGGCTCCGTCGTCAGGATCAT |
| 168 | Bxb1 attB [GT] | GGCTTGTCGACGACGGCGGTCTCCGTCGTCAGGATCAT |
| 169 | Bxb1 attB [TA] | GGCTTGTCGACGACGGCGTACTCCGTCGTCAGGATCAT |
| 170 | Bxb1 attB [TC] | GGCTTGTCGACGACGGCGTCCTCCGTCGTCAGGATCAT |
| 171 | Bxb1 attB [TG] | GGCTTGTCGACGACGGCGTGCTCCGTCGTCAGGATCAT |
| 172 | Bxb1 attB [TT] | GGCTTGTCGACGACGGCGTTCTCCGTCGTCAGGATCAT |
| 173 | Bxb1 attP [AA] | GGTTTGTCTGGTCAACCACCGCGAACTCAGTGGTGTACGG |
| | | TACAAACC |
| 174 | Bxb1 attP [AC] | GGTTTGTCTGGTCAACCACCGCGACCTCAGTGGTGTACGG |
| | | TACAAACC |
| 175 | Bxb1 attP [AG] | GGTTTGTCTGGTCAACCACCGCGAGCTCAGTGGTGTACGG |
| | | TACAAACC |
| 176 | Bxb1 attP [AT] | GGTTTGTCTGGTCAACCACCGCGATCTCAGTGGTGTACGG |
| | | TACAAACC |
| 177 | Bxb1 attP [CA] | GGTTTGTCTGGTCAACCACCGCGCACTCAGTGGTGTACGG |
| | | TACAAACC |
| 178 | Bxb1 attP [CC] | GGTTTGTCTGGTCAACCACCGCGCCCTCAGTGGTGTACGG |
| | | TACAAACC |
| 179 | Bxb1 attP [CG] | GGTTTGTCTGGTCAACCACCGCGCGCTCAGTGGTGTACGG |
| | | TACAAACC |
| 180 | Bxb1 attP [CT] | GGTTTGTCTGGTCAACCACCGCGCTCTCAGTGGTGTACGG |
| | | TACAAACC |
| 181 | Bxb1 attP [GA] | GGTTTGTCTGGTCAACCACCGCGGACTCAGTGGTGTACGG |
| | | TACAAACC |
| 182 | Bxb1 attP [GC] | GGTTTGTCTGGTCAACCACCGCGGCCTCAGTGGTGTACGG |
| | | TACAAACC |
| 183 | Bxb1 attP [GG] | GGTTTGTCTGGTCAACCACCGCGGGCTCAGTGGTGTACGG |
| | | TACAAACC |
| 184 | Bxb1 attP [GT] | GGTTTGTCTGGTCAACCACCGCGGTCTCAGTGGTGTACGG |
| | | TACAAACC |
| 185 | Bxb1 attP [TA] | GGTTTGTCTGGTCAACCACCGCGTACTCAGTGGTGTACGG |
| | | TACAAACC |
| 186 | Bxb1 attP [TC] | GGTTTGTCTGGTCAACCACCGCGTCCTCAGTGGTGTACGG |
| | | TACAAACC |
| 187 | Bxb1 attP [TG] | GGTTTGTCTGGTCAACCACCGCGTGCTCAGTGGTGTACGG |
| | | TACAAACC |
| 188 | Bxb1 attP [TT] | GGTTTGTCTGGTCAACCACCGCGTTCTCAGTGGTGTACGG |
| | | TACAAACC |
| 189 | PhiC31 attB [AA] | GTGCGGGTGCCAGGGCGTGCCCAAGGGCTCCCCGGGCGC |
| | | GTACTCC |
| 190 | PhiC31 attB [AC] | GTGCGGGTGCCAGGGCGTGCCCACGGGCTCCCCGGGCGC |
| | | GTACTCC |
| 191 | PhiC31 attB [AG] | GTGCGGGTGCCAGGGCGTGCCCAGGGGCTCCCCGGGCGC |
| | | GTACTCC |
| 192 | PhiC31 attB [AT] | GTGCGGGTGCCAGGGCGTGCCCATGGGCTCCCCGGGCGC |
| | | GTACTCC |
| 193 | PhiC31 attB [CA] | GTGCGGGTGCCAGGGCGTGCCCCAGGGCTCCCCGGGCGC |
| | | GTACTCC |
| 194 | PhiC31 attB [CC] | GTGCGGGTGCCAGGGCGTGCCCCCGGGCTCCCCGGGCGC |
| | | GTACTCC |
| 195 | PhiC31 attB [CG] | GTGCGGGTGCCAGGGCGTGCCCCGGGGCTCCCCGGGCGC |
| | | GTACTCC |
| 196 | PhiC31 attB [CT] | GTGCGGGTGCCAGGGCGTGCCCCTGGGCTCCCCGGGCGC |
| | | GTACTCC |
| 197 | PhiC31 attB [GA] | GTGCGGGTGCCAGGGCGTGCCCGAGGGCTCCCCGGGCGC |
| | | GTACTCC |
| 198 | PhiC31 attB [GC] | GTGCGGGTGCCAGGGCGTGCCCGCGGGCTCCCCGGGCGC |
| | | GTACTCC |
| 199 | PhiC31 attB [GG] | GTGCGGGTGCCAGGGCGTGCCCGGGGGCTCCCCGGGCGC |
| | | GTACTCC |
| 200 | PhiC31 attB [GT] | GTGCGGGTGCCAGGGCGTGCCCGTGGGCTCCCCGGGCGC |
| | | GTACTCC |
| 201 | PhiC31 attB [TA] | GTGCGGGTGCCAGGGCGTGCCCTAGGGCTCCCCGGGCGC |
| | | GTACTCC |
| 202 | PhiC31 attB [TC] | GTGCGGGTGCCAGGGCGTGCCCTCGGGCTCCCCGGGCGC |
| | | GTACTCC |
| 203 | PhiC31 attB [TG] | GTGCGGGTGCCAGGGCGTGCCCTGGGGCTCCCCGGGCGC |
| | | GTACTCC |
| 204 | PhiC31 attB [TT] | GTGCGGGTGCCAGGGCGTGCCCTTGGGCTCCCCGGGCGC |
| | | GTACTCC |
| 205 | PhiC31 attP [AA] | AGTGCCCCAACTGGGGTAACCTAAGAGTTCTCTCAGTTGG |
| | | GGGCGT |
| 206 | PhiC31 attP [AC] | AGTGCCCCAACTGGGGTAACCTACGAGTTCTCTCAGTTGG |
| | | GGGCGT |
| 207 | PhiC31 attP [AG] | AGTGCCCCAACTGGGGTAACCTAGGAGTTCTCTCAGTTGG |
| | | GGGCGT |
| 208 | PhiC31 attP [AT] | AGTGCCCCAACTGGGGTAACCTATGAGTTCTCTCAGTTGG |
| | | GGGCGT |
| 209 | PhiC31 attP [CA] | AGTGCCCCAACTGGGGTAACCTCAGAGTTCTCTCAGTTGG |
| | | GGGCGT |
| 210 | PhiC31 attP [CC] | AGTGCCCCAACTGGGGTAACCTCCGAGTTCTCTCAGTTGG |
| | | GGGCGT |
| 211 | PhiC31 attP [CG] | AGTGCCCCAACTGGGGTAACCTCGGAGTTCTCTCAGTTGG |
| | | GGGCGT |
| 212 | PhiC31 attP [CT] | AGTGCCCCAACTGGGGTAACCTCTGAGTTCTCTCAGTTGG |
| | | GGGCGT |
| 213 | PhiC31 attP [GA] | AGTGCCCCAACTGGGGTAACCTGAGAGTTCTCTCAGTTGG |
| | | GGGCGT |
| 214 | PhiC31 attP [GC] | AGTGCCCCAACTGGGGTAACCTGCGAGTTCTCTCAGTTGG |
| | | GGGCGT |
| 215 | PhiC31 attP [GG] | AGTGCCCCAACTGGGGTAACCTGGGAGTTCTCTCAGTTGG |
| | | GGGCGT |
| 216 | PhiC31 attP [GT] | AGTGCCCCAACTGGGGTAACCTGTGAGTTCTCTCAGTTGG |
| | | GGGCGT |
| 217 | PhiC31 attP [TA] | AGTGCCCCAACTGGGGTAACCTTAGAGTTCTCTCAGTTGG |
| | | GGGCGT |
| 218 | PhiC31 attP [TC] | AGTGCCCCAACTGGGGTAACCTTCGAGTTCTCTCAGTTGG |
| | | GGGCGT |
| 219 | PhiC31 attP [TG] | AGTGCCCCAACTGGGGTAACCTTGGAGTTCTCTCAGTTGG |
| | | GGGCGT |
| 220 | PhiC31 attP [TT] | AGTGCCCCAACTGGGGTAACCTTTGAGTTCTCTCAGTTGG |
| | | GGGCGT |
| 221 | lox66 | ATAACTTCGTATAGCATACATTATACGAACGGTA |
| 222 | lox71 | TACCGttcgtataGCATACATtatacgaagttat |
| 223 | lox511 | ataacttcgtataatgtatActatacgaagttat |
| 224 | lox2272 | ataacttcgtataGgATACtTtatacgaagttat |
| 225 | lox5171 | ataacttcgtataatgtGtActatacgaagttat |
| 226 | loxKR3 | ataacttcgtataGCATACATtatacCTTgttat |
| 227 | loxM2/71 | TACCGTTCGTATATGGTTTCTTATACGAAGTTAT |
| 228 | loxN | ATAACTTCGTATAgtatacctTATACGAAGTTAT |
| 229 | loxP | ataacttcgtatagcatacattatacgaagttat |
| 230 | VloxP | TCAATTTCTGAGAACTGTCATTCTCGGAAATTGA |
| 231 | Vlox2272 | TCAATTTCTGAGAAGTGTCTTTCTCGGAAATTGA |
| 232 | VloxM1 | TCAATTTCCGAGAACTGTCATTCTCGGAAATTGA |
| 233 | VloxM1 | TCAATTTCTGAGAACTGTCATTCTCAGAAATTGA |
| 234 | Vlox43R | TCAATTTCTGAGAACTGTCATTCTCGGAATACCT |
| 235 | Vlox43L | CGTGATTCTGAGAACTGTCATTCTCGGAAATTGA |

What is claimed is:

1. A polynucleic acid molecule encoding a polypeptide dimer having recombinase activity, wherein the nucleic acid sequence encoding the polypeptide dimer comprises, from 5′ to 3′: (i) a sequence encoding for a first polypeptide comprising a first portion of a recombinase and a first dimerization domain; (ii) a sequence encoding for a viral 2A peptide and/or an internal ribosomal entry site (IRES); and (iii) a sequence encoding for a second polypeptide comprising a second portion of a recombinase and a second dimerization domain; wherein the first polypeptide and the second polypeptide lack recombinase activity in the absence of dimerization; and wherein a recombinase dimer comprising the first polypeptide and the second polypeptide has recombinase activity.

2. The polynucleic acid molecule of claim 1, wherein the polypeptide dimer is derived from a Flp recombinase, a Bxb1 recombinase, a PhiC31 recombinase, a TP901 recombinase, a Cre recombinase, a Vcre recombinase, a R4 recombinase, a Dre recombinase, an Int1 recombinase, an Int2 recombinase, an Int3 recombinase, an Int4 recombinase, an Int5 recombinase, an Int6 recombinase, an Int7 recombinase, an Int8 recombinase, an Int9 recombinase, an Int10 recombinase, an Int11 recombinase, an Int 12 recombinase, an Int13 recombinase, an Int14 recombinase, an Int15 recombinase, an Int16 recombinase, an Int17 recombinase, an Int18 recombinase, an Int19 recombinase, an Int20 recombinase, an Int21 recombinase, an Int22 recombinase, an Int23 recombinase, an Int24 recombinase, an Int25 recombinase, an Int26 recombinase, an Int27 recombinase, an Int28 recombinase, an Int29 recombinase, an Int30 recombinase, an Int31 recombinase, an Int32 recombinase, an Int33 recombinase, or an Int34 recombinase.

3. The polynucleic acid molecule of claim 2, wherein the polypeptide dimer is derived from Flp recombinase.

4. The polynucleic acid molecule of claim 3, wherein the first portion of the recombinase corresponds to an N-terminal portion of Flp recombinase and consists of an amino acid sequence having at least 85% identity to SEQ ID NO: 41 and the second portion of the recombinase corresponds to a C-terminal portion of Flp recombinase and consists of an amino acid sequence having at least 85% identity to SEQ ID NO: 42.

5. The polynucleic acid molecule of claim 3, wherein the first portion of the recombinase corresponds to a C-terminal portion of Flp recombinase and consists of an amino acid sequence having at least 85% identity to SEQ ID NO: 42 and the second portion of the recombinase corresponds to an N-terminal portion of Flp recombinase and consists of an amino acid sequence having at least 85% identity to SEQ ID NO: 41.

6. The polynucleic acid molecule of claim 3, wherein the first portion of the recombinase corresponds to an N-terminal portion of Flp recombinase and consists of an amino acid sequence having at least 85% identity to SEQ ID NO: 43 and the second portion of the recombinase corresponds to a C-terminal portion of Flp recombinase and consists of an amino acid sequence having at least 85% identity to SEQ ID NO: 44.

7. The polynucleic acid molecule of claim 3, wherein the first portion of the recombinase corresponds to a C-terminal portion of Flp recombinase and consists of an amino acid sequence having at least 85% identity to SEQ ID NO: 44 and the second portion of the recombinase corresponds to an N-terminal portion of Flp recombinase and consists of an amino acid sequence having at least 85% identity to SEQ ID NO: 43.

8. The polynucleic acid molecule of claim 2, wherein the polypeptide dimer is derived from Bxb1 recombinase.

9. The polynucleic acid molecule of claim 8, wherein the first portion of the recombinase corresponds to an N-terminal portion of Bxb1 recombinase and consists of an amino acid sequence having at least 85% identity to SEQ ID NO: 65 and the second portion of the recombinase corresponds to a C-terminal portion of Bxb1 recombinase and consists of an amino acid sequence having at least 85% identity to SEQ ID NO: 66.

10. The polynucleic acid molecule of claim 8, wherein the first portion of the recombinase corresponds to a C-terminal portion of Bxb1 recombinase and consists of an amino acid sequence having at least 85% identity to SEQ ID NO: 66 and the second portion of the recombinase corresponds to an N-terminal portion of Bxb1 recombinase and consists of an amino acid sequence having at least 85% identity to SEQ ID NO: 65.

11. The polynucleic acid molecule of claim 8, wherein the first portion of the recombinase corresponds to an N-terminal portion of Bxb1 recombinase and consists of an amino acid sequence having at least 85% identity to SEQ ID NO: 47 and the second portion of the recombinase corresponds to a C-terminal portion of Bxb1 recombinase and consists of an amino acid sequence having at least 85% identity to SEQ ID NO: 48.

12. The polynucleic acid molecule of claim 8, wherein the first portion of the recombinase corresponds to a C-terminal portion of Bxb1 recombinase and consists of an amino acid sequence having at least 85% identity to SEQ ID NO: 48 and the second portion of the recombinase corresponds to an N-terminal portion of Bxb1 recombinase and consists of an amino acid sequence having at least 85% identity to SEQ ID NO: 47.

13. The polynucleic acid molecule of claim 8, wherein the first portion of the recombinase corresponds to an N-terminal portion of Bxb1 recombinase and consists of an amino acid sequence having at least 85% identity to SEQ ID NO: 49 and the second portion of the recombinase corresponds to a C-terminal portion of Bxb1 recombinase and consists of an amino acid sequence having at least 85% identity to SEQ ID NO: 50.

14. The polynucleic acid molecule of claim 8, wherein the first portion of the recombinase corresponds to a C-terminal portion of Bxb1 recombinase and consists of an amino acid sequence having at least 85% identity to SEQ ID NO: 50 and the second portion of the recombinase corresponds to an N-terminal portion of Bxb1 recombinase and consists of an amino acid sequence having at least 85% identity to SEQ ID NO: 49.

15. The polynucleic acid molecule of claim 8, wherein the first portion of the recombinase corresponds to an N-terminal portion of Bxb1 recombinase and consists of an amino acid sequence having at least 85% identity to SEQ ID NO: 53 and the second portion of the recombinase corresponds to a C-terminal portion of Bxb1 recombinase and consists of an amino acid sequence having at least 85% identity to SEQ ID NO: 54.

16. The polynucleic acid molecule of claim 8, wherein the first portion of the recombinase corresponds to a C-terminal portion of Bxb1 recombinase and consists of an amino acid sequence having at least 85% identity to SEQ ID NO: 54 and the second portion of the recombinase corresponds to an N-terminal portion of Bxb1 recombinase and consists of an amino acid sequence having at least 85% identity to SEQ ID NO: 53.

17. The polynucleic acid molecule of claim 8, wherein the first portion of the recombinase corresponds to an N-terminal portion of Bxb1 recombinase and consists of an amino acid sequence having at least 85% identity to SEQ ID NO: 59 and the second portion of the recombinase corresponds to a C-terminal portion of Bxb1 recombinase and consists of an amino acid sequence having at least 85% identity to SEQ ID NO: 60.

18. The polynucleic acid molecule of claim 8, wherein the first portion of the recombinase corresponds to a C-terminal portion of Bxb1 recombinase and consists of an amino acid sequence having at least 85% identity to SEQ ID NO: 60 and the second portion of the recombinase corresponds to an N-terminal portion of Bxb1 recombinase and consists of an amino acid sequence having at least 85% identity to SEQ ID NO: 59.

19. The polynucleic acid molecule of claim 2, wherein the polypeptide dimer is derived from PhiC31 recombinase.

20. The polynucleic acid molecule of claim 19, wherein the first portion of the recombinase corresponds to an N-terminal portion of PhiC31 recombinase and consists of an amino acid sequence having at least 85% identity to SEQ ID NO: 67 and the second portion of the recombinase corresponds to a C-terminal portion of PhiC31 recombinase and consists of an amino acid sequence having at least 85% identity to SEQ ID NO: 68.

21. The polynucleic acid molecule of claim 19, wherein the first portion of the recombinase corresponds to a C-terminal portion of PhiC31 recombinase and consists of an amino acid sequence having at least 85% identity to SEQ ID NO: 68 and the second portion of the recombinase corresponds to an N-terminal portion of PhiC31 recombinase and consists of an amino acid sequence having at least 85% identity to SEQ ID NO: 67.

22. The polynucleic acid molecule of claim 19, wherein the first portion of the recombinase corresponds to an N-terminal portion of PhiC31 recombinase and consists of an amino acid sequence having at least 85% identity to SEQ ID NO: 69 and the second portion of the recombinase corresponds to a C-terminal portion of PhiC31 recombinase and consists of an amino acid sequence having at least 85% identity to SEQ ID NO: 70.

23. The polynucleic acid molecule of claim 19, wherein the first portion of the recombinase corresponds to a C-terminal portion of PhiC31 recombinase and consists of an amino acid sequence having at least 85% identity to SEQ ID NO: 70 and the second portion of the recombinase corresponds to an N-terminal portion of PhiC31 recombinase and consists of an amino acid sequence having at least 85% identity to SEQ ID NO: 69.

24. The polynucleic acid molecule of claim 2, wherein the polypeptide dimer is derived from TP901 recombinase.

25. The polynucleic acid molecule of claim 24, wherein the first portion of the recombinase corresponds to an N-terminal portion of TP901 recombinase and consists of an amino acid sequence having at least 85% identity to SEQ ID NO: 71 and the second portion of the recombinase corresponds to a C-terminal portion of TP901 recombinase and consists of an amino acid sequence having at least 85% identity to SEQ ID NO: 72.

26. The polynucleic acid molecule of claim 24, wherein the first portion of the recombinase corresponds to a C-terminal portion of TP901 recombinase and consists of an amino acid sequence having at least 85% identity to SEQ ID NO: 72 and the second portion of the recombinase corresponds to an N-terminal portion of TP901 recombinase and consists of an amino acid sequence having at least 85% identity to SEQ ID NO: 71.

27. The polynucleic acid molecule of claim 2, wherein the polypeptide dimer is derived from Cre recombinase.

28. The polynucleic acid molecule of claim 27, wherein the first portion of the recombinase corresponds to an N-terminal portion of Cre recombinase and consists of an amino acid sequence having at least 85% identity to SEQ ID NO: 73 and the second portion of the recombinase corresponds to a C-terminal portion of Cre recombinase and consists of an amino acid sequence having at least 85% identity to SEQ ID NO: 74.

29. The polynucleic acid molecule of claim 27, wherein the first portion of the recombinase corresponds to a C-terminal portion of Cre recombinase and consists of an amino acid sequence having at least 85% identity to SEQ ID NO: 74 and the second portion of the recombinase corresponds to an N-terminal portion of Cre recombinase and consists of an amino acid sequence having at least 85% identity to SEQ ID NO: 73.

30. The polynucleic acid molecule of claim 2, wherein the polypeptide dimer is derived from Vcre recombinase.

31. The polynucleic acid molecule of claim 30, wherein the first portion of the recombinase corresponds to an N-terminal portion of Vcre recombinase and consists of an amino acid sequence having at least 85% identity to SEQ ID NO: 77 and the second portion of the recombinase corresponds to a C-terminal portion of Vcre recombinase and consists of an amino acid sequence having at least 85% identity to SEQ ID NO: 78.

32. The polynucleic acid molecule of claim 30, wherein the first portion of the recombinase corresponds to a C-terminal portion of Vcre recombinase and consists of an amino acid sequence having at least 85% identity to SEQ ID NO: 78 and the second portion of the recombinase corresponds to an N-terminal portion of Vcre recombinase and consists of an amino acid sequence having at least 85% identity to SEQ ID NO: 77.

33. The polynucleic acid molecule of any one of claims 1-32, wherein, in the first polypeptide, the first dimerization domain is N-terminal to the first portion of the recombinase.

34. The polynucleic acid molecule of any one of claims 1-32, wherein, in the first polypeptide, the first dimerization domain is C-terminal to the first portion of the recombinase.

35. The polynucleic acid molecule of any one of claims 1-34, wherein, in the second polypeptide, the second dimerization domain is N-terminal to the second portion of the recombinase.

36. The polynucleic acid molecule of any one of claims 1-34, wherein, in the second polypeptide, the second dimerization domain is C-terminal to the second portion of the recombinase.

37. The polynucleic acid molecule of any one of claims 1-36, wherein the dimerization of the first polypeptide and the second polypeptide is dependent on the presence of a small molecule inducer.

38. The polynucleic acid molecule of claim 37, wherein the small molecule inducer is selected from the group consisting of gibberellic acid, abscisic acid, and rapalog.

39. The polynucleic acid molecule of claim 37 or claim 38, wherein:
the first dimerization domain comprises an amino acid sequence having at least 85% identity to SEQ ID NO: 80 and the second dimerization domain comprises an amino acid sequence having at least 85% identity to SEQ ID NO: 79;
the first dimerization domain comprises an amino acid sequence having at least 85% identity to SEQ ID NO: 79 and the second dimerization domain comprises an amino acid sequence having at least 85% identity to SEQ ID NO: 80;
the first dimerization domain comprises an amino acid sequence having at least 85% identity to SEQ ID NO: 81 and the second dimerization domain comprises an amino acid sequence having at least 85% identity to SEQ ID NO: 82;
the first dimerization domain comprises an amino acid sequence having at least 85% identity to SEQ ID NO: 82 and the second dimerization domain comprises an amino acid sequence having at least 85% identity to SEQ ID NO: 81;
the first dimerization domain comprises an amino acid sequence having at least 85% identity to SEQ ID NO: 83 and the second dimerization domain comprises an amino acid sequence having at least 85% identity to SEQ ID NO: 84; or
the first dimerization domain comprises an amino acid sequence having at least 85% identity to SEQ ID NO: 84 and the second dimerization domain comprises an amino acid sequence having at least 85% identity to SEQ ID NO: 83.

40. The polynucleic acid molecule of any one of claims 1-39, wherein the nucleic acid sequence encoding the polypeptide dimer comprises a sequence encoding for a viral 2A peptide.

41. The polynucleic acid molecule of claim 40, wherein the viral 2A peptide comprises an amino acid sequence having at least 85% identity to any one of SEQ ID NOs: 88-89 and 236-237.

42. The polynucleic acid molecule of any one of claims 1-39, wherein the nucleic acid sequence encoding the polypeptide dimer comprises a sequence encoding for an IRES.

43. The polynucleic acid molecule of claim 42, wherein the TRES comprises a nucleic acid sequence having at least 85% identity to any one of SEQ ID NOs: 85-87.

44. The polynucleic acid molecule of any one of claims 1-39, wherein the nucleic acid sequence encoding the polypeptide dimer comprises a sequence having at least 85% identity to any one of SEQ ID NOs: 90-110.

45. The polynucleic acid molecule of any one of claims 1-44, wherein the polynucleic acid molecule encodes a polycistronic mRNA operably linked to a promoter, wherein the polycistronic mRNA comprises the nucleic acid sequence encoding the polypeptide dimer.

46. The polynucleic acid molecule of claim 45, wherein the nucleic acid sequence encoding for the polycistronic mRNA is operably linked to a constitutive promoter.

47. The polynucleic acid molecule of claim 45, wherein the nucleic acid sequence encoding for the polycistronic mRNA is operably linked to an inducible promoter.

48. The polynucleic acid molecule of claim 45, wherein the polynucleic acid molecule comprises an expression cassette comprising the nucleotide encoding for the polycistronic mRNA operably linked to a promoter, wherein the expression cassette comprises a nucleic acid sequence having at least 85% identity to any one of SEQ ID NO: 132-143.

49. An engineered cell comprising the polynucleic acid molecule of any one of claims 1-48.

50. An engineered cell comprising:
(a) a first polynucleic acid molecule according to any one of claims 45-48; and
(b) a second polynucleic acid molecule comprising a nucleic acid sequence encoding, from 5′ to 3′: (i) a first recombinase site; (ii) a gene coding segment; and
(iii) a second recombinase site; wherein the first and second recombinase sites correspond to the polypeptide dimer having recombinase activity encoded by the polycistronic mRNA of the first polynucleic acid of (a).

51. The engineered cell of claim 50, wherein the first recombinase site of the second polynucleic acid molecule comprises a nucleic acid sequence having at least 85% identity to any one of SEQ ID NOs: 144-235 and the second recombinase site of the second polynucleic acid molecule comprises the nucleic acid sequence having at least 85% identity to any one of SEQ ID NOs: 144-235.

52. The engineered cell of claim 50 or claim 51, wherein the gene coding segment comprises a nucleic acid sequence encoding for at least a portion of Rep52, at least a portion of Rep40, at least a portion of Rep78, at least a portion of Rep68, at least a portion of E2A, at least a portion of E4Orf6, at least a portion of VARNA, at least a portion of VP1, at least a portion of VP2, at least a portion of VP3, at least a portion of AAP, or a combination thereof.

53. The engineered cell of claim 52, wherein the engineered cell comprises a stable integration of one or more polynucleic acid molecules collectively comprising nucleic acid sequences encoding for: Rep52 or Rep40; Rep78 or Rep68; E2A; E4Orf6; VARNA; VP1;
VP2; VP3; and AAP.

54. The engineered cell of claim 52 or claim 53, wherein the engineered cell comprises one or more polynucleic acid molecules collectively comprising nucleic acid sequences encoding for: UL5, UL8, UL29, UL30, UL42, UL52, UL12, ICP10, ICP4, and ICP22.
